(19) United States
(12) Patent Application Publication
(10) Pub. No.: US 2025/0084179 A1
(43) Pub. Date: Mar. 13, 2025

(54) ANTI-CD28 X ANTI-TROP2 ANTIBODIES

(71) Applicant: Xencor, Inc., Pasadena, CA (US)

(72) Inventors: Matthew J. Bernett, Monrovia, CA (US); Yoon Kyung Kim, Whittier, CA (US); ChaoFang Tan, Arcadia, CA (US); Patrick Arpp, Pasadena, CA (US); Erica Rodriguez, Anaheim, CA (US); Todd Anzelon, La Crescenta, CA (US); Gregory Moore, Azusa, CA (US); Dong Hyun Nam, Monterey Park, CA (US); Kendra N. Avery, Hawthorne, CA (US); Thuy B. Truong, Baldwin Park, CA (US); William Y. Yeh, Glendale, CA (US); Jonathan Jacinto, Lancaster, CA (US); Juan Diaz, Anaheim Hills, CA (US)

(21) Appl. No.: 18/634,848

(22) Filed: Apr. 12, 2024

Related U.S. Application Data
(60) Provisional application No. 63/496,372, filed on Apr. 14, 2023.
(60) Provisional application No. 63/593,942, filed on Oct. 27, 2023.

(51) Int. Cl. C07K 16/30; C07K 16/28

(52) U.S. Cl. CPC C07K 16/30; C07K 16/2818; C07K 2317/31; C07K 2317/33; C07K 2317/51; C07K 2317/515; C07K 2317/52; C07K 2317/55; C07K 2317/622; C07K 2317/73; C07K 2317/74; C07K 2317/92; C07K 2317/94

(57) ABSTRACT

Provided herein are novel anti-CD28×anti-TROP2 antibodies and methods of using such antibodies for the treatment of TROP2-associated cancers. Subject anti-CD28×anti-TROP2 antibodies are capable of agonistically binding to CD28 costimulatory molecules on T cells and TROP2 on tumor cells. Thus, such antibodies selectively enhance anti-tumor activity at tumor sites while minimizing peripheral toxicity. The subject antibodies provided herein are particularly useful in combination with other anti-cancer therapies, including, for example, bispecific antibodies for the treatment of TROP2-associated cancers.

### PRIORITY CLAIM

This application claims priority to and the benefit of United States Provisional Application Nos. 63/496,372, filed Apr. 14, 2023, and 63/593,942, filed Oct. 27, 2023, the contents of which are hereby incorporated by reference in their entirety.

### SEQUENCE LISTING

The instant application contains a Sequence Listing which has been submitted electronically in XML format and is hereby incorporated by reference in its entirety. Said XML copy, created on Jun. 10, 2024, is named 067461-5314-US_SL.xml and is 1,096,048 bytes in size.

### BACKGROUND

Tumor-associated calcium signal transducer 2 (TROP2) is a transmembrane protein overexpressed in various malignant tumors, and is an oncogene related to the occurrence, invasion and metastasis of malignant tumors. Exemplary cancers that overexpress TROP2 include endometrial cancer, pancreatic cancer, colon cancer, esophageal cancer, cholangiocarcinoma, oral cancer, and glioma. As it is overexpressed on a variety of tumors, TROP2 is a candidate for targeted therapeutic development.

Antibody-based therapeutics have been used successfully to treat a variety of diseases, including cancer. An increasingly prevalent avenue being explored is the engineering of single immunoglobulin molecules that co-engage two different antigens. Such alternate antibody formats that engage two different antigens are often referred to as bispecific antibodies. One particular approach for bispecific antibodies is to engineer a first binding domain which engages CD3 and a second binding domain which engages an antigen associated with or upregulated on cancer cells (e.g., TROP2) so that the bispecific antibody redirects CD3⁺ T cells to destroy the cancer cells.

TILs, however, lose their cytotoxic ability over time due to upregulation of inhibitory immune checkpoints. While checkpoint blockade has demonstrated increased clinical response rates relative to other treatment options, many patients still fail to achieve a response to checkpoint blockade. Engagement of costimulatory receptors on TILs could provide a positive signal capable of overcoming negative signals of immune checkpoints. Preclinical and clinical studies of agonistic costimulatory receptor antibodies have indeed demonstrated that agonism of costimulatory receptors can result in impressive anti-tumor responses, activating T cells to attack tumor cells.

It is also important for cancer therapy to enhance anti-tumor activity by specifically destroying tumor cells while minimizing peripheral toxicity. In this context, it is crucial that only T cells in the presence of the target tumor cells are provided a costimulatory signal. However, agonism of costimulatory receptors with monospecific full-length antibodies is likely nondiscriminatory with regards to TILs vs. peripheral T cells vs. autoantigen-reactive T cells that contribute to autoimmune toxicities.

While immunotherapies targeting TROP2 have been tried, there still remains a need for novel immune response enhancing compositions for the treatment of TROP2-associated cancers.

### SUMMARY

Provided herein are novel anti-CD28×anti-TROP2 antibodies and methods of using such antibodies for the treatment of TROP2-associated cancers. Subject anti-CD28×anti-TROP2 antibodies are capable of agonistically binding to CD28 costimulatory molecules on T cells and TROP2 on tumor cells. Thus, such antibodies selectively enhance anti-tumor activity at tumor sites while minimizing peripheral toxicity. The subject antibodies provided herein are particularly useful in combination with other anti-cancer therapies, including, for example, bispecific antibodies for the treatment of TROP2-associated cancers.

In a first aspect, provided herein is an anti-CD28×anti-TROP2 heterodimeric antibody that comprises a) a first monomer; b) a second monomer; and c) a light chain. The first monomer comprises: i) a single chain variable fragment (scFv); and ii) a first Fc domain, wherein the scFv is covalently attached to the N-terminus of the first Fc domain using a domain linker. The second monomer comprises, from N-terminal to C-terminal, a VH1-CH1-hinge-CH2-CH3, wherein VH1 is a first variable heavy domain and CH2-CH3 is a second Fc domain. The light chain comprises, from N-terminal to C-terminal, VL1-CL, wherein VL1 is a first variable light domain and CL is a constant light domain. The scFv comprises a second VH domain (VH2), a scFv linker, and a second variable light domain (VL2). The VH1 and the VL1 together form a first antigen binding domain (ABD) and the VH2 and the VL2 together form a second ABD. Further, one of the first ABD and second ABD is a CD28 binding domain and the other of the first ABD and second ABD is a tumor-associated calcium signal transducer 2 (TROP2) binding domain.

In some embodiments, the scFv comprises, from N-terminal to C-terminal, VH2-scFv linker-VL2. In some embodiments, the scFv comprises, from N-terminal to C-terminal, VL2-scFv linker-VH2.

In exemplary embodiments, the first ABDs is the TROP2 binding domain and the second ABD is the CD28 binding domain. In some embodiments, VH1 and VL1 are a VH and VL of any of the TROP2 binding domains from FIGS. 23-26 and 52-56 or a variant thereof. In some embodiments, VH2 and VL2 are selected from one of the following: 1) a VH and VL of any of the CD28 binding domains from FIGS. 16, 19, and 22 or a variant thereof; or 2) (i) a VH from FIG. 16, 17, or 34 or a variant thereof; and (ii) a VL from FIG. 16, 18, or 36 or a variant thereof.

In some embodiments, the first Fc domain and second Fc domain are each variant Fc domains.

In some embodiments, the first and second Fc domains comprise a set of heterodimerization skew variants selected from the following heterodimerization variants: S364K/E357Q:L368D/K370S; S364K:L 368D/K370S; S364K:L368E/K370S; D401K:T411E/K360E/Q362E; and T366W:T 366S/L368A/Y407V, wherein numbering is according to EU numbering. In some embodiments, the first and second Fc domains comprise heterodimerization skew variants S364K/E357Q:L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421D, the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In some embodiments, the scFv linker is GKPGSGKPGSGKPGSGKPGS (SEQ ID NO:24).

In another aspect, provided herein is an anti-CD28×anti-TROP2 heterodimeric antibody that comprises a) a first monomer; b) a second monomer; c) a first light chain; and d) a second light chain. The first monomer comprises, from N-terminal to C-terminal, VH1-CH1-first domain linker-scFv-second domain linker-CH2-CH3, wherein VH1 is a first variable heavy domain, and CH2-CH3 is a first Fc domain. The second monomer comprises, from N-terminal to C-terminal, a VH1-CH1-hinge-CH2-CH3, wherein CH2-CH3 is a second Fc domain. The first and second light chains each comprise, from N-terminal to C-terminal, VL1-CL, wherein VL1 is a first variable light domain and CL is a constant light domain. The scFv comprises a second VH domain (VH2), a scFv linker, and a second variable light domain (VL2). The VH1 of the first monomer and the VL1 of the first light chain and the VH1 of the second monomer and the VL1 of the second light chain each form a first antigen binding domain (ABD), and the VH2 and the VL2 form a second ABD. Further, one of the first ABDs and second ABD is a CD28 binding domain and the other of the first ABDs and second ABD is a tumor-associated calcium signal transducer 2 (TROP2) binding domain.

In some embodiments, the scFv comprises, from N-terminal to C-terminal, VH2-scFv linker-VL2. In some embodiments, the scFv comprises, from N-terminal to C-terminal, VL2-scFv linker-VH2.

In exemplary embodiments, the first ABDs are the TROP2 binding domains and the second ABD is the CD28 binding domain. In some embodiments, VH1 and VL1 are selected from one of the following: 1) a VH and VL of any of the TROP2 binding domains from FIGS. 23-26 and 52-56 or a variant thereof. In some embodiments, VH2 and VL2 are selected from one of the following: 1) a VH and VL of any of the CD28 binding domains from FIGS. 16, 19, and 22 or a variant thereof; or 2) (i) a VH from FIG. 16, 17, or 34 or a variant thereof; and (ii) a VL from FIG. 16, 18, or 36 or a variant thereof.

In some embodiments, the first Fc domain and second Fc domain are each variant Fc domains.

In some embodiments, the first and second Fc domains comprise a set of heterodimerization skew variants selected from the following heterodimerization variants: S364K/E357Q:L368D/K370S; S364K:L 368D/K370S; S364K:L368E/K370S; D401K:T411E/K360E/Q362E; and T366W:T 366S/L368A/Y407V, wherein numbering is according to EU numbering. In some embodiments, the first and second Fc domains comprise heterodimerization skew variants S364K/E357Q:L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421D, the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In some embodiments, the scFv linker is GKPGSGKPGSGKPGSGKPGS (SEQ ID NO:24).

In another aspect, provided herein is an anti-CD28×anti-TROP2 heterodimeric antibody that comprises a) a first monomer; b) a second monomer; c) a first light chain; and d) a second light chain. The first monomer comprises, from N-terminal to C-terminal, VH1-CH1-hinge-CH2-CH3-domain linker-scFv, wherein VH1 is a first variable heavy domain, and CH2-CH3 is a first Fc domain. The second monomer comprises, from N-terminal to C-terminal, a VH1-CH1-hinge-CH2-CH3, wherein VH1 is a first variable heavy domain and CH2-CH3 is a second Fc domain. The first and second light chains each comprise, from N-terminal to C-terminal, VL1-CL, wherein VL1 is a first variable light domain and CL is a constant light domain. The scFv comprises a second VH domain (VH2), a scFv linker, and a second variable light domain (VL2). The VH1 of the first monomer and the VL1 of the first light chain, and the VH1 of the second monomer and the VL1 of the second light chain each form a first antigen binding domain (ABD), and the VH2 and the VL2 form a second ABD. Further, one of the first and second ABDs bind human CD28 and the other of the first and second ABDs binds TROP2.

In some embodiments, the scFv comprises, from N-terminal to C-terminal, VH2-scFv linker-VL2. In some embodiments, the scFv comprises, from N-terminal to C-terminal, VL2-scFv linker-VH2.

In exemplary embodiments, the first ABDs are the TROP2 binding domains and the second ABD is the CD28 binding domain. In some embodiments, VH1 and VL1 are selected from one of the following: 1) a VH and VL of any of the TROP2 binding domains from FIGS. 23-26 and 52-56 or a variant thereof. In some embodiments, VH2 and VL2 are selected from one of the following: 1) a VH and VL of any of the CD28 binding domains from FIGS. 16, 19, and 22 or a variant thereof; or 2) (i) a VH from FIG. 16, 17, or 34 or a variant thereof; and (ii) a VL from FIG. 16, 18, or 36 or a variant thereof.

In some embodiments, the first Fc domain and second Fc domain are each variant Fc domains.

In some embodiments, the first and second Fc domains comprise a set of heterodimerization skew variants selected from the following heterodimerization variants: S364K/E357Q:L368D/K370S; S364K L368D/K370S; S364K:L368E/K370S; D401K:T411E/K360E/Q362E; and T366W:T 366S/L368A/Y407V, wherein numbering is according to EU numbering. In some embodiments, the first and second Fc domains comprise heterodimerization skew variants S364K/E357Q:L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421D, the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In some embodiments, the scFv linker is GKPGSGKPGSGKPGSGKPGS (SEQ ID NO:24).

In another aspect, provided herein is a bispecific antibody that comprises: a) a TROP2 binding domain comprising i) a first variable heavy domain (VH1), and ii) a first variable light domain (VL1); and b) an anti-CD28 binding domain comprising i) a second variable heavy domain (VH2), and ii) a second variable light domain (VL2). In some embodiments, VH1 and VL1 are selected from one of the following: 1) a VH and VL of any of the TROP2 binding domains from FIGS. 23-26 and 52-56 or a variant thereof. In some embodiments, VH2 and VL2 are selected from one of the following: 1) a VH and VL of any of the CD28 binding domains from FIGS. 16, 19, and 22 or a variant thereof; or 2) (i) a VH from FIG. 16, 17, or 34 or a variant thereof; and (ii) a VL from FIG. 16, 18, or 36 or a variant thereof.

In some embodiments of the bispecific antibodies, the first Fc domain and second Fc domain are each variant Fc domains.

In some embodiments of the bispecific antibodies, the first and second Fc domains comprise a set of heterodimerization skew variants selected from the following heterodimerization variants: S364K/E357Q:L368D/K370S; S364K:L368D/K370S; S364K:L368E/K370S; D401K: T411E/K360E/Q362E; and T366W:T366S/L368A/Y407V, wherein numbering is according to EU numbering. In some embodiments, the first and second Fc domains comprise heterodimerization skew variants S364K/E357Q:L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In one aspect provided herein is a multivalent antibody that comprises a first monomer, a second monomer, a first common light chain, and a second common light chain. The first monomer comprises, from N-terminal to C-terminal, a VH1-CH1-hinge-CH2-CH3, wherein VH1 is a first variable heavy domain and CH2-CH3 is a first Fc domain. The second monomer comprises, from N-terminal to C-terminal, a VH2-CH1-hinge-CH2-CH3, wherein VH2 is a second variable heavy domain and CH2-CH3 is a second Fc domain. The first common light chain and second common light chain each comprise a VL-CL, wherein VL is a variable domain and CL is a constant light domain. The VH1 and the VL of the first common light chain form a first antigen binding domain (ABD) and the VH2 and the VL of the second common light chain together form a second ABD, and the first common light chain and second common light chain have the same amino acid sequence. Further, one of the first ABD and second ABD is a CD28 binding domain and the other of the first ABD and second ABD is a tumor-associated calcium signal transducer 2 (TROP2) binding domain.

In some embodiments, the first ABD is the TROP2 binding domain, and the second ABD is the CD28 binding domain. In some embodiments, the VH1 is a variable heavy domain from FIG. 36 or 52-56, VH2 is a variable heavy domain from FIG. 34, and the VL of first and second common light chain is the 1F11-1A3.315[TROP2]_LI variable light domain (see FIG. 36A).

In some embodiments, the first Fc domain and second Fc domain are each variant Fc domains.

In some embodiments, the first and second Fc domains comprise a set of heterodimerization skew variants selected from the following heterodimerization variants: S364K/E357Q:L368D/K370S; S364K:L 368D/K370S; S364K:L368E/K370S; D401K:T411E/K360E/Q362E; and T366W:T 366S/L368A/Y407V, wherein numbering is according to EU numbering. In some embodiments, the first and second Fe domains comprise heterodimerization skew variants S364K/E357Q. L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421D, the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

In another aspect, provided herein is a multivalent antibody that comprises a first monomer, a second monomer, a first common light chain, a second common light chain, and a third common light chain. The first monomer comprises, from N-terminal to C-terminal, a VH1-CH1-linker-VH1-CH1-hinge-CH2-CH3, wherein the VHls are each a first variable heavy domain and CH2-CH3 is a first Fc domain. The second monomer comprises, from N-terminal to C-terminal, a VH2-CH1-hinge-CH2-CH3, wherein VH2 is a second variable heavy domain and CH2-CH3 is a second Fc domain. The first common light chain, second common light chain, and third common light chain each comprise a VL-CL, wherein VL is a variable domain and CL is a constant light domain. The VHls of the first monomer each pair with a VL of the first common light chain or second common light to form two first antigen binding domains (ABDs) and the VH2 and the VL of the third common light chain form a second ABD, and the first common light chain, second common light chain, and third common light chain each have the same amino acid sequence. Further, the first ABDs are each a CD28 binding domain and the second ABD is a tumor-associated calcium signal transducer 2 (TROP2) binding domain, or the first ABDs are each a TROP2 binding domain and the second ABD is a CD28 binding domain.

In some embodiments, the first ABDs are each a TROP2 binding domain and the second ABD is the CD28 binding domain. In some embodiments, the VH1s are each a variable heavy domain from FIG. 36 or 52-56, VH2 is a variable heavy domain from FIG. 34, and the VL of first, second, and third common light chain is the 1F11-1A3.315[TROP2]_L1 variable light domain (see FIG. 36A).

In some embodiments, the first Fc domain and second Fc domain are each variant Fc domains.

In some embodiments, the first and second Fc domains comprise a set ofheterodimerization skew variants selected from the following heterodimerization variants: S364K/E357Q:L368D/K370S; S364K:L 368D/K370S; S364K:L368E/K370S; D401K:T411E/K360E/Q362E; and T366W:T 366S/L368A/Y407V, wherein numbering is according to EU numbering. In some embodiments, the first and second Fc domains comprise heterodimerization skew variants S364K/E357Q:L368D/K370S, wherein numbering is according to EU numbering.

In certain embodiments, the first and second Fc domains each comprise one or more ablation variants. In exemplary embodiments, the one or more ablation variants comprise E233P/L234V/L235A/G236del/S267K, wherein numbering is according to EU numbering.

In some embodiments, the one of the first or second monomer further comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the CH1-hinge-CH2-CH3 of the second monomer comprises amino acid variants E233P/L234V/L235A/G236del/S267K/L368D/K370S/N208D/Q295E/N384D/Q418E/N421D, the first Fc domain comprises amino acid variants E233P/L234V/L235A/G236del/S267K/S364K/E357Q, wherein numbering is according to EU numbering. In exemplary embodiments, the first and second variant Fc domains each further comprise amino acid variants 428L/434S.

Also provided herein are nucleic acid compositions comprising nucleic acids encoding the antibodies described herein, expression vector compositions that include such nucleic acids, host cells for making the antibodies that comprise the expression vector compositions, and methods of making the antibodies.

In another aspect, provided herein is a method of treating a TROP2-associated cancer in a patient in need thereof, comprising administering to the patient according an anti-CD28×anti-TROP2 bispecific antibody as described herein.

In another aspect, provided herein is a method of treating a TROP2-associated cancer in a patient in need thereof, comprising administering to the patient an anti-CD28×anti-TROP2 bispecific antibody as described herein; and an anti-CD3×anti-TROP2 bispecific antibody.

In one aspect, provided herein is a TROP-2 binding construct comprising a variable heavy domain and a variable light domain selected from the following: 1) a VH and VL of any of the TROP2 binding domain VHs and VLs from FIGS. 23-26 and 52-56 or a variant thereof; and 2) a VH depicted in FIG. 36 or a variant thereof, and a 1F11-1A3.315 L1 VL (see FIG. 36A) or a variant thereof.

### BRIEF DESCRIPTION OF THE DRAWINGS

FIGS. 1A-1B depict the sequences for human, mouse, and cynomolgus CD28. Such CD28 are useful for the development of cross-reactive CD28 antigen binding domains for ease of clinical development.

FIG. 2 depicts the sequences for human, mouse, and cynomolgus TROP2. Such TROP2 are useful for the development of cross-reactive TROP2 antigen binding domains for ease of clinical development.

FIG. 3A-3F depict useful pairs of heterodimerization variant sets (including skew and pI variants). In FIG. 3F, there are variants for which there are no corresponding “monomer 2” variants. Such variants are pI variants that can be used alone on either monomer of a bispecific antibody (e.g., TROP2×CD28 bsAb), or included, for example, on the non-scFv side of a format that utilizes an scFv as a component and an appropriate charged scFv linker can be used on the second monomer that utilizes an scFv as the CD28 binding domain. Suitable charged linkers are shown in FIG. 6.

FIG. 4 depicts a list of isosteric variant antibody constant regions and their respective substitutions. pI_(−) indicates lower pI variants, while pI_(+) indicates higher pI variants. These variants can be optionally and independently combined with other variants, including heterodimerization variants, outlined herein.

FIG. 5 depict useful ablation variants that ablate FcγR binding (also referred to as “knockouts” or “KO” variants). In some embodiments, such ablation variants are included in the Fc domain of both monomers of the subject antibody described herein. In other embodiments, the ablation variants are only included on only one variant Fc domain.

FIGS. 6A-6B depicts a number of charged scFv linkers that find use in increasing or decreasing the pI of the subject heterodimeric bispecific antibodies that utilize one or more scFv as a component, as described herein (e.g., TROP2×CD28 bsAbs). The (+H) positive linker finds particular use herein, particularly with anti-CD28 VL and VH sequences shown herein. A single prior art scFv linker with a single charge is referenced as “Whitlow,” from Whitlow et al., Protein Engineering 6(8):989-995 (1993).

It should be noted that this linker was used for reducing aggregation and enhancing proteolytic stability in scFvs. Such charged scFv linkers can be used in any of the subject antibody formats disclosed herein that include scFvs (e.g., 1+1 Fab-scFv-Fc and 2+1 Fab2-scFv-Fc formats).

FIG. 7 depicts a number of exemplary domain linkers. In some embodiments, these linkers find use linking a single-chain Fv to an Fc chain. In some embodiments, these linkers may be combined in any orientation. For example, a GGGGS linker (SEQ ID NO:37) may be combined with a “lower half hinge” linker at the N-terminus or at the C-terminus.

FIG. 8 shows a particularly useful bispecific antibody platform for the TROP2×CD28 bsAbs of the invention. Although the platform is described in the context of the 1+1 Fab-scFv-Fc format, it can be adapted for use in other bispecific antibody formats.

FIG. 9 depicts various heterodimeric skew variant amino acid substitutions that can be used with the heterodimeric antibodies described herein.

FIGS. 10A-10D shows the sequences of several useful heterodimeric TROP2×CD28 bsAb backbones based on human IgG1, without the cytokine sequences. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain, and the E233P/L234V/L235A/G236del/S267K ablation variants on both chains. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297S variant that removes glycosylation on both chains. Heterodimeric Fc backbone 8 is based on human IgG4, and includes the L368D/K370S skew variants and the Q295E/N384D/Q418E/N421D pI variants on a first heterodimeric Fc chain, the S364K/E357Q skew variants on a second heterodimeric Fc chain, and the S228P (according to EU numbering, S241P in Kabat) variant that ablates Fab arm exchange (as is known in the art) on both chains. Heterodimeric Fc backbone 9 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 10 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain, and the S267K ablation variant on both chains. Heterodimeric Fc backbone 11 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 12 is based on human IgG1 (356E/358M allotype), and includes the L368D/K370S skew variants on a first heterodimeric Fc chain, the S364K/E357Q skew variants and P217R/P229R/N276K pI variants on a second heterodimeric Fc chain, and the E233P/L234V/L235A/G236del/S267K ablation variants on both chains. Heterodimeric Fc backbone 13 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 14 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains. Heterodimeric Fc backbone 15 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage.

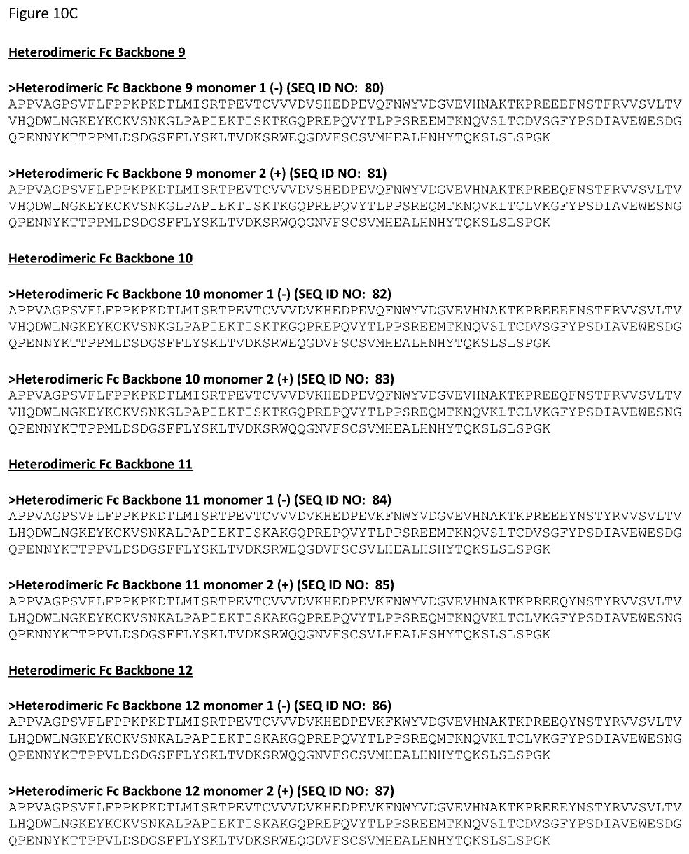

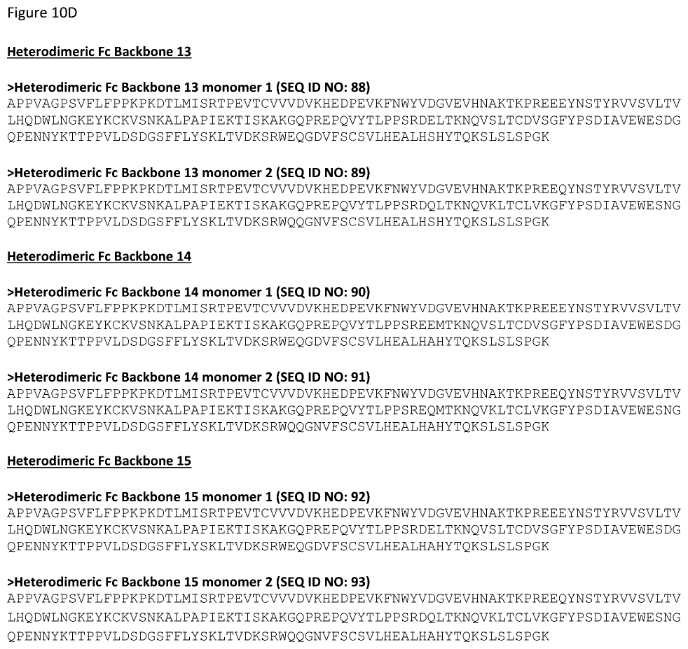

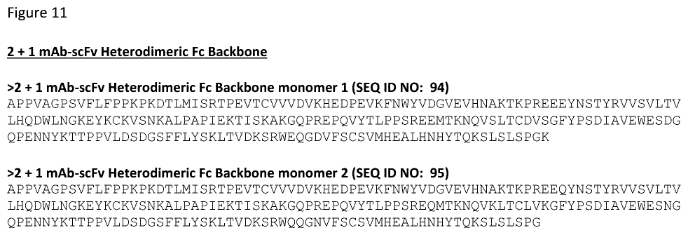

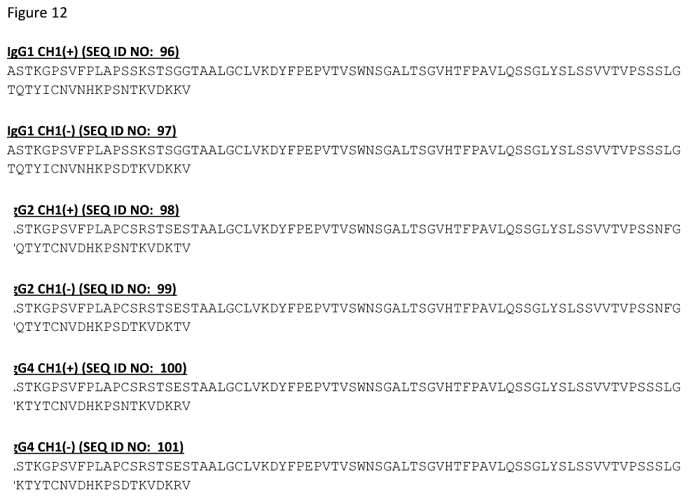

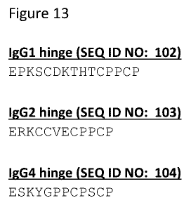

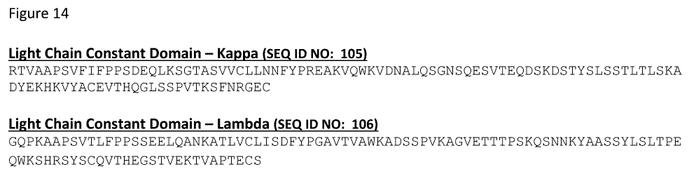

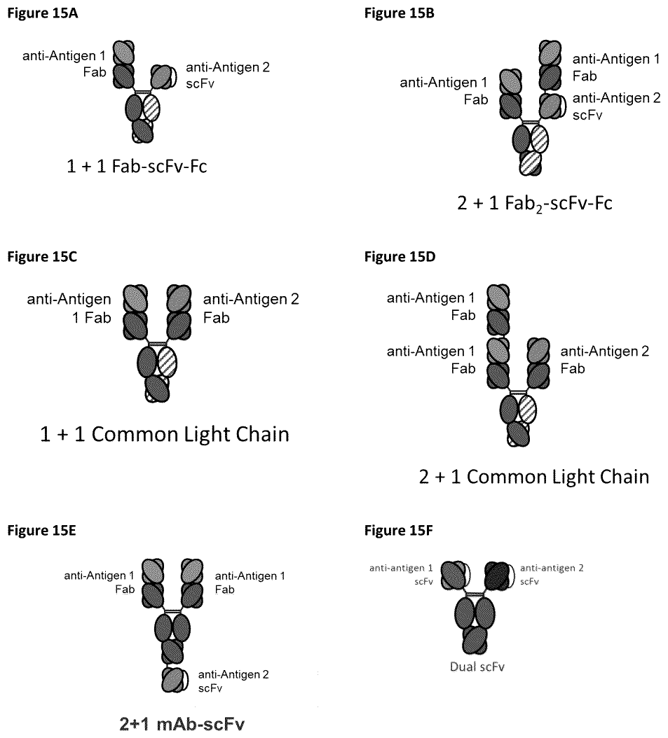

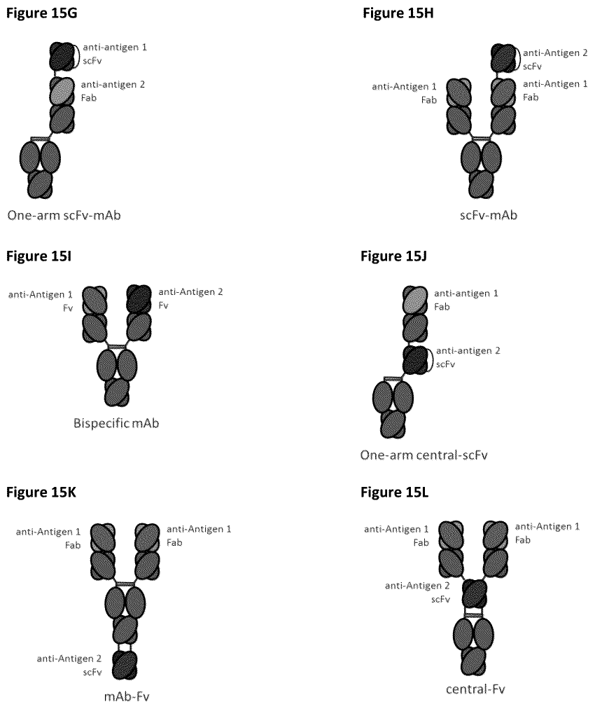

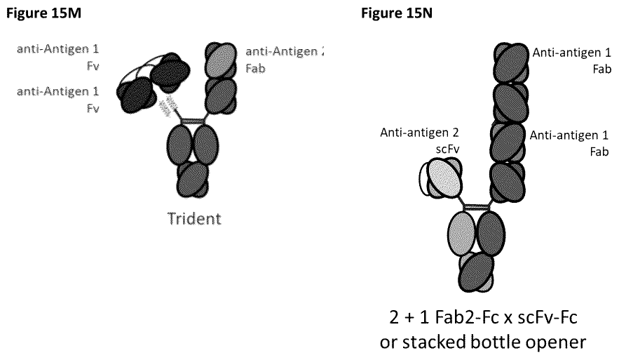

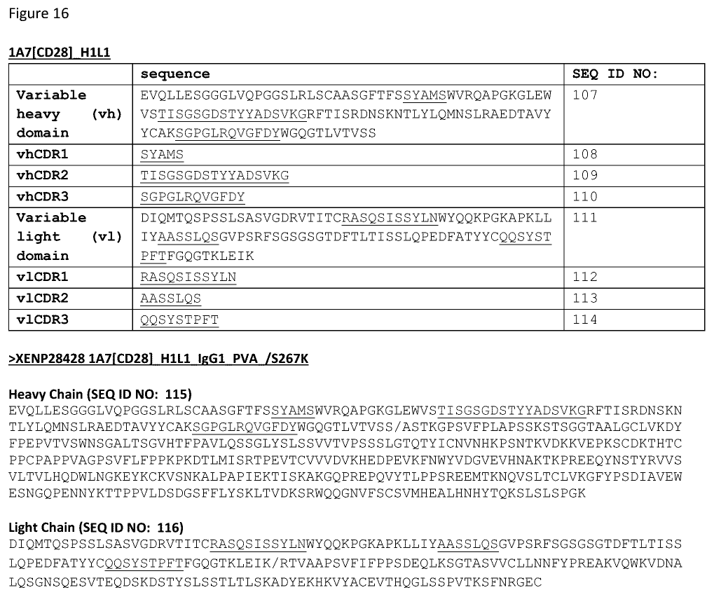

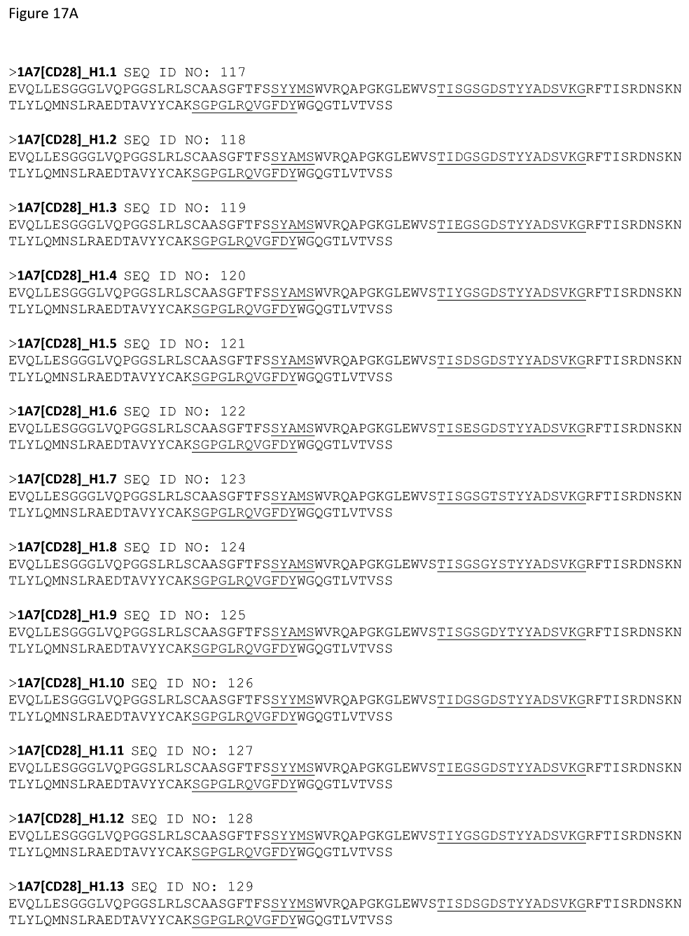

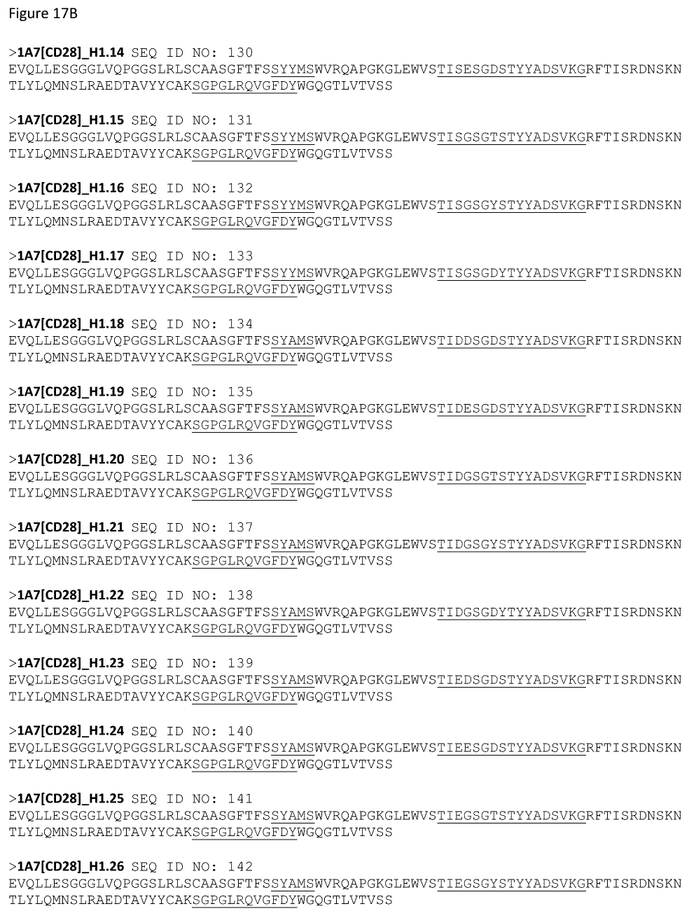

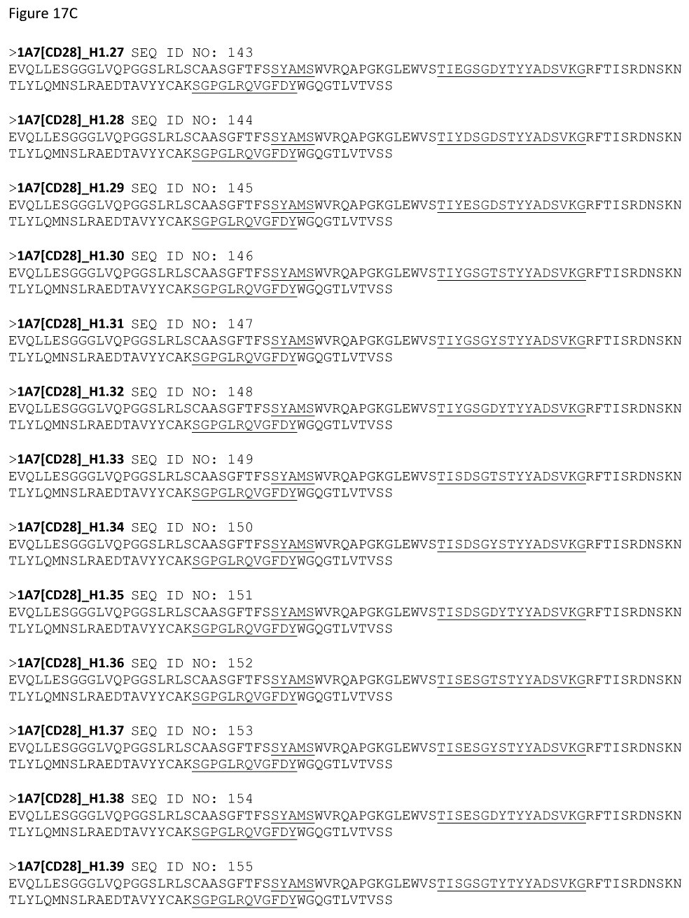

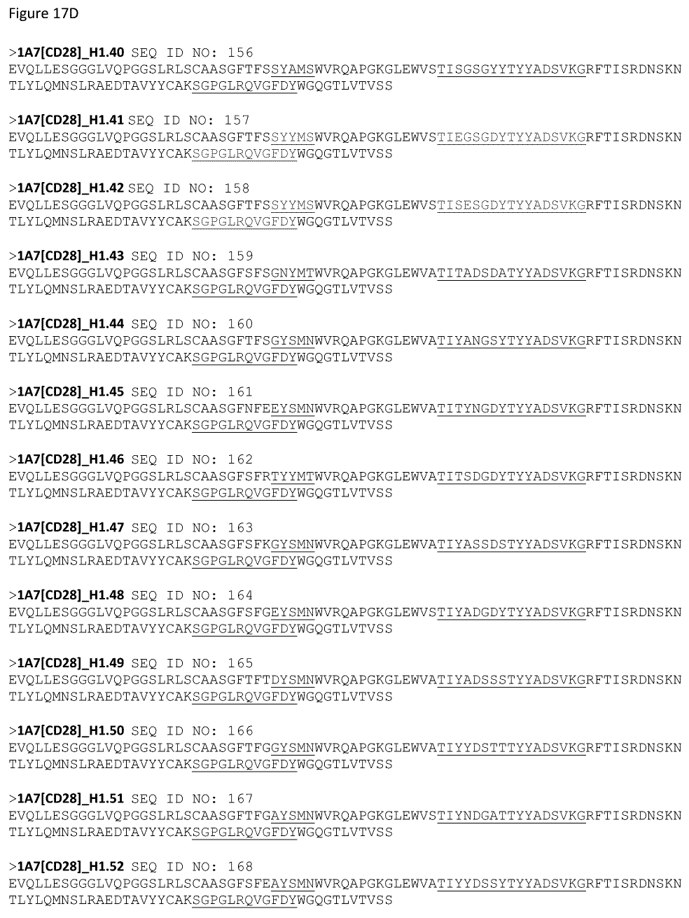

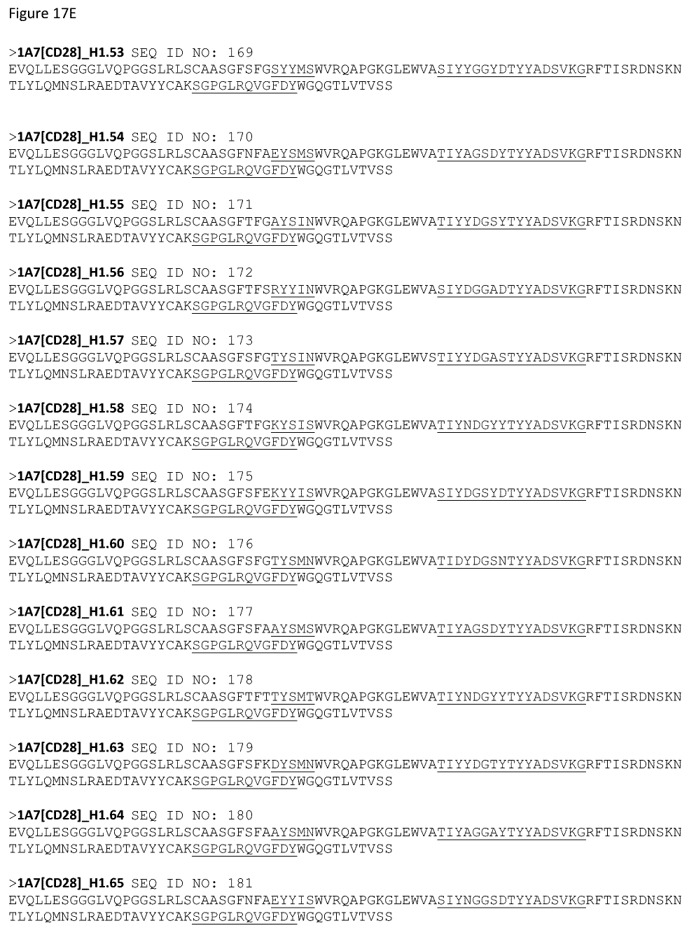

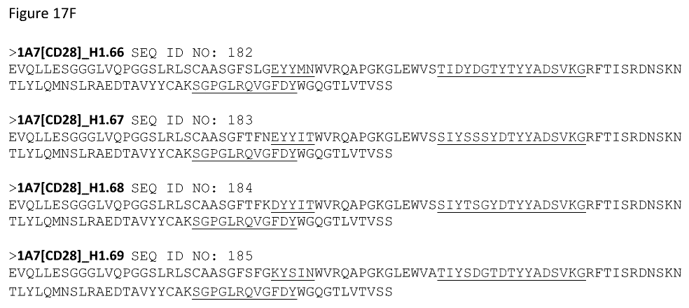

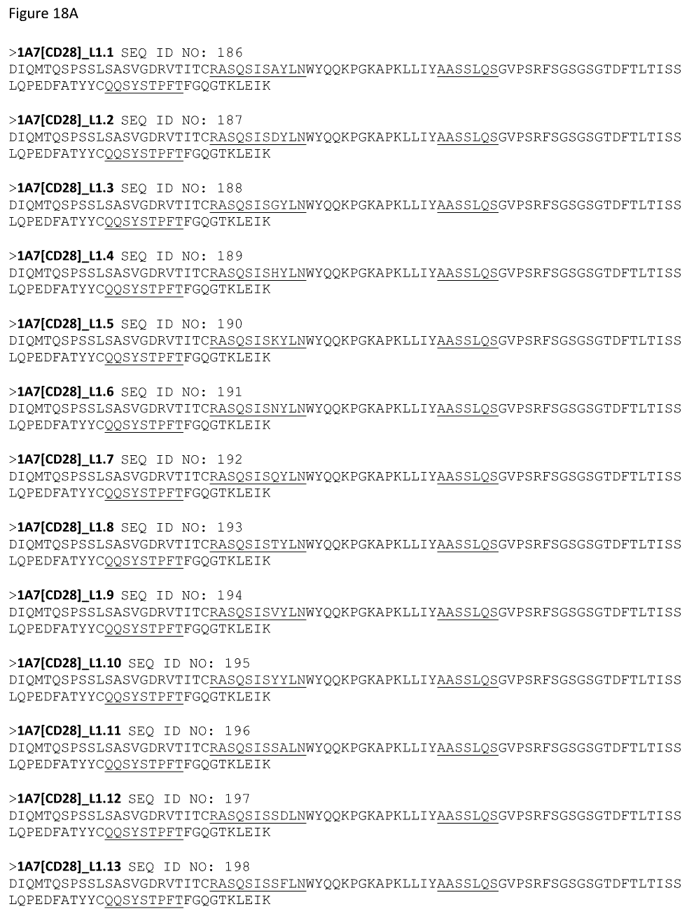

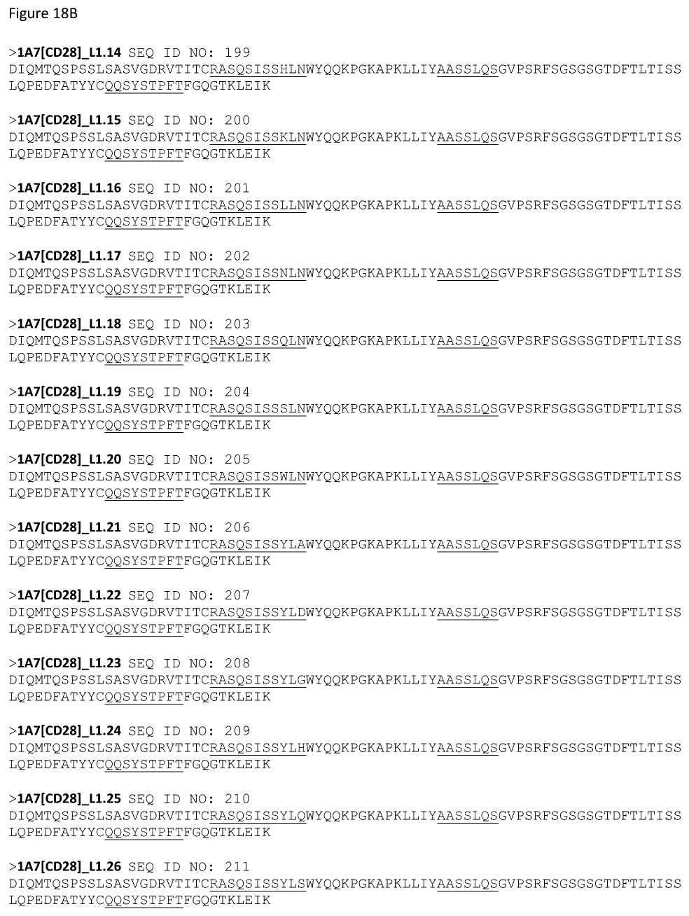

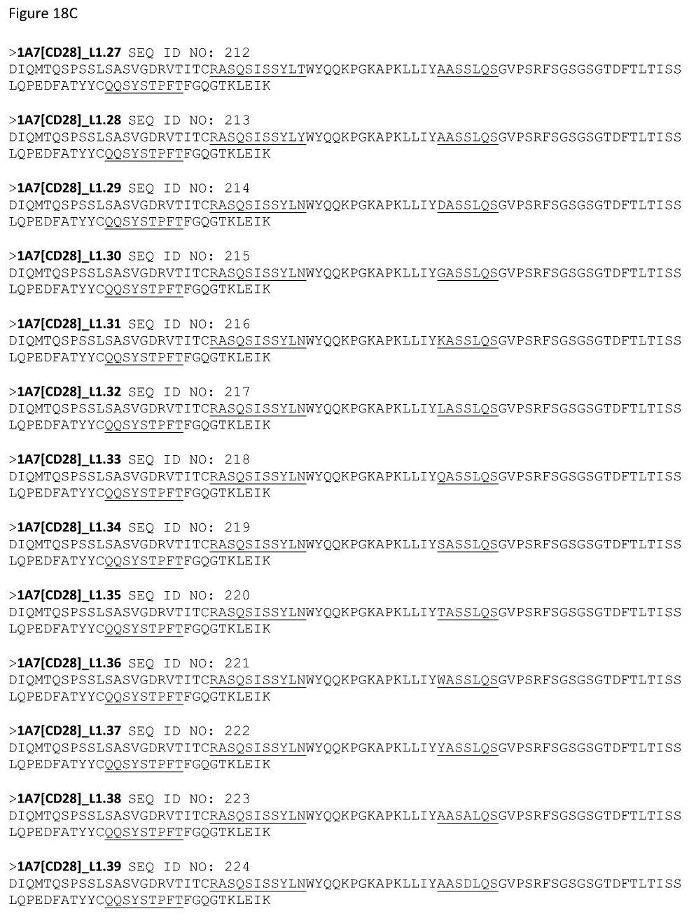

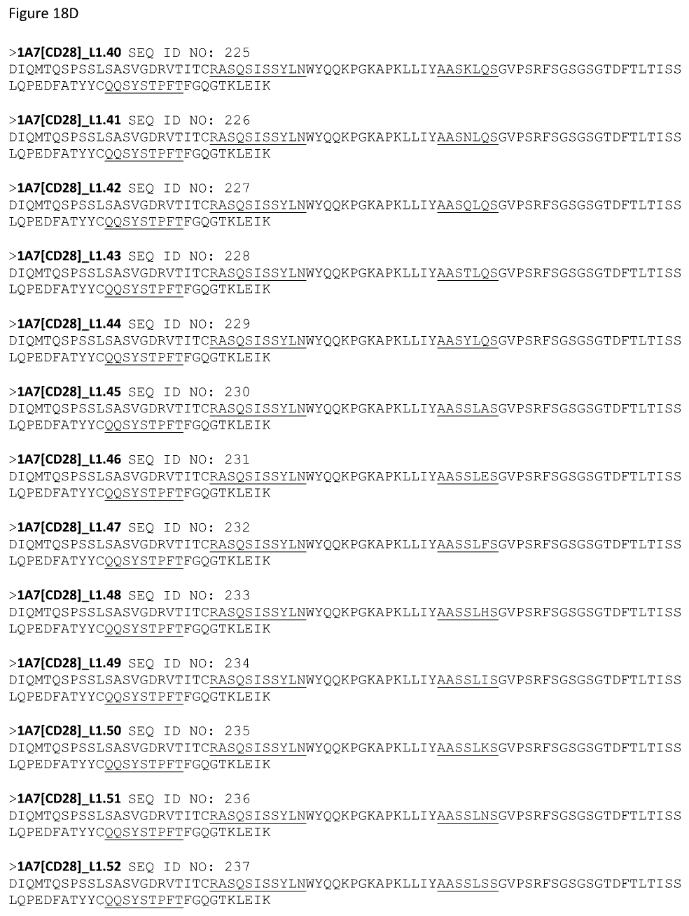

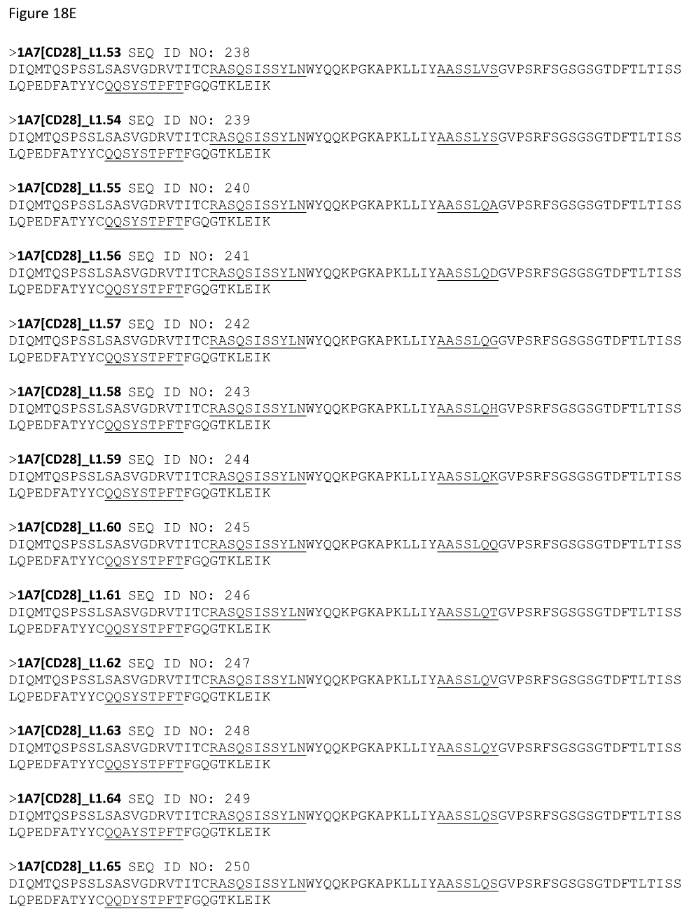

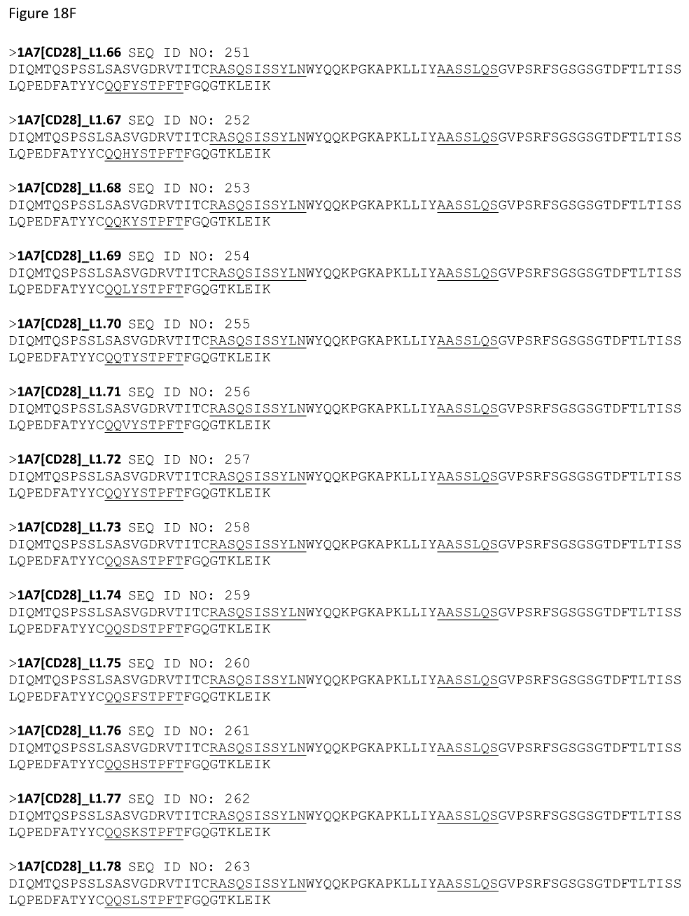

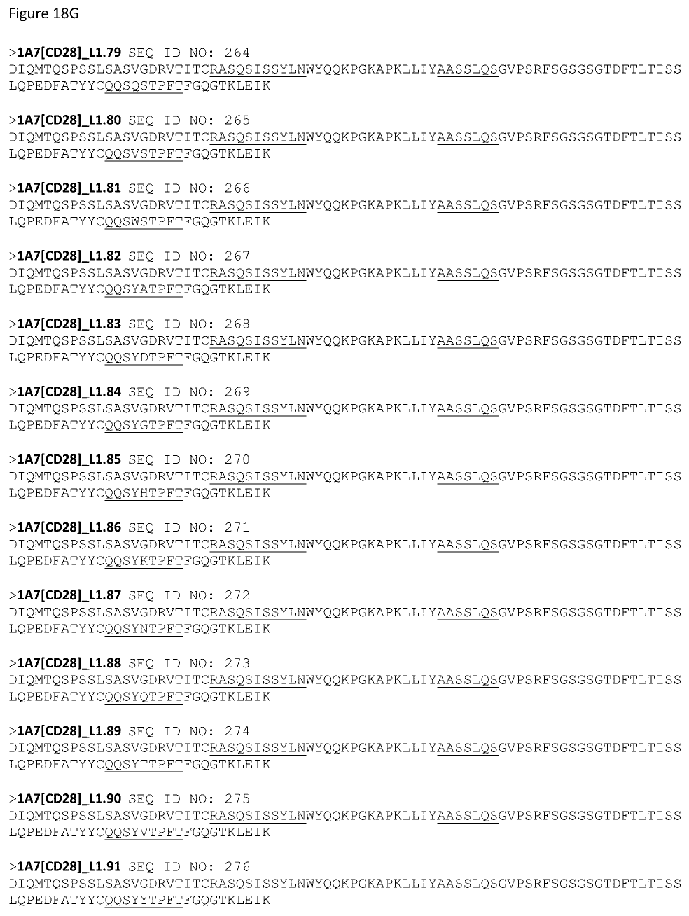

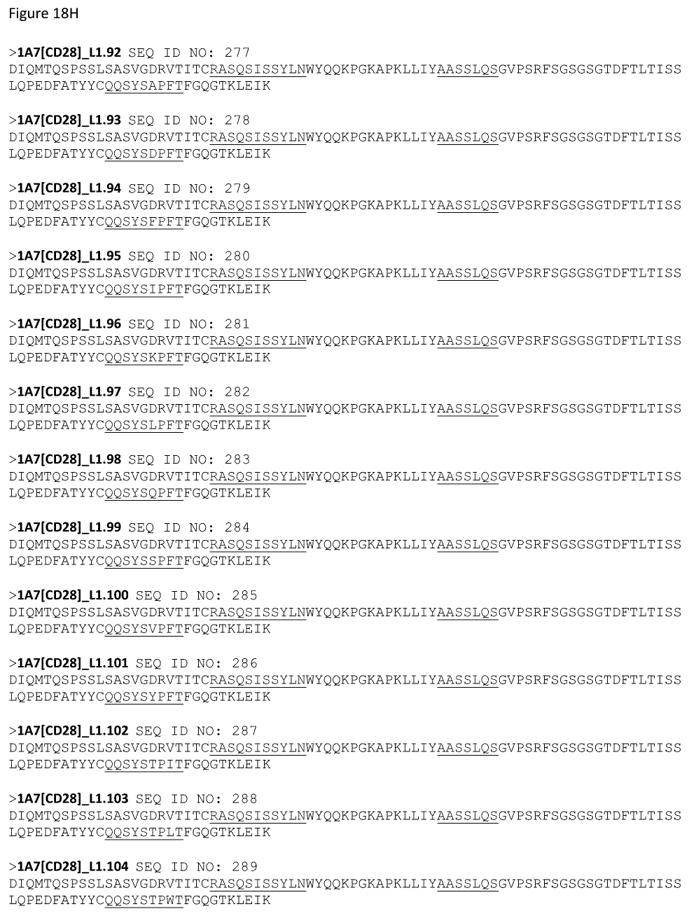

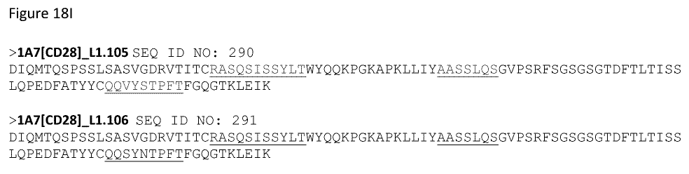

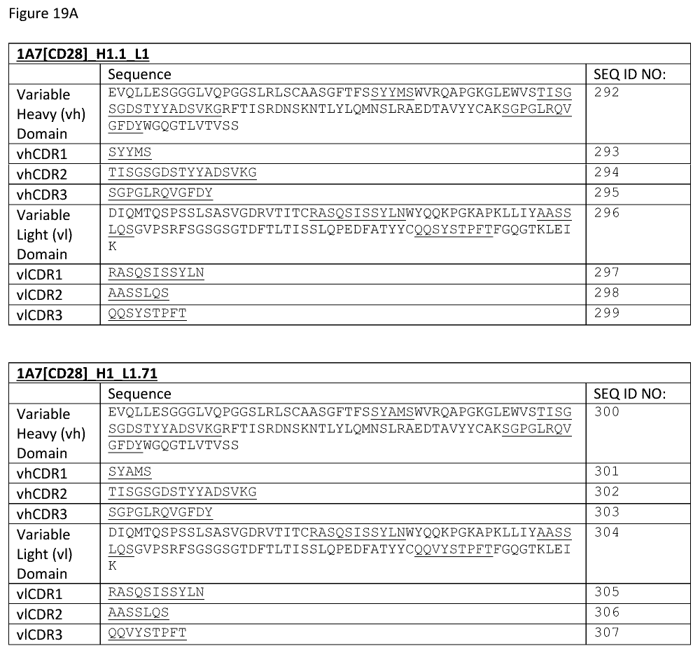

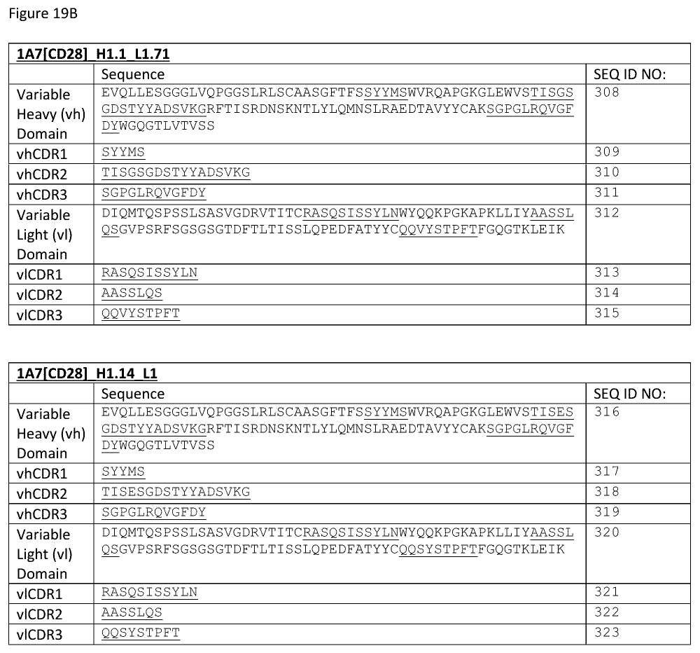

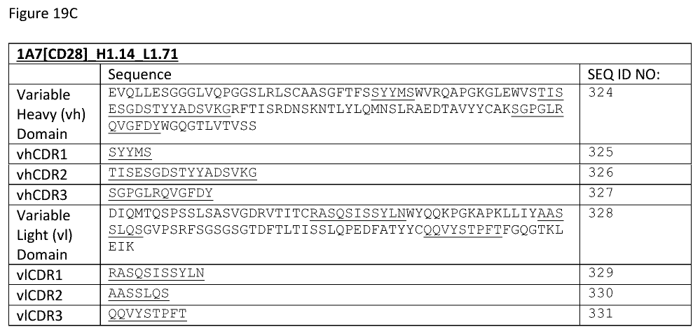

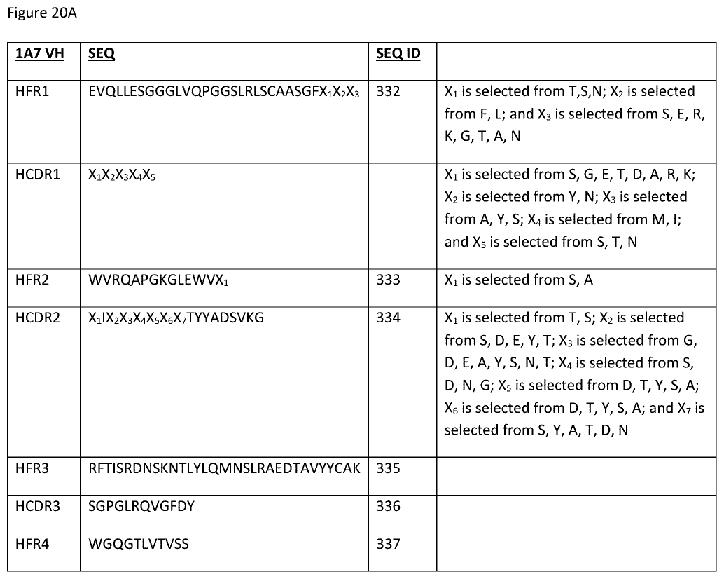

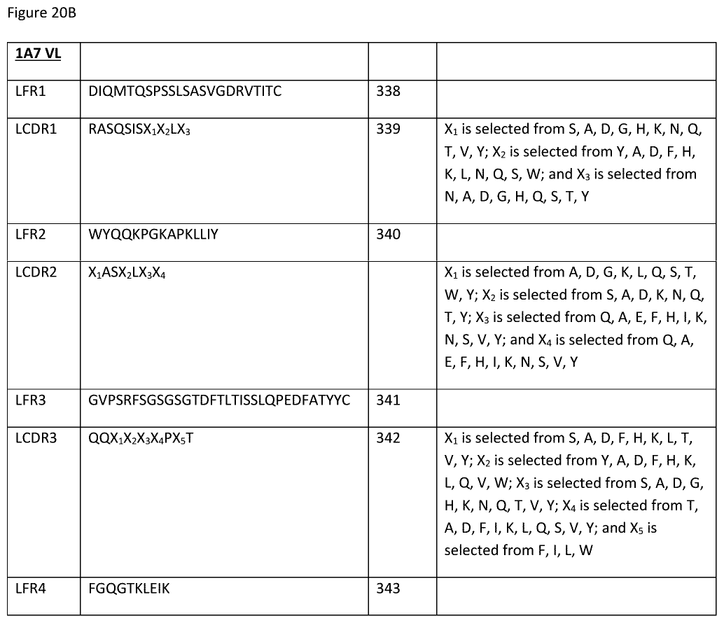

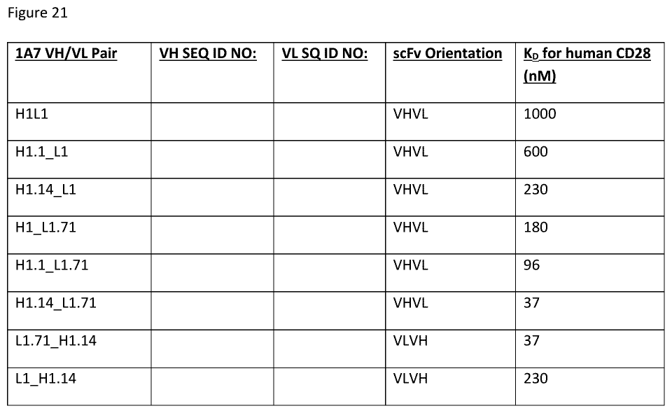

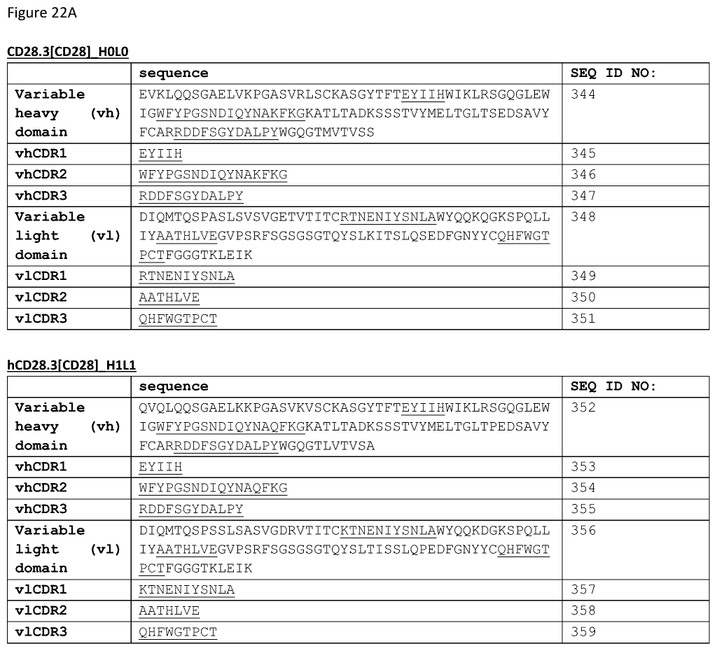

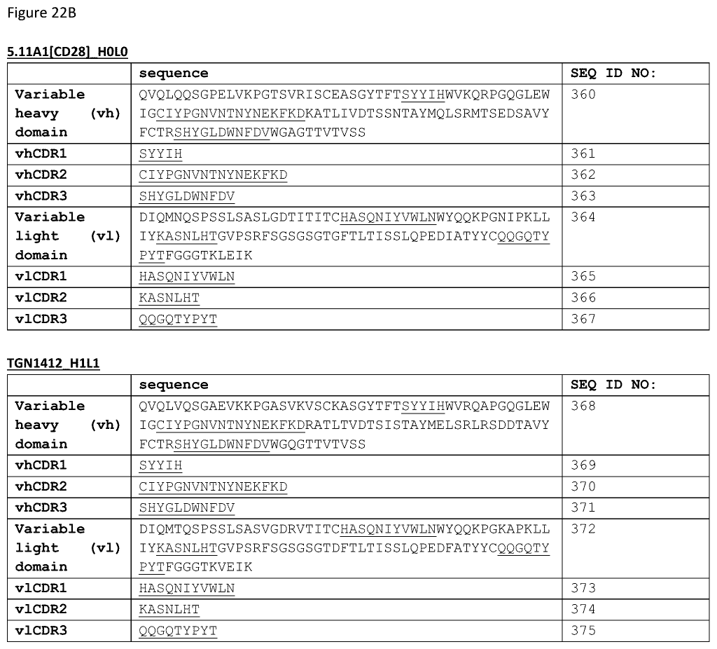

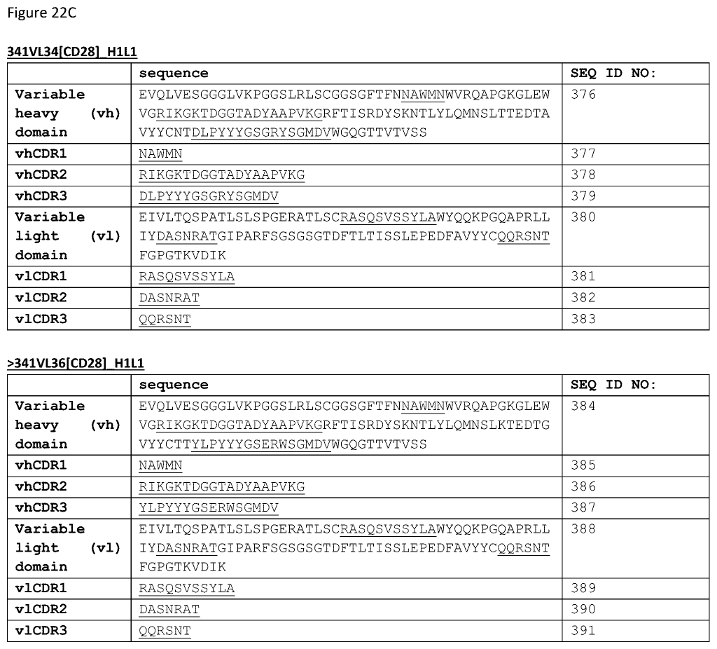

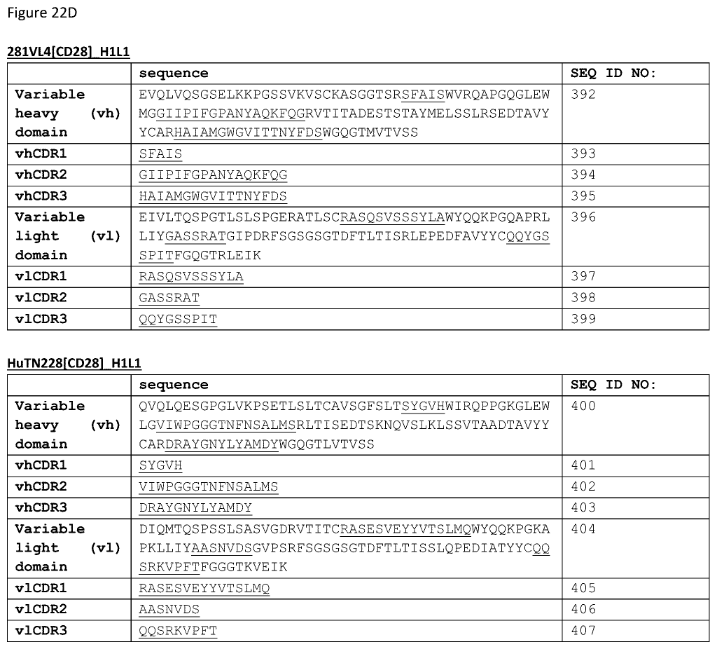

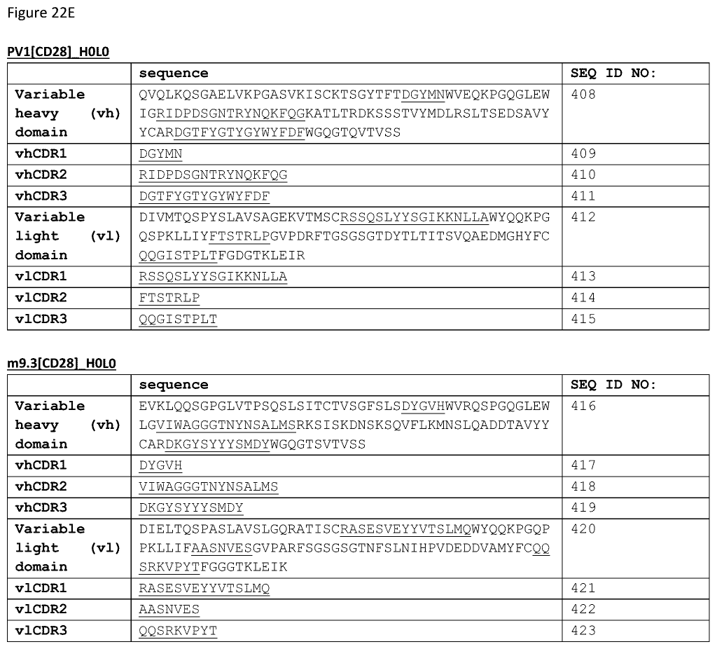

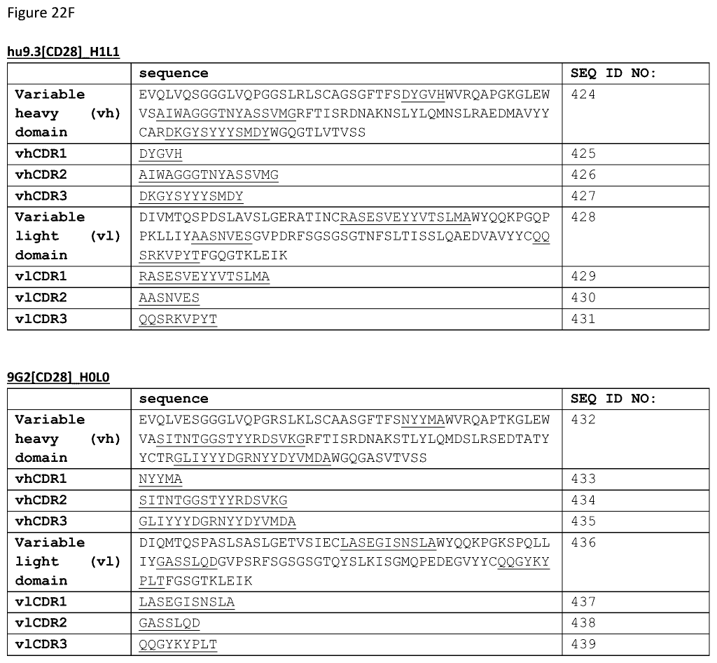

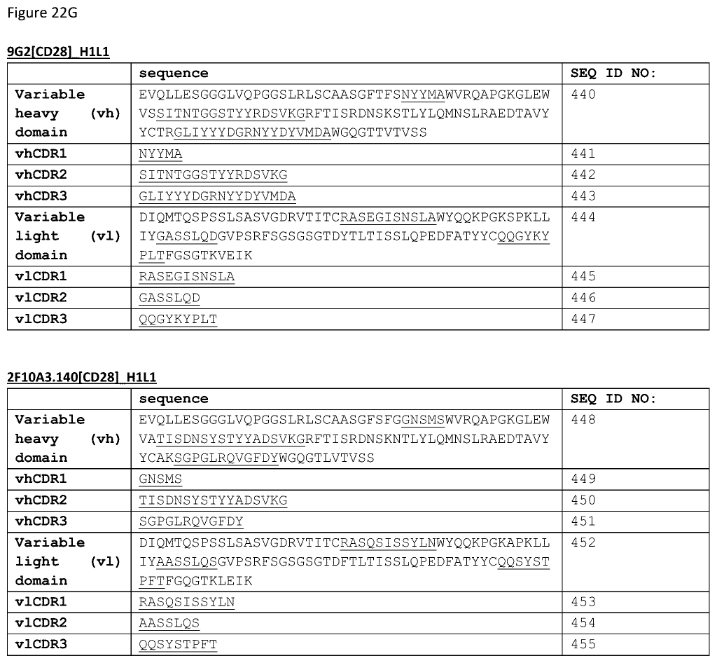

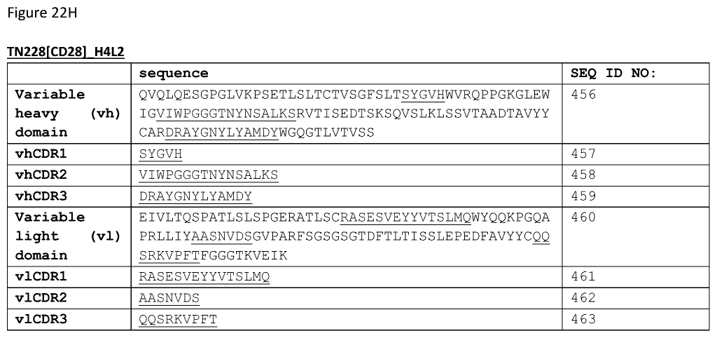

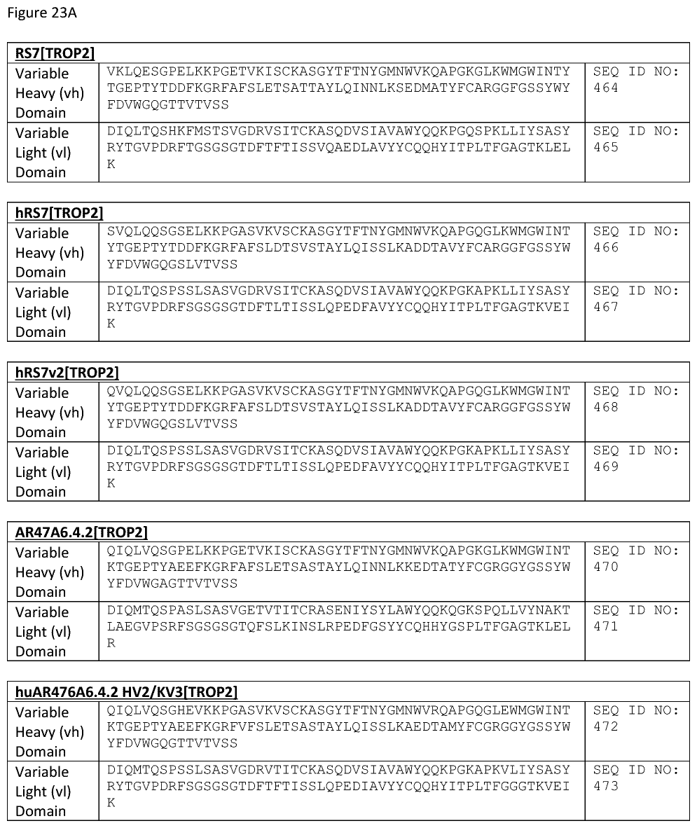

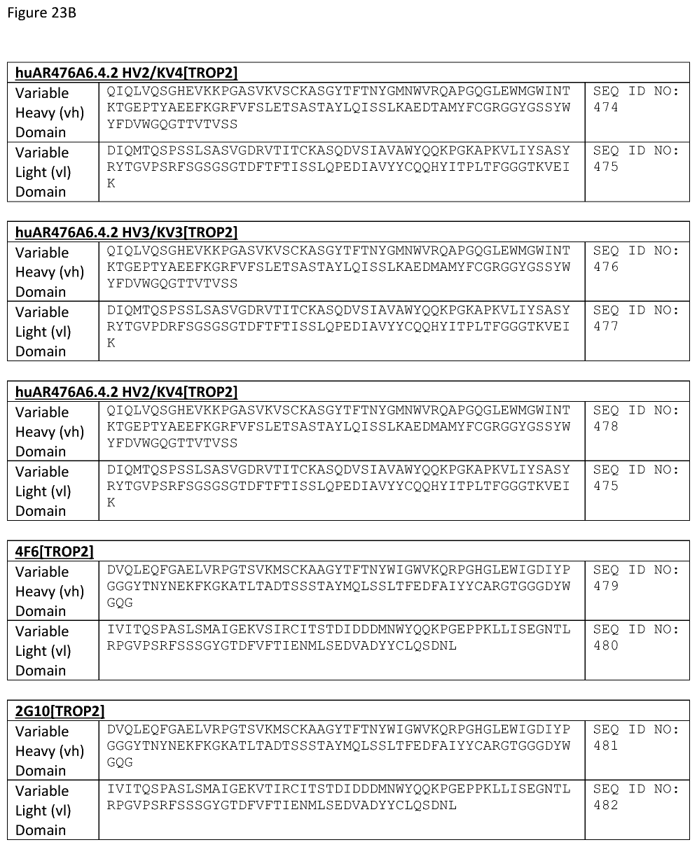

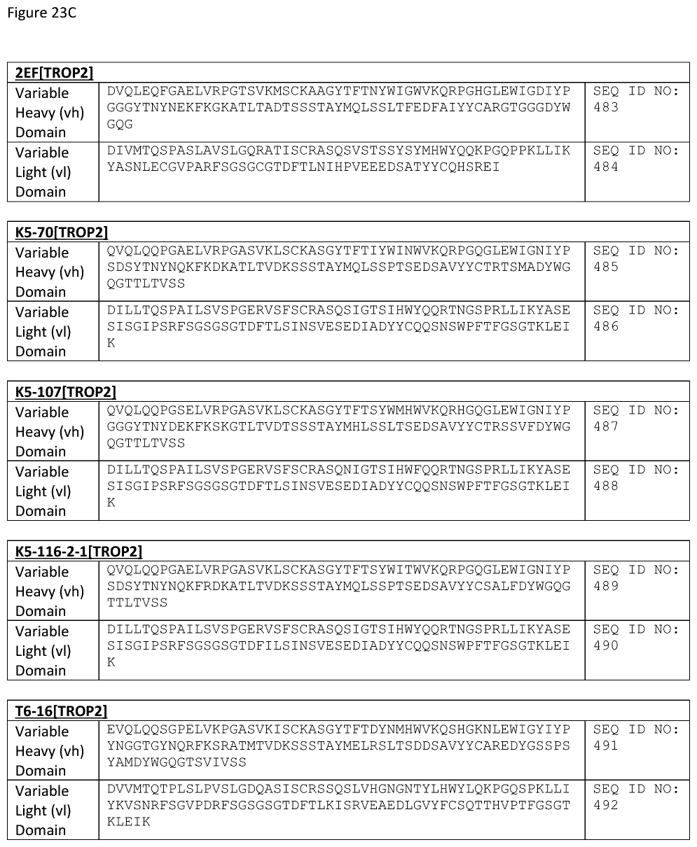

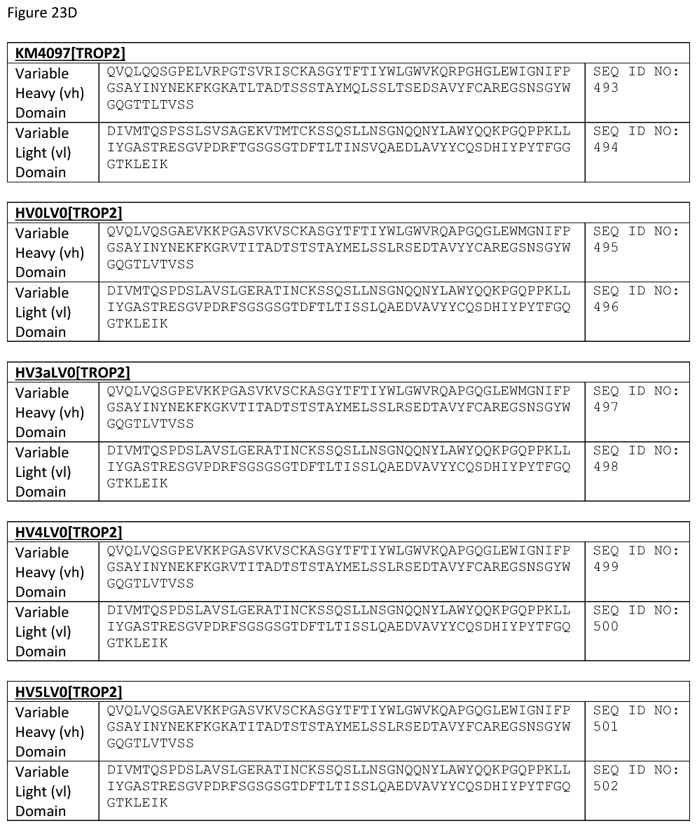

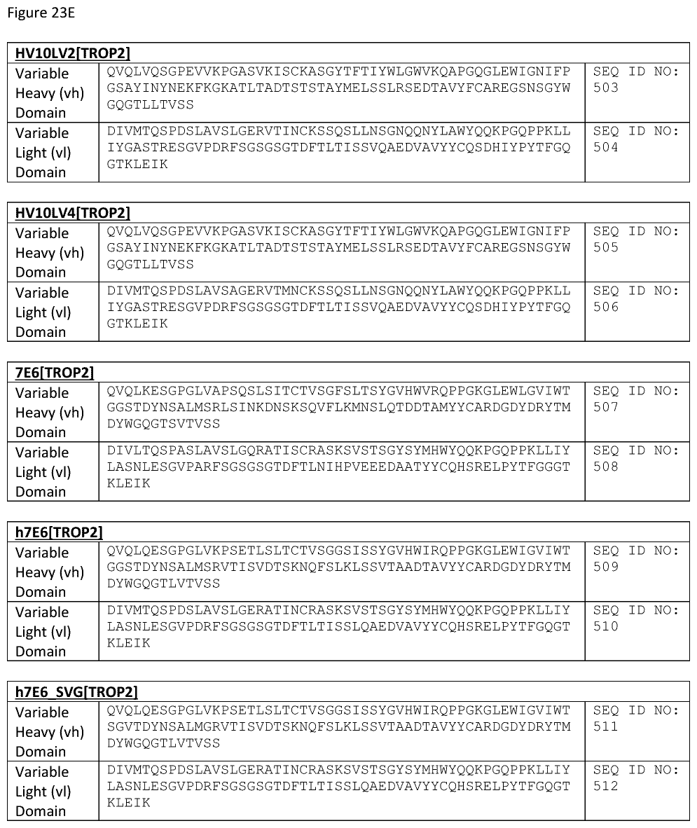

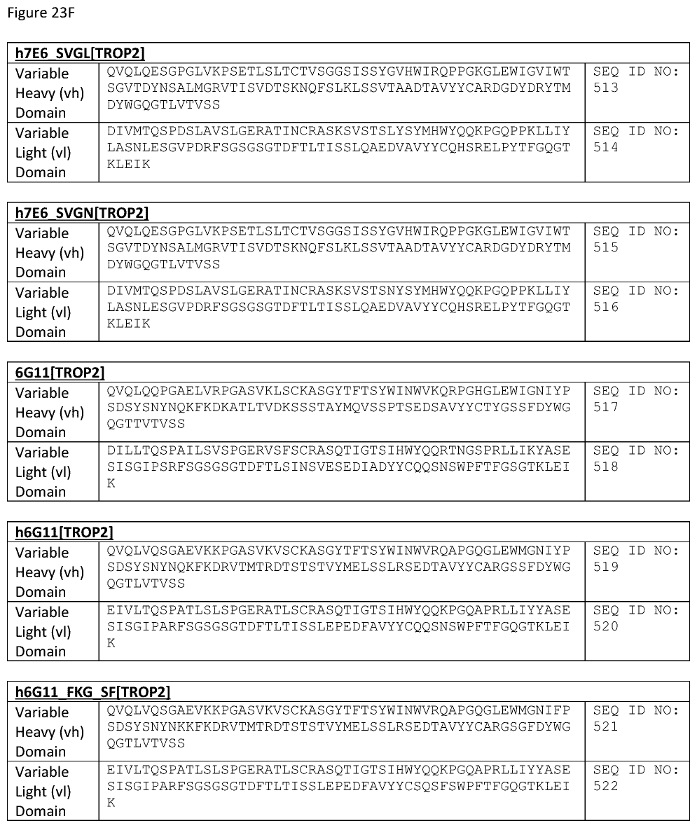

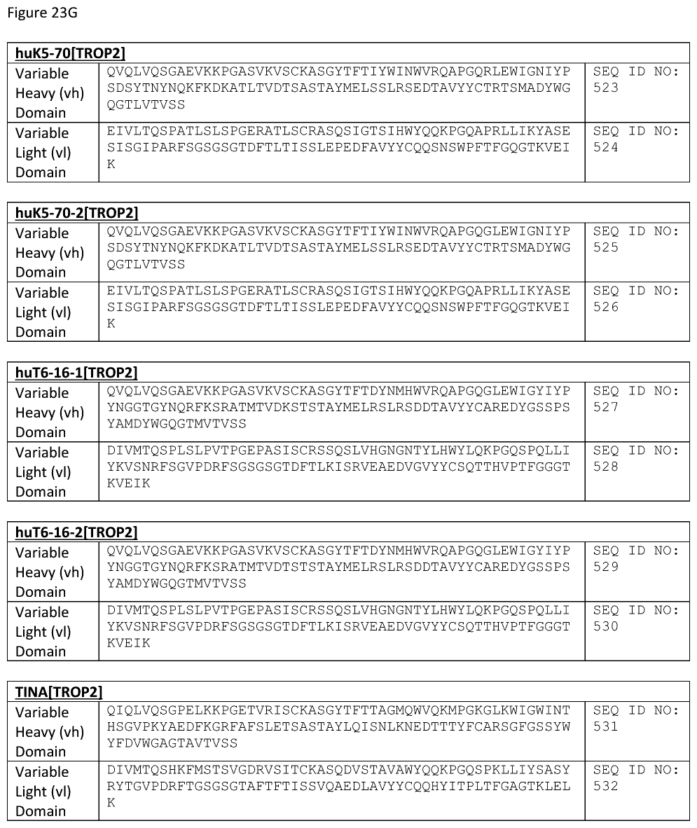

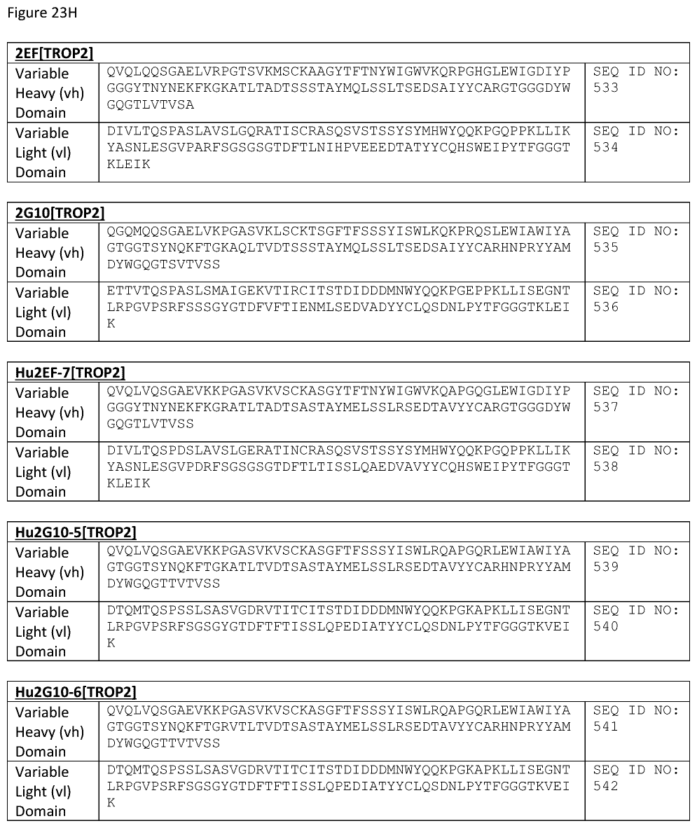

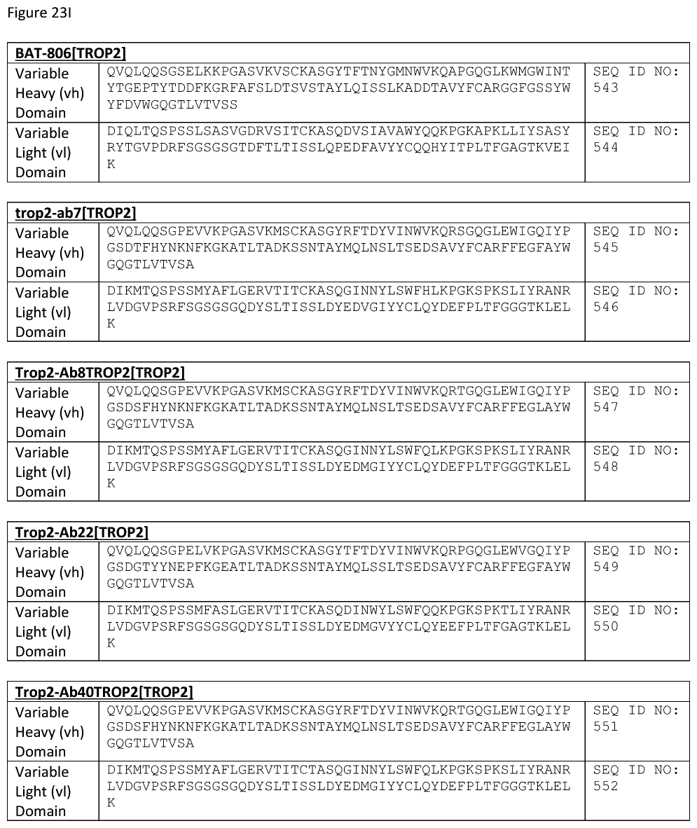

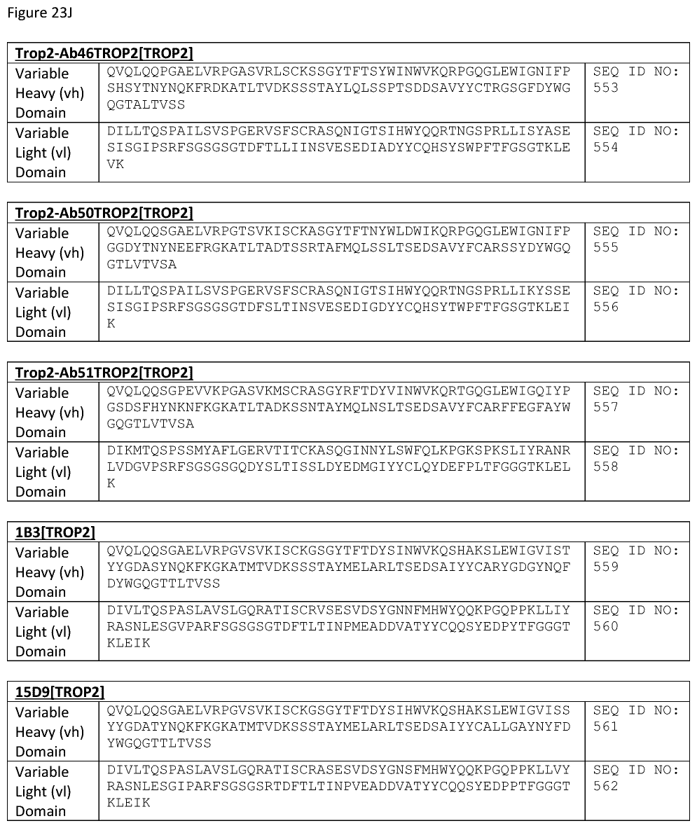

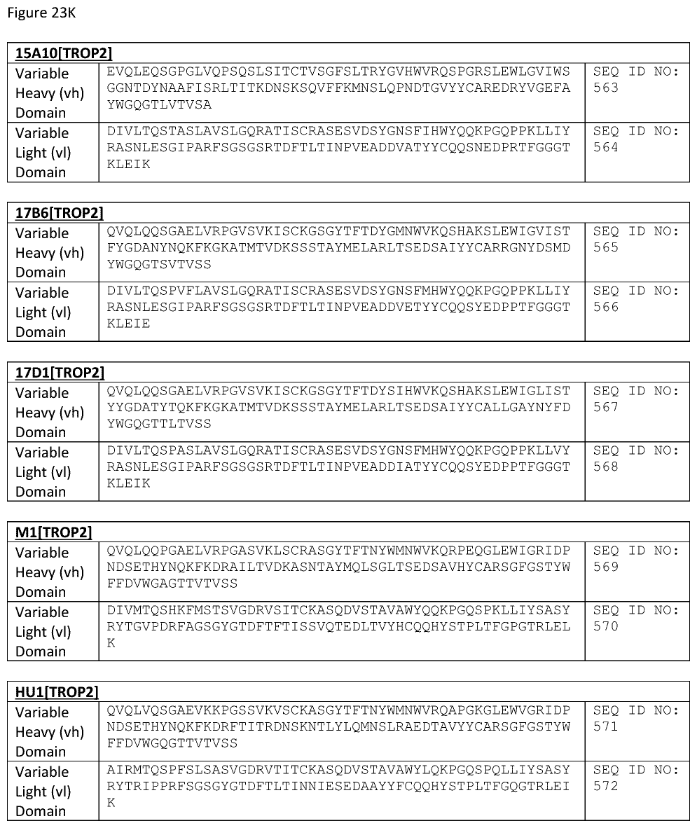

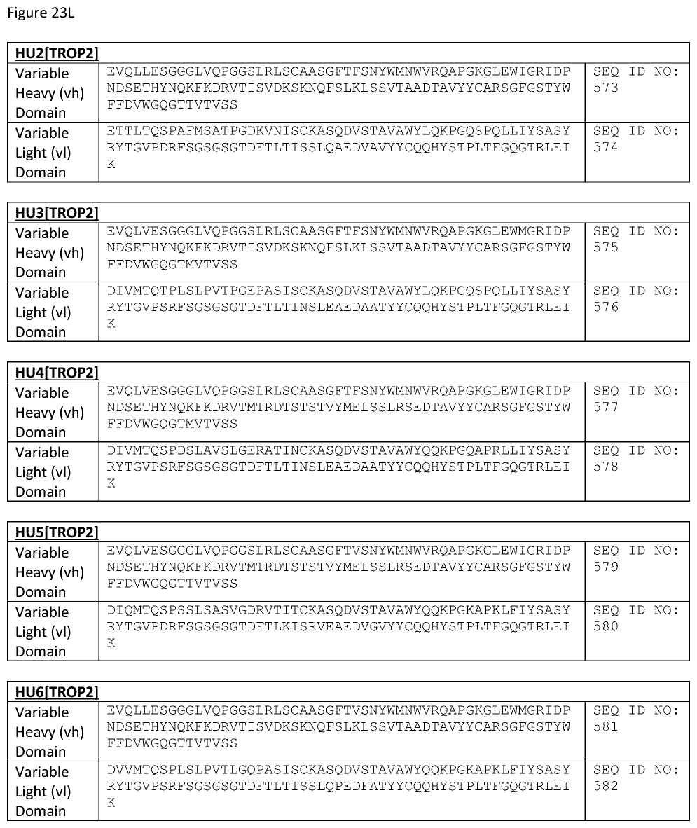

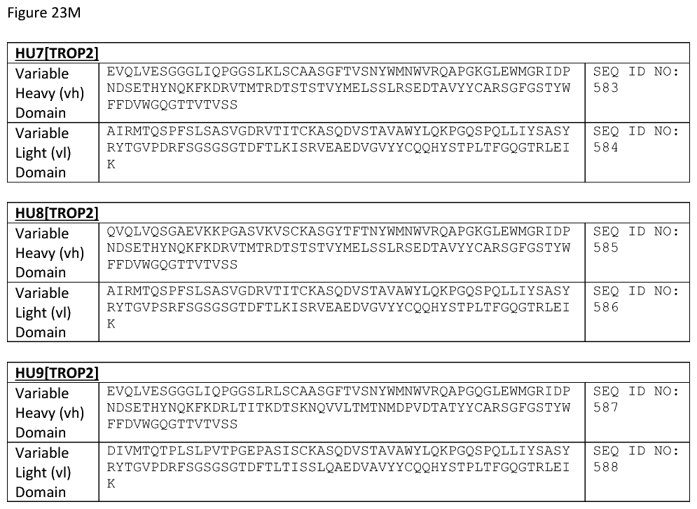

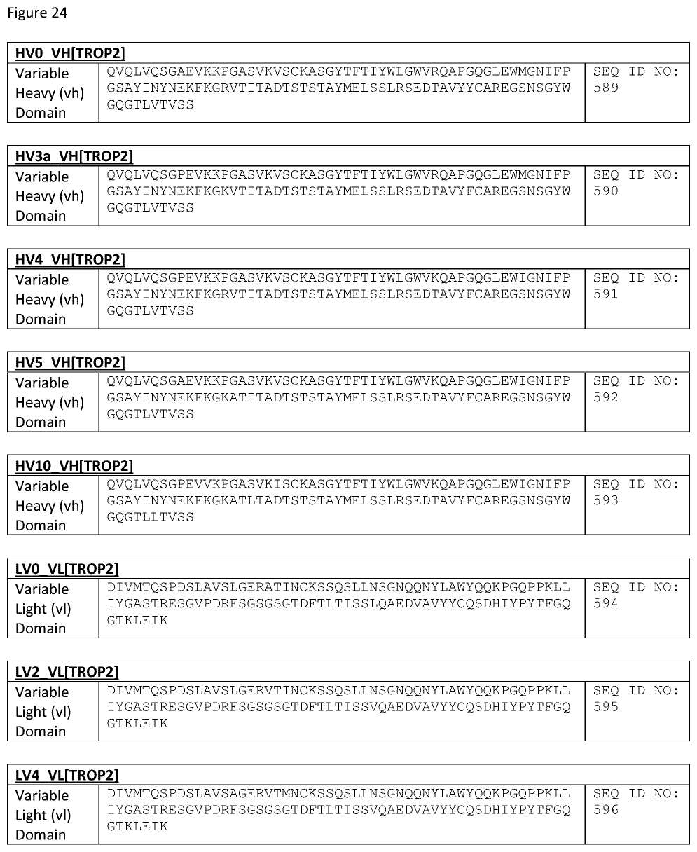

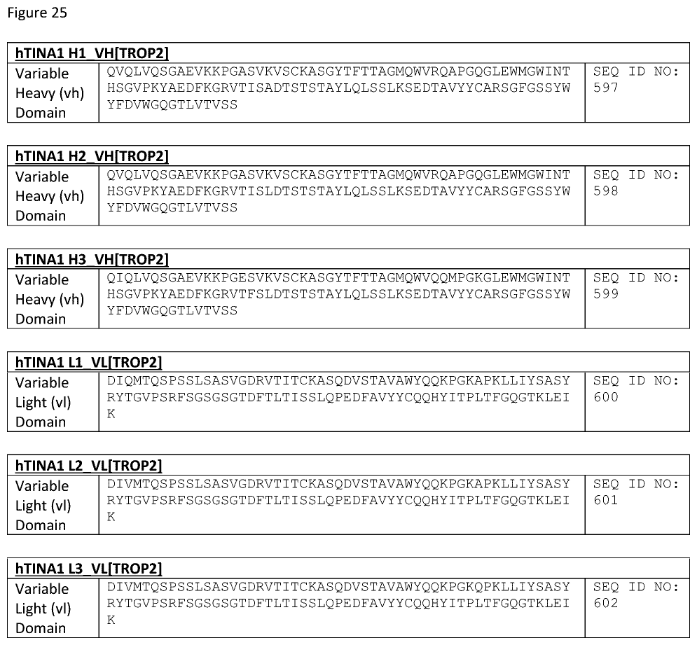

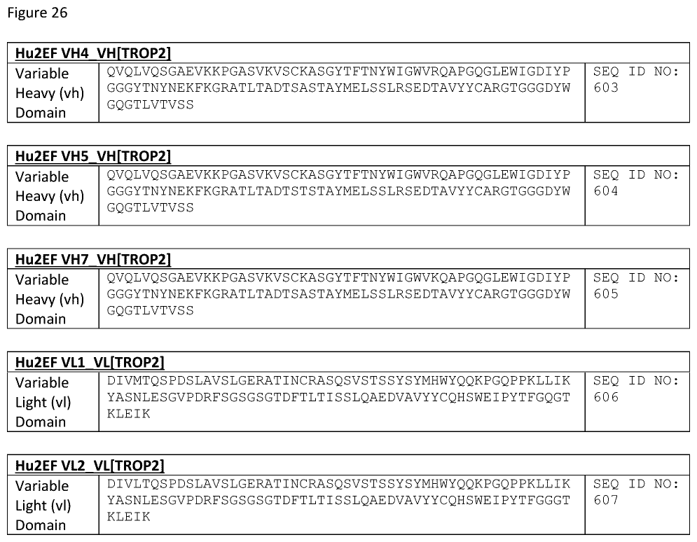

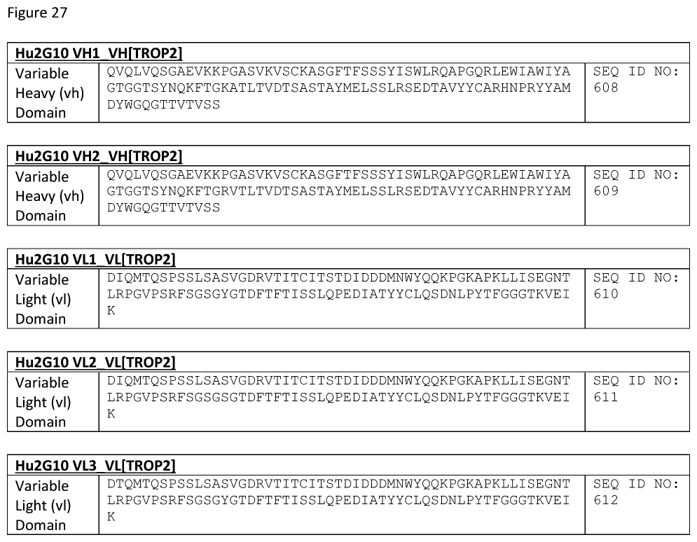

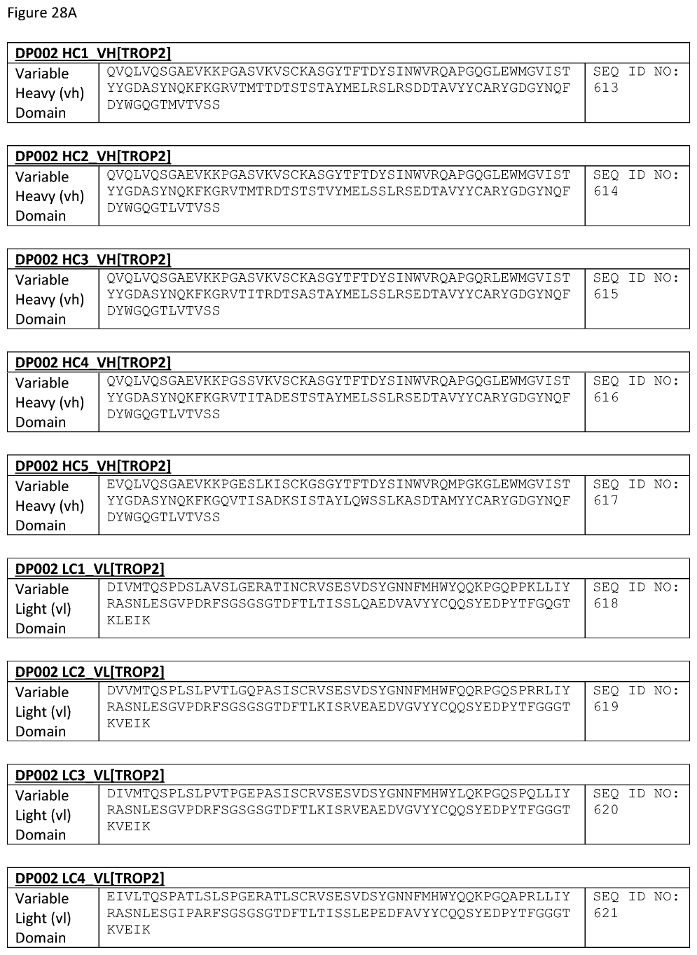

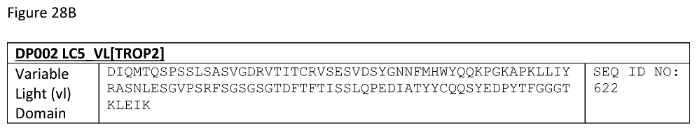

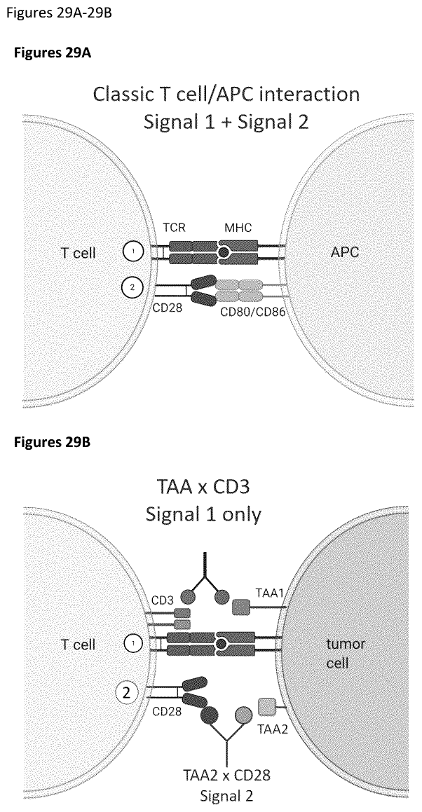

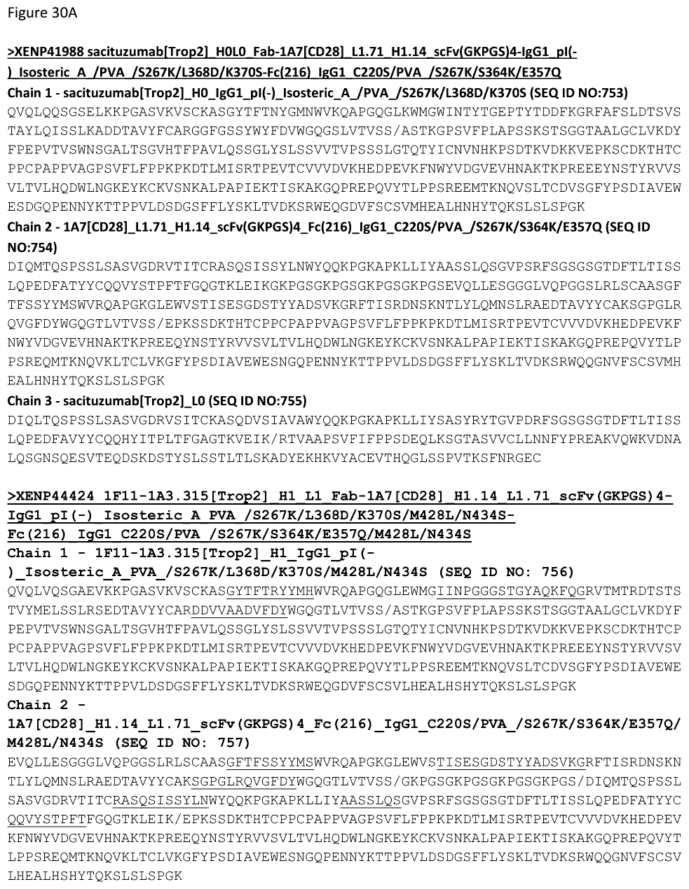

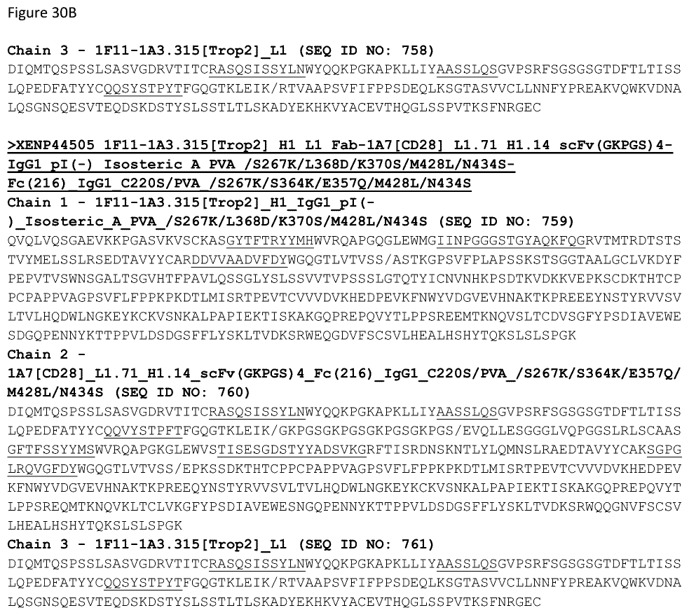

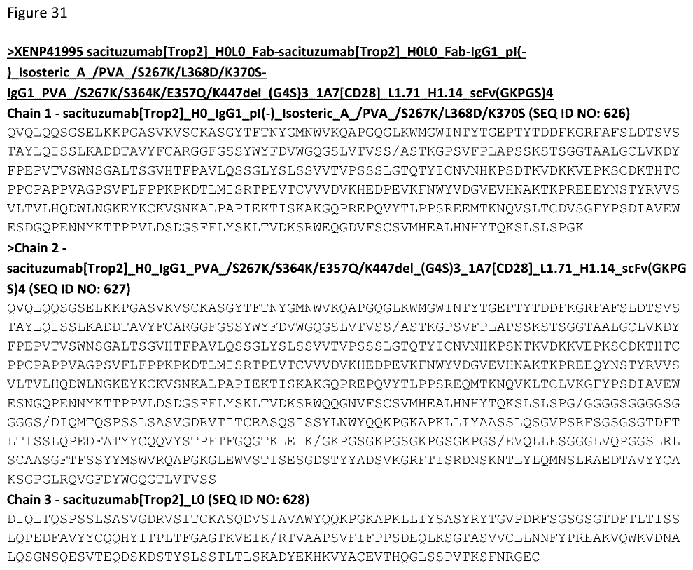

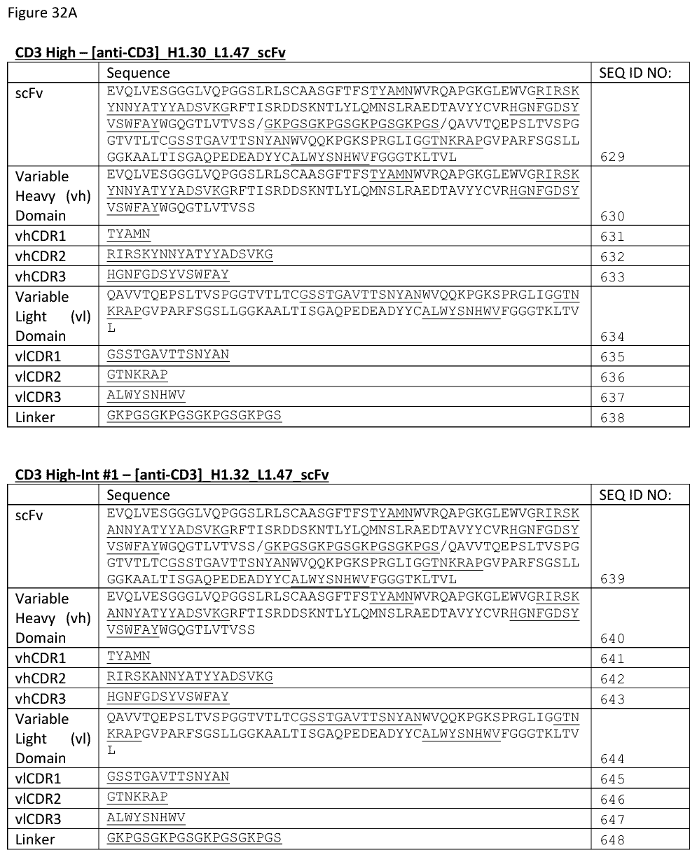

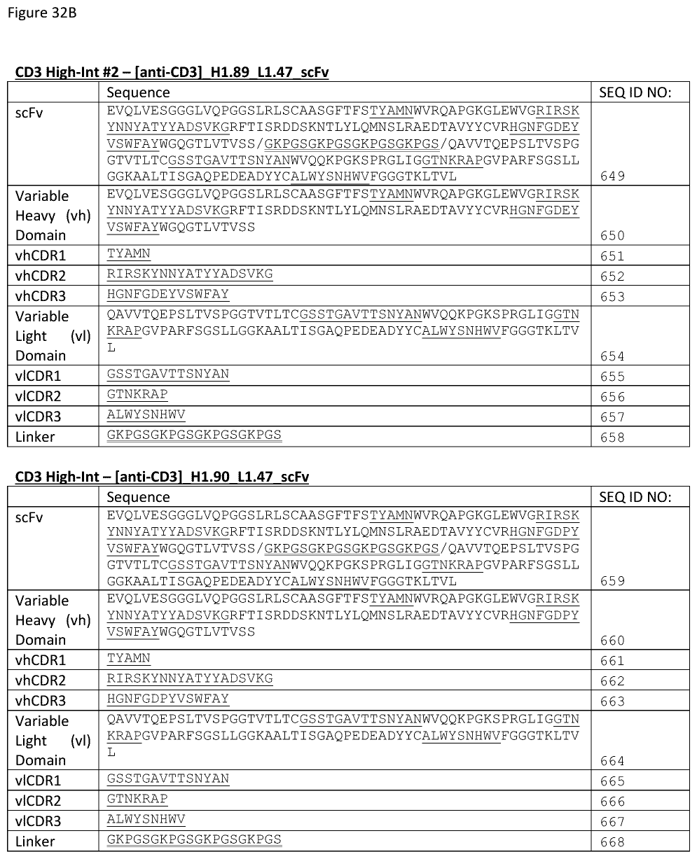

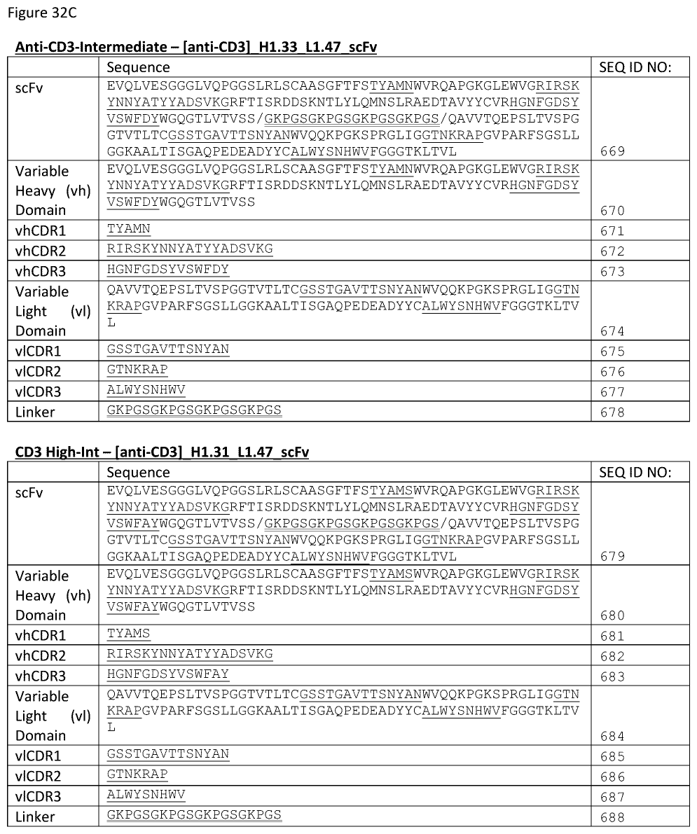

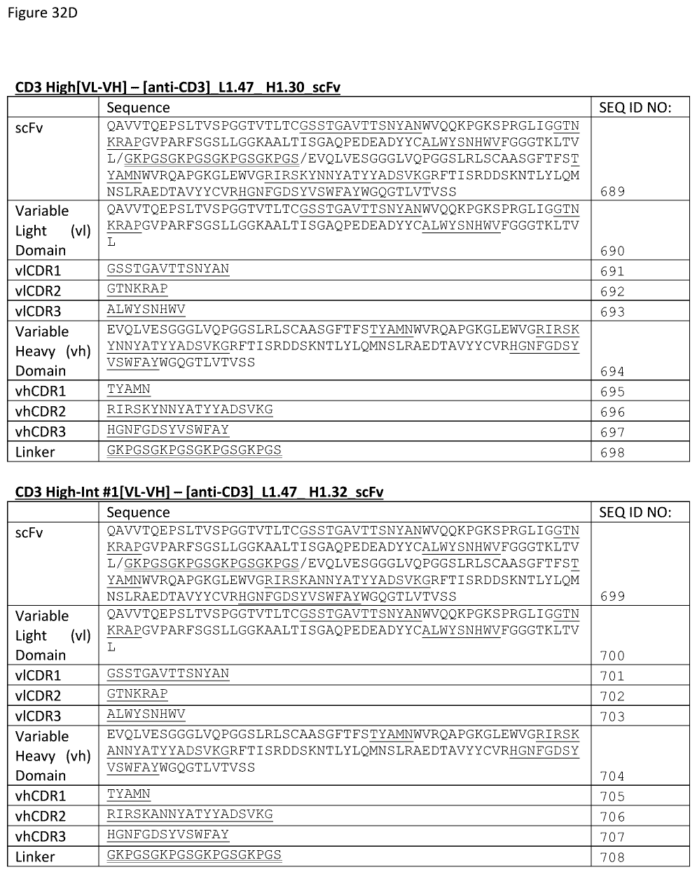

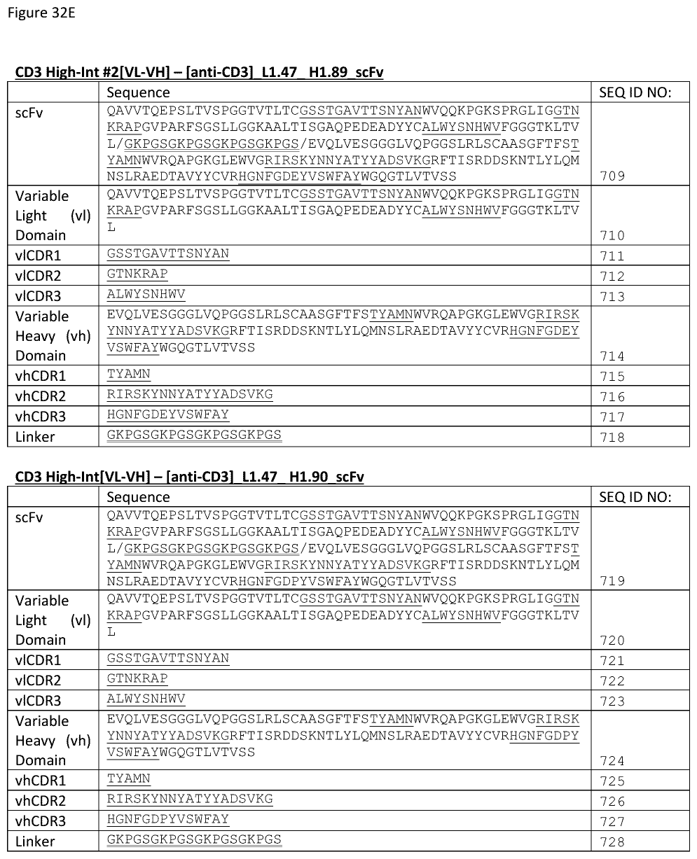

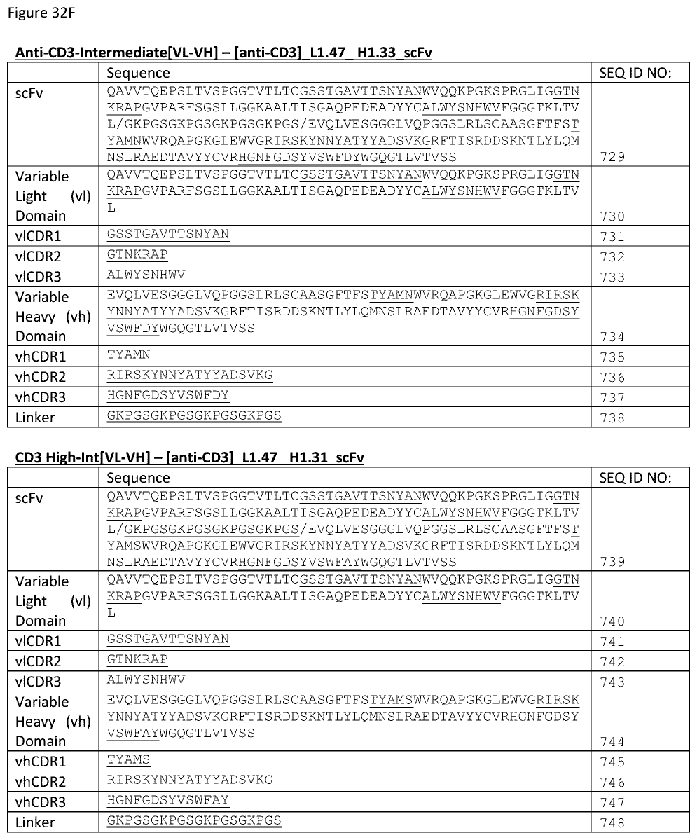

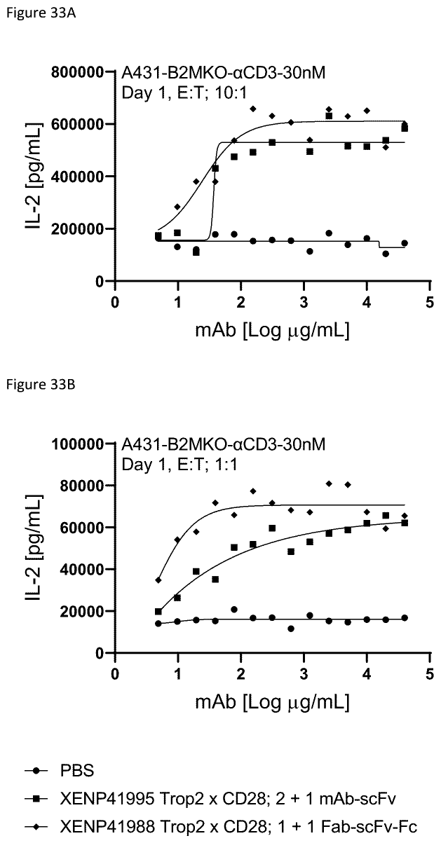

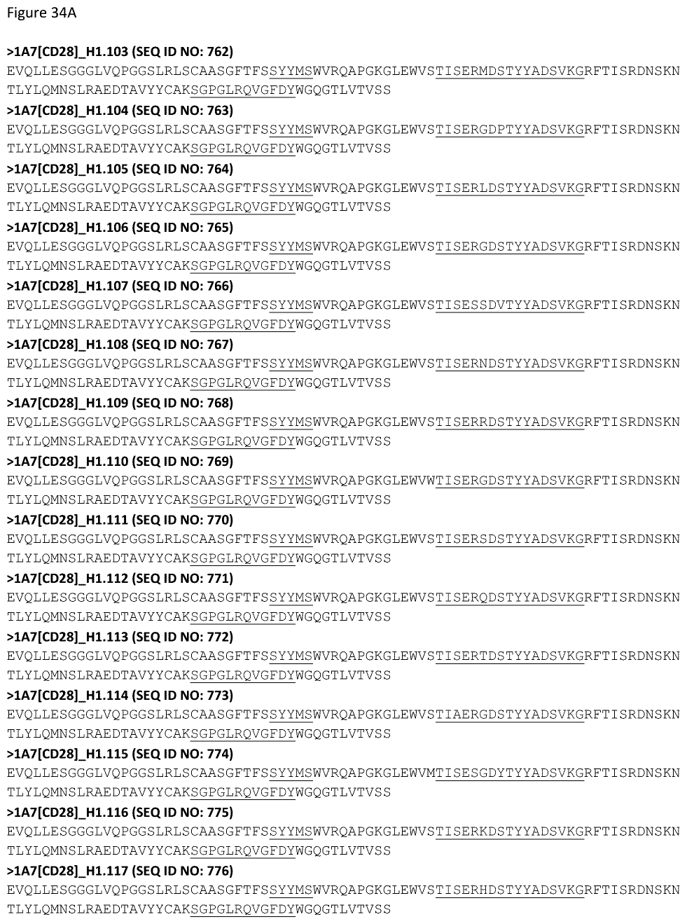

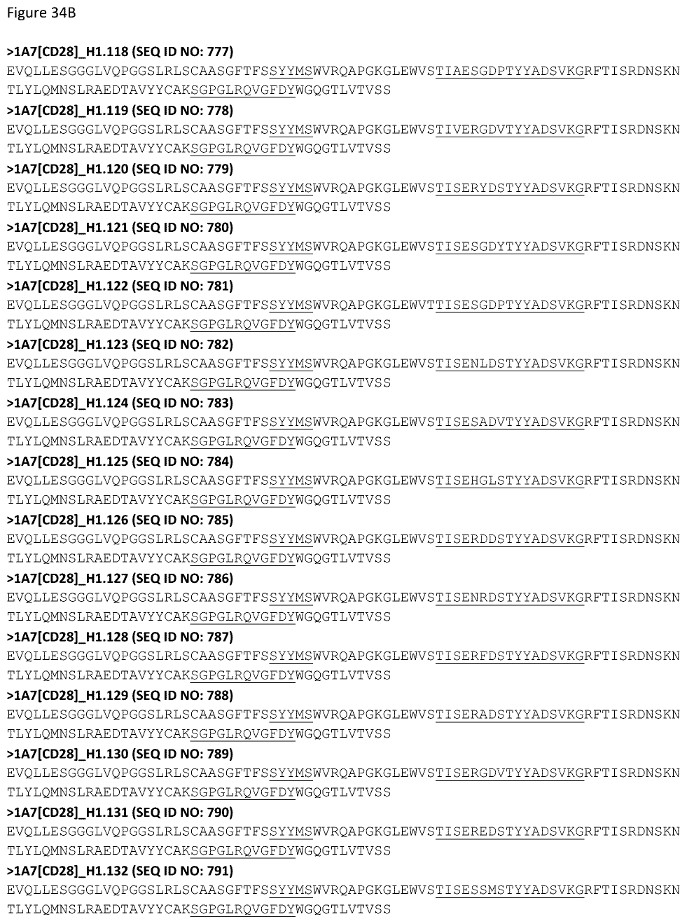

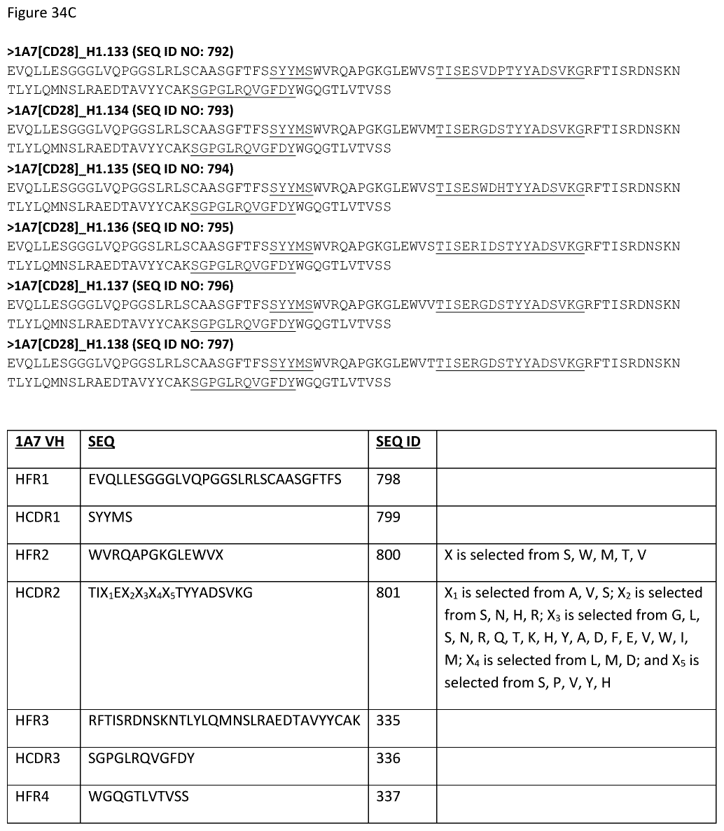

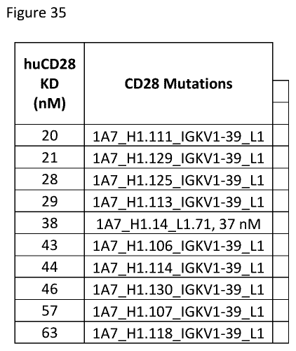

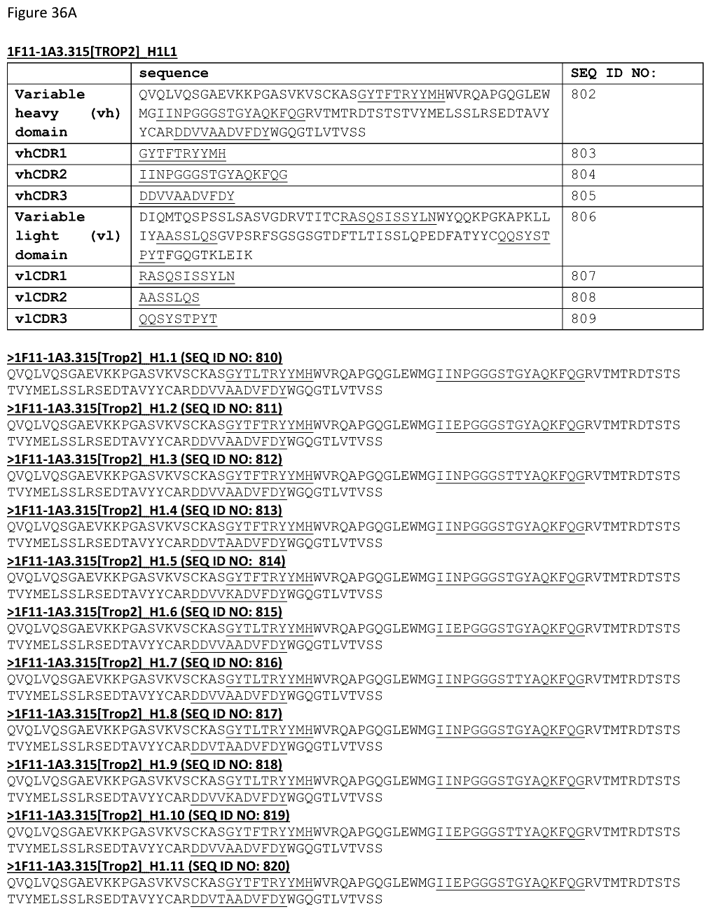

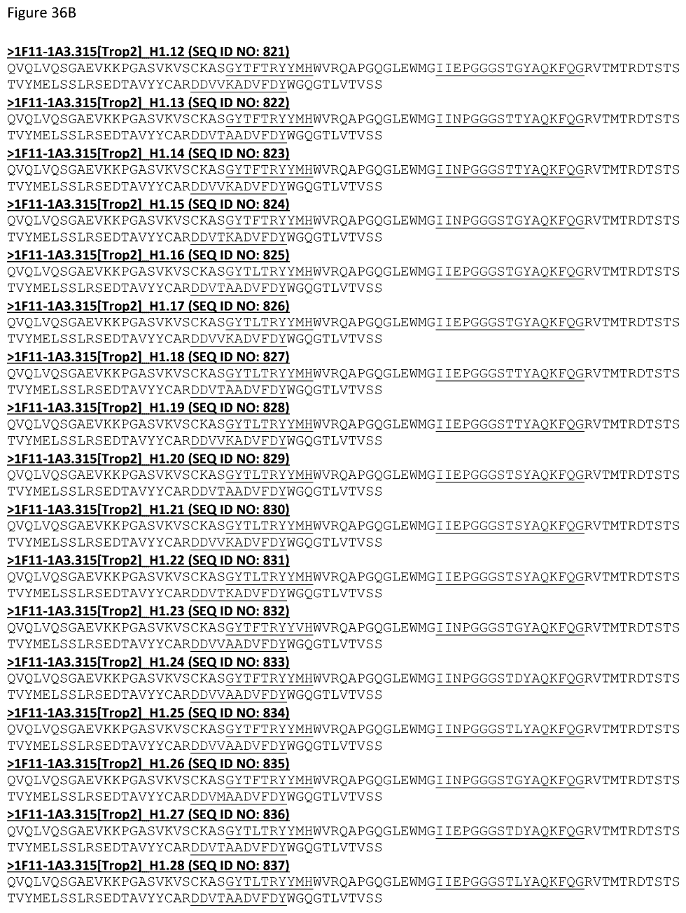

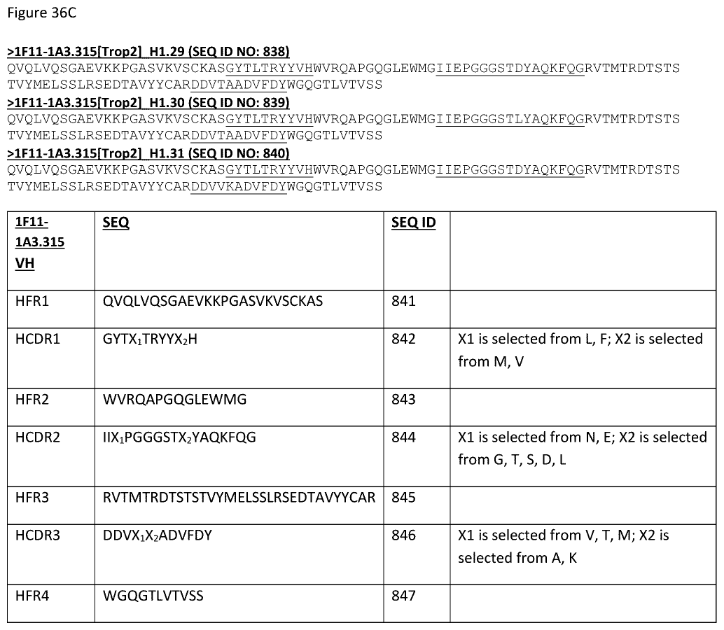

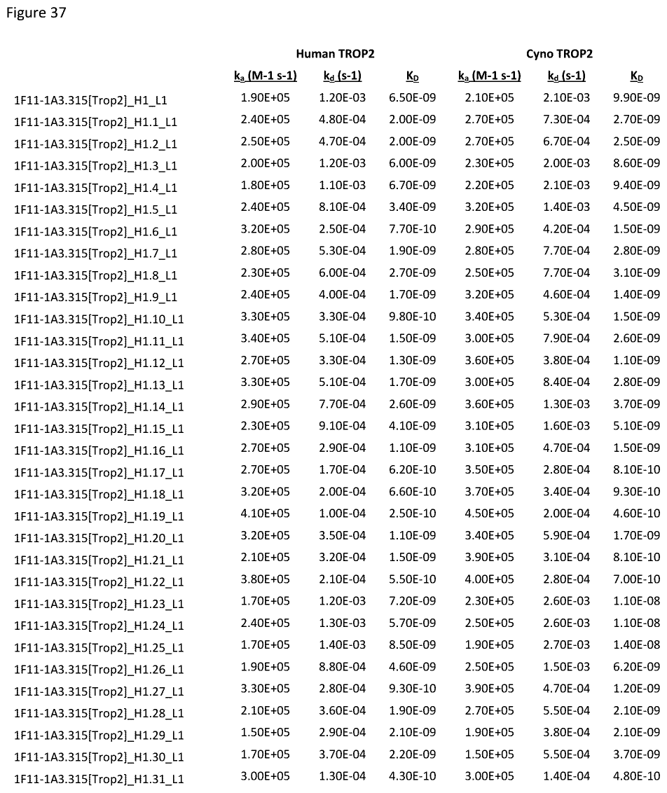

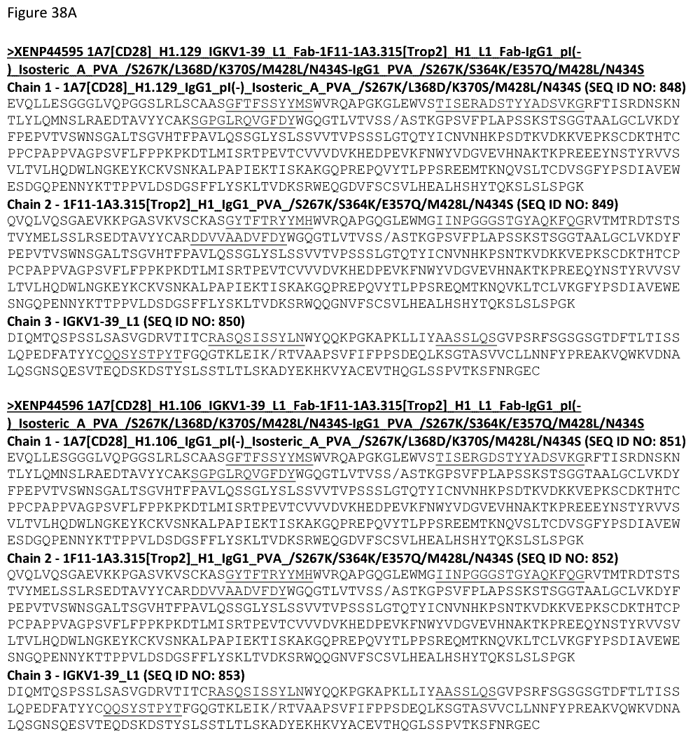

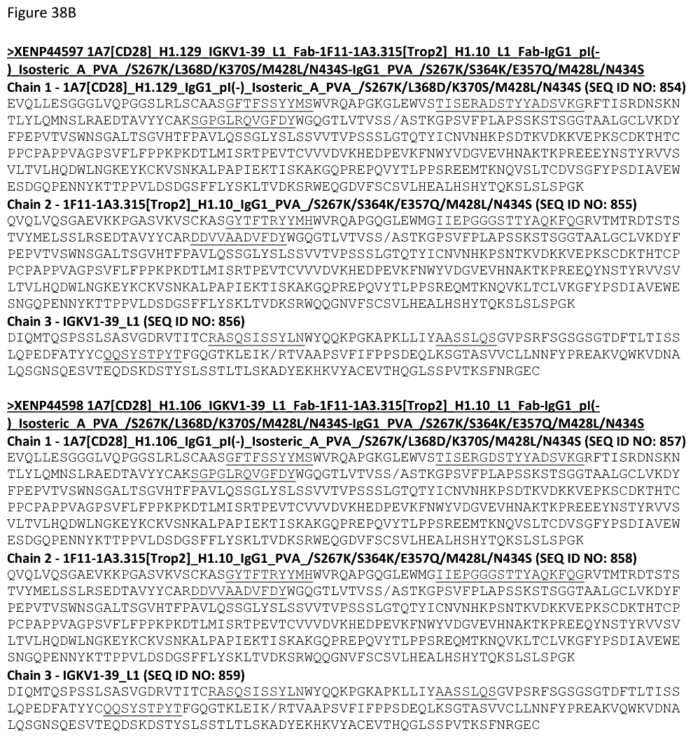

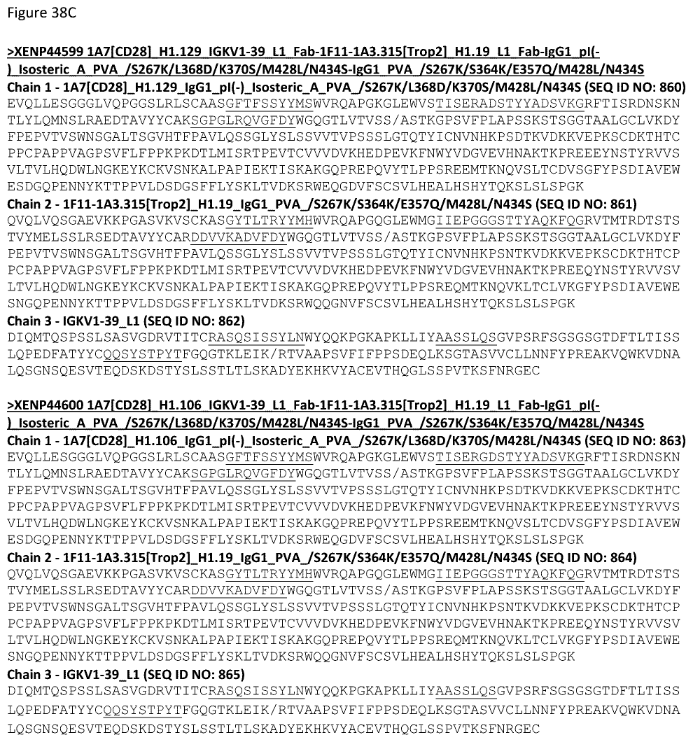

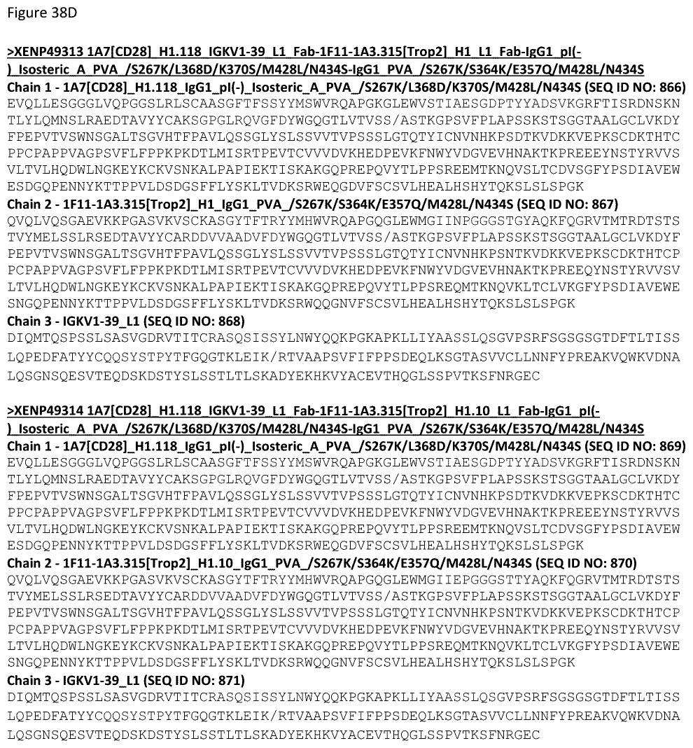

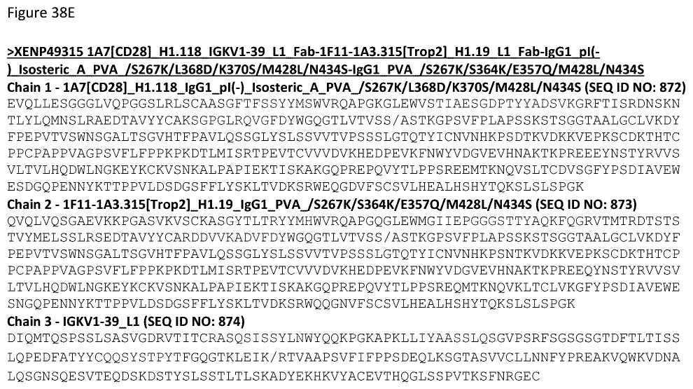

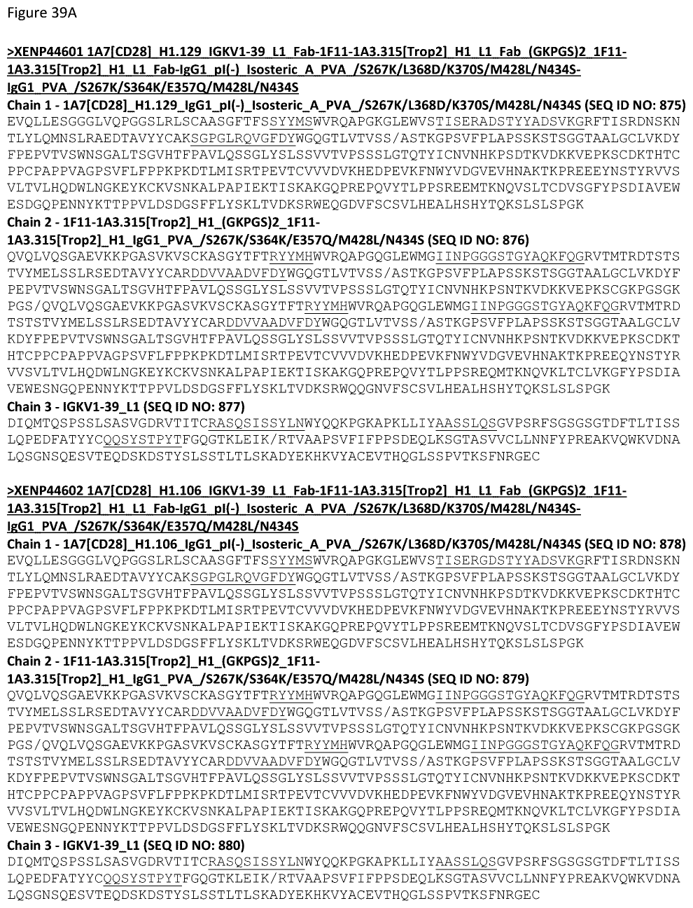

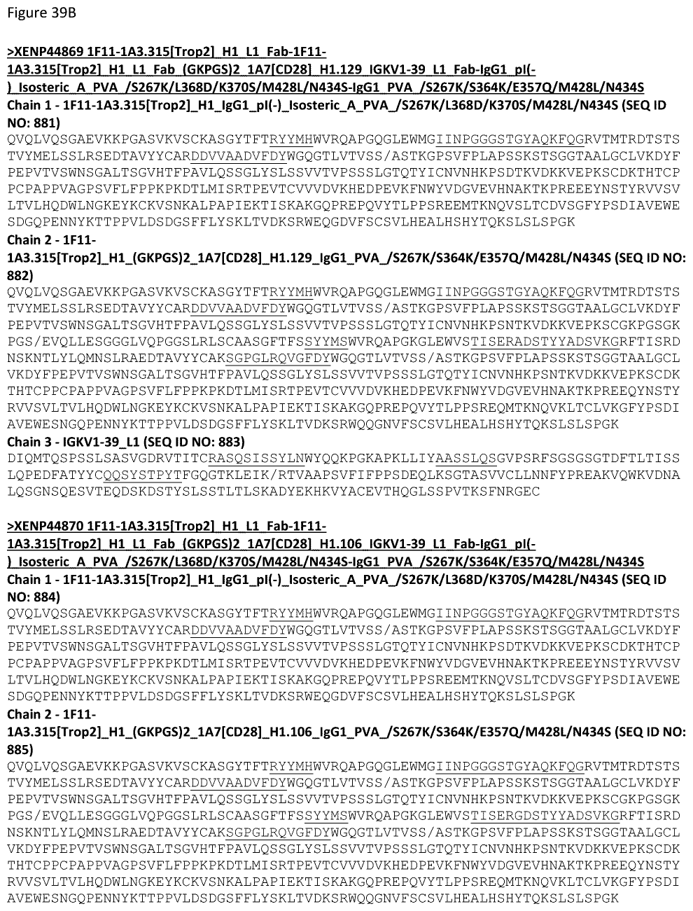

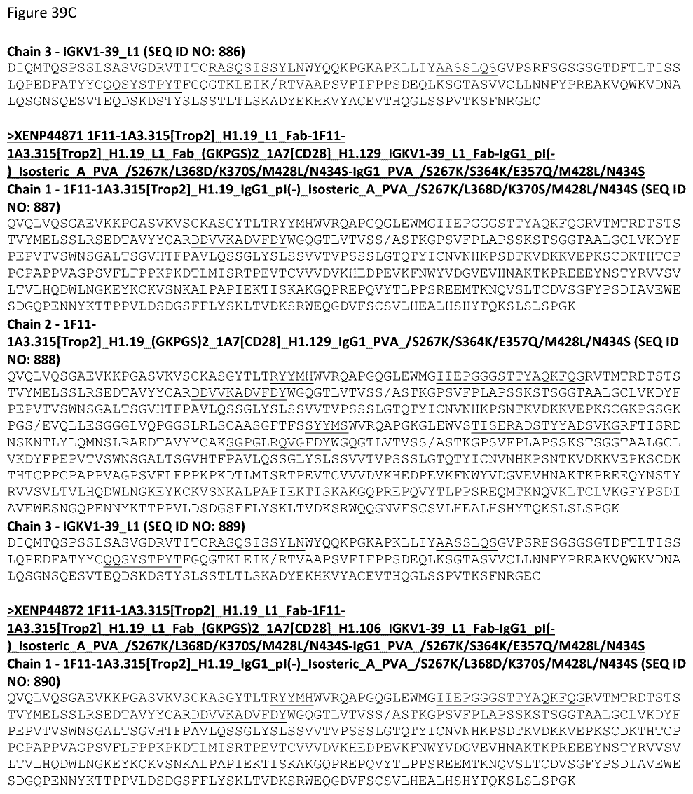

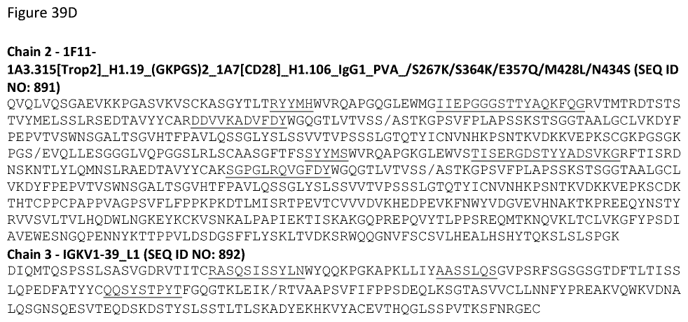

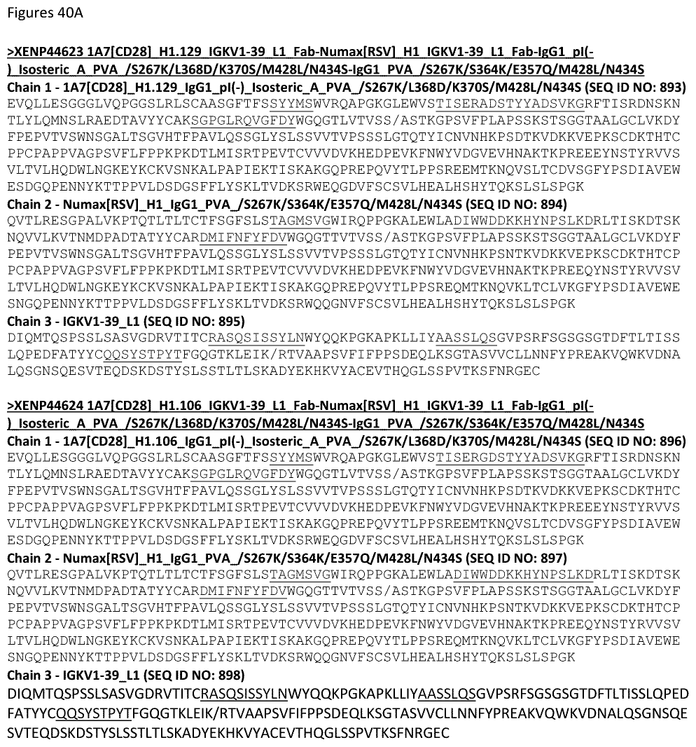

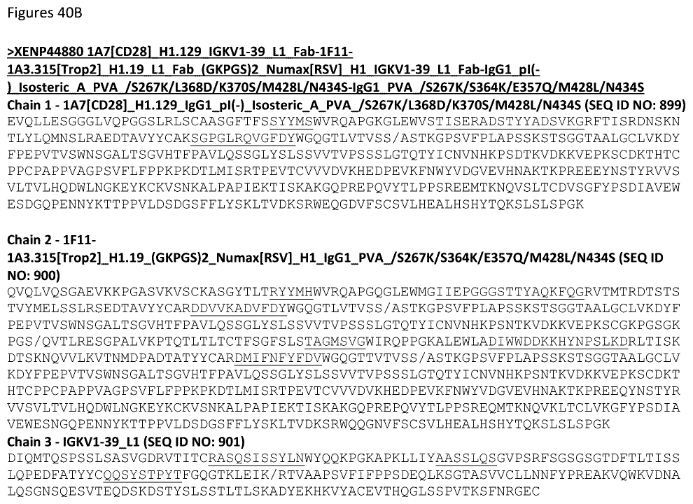

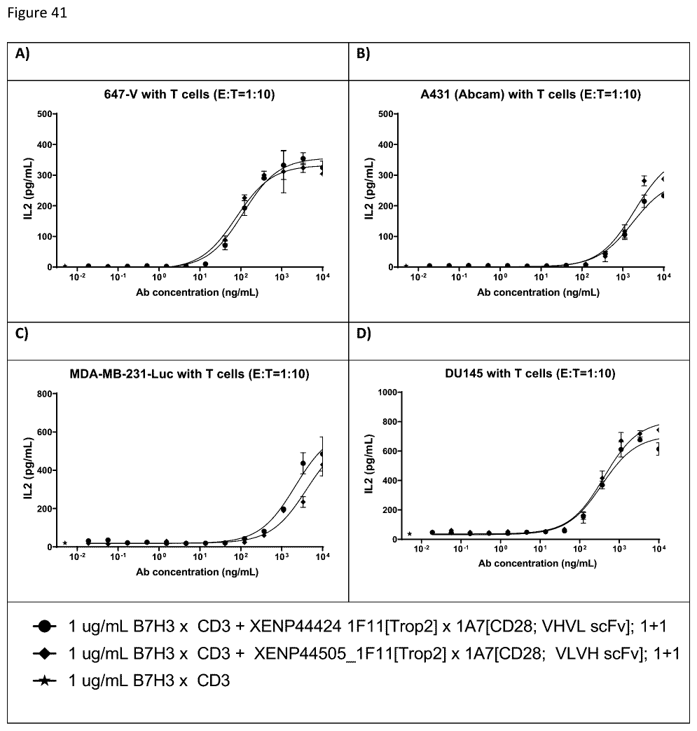

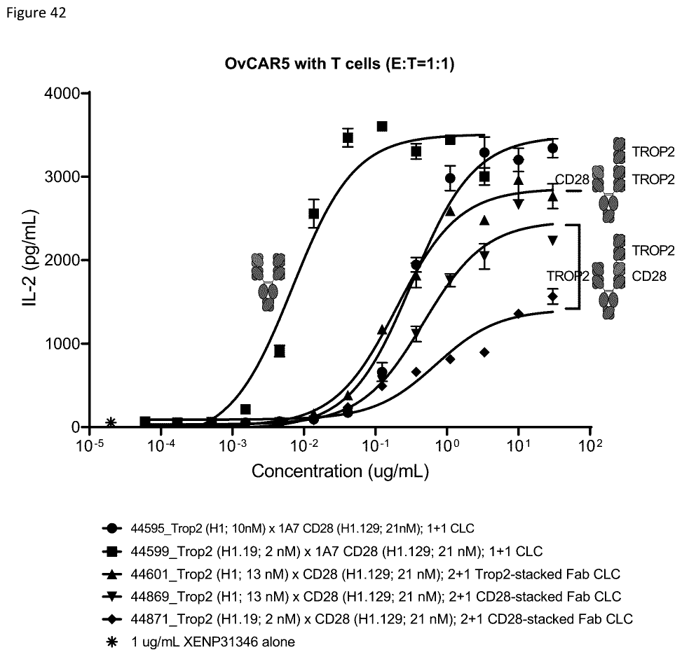

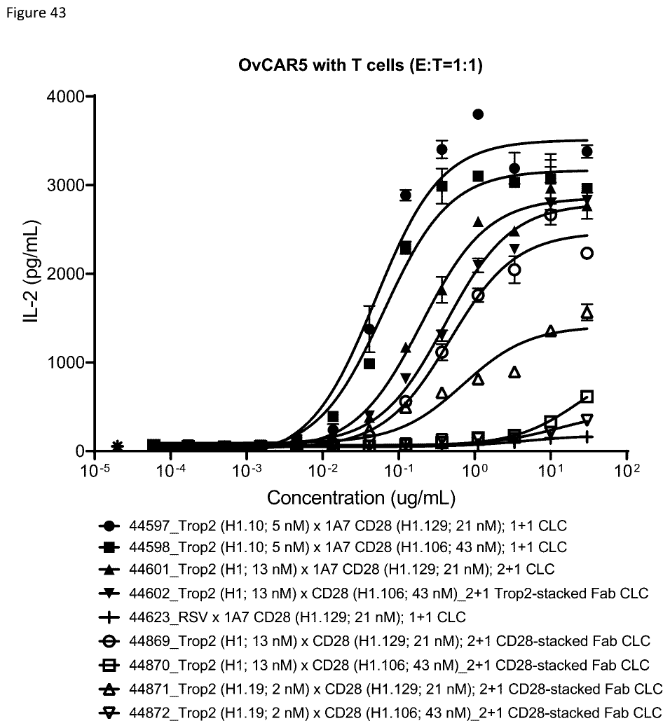

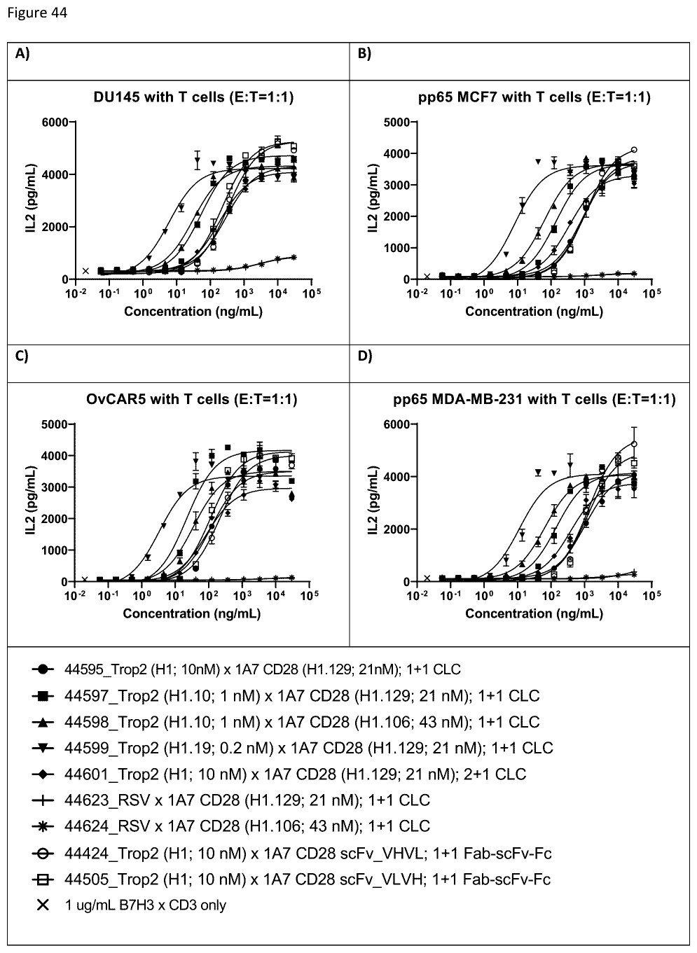

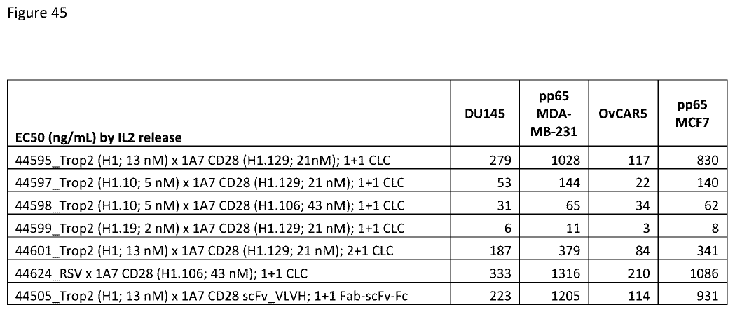

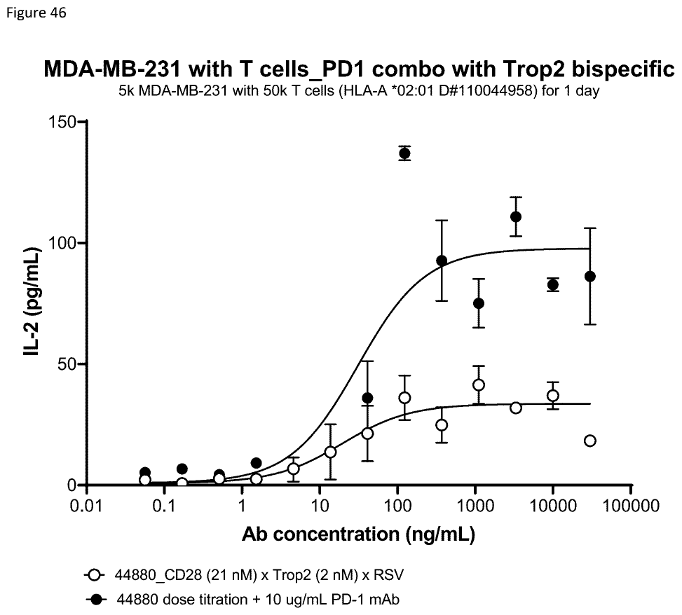

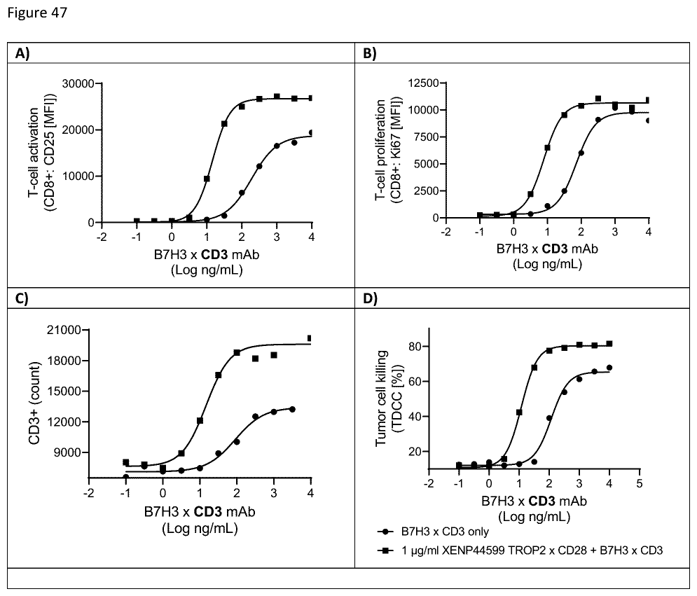

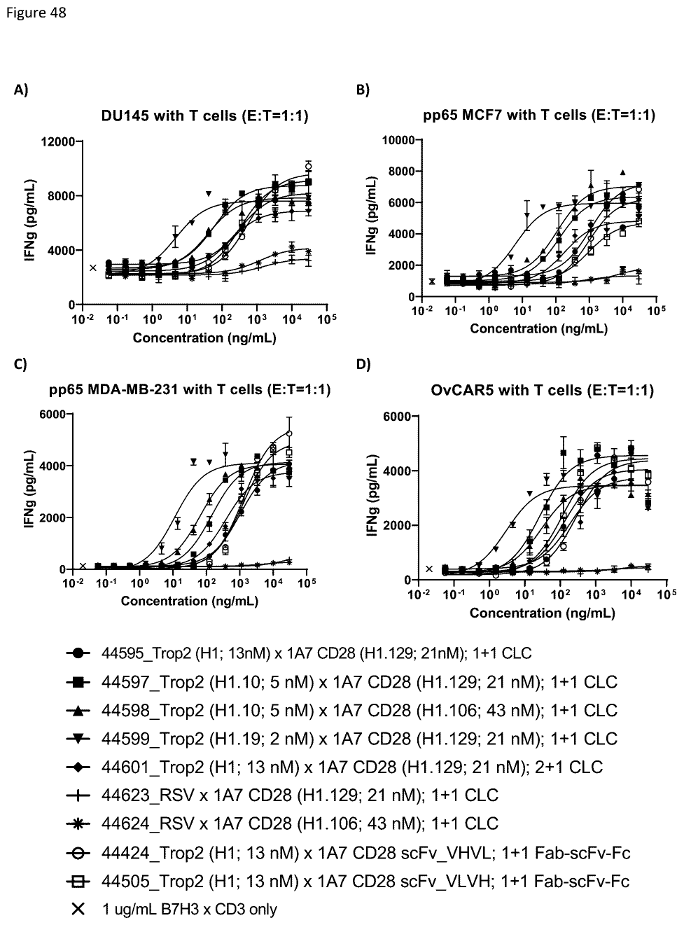

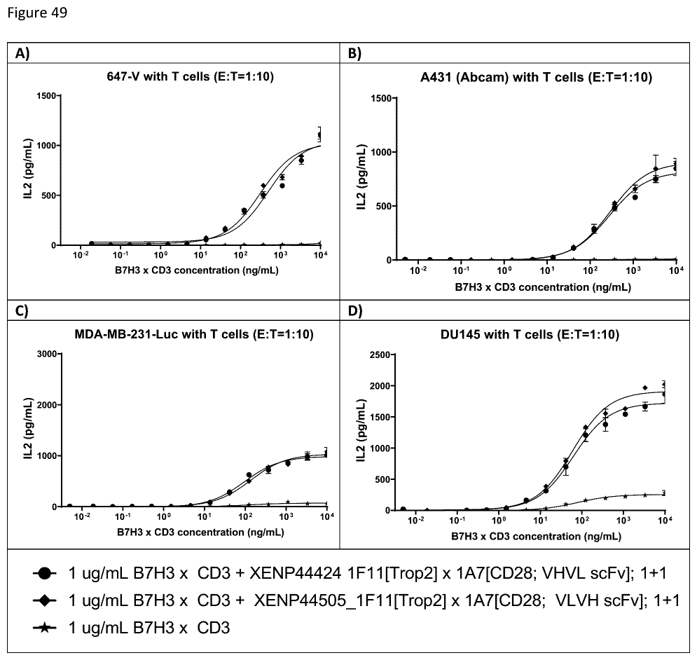

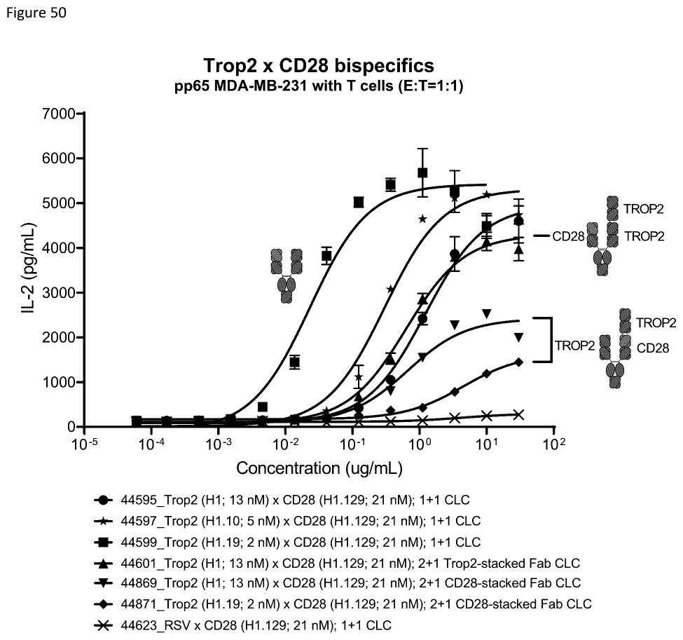

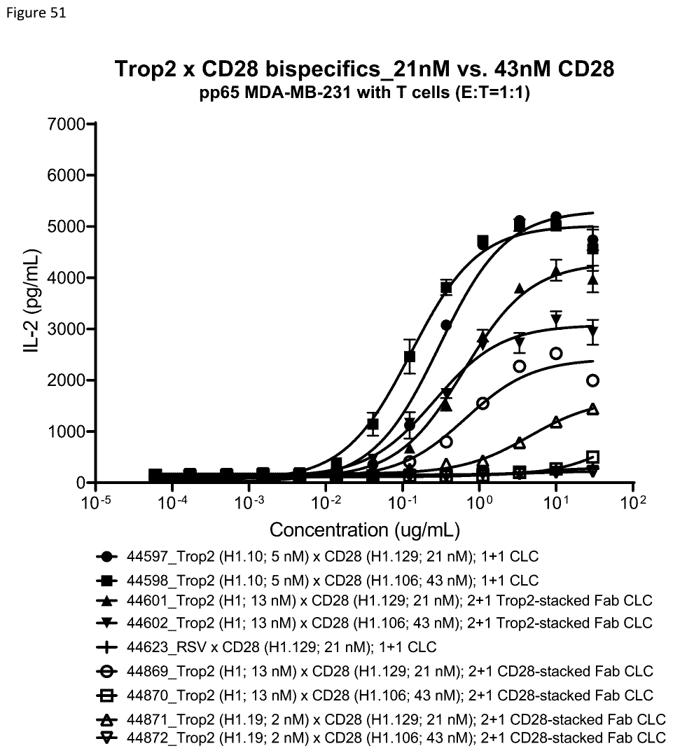

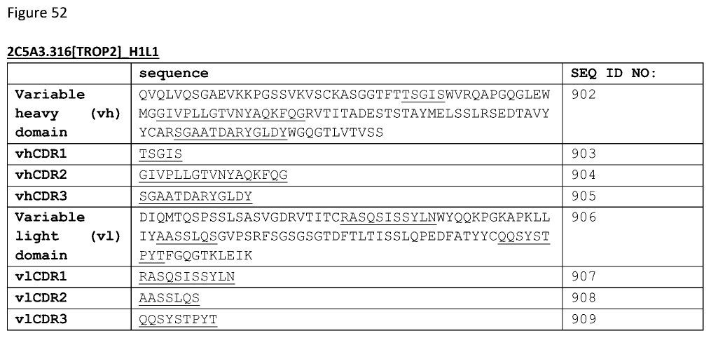

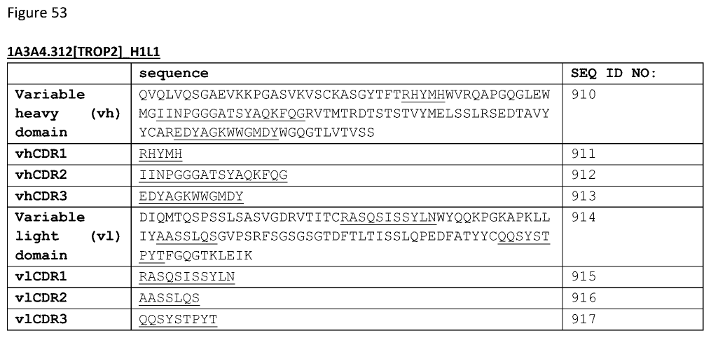

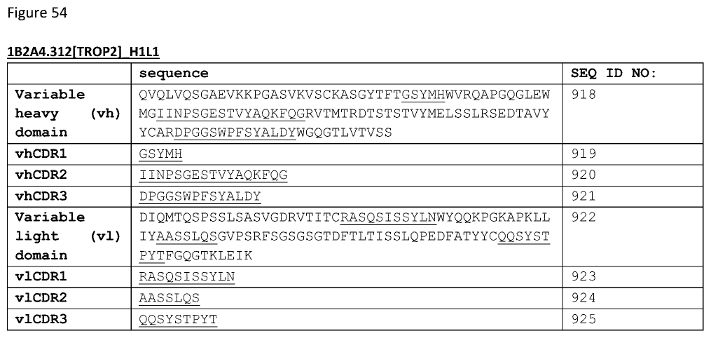

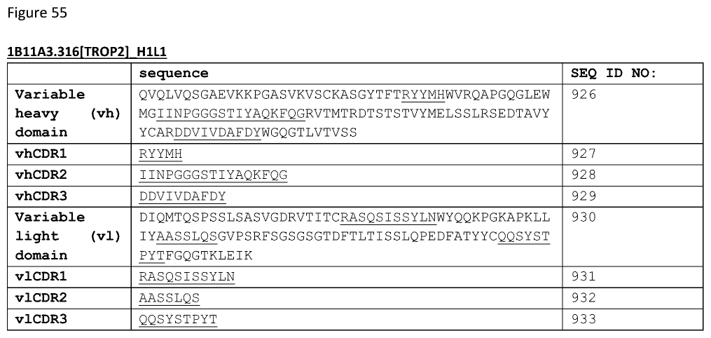

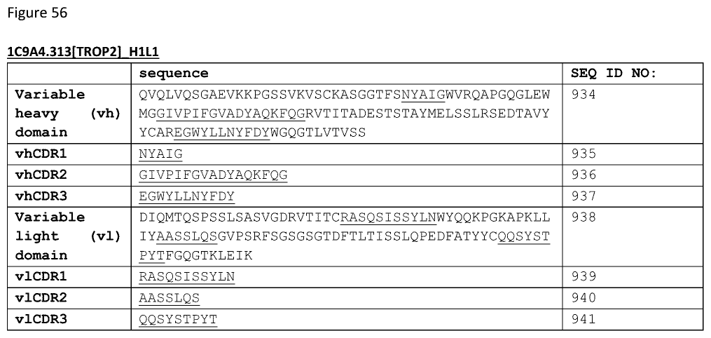

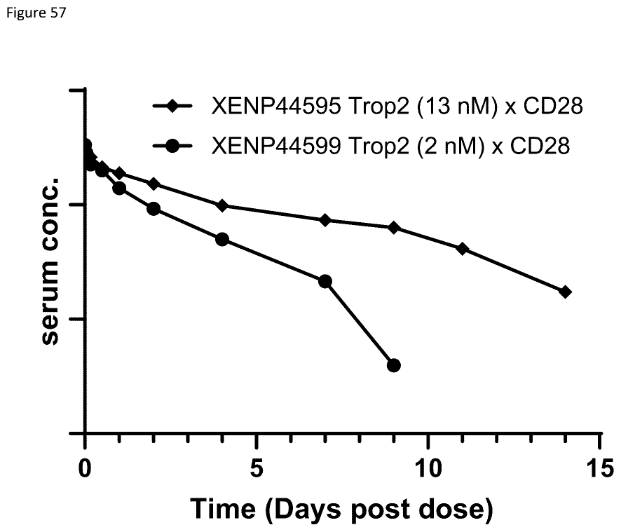

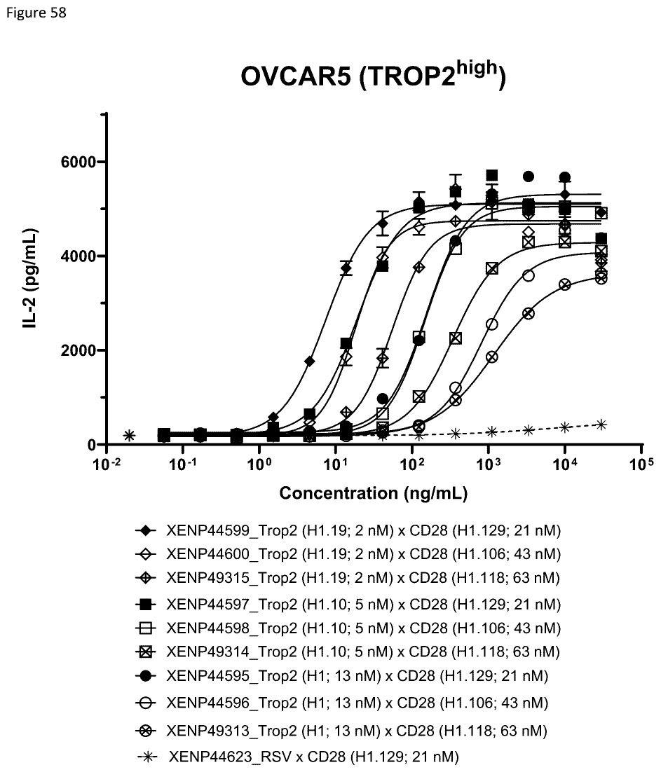

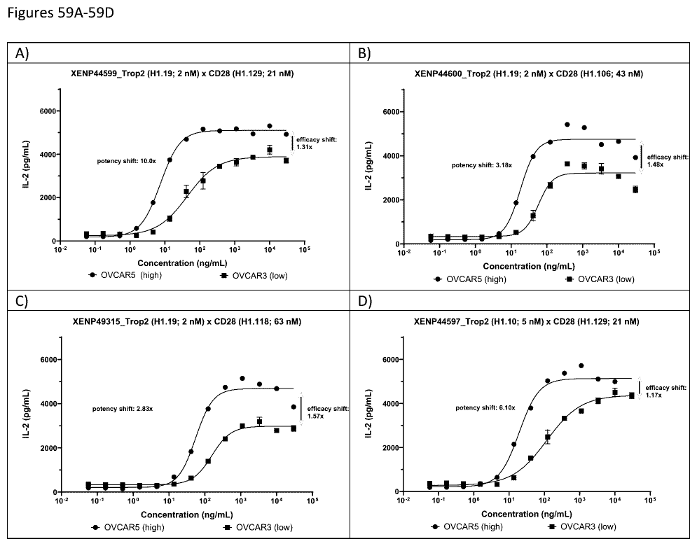

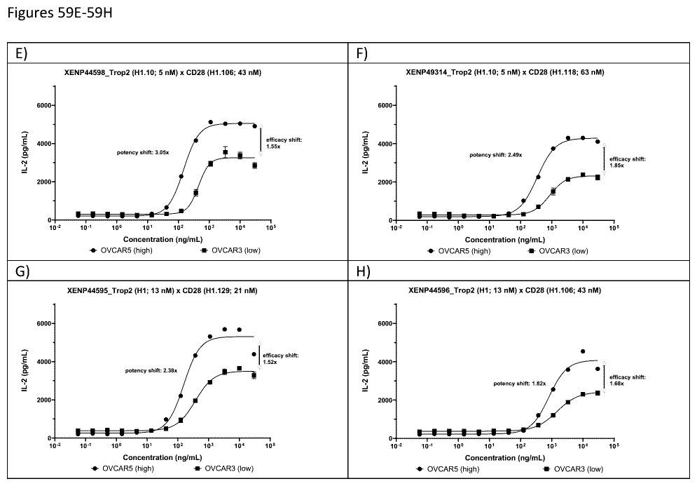

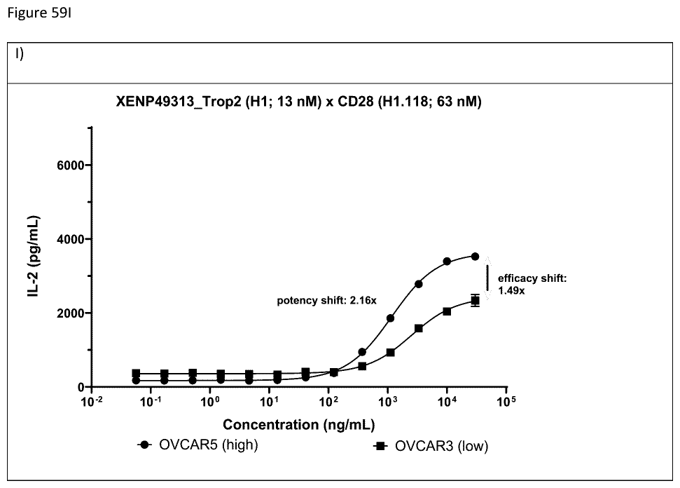

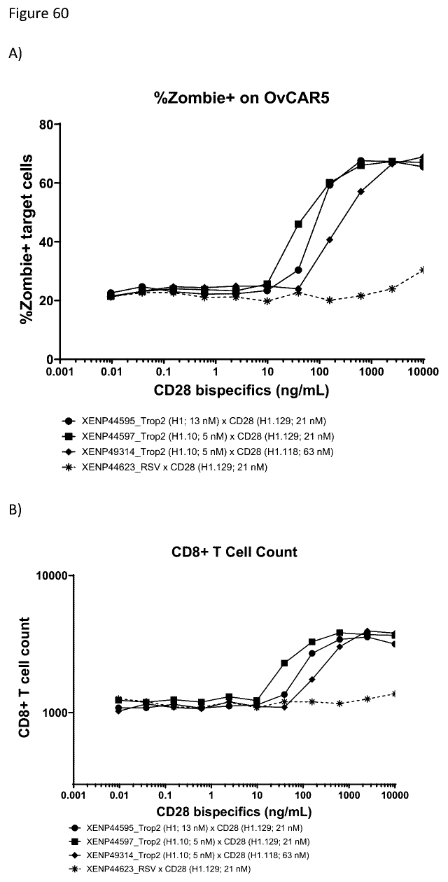

FIG. 11 depicts illustrative sequences of heterodimeric TROP2×CD28 bsAb backbone for use in the 2+1 mAb-scFv format. The format depicted here is based on heterodimeric Fc backbone 1 as depicted in FIG. 10, except further including G446_on monomer 1 (−) and G446_/K447_on monomer 2 (+). It should be noted that any of the additional backbones depicted in FIG. 10 may be adapted for use in the 2+1 mAb-scFv format with or without including K447_on one or both chains. It should be noted that these sequences may further include the M428L/N434S variants.

FIG. 12 depicts sequences for “CHi” that find use in embodiments of TROP2×CD28 bsAbs.

FIG. 13 depicts sequences for “hinge” that find use in embodiments of TROP2×CD28 bsAbs.

FIG. 14 depicts the constant domain of the cognate light chains that find use in the subject TROP2×CD28 bsAbs that utilize a Fab binding domain.

FIGS. 15A-15N depicts bispecific formats of the present invention. FIG. 15A depicts the “1+1 Fab-scFv-Fc” format, with a first Fab arm binding a first antigen and a second scFv arm binding second antigen. The 1+1 Fab-scFv-Fc format comprises a first monomer comprising a first heavy chain variable region (VH1) covalently attached to the N-terminus of a first heterodimeric Fc backbone (optionally via a linker), a second monomer comprising a single-chain Fv covalently attached to the N-terminus of a second corresponding heterodimeric Fc backbone (optionally via a linker), and a third monomer comprising a light chain variable region covalently to a light chain constant domain, wherein the light chain variable region is complementary to the VH1. FIG. 15B depicts the “2+1 Fab2-scFv-Fc” format, with a first Fab arm and a second Fab-scFv arm, wherein the Fab binds a first antigen and the scFv binds second antigen. The 2+1 Fab2-scFv-Fc format comprises a first monomer comprising a first heavy chain variable region (VH1) covalently attached to the N-terminus of a first heterodimeric Fc backbone (optionally via a linker), a second monomer comprising the VH1 covalently attached (optionally via a linker) to a single-chain Fv covalently attached (optionally via a linker) to the N-terminus of a second corresponding heterodimeric Fc backbone, and a third monomer comprising a light chain variable region covalently to a light chain constant domain, wherein the light chain variable region is complementary to the VH1. FIG. 15C depicts the “1+1 Common Light Chain” or “1+1 CLC” format, with a first Fc comprising a first Fab arm binding a first antigen and a second Fc comprising a second Fab arm binding second antigen. The 1+1 CLC format comprises a first monomer comprising VH1-CH1-hinge-CH2-CH3, a second monomer comprising VH2-CH1-hinge-CH2-CH3, and a third monomer comprising VL-CL. The VL pairs with the VH1 to form a binding domain with a first antigen binding specificity; and the VL pairs with the VH2 to form a binding domain with a second antigen binding specificity. FIG. 15D depicts the “2+1 Common Light Chain” or “2+1 CLC” format, with a first Fc comprising 2 Fab arms each binding a first antigen and a second Fc comprising 1 Fab arm binding a second antigen. The 2+1 CLC format comprises a first monomer comprising VH1-CH1-linker-VH1-CH1-hinge-CH2-CH3, a second monomer comprising VH2-CH1-hinge-CH2-CH3, and a third monomer comprising VL-CL. The VL pairs with the first and second VH1 to form binding domains with a first antigen binding specificity; and the VL pairs with the VH2 to form a binding domain with a second antigen binding specificity. FIG. 15E depicts the “2+1 mAb-scFv” format, with a first Fc comprising an N-terminal Fab arm binding a first antigen and a second Fc comprising an N-terminal Fab arm binding the first antigen and a C-terminal scFv binding a second antigen. The 2+1 mAb-scFv format comprises a first monomer comprising VH1-CH1-hinge-CH2-CH3, a second monomer comprising VH1-CH1-hinge-CH2-CH3-scFv, and a third monomer comprising VL-CL. The VL pairs with the first and second VH1 to form binding domains with binding specificity for the first antigen. FIG. 15N depicts the 2+1 Fab2-Fc×scFv-Fc format (also referred to as “stacked bottle opener”). This format includes a first monomer comprising VH1-CH1-domain linker-VH1-CH1-hinge-CH2-CH3, a second monomer comprising an scFv covalently attached to an Fc domain (CH2-CH3), and a third monomer comprising VL-CL. The VL pairs with the first and second VH1 of the first monomer to form binding domains with binding specificity for the first antigen, and the scFv has binding specificity for a second antigen. Additional bispecific formats include F) dual scFv, G) one-arm scFv-mAb, H) scFv-mAb, I) bispecific mAb, J) one-arm central-scFv, K) mAb-Fv, L) central-Fv, and M) trident.

FIG. 16 depicts the variable heavy and variable light chain sequences for 1A7, an exemplary phage-derived CD28 binding domain, as well as the sequences for XENP28428, an anti-CD28 mAb based on 1A7 and IgG1 backbone with E233P/L234V/L235A/G236del/S267K ablation variant. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 17A-17F depicts the sequences for affinity-optimized variable heavy domains from anti-CD28 clone 1A7. It should be noted that the variable heavy domains can be paired with any of the other variable light domains depicted in FIGS. 16 and 18.

FIGS. 18A-18I depicts the sequences for affinity-optimized variable light domain from anti-CD28 clone 1A7. It should be noted that the variable heavy domains can be paired with any of the other variable heavy domains depicted in FIGS. 16 and 17.

FIGS. 19A-19C depicts the sequence for illustrative affinity-optimized 1A7 VH/VL pairs. It should be noted that these pairs may be formatted as Fabs or as scFvs. Additionally, in the scFv format, these pairs may be formatted in the VHVL orientation or the VLVH orientation.

FIGS. 20A-20B depicts consensus framework regions (FR) and complementarity determining regions (CDRs) (as in Kabat) for anti-CD28 clone 1A7 variable heavy and variable light domain variants.

FIG. 21 depicts illustrative affinity-engineered 1A7 VH/VL pairs and their binding affinities in the context of scFvs (in the context of 1+1 Fab-scFv-Fc bsAb format).

FIGS. 22A-22H depicts the variable heavy and variable light chain sequences for additional CD28 binding domains which find use in the TROP2×CD28 bsAbs of the invention. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 23A-23M depicts the variable heavy and variable light chain sequences for exemplary TROP2 binding domains which find use in the TROP2×CD28 bsAbs of the invention. As noted herein and is true for every sequence herein containing CDRs, the identification of the CDR locations may be determined according to the numbering schemes as are shown in Table 2. Furthermore, as for all the sequences in the Figures, these VH and VL sequences can be used either in a scFv format or in a Fab format.

FIG. 24 depicts the variable heavy and variable light chain sequences for exemplary TROP2 binding domains which find use in the TROP2×CD28 bsAbs of the invention. Each of the variable heavy domains in this figure may be paired with any of the variable light domains in this figure. As noted herein and is true for every sequence herein containing CDRs, the identification of the CDR locations may be determined according to the numbering schemes as are shown in Table 2. Furthermore, as for all the sequences in the Figures, these VH and VL sequences can be used either in a scFv format or in a Fab format.

FIG. 25 depicts the variable heavy and variable light chain sequences for exemplary TROP2 binding domains which find use in the TROP2×CD28 bsAbs of the invention. Each of the variable heavy domains in this figure may be paired with any of the variable light domains in this figure. As noted herein and is true for every sequence herein containing CDRs, the identification of the CDR locations may be determined according to the numbering schemes as are shown in Table 2. Furthermore, as for all the sequences in the Figures, these VH and VL sequences can be used either in a scFv format or in a Fab format.

FIG. 26 depicts the variable heavy and variable light chain sequences for exemplary TROP2 binding domains which find use in the TROP2×CD28 bsAbs of the invention. Each of the variable heavy domains in this figure may be paired with any of the variable light domains in this figure. As noted herein and is true for every sequence herein containing CDRs, the identification of the CDR locations may be determined according to the numbering schemes as are shown in Table 2. Furthermore, as for all the sequences in the Figures, these VH and VL sequences can be used either in a scFv format or in a Fab format.

FIG. 27 depicts the variable heavy and variable light chain sequences for exemplary TROP2 binding domains which find use in the TROP2×CD28 bsAbs of the invention. Each of the variable heavy domains in this figure may be paired with any of the variable light domains in this figure. As noted herein and is true for every sequence herein containing CDRs, the identification of the CDR locations may be determined according to the numbering schemes as are shown in Table 2. Furthermore, as for all the sequences in the Figures, these VH and VL sequences can be used either in a scFv format or in a Fab format.

FIGS. 28A-28B depicts the variable heavy and variable light chain sequences for exemplary TROP2 binding domains which find use in the TROP2×CD28 bsAbs of the invention. Each of the variable heavy domains in this figure may be paired with any of the variable light domains in this figure. As noted herein and is true for every sequence herein containing CDRs, the identification of the CDR locations may be determined according to the numbering schemes as are shown in Table 2. Furthermore, as for all the sequences in the Figures, these VH and VL sequences can be used either in a scFv format or in a Fab format.

FIGS. 29A-29B depicts A) classic T cell/APC interaction and B) replication of the classic T cell/APC interaction by combining CD3 bispecific antibodies with CD28 bispecific antibodies. In classic T cell/APC interaction, there is a first signal provided by TCR reactivity with peptide-MHC (Signal 1) and a second signal provided by CD28 crosslinking by CD80/CD86 being expressed on APCs (Signal 2) which together fully activate T cells. In contrast in treatment with CD3 bispecifics, only the first signal is provided. The CD28 signal may be provided by a CD28 bispecific with the idea to promote activation and proliferation through CD28 costimulation. In some embodiments, TAA1 and TAA2 may be different antigens. In some embodiments, TAA1 and TAA2 may be same antigen but different epitopes. In some embodiments, TAA1 and TAA2 may be same antigen and same epitope.

FIGS. 30A-30B depict the sequences for illustrative TROP2×CD28 bsAbs in the 1+1 Fab-scFv-Fc format. Slashes indicate the border(s) of the variable domains and other domains e.g., domain linkers and constant domains. In addition, the naming convention illustrates the orientation of the scFv from N- to C-terminus. As noted herein and is true for every sequence herein containing CDRs, the identification of the CDR locations may be determined according to the numbering schemes as are shown in Table 2. It should be noted that the TROP2×CD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which including M428L/N434S results in longer half-life in serum.

FIG. 31 depict the sequences for illustrative TROP2×CD28 bsAbs in the 2+1 mAb-scFv format. Slashes indicate the border(s) of the variable domains and other domains e.g., domain linkers and constant domains. In addition, the naming convention illustrates the orientation of the scFv from N- to C-terminus. As noted herein and is true for every sequence herein containing CDRs, the identification of the CDR locations may be determined according to the numbering schemes as are shown in Table 2. It should be noted that the TROP2×CD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which including M428L/N434S results in longer half-life in serum.

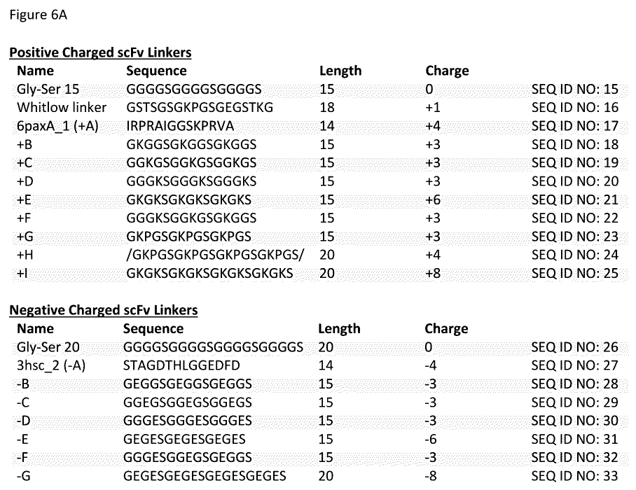

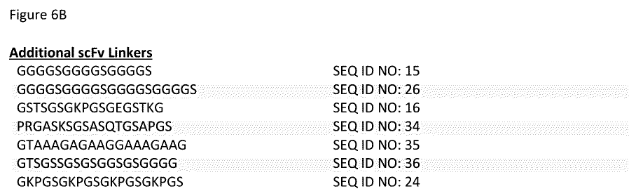

FIGS. 32A-32F depict sequences for exemplary anti-CD3 binding domains suitable for use in CD3 bispecific antibodies which may be combined with the CD28 bispecific antibodies of the invention. The CDRs are underlined, the scFv linker is double underlined (in the sequences, the scFv linker is a positively charged scFv (GKPGS)4 linker (SEQ ID NO: 24), although as will be appreciated by those in the art, this linker can be replaced by other linkers, including uncharged or negatively charged linkers, some of which are depicted in FIG. 6), and the slashes indicate the border(s) of the variable domains. In addition, the naming convention illustrates the orientation of the scFv from N- to C-terminus. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 33A-33B depicts induction of IL2 secretion from purified T cells incubated with αCD3 transfected A431-ß21-null cells at A) 10:1 or B) 1:1 effector:target ratio.

FIGS. 34A-34C depicts novel 1A7 VHs affinity-engineered to be paired with IGKV1-39 germline sequence, as well as consensus framework regions (FR) and complementarity determining regions (CDRs).

FIG. 35 depicts affinity of novel 1A7 variants paired with IGKV1-39 human germline VL for human CD28.

FIGS. 36A-36C depicts novel TROP2 binding domain clone 1F11-1A3.315, as well as illustrative affinity-engineered VH variants which may be paired with the L1 (it should be noted that the L1 is the IGKV1-39 germline sequence). Additionally, consensus framework regions (FR) and complementarity determining regions (CDRs) for 1F11-1A3.315 VH are depicted. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 37 depicts K_{D} binding constant, association constant (kₐ), and dissociation constant (k_{d}) for affinity-engineered anti-TROP2 clone 1F11-1A3.315 against human and cynomolgus TROP2.

FIGS. 38A-38E depicts the sequences for illustrative TROP2×CD28 bsAbs in the 1+1 CLC format. CDRs are underlined and slashes indicate the border(s) between the variable regions, linkers, Fc regions, and constant domains. It should be noted that the TROP2×CD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIGS. 39A-39D depicts the sequences for illustrative TROP2×CD28 bsAbs in the 2+1 CLC format. CDRs are underlined and slashes indicate the border(s) between the variable regions, linkers, Fc regions, and constant domains. It should be noted that the TROP2×CD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIGS. 40A-40B depicts the sequences for RSV controls (i.e. bsAbs swapping a TROP2 or CD28 binding domain with a “silent” RSV binding domain). These controls are used for a variety of experimental purposes including acting as surrogates for how the molecule would behave on TROP2 negative cells.

FIG. 41 depicts induction of IL-2 secretion by 1 μg/mL illustrative B7H3×CD3 bsAb in combination with TROP2×CD28 bsAbs having varying TROP2 and CD28 binding affinities in the presence of A) 647-V (TROP^{high}), B) A431 (TROP^{med}), C) MDA-MB-231 (TROP^{low-med}) and D) DU145 (TROP^{low-med}) tumor cells (1:10 effector:target).

FIG. 42 depicts induction of IL-2 secretion by 1 μg/mL illustrative B7H3×CD3 bsAb in combination with TROP2×CD28 bsAbs having varying TROP2 binding affinities and in various formats in the presence of OVCAR5 tumor cells (1:1 effector:target).

FIG. 43 depicts induction of IL-2 secretion by 1 μg/mL illustrative B7H3×CD3 bsAb in combination with TROP2×CD28 bsAbs having varying TROP2 and CD28 binding affinities and in various formats in the presence of OVCAR5 tumor cells (1:1 effector:target).

FIG. 44 depicts induction of IL-2 secretion by 1 μg/mL illustrative B7H3×CD3 bsAb in combination with TROP2×CD28 bsAbs having varying TROP2 and CD28 binding affinities in the presence of A) DU145, B) MCF7, C) OVCAR5, and D) MDA-MB-231 tumor cells (1:1 effector:target).

FIG. 45 depicts EC50 of induction of IL-2 secretion by 1 μg/mL illustrative B7H3×CD3 bsAb in combination with TROP2×CD28 bsAbs having varying TROP2 and CD28 binding affinities in the presence of DUl45, MDA-MB-231, OVCAR5, or MCF7 tumor cells (1:1 effector:target).

FIG. 46 depicts induction of IL-2 secretion by TROP2×CD28 bsAbs alone or in combination with 10 μg/ml PD-1 mAb in the presence of MDA-MB-231 tumor cells (1:10 effector:target). The data show that combining with PD-1 mAb enhances activity of TROP×CD28 bsAb.

FIG. 47 depicts induction of T cell activation (A), T cell proliferation (as indicated by ki67 (B) and T cell count (C)) and tumor cell kill (D) by 1 μg/ml TROP2×CD28 bsAb XENP44599 incubated with cocultures of T cells and OVCAR5 cells and dose titration of illustrative B7H3×CD3 bsAb.

FIG. 48 depicts induction of IFNγ secretion by 1 μg/mL illustrative B7H3×CD3 bsAb in combination with TROP2×CD28 bsAbs having varying TROP2 and CD28 binding affinities in the presence of A) DU145, B) MCF7, C) OVCAR5, and D) MDA-MB-231 tumor cells (1:1 effector:target).

FIG. 49 depicts induction of IL-2 secretion by dose titrating illustrative B7H3×CD3 bsAb in combination with 1 μg/ml TROP2×CD28 bsAbs having varying TROP2 and CD28 binding affinities in the presence of A) 647-V (TROP^{high}), B) A431 (TROP^{med}), C) MDA-MB-231 (TROP^{low-med}) and D) DU145 (TROP^{low-med}) tumor cells (1:10 effector:target).

FIG. 50 depicts induction of IL-2 secretion by 1 μg/mL illustrative B7H3×CD3 bsAb in combination with TROP2×CD28 bsAbs having varying TROP2 binding affinities and in various formats in the presence of pp65-MDA-MB-231 tumor cells (1:1 effector:target).

FIG. 51 depicts induction of IL-2 secretion by 1 μg/mL illustrative B7H3×CD3 bsAb in combination with TROP2×CD28 bsAbs having varying TROP2 and CD28 binding affinities and in various formats in the presence of pp65-MDA-MB-231 tumor cells (1:1 effector:target).

FIG. 52 depicts novel TROP2 binding domain clone 2C5A3.316 (it should be noted that the L1 is the IGKV1-39 germline sequence). As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 53 depicts novel TROP2 binding domain clone 1A3A4.312 (it should be noted that the L1 is the IGKV1-39 germline sequence). As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 54 depicts novel TROP2 binding domain clone 1B2A4.312 (it should be noted that the L1 is the IGKV1-39 germline sequence). As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 55 depicts novel TROP2 binding domain clone 1B11A3.316 (it should be noted that the L1 is the IGKV1-39 germline sequence). As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 56 depicts novel TROP2 binding domain clone 1C9A4.313 (it should be noted that the L1 is the IGKV1-39 germline sequence). As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 57 depicts serum concentration of XENP44599 and XENP44595 overtime in cynomolgus monkeys.

FIG. 58 depicts induction of IL-2 secretion by purified T cells incubated with 1 μg/ml illustrative EpCAM×CD3 bsAb in combination with TROP2×CD28 bsAbs having varying TROP2 and CD28 binding affinities in the presence of OVCAR5 TROP2high tumor cells (1:1 effector:target ratio).

FIGS. 59A-59I depicts induction of IL-2 secretion by purified T cells incubated with 1 μg/ml illustrative EpCAM×CD3 bsAb in combination with TROP2×CD28 bsAbs having varying TROP2 and CD28 binding affinities in the presence of OVCAR5 TROP2^{high} or OVCAR3 TROP2^{low} tumor cells (1:1 effector:target ratio) along with change in potency and efficacy on each cell line.

FIG. 60 depicts A) target cell kill and B) T cell proliferation upon incubating purified T cells with 2.5 ng/mL illustrative EpCAM×CD3 bsAb in combination with dose titration TROP2×CD28 bsAbs in the presence of OVCAR5 TROP2^{high} tumor cells (1:1 E:T ratio; 72 hours).

### DETAILED DESCRIPTION

### I. Overview

TROP2 is a type I transmembrane glycoprotein that has signal transduction function and plays a regulatory role in cell self-renewal, proliferation and transformation. TROP 2 is overexpressed in several cancers, including endometrial cancer, pancreatic cancer, colon cancer, esophageal cancer, cholangiocarcinoma, oral cancer, and glioma. Thus, TROP2 is a potential target for treating such cancers.

The activation of T cells in the treatment of cancer is being widely investigated. T cells require multiple signals for complete activation and differentiation. As shown in FIG. 29A, Signal 1, promoted by recognition of a peptide-MHC (pMHC) complex by the T cell receptor (TCR), is absolutely required for T cell activation. Signal 2, which synergizes with, and amplifies signal 1, is typically provided by the interaction of the CD28 ligands CD80 and CD86 with CD28 itself. Although CD28 engagement alone is typically inert, when combined with signal 1 activation, it promotes additional activation, survival, and proliferative signals, including IL-2 secretion. As CD80 and CD86 are only naturally expressed by professional antigen-presenting cells (APC), the extent of CD28 costimulation in the tumor setting can be highly variable. Accordingly, the present invention is directed to a novel class of tumor-targeted anti-CD28×anti-TROP2 bispecific antibodies, the CD80/CD86 engagement of CD28 that mimic the CD80/CD86 engagement of CD28, thereby providing an artificial source of signal 2 (FIG. 29B). Notably, signal 1 can either be provided by the natural TCR:pMHC recognition of tumor cells, or it can be provided by combination of the CD28 bispecific with a CD3 bispecific (e.g., anti-CD3×anti-TROP2), which can mimic signal 1.

Accordingly, provided herein are novel anti-CD28×anti-TROP2 (also referred to as “αCD28×αTROP2” and sometimes “CD28×TROP2”) bispecific antibodies and methods of using such antibodies for the treatment of TROP2-associated cancers. In many cases, these bispecific antibodies are heterodimeric. Subject αCD28×αTROP2 antibodies are capable of agonistically binding to CD28 costimulatory molecules on T cells and targeting to TROP2 on TROP2-expressing tumor cells. Thus, such antibodies selectively enhance anti-tumor activity at TROP2-expressing tumor sites while minimizing peripheral toxicity. The subject antibodies provided herein are particularly useful for enhancing anti-tumor activity either alone, as a monotherapy, or when used in combination with other anti-cancer therapies as more fully described herein.

Accordingly, in one aspect, provided herein are heterodimeric antibodies that bind to two different antigens, e.g., the antibodies are “bispecific,” in that they bind two different target antigens, generally CD28 and TROP2 as described below. These heterodimeric antibodies can bind each of the target antigens either monovalently (e.g., there is a single antigen binding domain such as a variable heavy and variable light domain pair) or bivalently (there are two antigen binding domains that each independently bind the antigen). In some embodiments, the heterodimeric antibody provided herein includes one CD28 binding domain and one TROP2 binding domain (e.g., heterodimeric antibodies in the “1+1 Fab-scFv-Fc” format described herein, which are thus bispecific and bivalent). In other embodiments, the heterodimeric antibody provided herein includes one CD28 binding domain and two TROP2 binding domains (e.g., heterodimeric antibodies in the “2+1 Fab2-scFv-Fc” formats described herein, which are thus bispecific but trivalent, as they contain three antigen binding domains (ABDs)). The heterodimeric antibodies provided herein are based on the use of different monomers that contain amino acid substitutions (i.e., skew variants”) that “skew” formation of heterodimers over homodimers, as is more fully outlined below. In some embodiments, the heterodimer antibodies are also coupled with purification variants (e.g., “pI variants”) that allow simple purification of the heterodimers away from the homodimers, as is similarly outlined below. The heterodimeric bispecific antibodies provided generally rely on the use of engineered or variant Fc domains that can self-assemble in production cells to produce heterodimeric proteins, and methods to generate and purify such heterodimeric proteins.

### II. Nomenclature

The naming nomenclature of particular antigen binding domains (e.g., TROP2 and CD28 binding domains) use a “Hx.xx_Ly.yy” type of format, with the numbers being unique identifiers to particular variable chain sequences. Thus, for example, the CD28 binding domain “1A7[CD28]_H1_L1” (FIG. 16) includes a variable heavy domain, H1, and a variable light domain L1. In the case that these sequences are used as scFvs, the designation “H1_L1”, indicates that the binding domain includes a variable heavy domain “Hi” combined with a variable light domain “L1,” and is in VH-linker-VL orientation, from N- to C-terminus. This molecule with the identical sequences of the heavy and light variable domains but in the reverse order (VL-linker-VH orientation, from N- to C-terminus) would be designated “L1_H1”. Similarly, different constructs may “mix and match” the heavy and light chains as will be evident from the sequence listing and the Figures.

### III. Definitions

In order that the application may be more completely understood, several definitions are set forth below. Such definitions are meant to encompass grammatical equivalents.

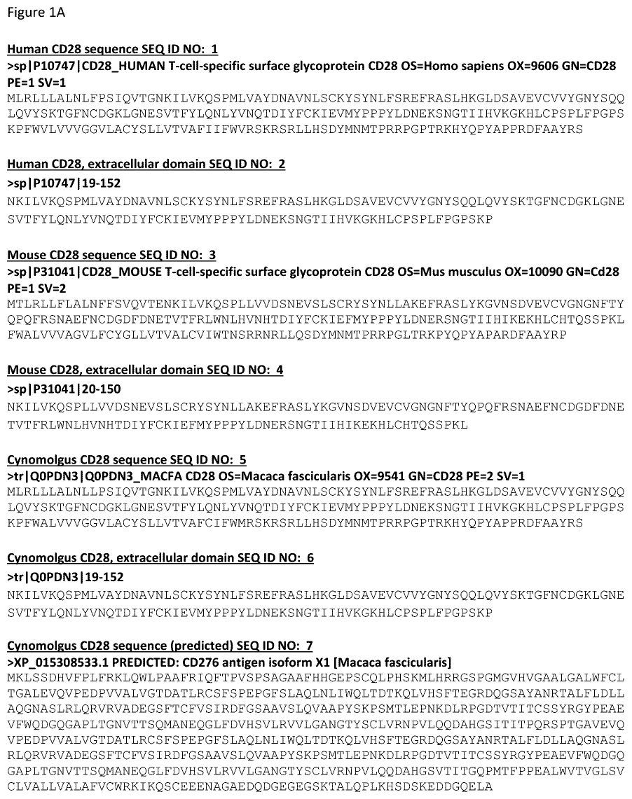

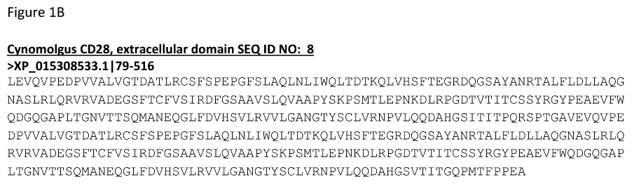

By “CD28,” “Cluster of Differentiation 28,” and “Tp44” (e.g., Genebank Accession Numbers NP_001230006 (human), NP_001230007 (human), NP_006130 (human), and NP_031668 (mouse)) herein is meant a B7 receptor expressed on T cells that provides co-stimulatory signals required for T cell activation and survival. T cell stimulation through CD28 in addition to the T cell receptor (TCR) provides a potent signal for the production of various interleukins. CD28 is the receptor for CD80 (B7.1) and CD86 (B7.2) proteins. CD28 includes an intercellular domain with a YMNM motif (SEQ ID NO:749) critical for the recruitment of SH2-domain containing proteins, particularly PI3K. CD28 also includes two proline-rich motifs that are able to bind SH3-containing proteins. Exemplary CD28 sequences are depicted in FIG. 1. Unless otherwise noted, references to CD28 are to the human CD28 sequence.

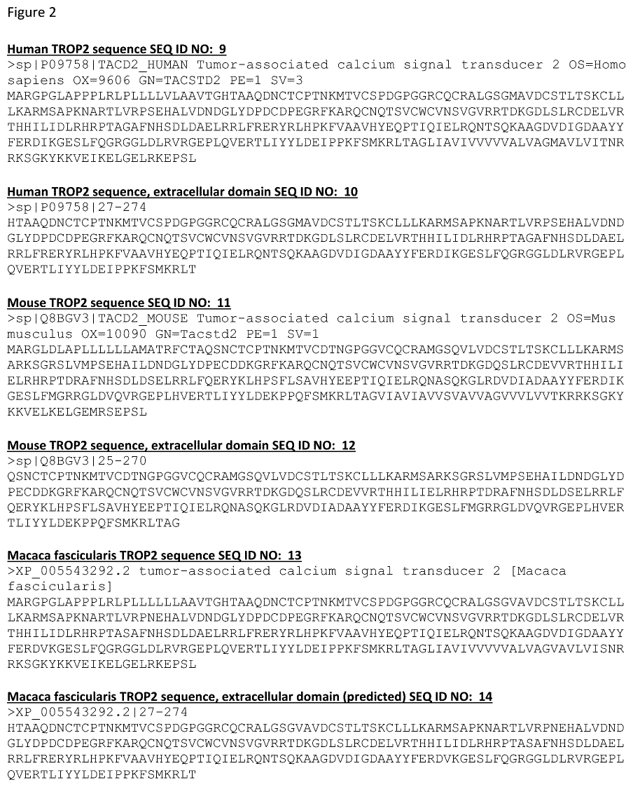

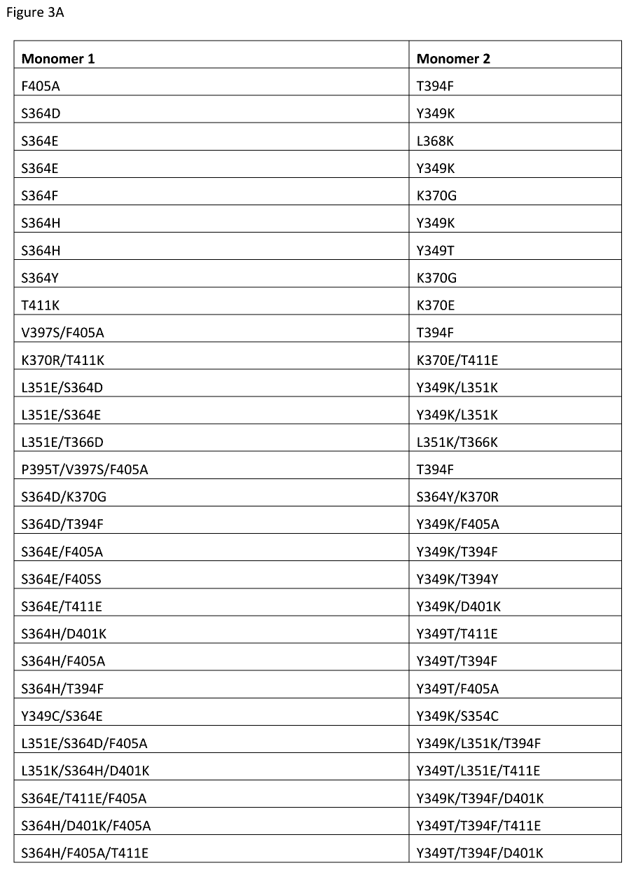

By “TROP2,” “tumor-associated calcium signal transducer 2,” “TACSTD2,” “epidermal glycoprotein 1,” and “EGP-1” herein is meant a transmembrane glycoprotein that is part of the tumor-associated calcium signal transducer (TACSTD) family. TROP2 that has signal transduction function and plays a regulatory role in cell self-renewal, proliferation and transformation. TROP2 sequences are depicted, for example, in FIG. 2. TROP2 is expressed in particular cancers, including endometrial cancer, pancreatic cancer, colon cancer, esophageal cancer, cholangiocarcinoma, oral cancer, and glioma.

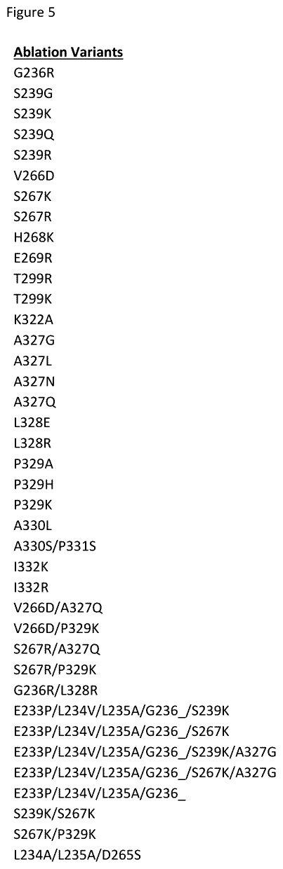

By “ablation” herein is meant a decrease or removal of activity. Thus, for example, “ablating FcγR binding” means the Fc region amino acid variant has less than 50% starting binding as compared to an Fc region not containing the specific variant, with more than 70-80-90-95-98% loss of activity being preferred, and in general, with the activity being below the level of detectable binding in a Biacore, SPR or BLI assay. Of particular use in the ablation of FcγR binding are those shown in FIG. 5, which generally are added to both monomers.

By “ADCC” or “antibody dependent cell-mediated cytotoxicity” as used herein is meant the cell-mediated reaction, wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

By “ADCP” or antibody dependent cell-mediated phagocytosis as used herein is meant the cell-mediated reaction wherein nonspecific phagocytic cells that express FcγRs recognize bound antibody on a target cell and subsequently cause phagocytosis of the target cell.

As used herein, the term “antibody” is used generally. Antibodies provided herein can take on a number of formats as described herein, including traditional antibodies as well as antibody derivatives, fragments and mimetics, described herein.

Traditional immunoglobulin (Ig) antibodies are “Y” shaped tetramers. Each tetramer is typically composed of two identical pairs of polypeptide chains, each pair having one “light chain” monomer (typically having a molecular weight of about 25 kDa) and one “heavy chain” monomer (typically having a molecular weight of about 50-70 kDa).

Other useful antibody formats include, but are not limited to, the “1+1 Fab-scFv-Fc,” and “2+1 Fab2-scFv-Fc” formats provided herein (see, e.g., FIG. 15). Additional useful antibody formats include, but are not limited to, “1+1 common light chain,” and “2+1 common light chain,” “mAb-Fv,” “mAb-scFv,” “central-Fv”, “one-armed scFv-mAb,” “scFv-mAb,” “dual scFv,” and “trident” format antibodies (FIG. 15). See also, US20180127501A1, which is incorporated by reference herein, particularly in pertinent part relating to antibody formats (see, e.g., FIG. 2 of US20180127501A1).

Antibody heavy chains typically include a variable heavy (VH) domain, which includes vhCDR1-3, and an Fc domain, which includes a CH2-CH3 monomer. In some embodiments, antibody heavy chains include a hinge and CHi domain. Traditional antibody heavy chains are monomers that are organized, from N- to C-terminus: VH-CH1-hinge-CH2-CH3. The CH1-hinge-CH2-CH3 is collectively referred to as the heavy chain “constant domain” or “constant region” of the antibody, of which there are five different categories or “isotypes”: IgA, IgD, IgG, IgE and IgM.

In some embodiments, the antibodies provided herein include IgG isotype constant domains, which has several subclasses, including, but not limited to IgG1, IgG2, IgG3, and IgG4. In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. As shown in Table 1, the exact numbering and placement of the heavy chain domains can be different among different numbering systems. As shown herein and described below, the pI variants can be in one or more of the CH regions, as well as the hinge region, discussed below.

It should be noted that IgG1 has different allotypes with polymorphisms at 356 (D or E) and 358 (L or M). The sequences depicted herein use the 356E/358M allotype, however the other allotype is included herein. That is, any sequence inclusive of an IgG1 Fc domain included herein can have 356D/358L replacing the 356E/358M allotype. It should be understood that therapeutic antibodies can also comprise hybrids of isotypes and/or subclasses. For example, as shown in US Publication 2009/0163699, incorporated by reference, the present antibodies, in some embodiments, include human IgG1/G2 hybrids.

By “Fc” or “Fc region” or “Fc domain” as used herein is meant the polypeptide comprising the constant region of an antibody, in some instances, excluding all of the first constant region immunoglobulin domain (e.g., CHi) or a portion thereof, and in some cases, optionally including all or part of the hinge. For IgG, the Fc domain comprises immunoglobulin domains CH2 and CH3 (Cy2 and Cy3), and optionally all or a portion of the hinge region between CHi (Cyl) and CH2 (Cy2). Thus, in some cases, the Fc domain includes, from N- to C-terminal, CH2-CH3 and hinge-CH2-CH3. In some embodiments, the Fc domain is that from IgG1, IgG2, IgG3 or IgG4, with IgG1 hinge-CH2-CH3 and IgG4 hinge-CH2-CH3 finding particular use in many embodiments. Additionally, in the case of human IgG1 Fc domains, the hinge may include a C220S amino acid substitution. Furthermore, in the case of human IgG4 Fc domains, the hinge may include a S228P amino acid substitution. Although the boundaries of the Fc region may vary, the human IgG heavy chain Fc region is usually defined to include residues E216, C226, or A231 to its carboxyl-terminal, wherein the numbering is according to the EU index as in Kabat. In some embodiments, as is more fully described below, amino acid modifications are made to the Fc region, for example to alter binding to one or more FcγR or to the FcRn.

By “heavy chain constant region” herein is meant the CH1-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

Another type of domain of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (P230 in IgG1), wherein the numbering is according to the EU index as in Kabat. In some cases, a “hinge fragment” is used, which contains fewer amino acids at either or both of the N- and C-termini of the hinge domain. As noted herein, pI variants can be made in the hinge region as well. Many of the antibodies herein have at least one the cysteines at position 220 according to EU numbering (hinge region) replaced by a serine. Generally, this modification is on the “scFv monomer” side (when 1+1 or 2+1 formats are used) for most of the sequences depicted herein, although it can also be on the “Fab monomer” side, or both, to reduce disulfide formation. Specifically included within the sequences herein are one or both of these cysteines replaced (C220S).

As will be appreciated by those in the art, the exact numbering and placement of the heavy chain constant region domains (i.e., CHi, hinge, CH2 and CH3 domains) can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The antibody light chain generally comprises two domains: the variable light domain (VL), which includes light chain CDRs vlCDR1-3, and a constant light chain region (often referred to as CL or CK). The antibody light chain is typically organized from N- to C-terminus: VL-CL.

By “antigen binding domain” or “ABD” herein is meant a set of six Complementary Determining Regions (CDRs) that, when present as part of a polypeptide sequence, specifically binds a target antigen (e.g., TROP2 or CD28) as discussed herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 variable heavy CDRs and vlCDR1, vlCDR2 and vlCDR3 vhCDR3 variable light CDRs. The CDRs are present in the variable heavy domain (vhCDR1-3) and variable light domain (vlCDR1-3). The variable heavy domain and variable light domain from an Fv region.

The present invention provides a large number of different CDR sets. In this case, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3). A useful comparison of CDR numbering is as below, see Lafranc et al., Dev. Comp. Immunol. 27(1):55-77 (2003):

TABLE 2

| | Kabat + | | | | | | |
|---|---|---|---|---|---|---|---|
| | Chothia | IMGT | Kabat | AbM | Chothia | Contact | Xencor |
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the van able domain (approximately, residues 1-107 of the light chain variable region and residues 1-113 of the heavy chain variable region) and the EU numbering system for Fc regions (e.g., Kabat et al., supra (1991)).

The CDRs contribute to the formation of the antigen-binding, or more specifically, epitope binding site of the antigen binding domains and antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

In some embodiments, the six CDRs of the antigen binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CHi domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used. In general, the C-terminus of the scFv domain is attached to the N-terminus of all or part of the hinge in the second monomer.

By “variable region” or “variable domain” as used herein is meant the region of an immunoglobulin that comprises one or more Ig domains substantially encoded by any of the VK, VW, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

By “Fab” or “Fab region” as used herein is meant the antibody region that comprises the VH, CH1, VL, and CL immunoglobulin domains, generally on two different polypeptide chains (e.g., VH-CH1 on one chain and VL-CL on the other). Fab may refer to this region in isolation, or this region in the context of a bispecific antibody of the invention. In the context of a Fab, the Fab comprises an Fv region in addition to the CHi and CL domains.

By “Fv” or “Fv fragment” or “Fv region” as used herein is meant the antibody region that comprises the VL and VH domains. Fv regions can be formatted as both Fabs (as discussed above, generally two different polypeptides that also include the constant regions as outlined above) and single chain Fvs (scFvs), where the vl and vh domains are included in a single peptide, attached generally with a linker as discussed herein.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g., H.X_L.Y means N- to C-terminal is vh-linker-vl, and L.Y_H.X is vl-linker-vh.

Some embodiments of the subject antibodies provided herein comprise at least one scFv domain, which, while not naturally occurring, generally includes a variable heavy domain and a variable light domain, linked together by a scFv linker. As outlined herein, while the scFv domain is generally from N- to C-terminus oriented as VH-scFv linker-VL, this can be reversed for any of the scFv domains (or those constructed using vh and vl sequences from Fabs), to VL-scFv linker-VH, with optional linkers at one or both ends depending on the format.

By “modification” or “variant” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution;” that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233 #, E233( ) or E233del designates a deletion of glutamic acid at position 233. Additionally, EDA233- or EDA233 #designates a deletion of the sequence GluAspAla that begins at position 233.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75, 80, 85, 90, 91, 92, 93, 94, 95, 96, 97, 98 or 99% identical to the parent protein, using the alignment programs described below, such as BLAST.

“Variant” as used herein also refers to particular amino acid modifications that confer particular function (e.g., a “heterodimerization variant,” “pI variant,” “ablation variant,” etc.).

As described below, in some embodiments the parent polypeptide, for example an Fc parent polypeptide, is a human wild-type sequence, such as the heavy constant domain or Fc region from IgG1, IgG2, IgG3 or IgG4, although human sequences with variants can also serve as “parent polypeptides”, for example the IgG1/2 hybrid of US Publication 2006/0134105 can be included. The protein variant sequence herein will preferably possess at least about 80% identity with a parent protein sequence, and most preferably at least about 90% identity, more preferably at least about 95-98-99% identity. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2 or IgG4.

“Fc variant” or “variant Fc” as used herein is meant a protein comprising an amino acid modification in an Fc domain. The modification can be an addition, deletion, or substitution. The Fc variants are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution.

In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Alternatively, the variant Fc domains can have up to 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Additionally, as discussed herein, the variant Fc domains described herein still retain the ability to form a dimer with another Fc domain as measured using known techniques as described herein, such as non-denaturing gel electrophoresis.

By “protein” as used herein is meant at least two covalently attached amino acids, which includes proteins, polypeptides, oligopeptides and peptides. In addition, polypeptides that make up the antibodies of the invention may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fe receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is an amino acid modification that contributes to increased binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “position” as used herein is meant a location in the sequence of a protein. Positions may be numbered sequentially, or according to an established format, for example the EU index for numbering of antibody domains (e.g., a CHi, CH2, CH3 or hinge domain).

By “target antigen” as used herein is meant the molecule that is bound specifically by the antigen binding domain comprising the variable regions of a given antibody.

By “strandedness” in the context of the monomers of the heterodimeric antibodies of the invention herein is meant that, similar to the two strands of DNA that “match,” heterodimerization variants are incorporated into each monomer so as to preserve the ability to “match” to form heterodimers. For example, if some pI variants are engineered into monomer A (e.g., making the pI higher) then steric variants that are “charge pairs” that can be utilized as well do not interfere with the pI variants, e.g., the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer one set of the pair will go, such that pI separation is maximized using the pI of the skews as well.

By “target cell” as used herein is meant a cell that expresses a target antigen.

By “host cell” in the context of producing a bispecific antibody according to the invention herein is meant a cell that contains the exogeneous nucleic acids encoding the components of the bispecific antibody and is capable of expressing the bispecific antibody under suitable conditions. Suitable host cells are discussed below.

By “wild type” or “WT” herein is meant an amino acid sequence or a nucleotide sequence that is found in nature, including allelic variations. A WT protein has an amino acid sequence or a nucleotide sequence that has not been intentionally modified.

Provided herein are a number of antibody domains (e.g., Fc domains) that have sequence identity to human antibody domains. Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol.48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore, SPR or BLI assay.

### IV. Anti-CD28×Anti-TROP2 Antibodies

In one aspect, provided herein are novel anti-CD28×anti-TROP2 antibodies In some embodiments, the anti-CD28×anti-TROP2 antibodies described herein are capable of agonistically binding to CD28 costimulatory molecules on T cells and TROP2 on tumor cells. Such antibodies selectively enhance anti-tumor activity at TROP2-associated tumor sites while minimizing peripheral toxicity. The subject antibodies provided herein are particularly useful in combination with other anti-cancer therapies, including, for example, bispecific antibodies for the treatment of TROP2-associated cancers.

The anti-CD28×anti-TROP2 antibodies are multivalent and include at least two antigen binding domains (ABDs), wherein at least one antigen binding domain is a CD28 binding domain and at least one antigen binding domain is a TROP2 binding domain. Any suitable CD28 binding domain and TROP2 binding domain can be included in the subject anti-CD28×anti-TROP2 antibodies, including, for example, the CD28 binding domains and TROP2 binding domains provided herein.

The antigen binding domains provided herein generally include a variable heavy domain (VH) having a VH-CDR1, VH-CDR-2, and VH-CDR-3; and a variable light domain (VL), and a variable light domain (VL) having a VL-CDR1, VL-CDR-2, and VL-CDR-3.

In addition, as discussed above, the numbering used in the sequence listing and figures for the identification of the CDRs is Kabat, however, different numbering can be used, which will change the amino acid sequences of the CDRs as shown in Table 2.

For all of the variable heavy and light domains listed herein, further variants can be made. As outlined herein, in some embodiments the set of 6 CDRs can have from 0, 1, 2, 3, 4 or 5 amino acid modifications (with amino acid substitutions finding particular use), as well as changes in the framework regions of the variable heavy and light domains, as long as the frameworks (excluding the CDRs) retain at least about 80, 85, 90, 95 or 99% identity to a human germline sequence selected from those listed in FIG. 1 of U.S. Pat. No. 7,657,380, which Figure and Legend is incorporated by reference in its entirety herein. Thus, for example, the identical CDRs as described herein can be combined with different framework sequences from human germline sequences, as long as the framework regions retain at least 80, 85 or 90% identity to a human germline sequence selected from those listed in FIG. 1 of U.S. Pat. No. 7,657,380. Alternatively, the CDRs can have amino acid modifications (e.g., from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g., there may be one change in vlCDR1, two in vhCDR2, none in vhCDR3, etc.)), as well as having framework region changes, as long as the framework regions retain at least 80, 85, 95 to 99% identity to a human germline sequence selected from those listed in FIG. 1 of U.S. Pat. No. 7,657,380.

As will be appreciated by those in the art, any set of 6 CDRs or VH and VL domains can be in the scFv format or in the Fab format, which is then added to the heavy and light constant domains, where the heavy constant domains comprise variants (including within the CHi domain as well as the Fc domain).

In addition, in embodiments wherein the subject antibody includes an scFv, the scFv can be in an orientation from N- to C-terminus of VH-scFv linker-VL or VL-scFv linker-VH. In some formats, one or more of the ABDs generally is a Fab that includes a VH domain on one protein chain (generally as a component of a heavy chain) and a VL on another protein chain (generally as a component of a light chain). Exemplary scFv linkers for use in the subject antibodies are depicted in FIG. 6.

In some embodiments, the anti-CD28×anti-TROP2 antibody is a bispecific antibody. In some embodiments, the anti-CD28×anti-TROP2 antibody is a bivalent antibody. In some embodiments, the anti-CD28×anti-TROP2 antibody is a trivalent antibody. In some embodiments, the anti-CD28×anti-TROP2 antibody is a bispecific, bivalent antibody. In some embodiments, the anti-CD28×anti-TROP2 antibodies include one CD28 binding domain and one TROP2 binding domain. In exemplary embodiments, the anti-CD28×anti-TROP2 antibody is a bispecific, trivalent antibody. In some embodiments, the anti-CD28×anti-TROP2 antibodies include one CD28 binding domain and two TROP2 binding domains.

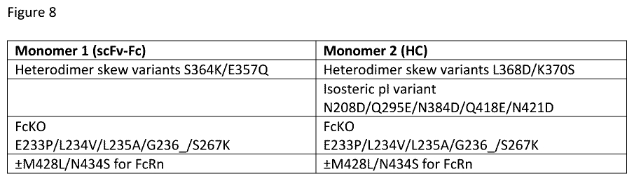

The anti-CD28×anti-TROP2 antibodies provided herein can be in any useful format, including, including, for example, canonical immunoglobulin, as well as the “1+1 Fab-scFv-Fc,” “2+1 “mAb-scFv,” and “2+1 Fab2-scFv-Fc,” formats described herein (FIG. 15). Additional useful formats include, but are not limited to: “mAb-Fv,” “central-Fv,” “one-armed scFv-mAb,” “scFv-mAb,” “dual scFv,” and “trident” formats provided herein (see, e.g., FIG. 15). See also, US20180127501A1, which is incorporated by reference herein, particularly in pertinent part relating to antibody formats (see, e.g., FIG. 2). In some embodiments, the anti-CD28×anti-TROP2 antibodies are heterodimeric bispecific antibodies that include variant Fc domains having any of the heterodimerization skew variants, pI variants and/or ablation variants described herein. See, e.g. FIG. 8.

Note that unless specified herein, the order of the antigen list in the name does not confer structure; that is an anti-TROP2×anti-CD28 1+1 Fab-scFv-Fc antibody can have the scFv bind to TROP2 or CD28, although in some cases, the order specifies structure as indicated.

The anti-CD28×anti-TROP2 antibodies provided herein further include different antibody domains. As described herein and known in the art, the antibodies described herein include different domains within the heavy and light chains, which can be overlapping as well. These domains include, but are not limited to, the Fc domain, the CHi domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

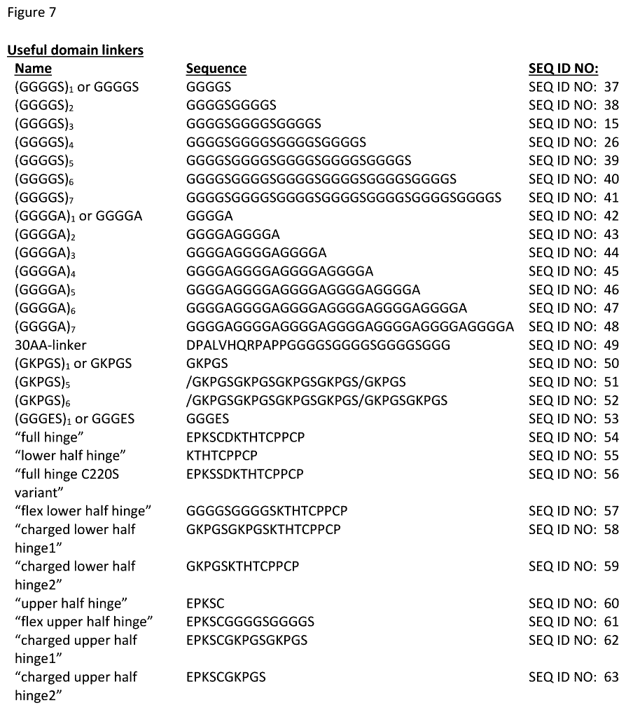

As shown herein, there are a number of suitable linkers (for use as either domain linkers or scFv linkers) that can be used to covalently attach the recited domains (e.g., scFvs, Fabs, Fc domains, VH domains, VL domains, etc.), including traditional peptide bonds, generated by recombinant techniques. Exemplary linkers to attach domains of the subject antibody to each other are depicted in FIG. 7. In some embodiments, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n (SEQ ID NO: 750), (GGGGS)n (SEQ ID NO: 751), and (GGGS)n (SEQ ID NO: 752), where n is an integer of at least one (and generally from 3 to 4), glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of CL/CH1 domain but not all residues of CL/CH1 domain; for example, the first 5-12 amino acid residues of the CL/CH1 domains. Linkers can be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR), hinge region-derived sequences, and other natural sequences from other proteins.

In some embodiments, the linker is a “domain linker,” used to link any two domains as outlined herein together. For example, in the 2+1 Fab2-scFv-Fc format, there may be a domain linker that attaches the C-terminus of the CH1 domain of the Fab to the N-terminus of the scFv, with another optional domain linker attaching the C-terminus of the scFv to the CH2 domain (although in many embodiments the hinge is used as this domain linker). While any suitable linker can be used, many embodiments utilize a glycine-serine polymer as the domain linker, including for example (GS)n, (GSGGS)n (SEQ ID NO: 750), (GGGGS)n (SEQ ID NO: 751), and (GGGS)n (SEQ ID NO: 752), where n is an integer of at least one (and generally from 3 to 4 to 5) as well as any peptide sequence that allows for recombinant attachment of the two domains with sufficient length and flexibility to allow each domain to retain its biological function. In some cases, and with attention being paid to “strandedness,” as outlined below, charged domain linkers, as used in some embodiments of scFv linkers can be used. Exemplary useful domain linkers are depicted in FIG. 7.

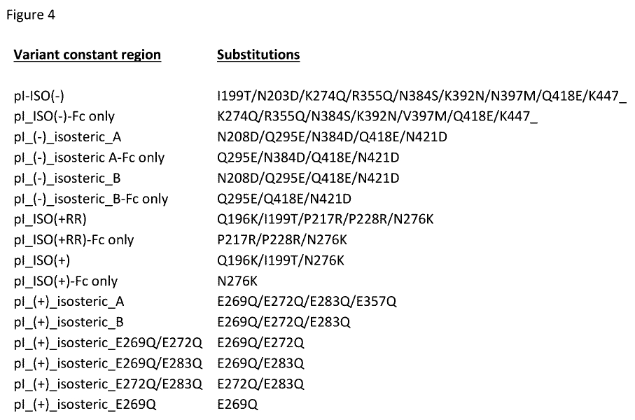

In some embodiments, the linker is a scFv linker that is used to covalently attach the VH and VL domains as discussed herein. In many cases, the scFv linker is a charged scFv linker, a number of which are shown in FIG. 6. Accordingly, provided herein are charged scFv linkers, to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains. These charged linkers can be substituted into any scFv containing standard linkers. Again, as will be appreciated by those in the art, charged scFv linkers are used on the correct “strand” or monomer, according to the desired changes in pI. For example, as discussed herein, to make 1+1 Fab-scFv-Fc format heterodimeric antibody, the original pI of the Fv region for each of the desired antigen binding domains are calculated, and one is chosen to make an scFv, and depending on the pI, either positive or negative linkers are chosen. Charged domain linkers can also be used to increase the pI separation of the monomers of the invention as well, and thus those included in FIG. 4 can be used in any embodiment herein where a linker is utilized.

Exemplary subject anti-CD28×anti-TROP2 antibodies are depicted, for example, in FIGS. 30 and 31. During the cell culture production of the anti-CD28×anti-TROP2 antibodies provided herein, the C-terminal lysine residue or C-terminal lysine and glycine residues may be cleaved from the heavy chain monomers, thereby leading to variants with C-terminal “clipping.” See, e.g., Jiang et al., Journal of Pharmaceutical Sciences 105:2066-2072 (2016). Thus, in some embodiments provided herein, the anti-CD28×anti-TROP2 antibody is a variant of one of the anti-CD28×anti-TROP2 antibodies includes a deletion of a C-terminal lysine (-K) terminal or C-terminal lysine and glycine (-GK) residues in one or both Fc domains of an anti-CD28×anti-TROP2 antibody described herein. In some embodiments, the deletion is G446del and/or K447del (EU numbering).

Aspects of the anti-CD28×anti-TROP2 antibodies are further described in detail below.

### A. CD28 Binding Domains

The anti-CD28×anti-TROP2 antibodies provided herein include at least one CD28 binding domain. Any suitable CD28 binding domain can be included in the anti-CD28×anti-TROP2 antibodies provided herein. In exemplary embodiments, the CD28 binding domain is an agonistic CD28 ABDs that advantageously provide T cell costimulatory activity.

As will be appreciated by those in the art, suitable CD28 binding domains can comprise a set of 6 CDRs as depicted in the figures, either as they are underlined or, in the case where a different numbering scheme is used, as described herein and as shown in Table 2, as the CDRs that are identified using other alignments within the variable heavy (VH) domain and variable light domain (VL) sequences of those CD28 binding domains depicted in FIGS. 16, 19 and 22. Additional VH and VL sequences of exemplary CD28 binding domains that can be used in the subject antibodies are depicted in FIGS. 17, 18, and 34. Suitable CD28 ABDs can also include the entire VH and VL sequences as depicted in these sequences and figures, used as scFvs or as Fabs.

In one embodiment, the CD28 antigen binding domain includes the 6 CDRs (i.e., vhCDR1-3 and vlCDR1-3) of any of the CD28 binding domains described herein, including, but not limited to those depicted in FIGS. 16, 19 and 22. In some embodiments, the CD28 ABD that binds human CD28 is one of the following CD28 ABDs: 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]H4L2 (FIGS. 16, 19 and 22). In some embodiments, the CD28 ABD includes a VH/VL pair selected from the VHs and VLs depicted in FIGS. 16-19, 22 and 34.

In addition to the parental CDR sets disclosed in the figures and sequence listing that form an ABD to CD28, provided herein are variant CD28 ABDS having CDRs that include at least one modification of the CD28 ABD CDRs disclosed herein (e.g., (FIGS. 16, 19 and 22, and the sequence listing). In one embodiment, the CD28 ABD of the subject anti-CD28×anti-TROP2 antibody includes a set of 6 CDRs with 1, 2, 3, 4, 5, 6, 7, 8, 9, 10 amino acid modifications as compared to the 6 CDRs of a CD28 ABD as described herein, including the figures and sequence listing. In exemplary embodiments, the CD28 ABD of the subject anti-CD28×anti-TROP2 antibody includes a set of 6 CDRs with 1, 2, 3, 4, 5, 6, 7, 8, 9, 10 amino acid modifications as compared to the 6 CDRs of one of the following CD28 ABDs: 1A7[CD28]_H1L1, 1A7[CD28]_H1.1_L1, 1A7[CD28]_H1_L1.71, 1A7[CD28]_H1.1_L1.71, 1A7[CD28]_H1.14_L1 11A7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]_H4L2 (FIGS. 16, 19 and 22). In certain embodiments, the CD28 ABD of the subject anti-CD28×anti-TROP2 antibody is capable of binding CD28 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In some embodiments, the CD28 ABD of the subject anti-CD28×anti-TROP2 antibody includes 6 CDRs that are at least 90, 95, 97, 98 or 99% identical to the 6 CDRs of a CD28 ABD as described herein, including the figures and sequence listing. In exemplary embodiments, the CD28 ABD of the subject anti-CD28×anti-TROP2 antibody includes 6 CDRs that are at least 90, 95, 97, 98 or 99% identical to the 6 CDRs of one of the following CD28 ABDs: 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, CD28.3[CD28]_H0L0, hCD28.3[CD28]_H1L1, 5.11A1[CD28]_H0L0, TGN1412_HIL1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]H4L2 (FIGS. 16, 19 and 22). In certain embodiments, the CD28 ABD is capable of binding to the CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In another exemplary embodiment, the CD28 ABD of the subject anti-CD28×anti-TROP2 antibody includes the variable heavy (VH) domain and variable light (VL) domain of any one of the CD28 ABDs described herein, including the figures and sequence listing. In exemplary embodiments, the CD28 ABD is one of the following CD28 ABDs: 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]H4L2 (FIGS. 16, 19 and 22). In some embodiments, the CD28 ABD includes a VH and VL pair selected from the VH and VLs depicted in FIGS. 16-19, 22 and 34.

In some embodiments, the anti-CD28×anti-TROP2 antibody includes a CD28 ABD that includes a variable heavy domain and/or a variable light domain that are variants of a CD28 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a CD28 ABD described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of one of the following CD28 ABDs: 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]H4L2 (FIGS. 16, 19 and 22). In some embodiments, the changes are in a VH domain depicted in FIGS. 16-19 and 22. In some embodiments, the changes are in a VL domain are depicted in FIGS. 16-19 and 22. In some embodiments, the changes are in a VH and VL domain are depicted in FIGS. 16-19 and 22. In some embodiments, the one or more amino acid changes are in the VH and/or VL framework regions (FRI, FR2, FR3, and/or FR4). In some embodiments, the one or more amino acid change(s) are in one or more CDRs. In certain embodiments, the CD28 ABD is capable of binding to CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In one embodiment, the variant VH and/or VL domain is at least 90, 95, 97, 98 or 99% identical to the VH and/or VL of a CD28 ABD as described herein, including the figures and sequence listing. In exemplary embodiments, the variant VH and/or VL domain is at least 90, 95, 97, 98 or 99% identical to the VH and/or VL of one of the following CD28 ABDs: 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]_H4L2 (FIGS. 16, 19 and 22). In some embodiments, the CD28 ABD includes a VH that is at least 90, 95, 97, 98 or 99% identical to VH domain depicted in FIGS. 16-19 and 22. In some embodiments, the CD28 ABD includes a VL that is at least 90, 95, 97, 98 or 99% identical to VL domain depicted in FIGS. 16-19 and 22. In some embodiments, the CD28 ABD includes a VH and a VL that is at least 90, 95, 97, 98 or 99% identical to a VH domain and a VL domain depicted in FIGS. 16-19 and 22. In certain embodiments, the CD28 ABD is capable of binding to CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In some embodiments, the CD28 binding domain of the subject anti-CD28×anti-TROP2 antibody includes a VH that includes any one of the VHCDR1-3 or HFR1-4 sequences depicted in FIG. 20A. In some embodiments, the CD28 binding domain includes a VL that includes any one of the VLCDRI-3 or LFR1-4 sequences depicted in FIG. 20B.

In some embodiments, the anti-CD28×anti-TROP2 antibody includes a CD28 binding domain that includes a VH and VL selected from the following:
(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 16, 19 and 22 or a variant thereof; and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 16, 19 and 22 or a variant thereof; or
(i) a VH comprising a vhCDR1, a vhCDR2, and a vhCDR3 having an amino acid sequence of a vhCDR1, a vhCDR2, and a vhCDR3, respectively, of a VH depicted in FIG. 17 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIG. 18 or a variant thereof.

In some embodiments, the anti-CD28×anti-TROP2 antibody includes a CD28 binding domain that includes a VH and VL selected from the following:
(i) a VH having an amino acid sequence of a VH of any of the CD28 binding domains depicted in FIGS. 16, 19 and 22 or a variant thereof; and (ii) a VL having an amino acid sequence of the VL of the CD28 binding domain depicted in FIGS. 16, 19 and 22 or a variant thereof; or
(i) a VH having an amino acid sequence of a VH depicted in FIG. 17 or 34 or a variant thereof; and (ii) a VL having an amino acid sequence of the VL of a VL depicted in FIG. 18 or a variant thereof.

### 1. Additional CD28 Binding Domains

In some embodiments, the anti-CD28×anti-TROP2 antibodies provided herein includes a CD28 binding domain that includes a common light chain, wherein the common light chain can also be used as a light chain for a TROP2 binding domain. In some embodiments, anti-CD28×anti-TROP2 antibodies that include such a CD28 binding domain, the CD28 binding domain and TROP2 binding domain each include the common light chain. Exemplary anti-CD28×anti-TROP2 antibody formats that utilize CD28 binding domains that include a common light chain include the 1+1 common light chain (CLC) and 2+1 common light chain formats described herein.

In one embodiment, the CD28 antigen binding domain includes the vhCDR1-3 of any of the CD28 variable heavy domains depicted in FIG. 34, and the vlCDR1-3 of the 1F11-1A3.315 L1 variable light domain (see FIG. 36A).

In one embodiment, the CD28 ABD of the subject anti-CD28×anti-TROP2 antibody includes a) a vhCDR1, vhCDR2, and/or vhCDR3 with 1, 2, 3, 4, 5, 6, 7, 8, 9, 10 amino acid modifications as compared to the vhCDR1, vhCDR2, and/or vhCDR3 of one of the CD28 variable heavy domains depicted in FIG. 34, respectively, and/or b) a vlCDR1, vlCDR2, and/or vlCDR3 with 1, 2, 3, 4, 5, 6, 7, 8, 9, 10 amino acid modifications as compared to the vlCDR1, vlCDR2, and/or vlCDR3 of the 1F11-1A3.315 L1 variable light domain (see FIG. 36A), respectively. In certain embodiments, the CD28 ABD of the subject anti-CD28×anti-TROP2 antibody is capable of binding CD28 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In one embodiment, the CD28 ABD of the subject anti-CD28×anti-TROP2 antibody includes a vhCDR1, vhCDR2, and/or vhCDR3 that is at least 90, 95, 97, 98 or 99% identical to the vhCDR1, vhCDR2, and/or vhCDR3 of one of the CD28 variable heavy domains depicted in FIG. 34, respectively, and/or a vlCDR1, vlCDR2, and/or vlCDR3 that is at least 90, 95, 97, 98 or 99% identical to the vlCDR1, vlCDR2, and/or vlCDR3 of the 1F11-1A3.315 L1 variable light domain (see FIG. 36A), respectively. In certain embodiments, the CD28 ABD is capable of binding to the CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In another exemplary embodiment, the CD28 ABD of the subject anti-CD28×anti-TROP2 antibody includes the variable heavy (VH) domain of one of the CD28 variable heavy domains depicted in FIG. 34, and the 1F11-1A3.315 L1 variable light domain (see FIG. 36A).

In some embodiments, the anti-CD28×anti-TROP2 antibody includes a CD28 ABD that includes a variable heavy domain that is a variant of one of the CD28 variable heavy domains depicted in FIG. 34, and/or a variable light domain that is a variant of the 1F11-1A3.315 L1 variable light domain (see FIG. 36A). In some embodiments, the variant VH domain has from 1, 2, 3, 4, 5, 6, 7, 8, 9 or 10 amino acid changes compared to one of the CD28 variable heavy domains depicted in FIG. 34 and/or the variable light domain has from 1, 2, 3, 4, 5, 6, 7, 8, 9 or 10 amino acid changes from the 1F11-1A3.315 L1 variable light domain (see FIG. 36A). In some embodiments, the one or more amino acid changes are in the VH and/or VL framework regions (FR1, FR2, FR3, and/or FR4). In some embodiments, the one or more amino acid change(s) are in one or more CDRs. In certain embodiments, the CD28 ABD is capable of binding to CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In one embodiment, the variant VH domain is at least 90, 95, 97, 98 or 99% identical to one of the CD28 variable heavy domains depicted in FIG. 34 and/or the variable light domain is at least 90, 95, 97, 98 or 99% identical to the 1F11-1A3.315 L1 variable light domain (see FIG. 36A). In certain embodiments, the CD28 ABD is capable of binding to CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In some embodiments, the CD28 binding domain of the subject anti-CD28×anti-TROP2 antibody includes a VH that includes any one of the VHCDR1-3 or HFR1-4 sequences depicted in FIG. 34C. In some embodiments, the CD28 binding domain includes any one of the VLCDR1-3 or the LFR1-4 sequences of the 1F11-1A3.315 L1 variable light domain (see FIG. 36A).

### B. TROP2 Binding Domains

The anti-CD28×anti-TROP2 antibodies provided herein include at least one TROP2 binding domain. Subject antibodies that include such TROP2 antigen binding domains (e.g., anti-TROP2×anti-CD3 bispecific antibodies) advantageously target cells that express high levels of TROP2 over those that express levels of TROP2 (e.g., normal cells).

As will be appreciated by those in the art, suitable TROP2 binding domains can comprise a set of 6 CDRs as depicted in the sequence listing and FIGS. 23-28, either as the CDRs are underlined or, in the case where a different numbering scheme is used as described herein and as shown in Table 2, as the CDRs that are identified using other alignments within the variable heavy (VH) domain and variable light domain (VL) sequences of those depicted in FIGS. 23-28 and the sequence listing (see Table 2). Suitable TROP2 ABDs can also include the entire VH and VL sequences as depicted in these sequences and figures, used as scFvs or as Fab domains.

In exemplary embodiments, the TROP2 ABD of the anti-CD28×anti-TROP2 antibody include the variable heavy (VH) domain and variable light (VL) domain of any one of the TROP2 ABDs described herein, including the figures and sequence listing. In exemplary embodiments, the TROP2 ABD is a TROP2 ABD depicted in FIG. 23. In exemplary embodiments, the TROP2 ABD comprises a VH and VL pair selected from those depicted in FIGS. 23-28.

In addition to the parental TROP2 variable heavy and variable light domains disclosed herein, provided herein are TROP2 ABDs that include a variable heavy domain and/or a variable light domain that are variants of a TROP2 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of a TROP2 ABD described herein, including the figures and sequence listing. In exemplary embodiments, the variant VH domain and/or VL domain has from 1, 2, 3, 4, 5, 6, 7, 8, 9 or 10 amino acid changes from a VH and/or VL domain of a TROP2 ABD depicted in FIG. 23. In exemplary embodiments, the variant VH domain and/or VL domain has from 1, 2, 3, 4, 5, 6, 7, 8, 9 or 10 amino acid changes from a VH and/or VL domain depicted in FIGS. 23-28. In some embodiments, the changes are in a VH domain depicted in FIGS. 23-28. In some embodiments, the changes are in a VL domain are depicted in FIGS. 23-28. In some embodiments, the changes are in a VH and VL domain are depicted in FIGS. 23-28. In some embodiments, the one or more amino acid changes are in the VH and/or VL framework regions (FR1, FR2, FR3, and/or FR4). In some embodiments, the one or more amino acid change(s) are in one or more of vhCDR1-3 and/or vlCDR1-3. In certain embodiments, the TROP2 ABD of the anti-CD28×anti-TROP2 antibody is capable of binding to TROP2, as measured at least one of a Biacore, surface plasmon resonance (SPR), flow cytometry, and/or BLI (biolayer interferometry, e.g., Octet assay) assay, with the latter finding particular use in many embodiments. In particular embodiments, the TROP2 ABD is capable of binding human TROP2 antigen (FIG. 2).

In one embodiment, the variant VH and/or VL domain is at least 90, 95, 97, 98 or 99% identical to the VH and/or VL of a TROP2 ABD as described herein, including the figures and sequence listing. In exemplary embodiments, the variant VH and/or VL domain is at least 90, 95, 97, 98 or 99% identical to the VH and/or VL of a TROP2 ABD depicted in FIG. 23. In exemplary embodiments, the variant VH and/or VL domain is at least 90, 95, 97, 98 or 99% identical to the VH and/or VL depicted in FIGS. 23-28. In some embodiments, the TROP2 ABD includes a VH that is at least 90, 95, 97, 98 or 99% identical to VH domain depicted in FIGS. 23-28. In some embodiments, the TROP2 ABD includes a VL that is at least 90, 95, 97, 98 or 99% identical to VL domain depicted in FIGS. 23-28. In some embodiments, the TROP2 ABD includes a VH and a VL that is at least 90, 95, 97, 98 or 99% identical to a VH domain and a VL domain depicted in FIGS. 23-28. In certain embodiments, the TROP2 ABD of the anti-CD28×anti-TROP2 antibody is capable of binding to the TROP2, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the TROP2 ABD is capable of binding human TROP2 antigen (FIG. 2).

In some embodiments, the anti-CD28×anti-TROP2 antibody is a bivalent antibody (e.g., 1+1 Fab-scFv-Fc format antibody) that includes one TROP2 binding domain. In other embodiments, the anti-CD28×anti-TROP2 antibody is a trivalent antibody (e.g., 2+1 mAb-scFv and 2+1 Fab2_scFv-Fc format antibodies) that includes two TROP2 binding domains.

### 1. Additional TROP2 Binding Domains

Provided herein are TROP2 binding domains compositions that include a common variable light domain, wherein the common variable light domain can advantageously also be included in particular CD28 and PDL1 binding domains described herein. In some embodiments, the TROP2 antigen binding domain includes one of the TROP2 variable heavy domains depicted in FIG. 36, and the 1F11-1A3.315 L1 variable light domain (see FIG. 36A, also referred to herein as the “IGKV1-39” variable light domain). In some embodiments, the TROP2 antigen binding domain includes the variable heavy domain and variable light domain of one of the TROP2 antigen binding domains depicted in FIGS. 52-56 or a variant thereof.

In some embodiments, the anti-CD28×anti-TROP2 antibodies provided herein includes a TROP2 binding domain that includes a common light chain, wherein the common light chain can also be used as a light chain for a CD28 binding domain. In some embodiments, anti-CD28×anti-TROP2 antibodies that include such a TROP2 binding domain, the CD28 binding domain and TROP2 binding domain each include the common light chain. Exemplary anti-CD28×anti-TROP2 antibody formats that utilize TROP2 binding domains that include a common light chain include the 1+1 common light chain (CLC) and 2+1 common light chain formats described herein.

In one embodiment, the TROP2 antigen binding domain includes the vhCDR1-3 of any of the TROP2 variable heavy domains depicted in FIG. 36, and the vlCDR1-3 of the 1F11-1A3.315 L1 variable light domain (see FIG. 36A). In some embodiments, the TROP2 antigen binding domain includes the vhCDR1-3 and vlCDR1-3 of any one of the TROP2 antigen binding domains depicted in FIGS. 52-56.

In one embodiment, the TROP2 ABD of the subject anti-CD28×anti-TROP2 antibody includes a) a vhCDR1, vhCDR2, and/or vhCDR3 with 1, 2, 3, 4, 5, 6, 7, 8, 9, 10 amino acid modifications as compared to the vhCDR1, vhCDR2, and/or vhCDR3 of one of the TROP2 variable heavy domains depicted in FIGS. 36 and 52-56, respectively, and/or b) a vlCDR1, vlCDR2, and/or vlCDR3 with 1, 2, 3, 4, 5, 6, 7, 8, 9, 10 amino acid modifications as compared to the vlCDR1, vlCDR2, and/or vlCDR3 of the 1F11-1A3.315 L1 variable light domain (see FIGS. 36A and 52-56), respectively. In certain embodiments, the TROP2 ABD of the subject anti-CD28×anti-TROP2 antibody is capable of binding TROP2 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the TROP2 ABD is capable of binding human TROP2 antigen (see FIG. 2).

In one embodiment, the TROP2 ABD of the subject anti-CD28×anti-TROP2 antibody includes a vhCDR1, vhCDR2, and/or vhCDR3 that is at least 90, 95, 97, 98 or 99% identical to the vhCDR1, vhCDR2, and/or vhCDR3 of one of the TROP2 variable heavy domains depicted in FIGS. 36 and 52-56, respectively, and/or a vlCDR1, vlCDR2, and/or vlCDR3 that is at least 90, 95, 97, 98 or 99% identical to the vlCDR1, vlCDR2, and/or vlCDR3 of the 1F11-1A3.315 L1 variable light domain (see FIGS. 36A and 52-56), respectively. In certain embodiments, the TROP2 ABD is capable of binding to TROP2 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the TROP2 ABD is capable of binding human TROP2 antigen (see FIG. 2).

In another exemplary embodiment, the TROP2 ABD of the subject anti-CD28×anti-TROP2 antibody includes the variable heavy (VH) domain of one of the TROP2 variable heavy domains depicted in FIGS. 36 and 52-56, and the 1F11-1A3.315 L1 variable light domain (see FIGS. 36A and 52-56).

In some embodiments, the anti-CD28×anti-TROP2 antibody includes a TROP2 ABD that includes a variable heavy domain that is a variant of one of the TROP2 variable heavy domains depicted in FIGS. 36 and 52-56, and/or a variable light domain that is a variant of the 1F11-1A3.315 L1 variable light domain (see FIG. 36A). In some embodiments, the variant VH domain has from 1, 2, 3, 4, 5, 6, 7, 8, 9 or 10 amino acid changes compared to one of the TROP2 variable heavy domains depicted in FIGS. 36 and 52-56 and/or the variable light domain has from 1, 2, 3, 4, 5, 6, 7, 8, 9 or 10 amino acid changes from the 1F11-1A3.315 L1 variable light domain (see FIGS. 36A and 52-56). In some embodiments, the one or more amino acid changes are in the VH and/or VL framework regions (FR1, FR2, FR3, and/or FR4). In some embodiments, the one or more amino acid change(s) are in one or more CDRs. In certain embodiments, the TROP2 ABD is capable of binding to TROP2, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the TROP2 ABD is capable of binding human TROP2 antigen (see FIG. 2).

In one embodiment, the variant VH domain is at least 90, 95, 97, 98 or 99% identical to one of the TROP2 variable heavy domains depicted in FIGS. 36 and 52-56 and/or the variable light domain is at least 90, 95, 97, 98 or 99% identical to the 1F11-1A3.315 L1 variable light domain (see FIGS. 36A and 52-56). In certain embodiments, the TROP2 ABD is capable of binding to TROP2, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the TROP2 ABD is capable of binding human TROP2 antigen (see FIG. 2).

In some embodiments, the TROP2 binding domain of the subject anti-CD28×anti-TROP2 antibody includes a VH that includes any one of the VHCDR1-3 or HFR1-4 sequences depicted in FIG. 36C. In some embodiments, the TROP2 binding domain includes any one of the VLCDR1-3 or the LFR1-4 sequences of the 1F11-1A3.315 L1 variable light domain (see FIG. 36A).

### C. Chimeric and Humanized Antibodies

In certain embodiments, the subject antibodies provided herein include a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene (prior to the introduction of any skew, pI and ablation variants herein; that is, the number of variants is generally low, prior to the introduction of the variants of the invention). In certain cases, the humanized antibody may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants of the invention).

In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

### D. Heterodimeric Antibodies

In exemplary embodiments, the anti-CD28×anti-TROP2 antibodies provided herein are heterodimeric bispecific antibodies that include two variant Fc domain sequences. Such variant Fc domains include amino acid modifications to facilitate the self-assembly and/or purification of the heterodimeric antibodies.

An ongoing problem in antibody technologies is the desire for “bispecific” antibodies that bind to two different antigens simultaneously, in general thus allowing the different antigens to be brought into proximity and resulting in new functionalities and new therapies. In general, these antibodies are made by including genes for each heavy and light chain into the host cells. This generally results in the formation of the desired heterodimer (A-B), as well as the two homodimers (A-A and B-B (not including the light chain heterodimeric issues)). However, a major obstacle in the formation of bispecific antibodies is the difficulty in biasing the formation of the desired heterodimeric antibody over the formation of the homodimers and/or purifying the heterodimeric antibody away from the homodimers.

There are a number of mechanisms that can be used to generate the subject heterodimeric antibodies. In addition, as will be appreciated by those in the art, these different mechanisms can be combined to ensure high heterodimerization. Amino acid modifications that facilitate the production and purification of heterodimers are collectively referred to generally as “heterodimerization variants.” As discussed below, heterodimerization variants include “skew” variants (e.g., the “knobs and holes” and the “charge pairs” variants described below) as well as “pI variants,” which allow purification of heterodimers from homodimers. As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

Heterodimerization variants that are useful for the formation and purification of the subject heterodimeric antibody (e.g., bispecific antibodies) are further discussed in detailed below.

### 1. Skew Variants

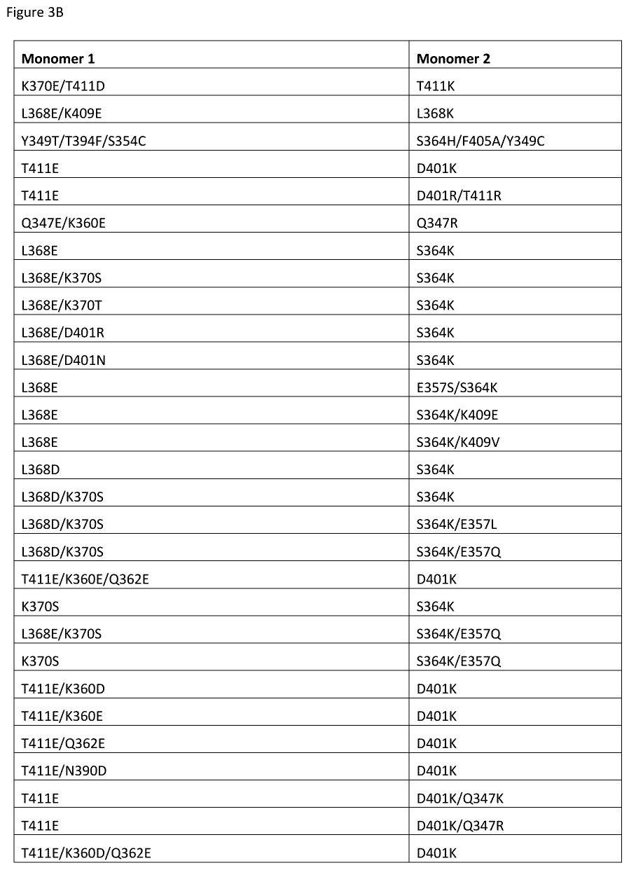

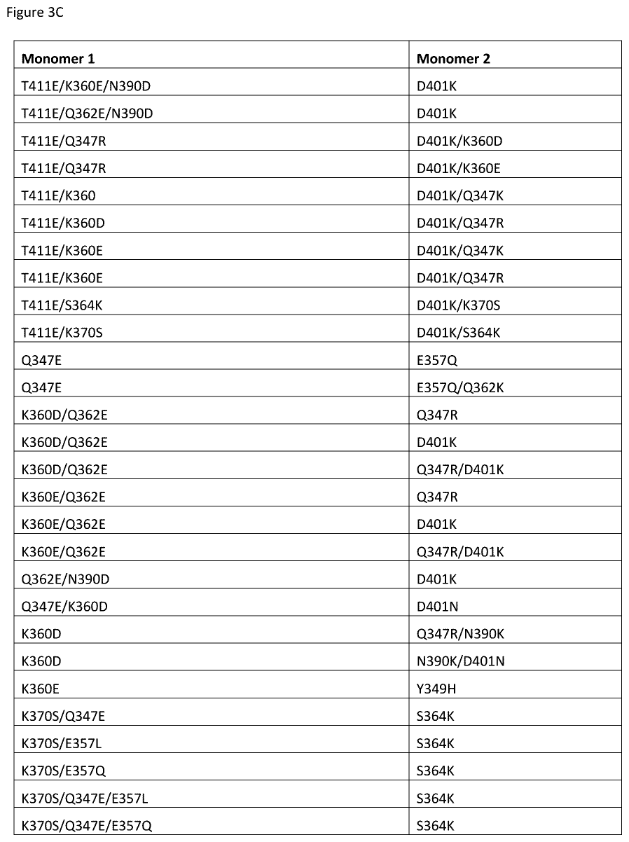

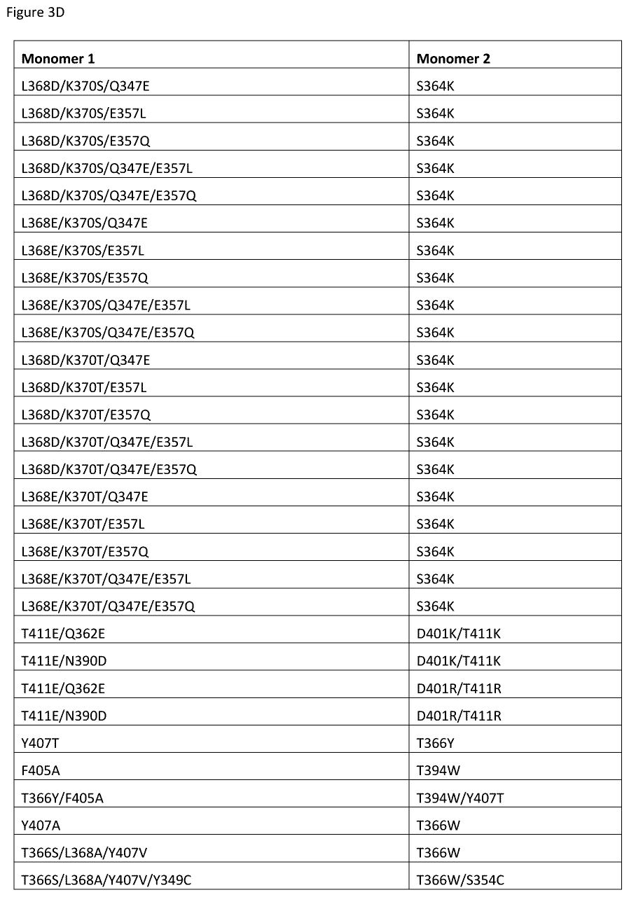

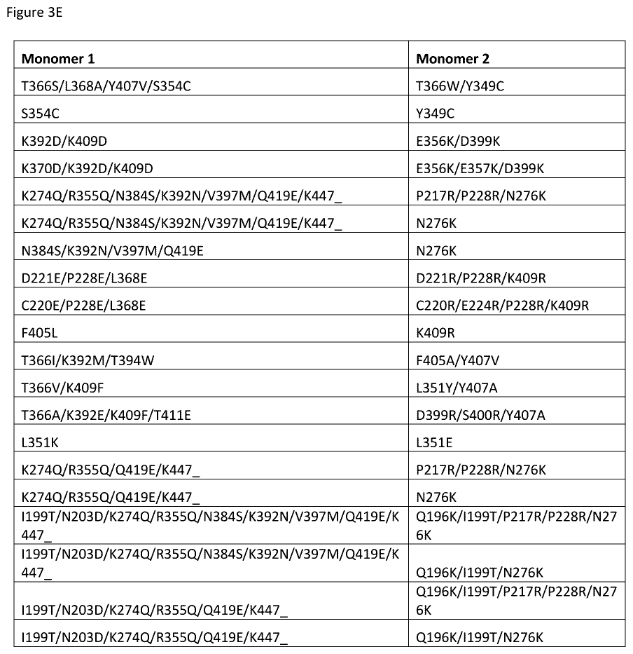

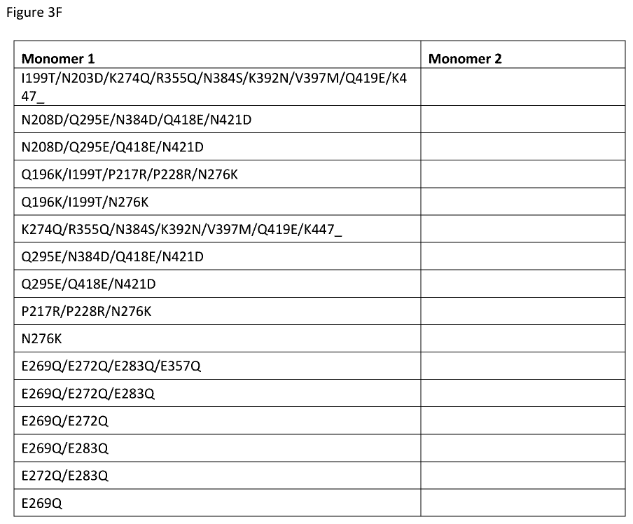

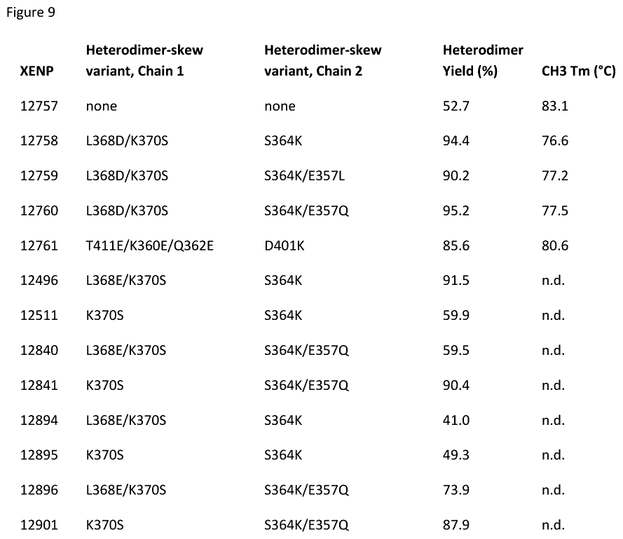

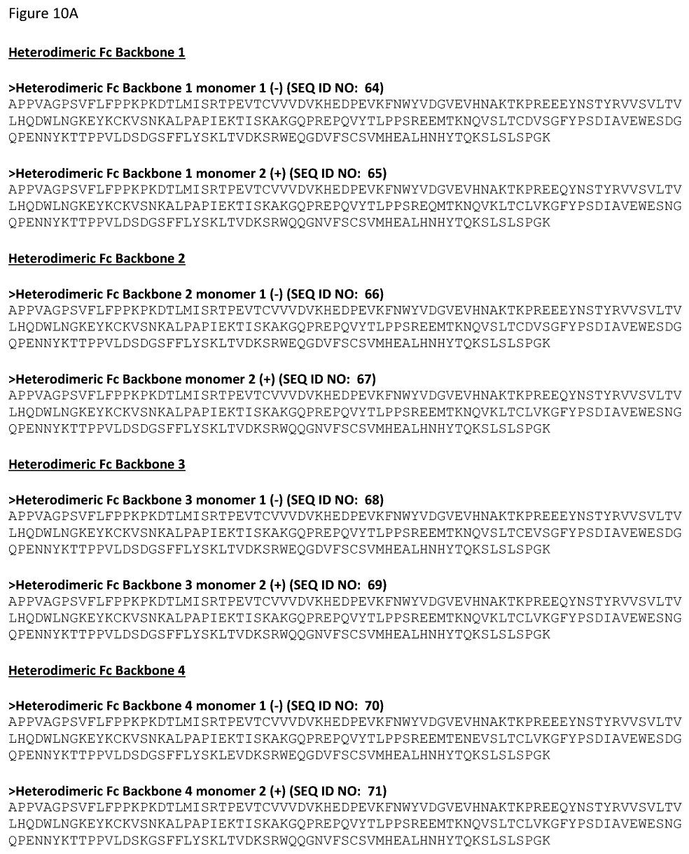

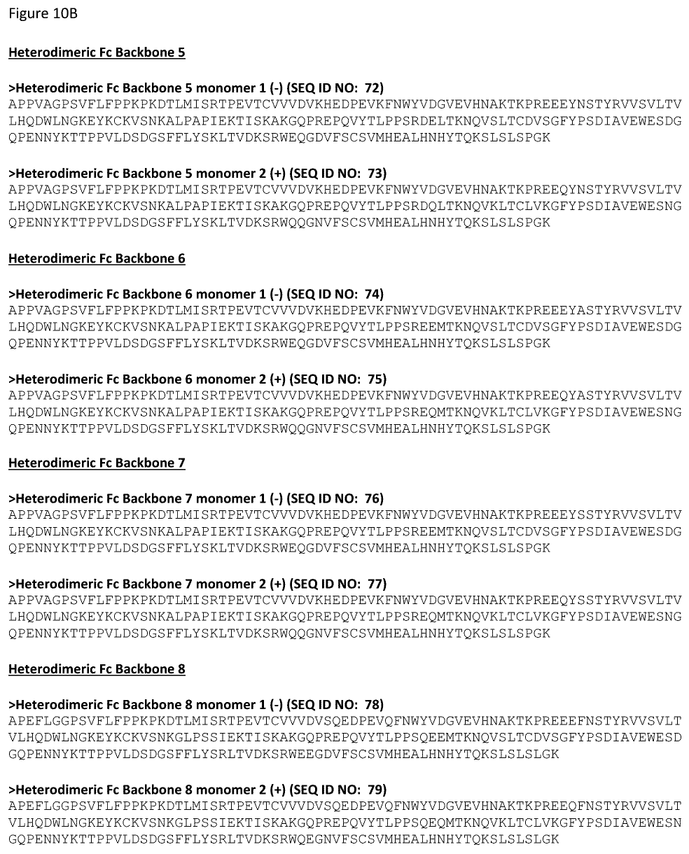

In some embodiments, the heterodimeric antibody includes skew variants which are one or more amino acid modifications in a first Fc domain (A) and/or a second Fc domain (B) that favor the formation of Fc heterodimers (Fc dimers that include the first and the second Fc domain; (A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in FIGS. 3 and 9.

One particular type of skew variants is generally referred to in the art as “knobs and holes,” referring to amino acid engineering that creates steric influences to favor heterodimeric formation and disfavor homodimeric formation, as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety and specifically for the disclosure of “knobs and holes” mutations. This is sometime referred to herein as “steric variants.” The figures identify a number of “monomer A-monomer B” pairs that rely on “knobs and holes.” In addition, as described in Merchant et al., Nature Biotech. 16:677 (1998), these “knobs and holes” mutations can be combined with disulfide bonds to further favor formation of Fc heterodimers.

Another method that finds use in the generation of heterodimers is sometimes referred to as “electrostatic steering” as described in Gunasekaran et al., J. Biol. Chem. 285(25):19637 (2010), hereby incorporated by reference in its entirety. This is sometimes referred to herein as “charge pairs.” In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “skew variants.” These include, but are not limited to, D221E/P228E/L368E paired with D221R/P228R/K409R (e.g., these are “monomer corresponding sets) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the heterodimeric antibody includes one or more sets of such heterodimerization skew variants. These variants come in “pairs” of “sets.” That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B). Exemplary heterodimerization “skew” variants are depicted in FIGS. 3 and 9. Such “skew” variants include, but are not limited to: S364K/E357Q:L368D/K370S; L368D/K370S:S364K; L368E/K370S:S364K; T411T/E360E/Q362E:D401K; L368D/K370S:S 364K/E357L; K370S:S364K/E357Q (EU numbering).

In exemplary embodiments, the heterodimeric antibody includes a S364K/E357Q:L 368D/K370S; L368D/K370S:S364K; L368E/K370S:S364K; T411T/E360E/Q362E:D401K; L368D/K370S:S364K/E357L; K370S:S364K/E357Q; or a T366S/L368A/Y407V:T366W (optionally including a bridging disulfide, T366S/L368A/Y407V/Y349C:T366W/S354C) “skew” variant amino acid substitution set (EU numbering). In an exemplary embodiment, the heterodimeric antibody includes a “S364K/E357Q:L368D/K370S” amino acid substitution set. In terms of nomenclature, the pair “S364K/E357Q:L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI.

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotpypic variants, FcRn variants, ablation variants, etc. into one or both of the first and second Fc domains of the heterodimeric antibody. Further, individual modifications can also independently and optionally be included or excluded from the subject the heterodimeric antibody.

In some embodiments, the skew variants outlined herein can be optionally and independently incorporated with any pI variant (or other variants such as Fc variants, FcRn variants, etc.) into one or both heavy chain monomers, and can be independently and optionally included or excluded from the subject heterodimeric antibodies.

### 2. Purification Variants

In some embodiments, the heterodimeric antibody includes purification variants that advantageously allow for the separation of heterodimeric proteins (e.g., anti-CD28×anti-TROP2 bispecific antibody) from homodimeric proteins.

There are several basic mechanisms that can lead to ease of purifying heterodimeric antibodies. For example, modifications to one or both of the antibody heavy chain monomers A and B such that each monomer has a different pI allows for the isoelectric purification of heterodimeric A-B antibody from monomeric A-A and B-B proteins. Alternatively, some scaffold formats, such as the “1+1 Fab-scFv-Fc” format, and the “2+1 Fab2-scFv-Fc” format, allows separation on the basis of size. As described above, it is also possible to “skew” the formation of heterodimers over homodimers using skew variants. Thus, a combination of heterodimerization skew variants and purification variants find particular use in the heterodimeric antibodies provided herein.

Additionally, as more fully outlined below, depending on the format of the heterodimeric antibody, purification variants either contained within the constant region and/or Fc domains of a monomer, and/or domain linkers can be used. In some embodiments, the heterodimeric antibody includes additional modifications for alternative functionalities that can also create pI changes, such as Fc, FcRn and KO variants.

In some embodiments, the subject heterodimeric antibodies provided herein include at least one monomer with one or more modifications that alter the pI of the monomer (i.e., a “pI variant”). In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Depending on the format of the heterodimer antibody, pI variants can be either contained within the constant and/or Fc domains of a monomer, or charged linkers, either domain linkers or scFv linkers, can be used. That is, antibody formats that utilize scFv(s) such as “1+1 Fab-scFv-Fc”, format can include charged scFv linkers (either positive or negative), that give a further pI boost for purification purposes. As will be appreciated by those in the art, some 1+1 Fab-scFv-Fc and 2+1 Fab2-scFv-Fc formats are useful with just charged scFv linkers and no additional pI adjustments, although the invention does provide pI variants that are on one or both of the monomers, and/or charged domain linkers as well. In addition, additional amino acid engineering for alternative functionalities may also confer pI changes, such as Fc, FcRn and KO variants.

In subject heterodimeric antibodies that utilizes pI as a separation mechanism to allow the purification of heterodimeric proteins, amino acid variants are introduced into one or both of the monomer polypeptides. That is, the pI of one of the monomers (referred to herein for simplicity as “monomer A”) can be engineered away from monomer B, or both monomer A and B change be changed, with the pI of monomer A increasing and the pI of monomer B decreasing. As is outlined more fully below, the pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (aspartic acid to lysine) or changing a charged residue to a neutral residue (e.g., loss of a charge; lysine to serine.). A number of these variants are shown in the FIG. 4.

Thus, in some embodiments, the subject heterodimeric antibody includes amino acid modifications in the constant regions that alter the isoelectric point (pI) of at least one, if not both, of the monomers of a dimeric protein to form “pI antibodies”) by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the present invention.

As will be appreciated by those in the art, the number of pI variants to be included on each or both monomer(s) to get good separation will depend in part on the starting pI of the components, for example in the 1+1 Fab-scFv-Fc, 2+1 Fab2-scFv-Fc, 1+1 CLC and 2+1 CLC formats, the starting pI of the scFv (1+1 Fab-scFv-Fc, 2+1 Fab2-scFv-Fc) and Fab(s) of interest. That is, to determine which monomer to engineer or in which “direction” (e.g., more positive or more negative), the Fv sequences of the two target antigens are calculated and a decision is made from there. As is known in the art, different Fvs will have different starting pIs which are exploited in the present invention. In general, as outlined herein, the pIs are engineered to result in a total pI difference of each monomer of at least about 0.1 logs, with 0.2 to 0.5 being preferred as outlined herein.

In the case where pI variants are used to achieve heterodimerization, by using the constant region(s) of the heavy chain(s), a more modular approach to designing and purifying bispecific proteins, including antibodies, is provided. Thus, in some embodiments, heterodimerization variants (including skew and pI heterodimerization variants) are not included in the variable regions, such that each individual antibody must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g., the minimization or avoidance of non-human residues at any particular position. Alternatively or in addition to isotypic substitutions, the possibility of immunogenicity resulting from the pI variants is significantly reduced by utilizing isosteric substitutions (e.g. Asn to Asp; and Gln to Glu).

As discussed below, a side benefit that can occur with this pI engineering is also the extension of serum half-life and increased FcRn binding. That is, as described in US Publ. App. No. US 2012/0028304 (incorporated by reference in its entirety), lowering the pI of antibody constant domains (including those found in antibodies and Fc fusions) can lead to longer serum retention in vivo. These pI variants for increased serum half-life also facilitate pI changes for purification.

In addition, it should be noted that the pI variants give an additional benefit for the analytics and quality control process of bispecific antibodies, as the ability to either eliminate, minimize and distinguish when homodimers are present is significant. Similarly, the ability to reliably test the reproducibility of the heterodimeric antibody production is important.

In general, embodiments of particular use rely on sets of variants that include skew variants, which encourage heterodimerization formation over homodimerization formation, coupled with pI variants, which increase the pI difference between the two monomers to facilitate purification of heterodimers away from homodimers.

Exemplary combinations of pI variants are shown in FIGS. 3 and 4, and FIG. 30 of US Publ. App. No. 2016/0355608, all of which are herein incorporated by reference in its entirety and specifically for the disclosure of pI variants. Preferred combinations of pI variants are shown in FIGS. 3 and 4. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)₄ (SEQ ID NO:24). However, as will be appreciated by those in the art, the first monomer includes a CHi domain, including position 208. Accordingly, in constructs that do not include a CHi domain (for example for antibodies that do not utilize a CHi domain on one of the domains), a preferred negative pI variant Fc set includes 295E/384D/418E/421D variants (Q295E/N384D/Q418E/N421D when relative to human IgG1).

Accordingly, in some embodiments, one monomer has a set of substitutions from FIG. 8 and the other monomer has a charged linker (either in the form of a charged scFv linker because that monomer comprises an scFv or a charged domain linker, as the format dictates, which can be selected from those depicted in FIG. 6).

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., an antibody provided herein may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389,392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

In some embodiments, the anti-CD28×anti-TROP2 antibody includes amino acid substitutions in one of its Fc domains that reduces binding to Protein A. Such purification variants produces heterodimers with asymmetric binding to Protein A, which can in turn be used for separation of heterodimeric from homodimeric populations by a pH gradient. Exemplary purification amino acid substitutions that reduce binding to Protein A include, but are not limited to H435R and Y436F (IgG1 CH3 domain, EU numbering). See, e.g., US2010331527, which is incorporated by reference in its entirety, and specifically for pertinent disclosures relating to Fc domain modifications to reduce Protein A binding.

### 3. Isotypic Variants

In addition, many embodiments of the subject heterodimeric antibodies rely on the “importation” of pI amino acids at particular positions from one IgG isotype into another, thus reducing or eliminating the possibility of unwanted immunogenicity being introduced into the variants. A number of these are shown in FIG. 21 of US Publ. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significant affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, etc. as is further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point.

### 4. Calculating pI

The pI of each monomer of the antibodies provided herein can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of US Pub. 2014/0370013. As discussed herein, which monomer to engineer is generally decided by the inherent pI of the Fv and scaffold regions. Alternatively, the pI of each monomer can be compared.

5. pI Variants that Also Confer Better FcRn In Vivo Binding

In the case where the pI variant decreases the pI of the monomer, the pI variant can have the added benefit of improving serum retention in vivo.

Although still under examination, Fc regions are believed to have longer half-lives in vivo, because binding to FcRn at pH 6 in an endosome sequesters the Fc (Ghetie and Ward, 1997 Immunol Today. 18(12): 592-598, entirely incorporated by reference). The endosomal compartment then recycles the Fc to the cell surface. Once the compartment opens to the extracellular space, the higher pH, ˜7.4, induces the release of Fc back into the blood. In mice, Dall' Acqua et al. showed that Fc mutants with increased FcRn binding at pH 6 and pH 7.4 actually had reduced serum concentrations and the same half-life as wild-type Fc (Dall' Acqua et al. 2002, J. Immunol. 169:5171-5180, entirely incorporated by reference). The increased affinity of Fc for FcRn at pH 7.4 is thought to forbid the release of the Fc back into the blood. Therefore, the Fc mutations that will increase Fe's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

Recently it has been suggested that antibodies with variable regions that have lower isoelectric points may also have longer serum half-lives (Igawa et al., 2010 PEDS. 23(5): 385-392, entirely incorporated by reference). However, the mechanism of this is still poorly understood. Moreover, variable regions differ from antibody to antibody. Constant region variants with reduced pI and extended half-life would provide a more modular approach to improving the pharmacokinetic properties of antibodies, as described herein.

### E. Additional Fc Variants for Additional Functionality

In addition to the heterodimerization variants discussed above, there are a number of useful Fc amino acid modification that can be made for a variety of reasons, including, but not limited to, altering binding to one or more FcγR receptors, altered binding to FcRn receptors, etc., as discussed below.

Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein.

### 1. FcγR and FcRn Variants

Accordingly, there are a number of useful Fc substitutions that can be made to alter binding to one or more of the FcγR receptors. In certain embodiments, the subject antibody includes modifications that alter the binding to one or more FcγR receptors (i.e., “FcγR variants”). Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the subject antibodies include those listed in U.S. Pat. No. 8,188,321 (particularly FIG. 41) and 8,084,582, and US Publ. App. Nos. 20060235208 and 20070148170, all of which are expressly incorporated herein by reference in their entirety and specifically for the variants disclosed therein that affect Fcγ receptor binding. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T. Such modification may be included in one or both Fc domains of the subject antibody.

In some embodiments, the subject antibody includes one or more Fc modifications that increase serum half-life. Fc substitutions that find use in increased binding to the FcRn receptor and increased serum half-life, as specifically disclosed in U.S. Ser. No. 12/341,769, hereby incorporated by reference in its entirety, including, but not limited to, 434S, 434A, 428L, 308F, 259I, 428L/434S, 259I/308F, 436I/428L, 4361 or V/434S, 436V/428L, 259I/308F/428L, and M252Y/S254T/T256E. Such modification may be included in one or both Fc domains of the subject antibody.

### 2. Ablation Variants

In some embodiments, the heterodimeric antibody includes one or more modifications that reduce or remove the normal binding of the Fc domain to one or more or all of the Fcγ receptors (e.g., FcγR1, FcγRIIa, FcγRIIb, FcγRIIIa, etc.) to avoid additional mechanisms of action. Such modifications are referred to as “FcγR ablation variants” or “Fc knock out (FcKO or KO)” variants. In these embodiments, for some therapeutic applications, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, in many embodiments, particularly in the use of bispecific antibodies that bind CD28 monovalently, it is generally desirable to ablate FcγRIIIa binding to eliminate or significantly reduce ADCC activity. In some embodiments, of the subject antibodies described herein, at least one of the Fc domains comprises one or more Fcγ receptor ablation variants. In some embodiments, of the subject antibodies described herein, both of the Fc domains comprises one or more Fcγ receptor ablation variants. These ablation variants are depicted in FIG. 5, and each can be independently and optionally included or excluded, with preferred aspects utilizing ablation variants selected from the group consisting of: L234A/L235A/D265S, G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del. It should be noted that the ablation variants referenced herein ablate FcγR binding but generally not FcRn binding.

As is known in the art, the Fc domain of human IgG1 has the highest binding to the Fcγ receptors, and thus ablation variants can be used when the constant domain (or Fc domain) in the backbone of the heterodimeric antibody is IgG1. Alternatively, or in addition to ablation variants in an IgG1 background, mutations at the glycosylation position 297 (generally to A or S) can significantly ablate binding to FeyRIIIa, for example. Human IgG2 and IgG4 have naturally reduced binding to the Fcγ receptors, and thus those backbones can be used with or without the ablation variants.

### F. Combination of Heterodimeric and Fc Variants

As will be appreciated by those in the art, all of the recited heterodimerization variants (including skew and/or purification variants) can be optionally and independently combined in any way, as long as they retain their “strandedness” or “monomer partition.” In addition, all of these variants can be combined into any of the heterodimerization formats.

In the case of pI variants, while embodiments finding particular use are shown in the figures, other combinations can be generated, following the basic rule of altering the pI difference between two monomers to facilitate purification.

In addition, any of the heterodimerization variants (skew and purification variants), are also independently and optionally combined with Fc ablation variants, Fc variants, FcRn variants, as generally outlined herein.

Exemplary combination of variants that are included in some embodiments of the heterodimeric 1+1 Fab-scFv-Fc, 2+1 mAb-Fc, and 2+1 Fab2-scFv-Fc format antibodies are included in FIG. 8. In some embodiments, the heterodimeric antibody includes a combination of variants as depicted in FIG. 8.

### G. Useful Antibody Formats

As will be appreciated by those in the art and discussed more fully below, the heterodimeric bispecific antibodies provided herein can take on several different configurations as generally depicted in FIG. 15.

As will be appreciated by those in the art, the heterodimeric formats of the invention can have different valencies as well as be bispecific. That is, heterodimeric antibodies of the invention can be bivalent and bispecific, or trivalent and bispecific, wherein the first antigen is bound by two binding domains and the second antigen by a second binding domain. As is outlined herein, when CD28 is one of the target antigens, it is preferable that the CD28 is bound only monovalently.

The present invention utilizes CD28 binding domains in combination with TROP2 binding domains. As will be appreciated by those in the art, any collection of anti-CD28 CDRs, anti-CD28 variable light and variable heavy domains, Fabs and scFvs as depicted in any of the Figures (see particularly FIGS. 16-19 and 22) or variants thereof can be used. Similarly, any of the TROP2 antigen binding domains can be used, whether CDRs, variable light and variable heavy domains, Fabs and scFvs as depicted in any of the Figures (e.g., FIGS. 23-28) or variants thereof can be used, optionally and independently combined in any combination.

### 1. 1+1 CLC Format

One heterodimeric antibody format that finds particular use in subject anti-CD28×anti-TROP2 antibodies provided herein is the “1+1 Common Light Chain” or “1+1 CLC” format, which is depicted in FIG. 15C. The 1+1 CLC format antibody includes a first monomer that includes a VH1-CH1-hinge-CH2-CH3, wherein VH1 is a first variable heavy domain and CH2-CH3 is a first Fc domain; a second monomer that includes a VH2-CH1-hinge-CH2-CH3, wherein VH2 is a second variable heavy domain and CH2-C3 is a second Fc domain; and a third monomer “common light chain” comprising VL-CL, wherein VL is a common variable light domain and CL is a constant light domain. In some embodiments, the 1+1 CLC includes two identical common light chains. In such embodiments, the VL of one of the common light chains pairs with the VH1 to form a first binding domain with a first antigen binding specificity (i.e., CD28 or TROP2); and the VL of the other common light chain pairs with the VH2 to form a second binding domain with a second antigen binding specificity (i.e., CD28 or TROP2). In some embodiments, the 1+1 CLC format antibody is a bivalent antibody.

In some embodiments, the first and second Fc domains of the 1+1 CLC format are variant Fc domains that include heterodimerization skew variants (e.g., a set of amino acid substitutions as shown in FIGS. 3 and 9). Particularly useful heterodimerization skew variants include S364K/E357Q:L 368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first or second monomer includes pI variants (including those shown in FIG. 4). In exemplary embodiments, the constant domain (CH1-hinge-CH2-CH3) of the first or second monomer includes pI variants N208D/Q295E/N384D/Q418E/N421D, wherein numbering is according to EU numbering.

In some embodiments, the 1+1 CLC format antibody provided herein includes FcRn variants M428L/N434S, wherein numbering is according to EU numbering.

In exemplary embodiments, the first variant Fc domain includes heterodimerization skew variants S364K/E357Q and the second variant Fc domain includes heterodimerization skew variants L368D/K370S; each of the first and second variant Fc domains include ablation variants E233P/L234V/L235A/G236/S267K; and the constant domain (CH1-hinge-CH2-CH3) of the second monomer includes pI variants N208D/Q295E/N384D/Q418E/N421D, wherein numbering is according to EU numbering.

In some embodiments, the CH1-hinge-CH2-CH3 of the second monomer comprises amino acid variants L368D/K370S/N208D/Q295E/N384D/Q418E/N421D/E233P/L234V/L235A/G236del/S267K, and the first Fc domain comprises amino acid variants S364K/E357Q/E233P/L234V/L235A/G236del/S267K, wherein numbering is according to EU numbering.

In some embodiments, the 1+1 CLC format antibody provided herein further includes FcRn variants M428L/N434S, wherein numbering is according to EU numbering.

In some embodiments, one of the first binding domain or the second binding domain binds CD28 and the other binding domain binds TROP2. Any suitable CD28 binding domain and TROP2 domain can be included in subject 1+1 CLC format antibody, including any of the CD28 binding domains and TROP2 binding domains provided herein or a variant thereof.

In some embodiments, the CD28 binding domain includes the vhCDR1-3 of one of the CD28 variable heavy domains depicted in FIG. 34, and the vlCDR1-3 of the 1F11-1A3.315 L1 variable light domain (see FIG. 36A). In some embodiments, the CD28 binding domain includes the vhCDR1-3 of one of the following CD28 variable heavy domains: 1A7[CD28]_H1.129, and 1A7[CD28]_H1.106 (FIG. 34), and the vlCDR1-3 of the 1F11-1A3.315 L1 variable light domain (see FIG. 36A). In another exemplary embodiment, the CD28 ABD of the subject anti-CD28×anti-TROP2 antibody includes the variable heavy (VH) domain of one of the CD28 variable heavy domains depicted in FIG. 34 or a variant thereof, and the 1F11-1A3.315 L1 variable light domain (see FIG. 36A) or a variant thereof. In some embodiments, the CD28 binding domain includes the variable heavy domain of one of the following CD28 variable heavy domains: 1A7[CD28]_H1.129, and 1A7[CD28]_H1.106 (FIG. 34) or a variant thereof, and the 1F11-1A3.315 L1 variable light domain (see FIG. 36A) or a variant thereof.

In some embodiments, the TROP2 binding domain includes the vhCDR1-3 of one of the TROP2 variable heavy domains depicted in FIGS. 36 and 52-56, and the vlCDR1-3 of the 1F11-1A3.315 L1 variable light domain (see FIG. 36A). In some embodiments, the TROP2 binding domain includes the vhCDR1-3 of one of the following TROP2 variable heavy domains: 1F11-IA3.315[Trop2]_H1, 1F11-1A3.315[Trop2]_H1.10, 1F11-1A3.315[Trop2]_H1.19, 2C5A3.316[TROP2]_H1, 1A3A4.312[TROP2]_H1, 1B2A4.312[TROP2]H1, 1B11A3.316[TROP2]_H1, and 1C9A4.313[TROP2]_H1 (FIGS. 36, and 52-56), and the vlCDR1-3 of the 1F11-1A3.315 L1 variable light domain (see FIG. 36A). IIn another exemplary embodiment, the TROP2 binding domain of the subject anti-CD28×anti-TROP2 antibody includes the variable heavy (VH) domain of one of the TROP2 variable heavy domains depicted in FIG. 36 or a variant thereof, and the 1F11-1A3.315 L1 variable light domain (see FIG. 36A) or a variant thereof. In some embodiments, the TROP2 binding domain includes the variable heavy domain of one of the following TROP2 variable heavy domains: 1F11-1A3.315[Trop2]_H1, 1F11-1A3.315[Trop2]_H1.1, 1F11-1A3.315[Trop2]_H1.19, 2C5A3.316[TROP2]_H1, 1A3A4.312[TROP2]_H1, 1B2A4.312[TROP2]_H1, 1B11A3.316[TROP2]_H1, and 1C9A4.313[TROP2]H1 (FIGS. 36, and 52-56) or a variant thereof, and the 1F11-1A3.315 L1 variable light domain (see FIG. 36A) or a variant thereof.

Exemplary subject anti-CD28×anti-TROP2 antibodies in the 1+1 CLC format are depicted in FIG. 38.

### 2. 2+1 CLC Format

Another heterodimeric antibody format that finds particular use in subject CD28×anti-TROP2 antibodies provided herein is the “2+1 Common Light Chain” or “2+1 CLC” format, which is depicted in FIG. 15D. The 2+1 CLC format includes a first monomer that includes a VH1-CH1-linker-VH1-CH1-hinge-CH2-CH3, wherein the VH1s are each a first variable heavy domain and CH2-CH3 is a first Fc domain; a second monomer that includes a VH2-CH1-hinge-CH2-CH3, wherein VH2 is a second variable heavy domain and CH2-CH3 is a second Fc domain; and a third monomer that includes a “common light chain” VL-CL, wherein VL is a common variable light domain and CL is a constant light domain. In some embodiments, the 2+1 CLC format includes three identical common light chains (i.e., a first, second, and third common light chain). In such embodiments, the VLs of the first and second common light chains each pairs with one of the VHis of the first monomer to form two first binding domains, each with a first antigen binding specificity; and the VL of the third common light chain pairs with the VH2 to form a second binding domain with a second antigen binding specificity. The linker of the first monomer can be any suitable linker, including any one of the domain linkers or combinations thereof described in FIG. 7. In some embodiments, the 2+1 CLC format antibody is a trivalent antibody.

In some embodiments, the first and second Fc domains of the 2+1 CLC format are variant Fc domains that include heterodimerization skew variants (e.g., a set of amino acid substitutions as shown in FIGS. 3 and 9). Particularly useful heterodimerization skew variants include S364K/E357Q:L 368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first or second monomer includes pI variants (including those shown in FIG. 4). In exemplary embodiments, the constant domain (CH1-hinge-CH2-CH3) of the first or second monomer includes pI variants N208D/Q295E/N384D/Q418E/N421D, wherein numbering is according to EU numbering.

In some embodiments, the 2+1 CLC format antibody provided herein further includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236/S267K; and the constant domain (CH1-hinge-CH2-CH3) of the first monomer includes pI variants N208D/Q295E/N384D/Q418E/N421D, wherein numbering is according to EU numbering. In some embodiments, the 2+1 CLC format antibody provided herein further includes FcRn variants M428L/N434S, wherein numbering is according to EU numbering.

In some embodiments, the CH1-hinge-CH2-CH3 of the second monomer comprises amino acid variants L368D/K370S/N208D/Q295E/N384D/Q418E/N421D/E233P/L234V/L235A/G236del/S267K, and the first Fc domain comprises amino acid variants S364K/E357Q/E233P/L234V/L235A/G236del/S267K, wherein numbering is according to EU numbering.

In some embodiments, each of the two first binding domains binds a tumor TROP2 and the second binding domain binds CD28. Any suitable CD28 binding domain and TROP2 domain can be included in the subject 2+1 CLC format antibody, including any of the CD28 binding domains and TROP2 binding domains provided herein or a variant thereof.

In some embodiments, the CD28 binding domain includes the vhCDR1-3 of one of the CD28 variable heavy domains depicted in FIG. 34, and the vlCDR1-3 of the 1F11-1A3.315 L1 variable light domain (see FIG. 36A). In some embodiments, the CD28 binding domain includes the vhCDR1-3 of one of the following CD28 variable heavy domains: 1A7[CD28]_H1.129, and 1A7[CD28]_H1.106 (FIG. 34), and the vlCDR1-3 of the 1F11-1A3.315 L1 variable light domain (see FIG. 36A). In another exemplary embodiment, the CD28 ABD of the subject anti-CD28×anti-TROP2 antibody includes the variable heavy (VH) domain of one of the CD28 variable heavy domains depicted in FIG. 34 or a variant thereof, and the 1F11-1A3.315 L1 variable light domain (see FIG. 36A) or a variant thereof. In some embodiments, the CD28 binding domain includes the variable heavy domain of one of the following CD28 variable heavy domains: 1A7[CD28]_H1.129, and 1A7[CD28]_H1.106 (FIG. 34) or a variant thereof, and the 1F11-1A3.315 L1 variable light domain (see FIG. 36A) or a variant thereof.

In some embodiments, the TROP2 binding domains include the vhCDR1-3 of one of the TROP2 variable heavy domains depicted in FIGS. 36 and 52-56, and the vlCDR1-3 of the 1F11-1A3.315 L1 variable light domain (see FIG. 36A). In some embodiments, the TROP2 binding domains each include the vhCDR1-3 of one of the following TROP2 variable heavy domains: 1F11-1A3.315[Trop2]_H1, and 1F11-1A3.315[Trop2]_H1.19, 2C5A3.316[TROP2]_H1, 1A3A4.312[TROP2]_H1, 1B2A4.312[TROP2]H1, 1B11A3.316[TROP2]_H1, and 1C9A4.313[TROP2]_H1 (FIGS. 36, and 52-56), and the vlCDR1-3 of the 1F11-1A3.315 L1 variable light domain (see FIG. 36A). In another exemplary embodiment, the TROP2 binding domains of the subject anti-CD28×anti-TROP2 antibody each include the variable heavy (VH) domain of one of the TROP2 variable heavy domains depicted in FIGS. 36 and 52-56 or a variant thereof, and the 1F11-IA3.315 L1 variable light domain (see FIG. 36A) or a variant thereof. In some embodiments, the TROP2 binding domains each include the variable heavy domain of one of the following TROP2 variable heavy domains: 1F11-1A3.315[Trop2]_H1, and 1F11-1A3.315[Trop2]_H1.19, 2C5A3.316[TROP2]_H1, 1A3A4.312[TROP2]_H1, 1B2A4.312[TROP2]H1, 1B11A3.316[TROP2]_H1, and IC9A4.313[TROP2]_H1 (FIGS. 36, and 52-56) or a variant thereof, and the 1F11-1A3.315 L1 variable light domain (see FIG. 36A) or a variant thereof.

Exemplary subject anti-CD28×anti-TROP2 antibodies in the 2+1 CLC format are depicted in FIG. 39.

### 3. 1+1 Fab-scFv-Fc Format

One heterodimeric antibody format that finds particular use in subject anti-CD28×anti-TROP2 antibodies provided herein is the “1+1 Fab-scFv-Fc” or “bottle opener” format as shown in FIG. 15A. The 1+1 Fab-scFv-Fc format antibody includes a first monomer that is a “regular” heavy chain (VH1-CH1-hinge-CH2-CH3), wherein VH1 is a first variable heavy domain and CH2-CH3 is a first Fc domain. The 1+1 Fab-scFv-Fc also includes a light chain that includes a first variable light domain VL1 and a constant light domain CL. The light chain interacts with the VH1-CH1 of the first monomer to form a first antigen binding domain that is a Fab. The second monomer of the antibody includes a second binding domain that is a single chain Fv (“scFv” as defined below) and a second Fc domain. The scFv includes a second variable heavy domain (VH2) and a second variable light domain (VL2), wherein the VH2 is attached to the VL2 using an scFv linker that can be charged (see, e.g., FIG. 6). The scFv is attached to the heavy chain using a domain linker (see, e.g., FIG. 7). The two monomers are brought together by the use of amino acid variants (e.g., heterodimerization variants, discussed above) in the constant regions (e.g., the Fc domain, the CHi domain and/or the hinge region) that promote the formation of heterodimeric antibodies as is described more fully below. This structure is sometimes referred to herein as the “bottle-opener” format, due to a rough visual similarity to a bottle-opener. In some embodiments, the 1+1 Fab-scFv-Fc format antibody is a bivalent antibody.

There are several distinct advantages to the present “1+1 Fab-scFv-Fc” format. As is known in the art, antibody analogs relying on two scFv constructs often have stability and aggregation problems, which can be alleviated in the present invention by the addition of a “regular” heavy and light chain pairing. In addition, as opposed to formats that rely on two heavy chains and two light chains, there is no issue with the incorrect pairing of heavy and light chains (e.g., heavy 1 pairing with light 2, etc.).

In some embodiments of the 1+1 Fab-scFv-Fc format antibody, one of the first or second antigen binding domain is a CD28 binding domain and the other binding domain is a TROP2 binding domain. In some embodiments where the 1+1 Fab-scFv-Fc, it is the scFv that binds to the CD28, and the Fab that binds TROP2. An exemplary anti-CD28×anti-TROP2 bispecific antibodies in the 1+1 Fab-scFv-Fc format is depicted in FIG. 30.

In some embodiments, the first and second Fc domains of the 1+1 Fab-scFv-Fc format antibody are variant Fc domains that include heterodimerization skew variants (e.g., a set of amino acid substitutions as shown in FIGS. 3 and 9). Particularly useful heterodimerization skew variants include S364K/E357Q:L368D/K370S; L368D/K370S:S364K; L368E/K370S:S364K; T411T/E360E/Q362E:D 401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIGS. 3 and 4). In exemplary embodiments, the constant domain (CH1-hinge-CH2-CH3) of the first monomer includes pI variants N208D/Q295E/N384D/Q418E/N421D, wherein numbering is according to EU numbering.

In exemplary embodiments, the 1+1 Fab-scFv-Fc format antibody includes a combination of amino acid modifications as depicted in FIG. 8. In such embodiments, the CH1-hinge-CH2-CH3 of the first monomer comprises amino acid variants L368D/K370S/N208D/Q295E/N384D/Q418E/N421D/E233P/L234V/L235A/G236del/S267K, the second Fc domain comprises amino acid variants S364K/E357Q/E233P/L234V/L235A/G236del/S267K, wherein numbering is according to EU numbering.

In some embodiments, the scFv of the 1+1 Fab-scFv-Fc format antibody provided herein includes a charged scFv linker (including those shown in FIG. 6). In some embodiments, the 1+1 Fab-scFv-Fc format antibody provided herein includes FcRn variants M428L/N434S, wherein numbering is according to EU numbering.

In exemplary embodiments 1+1 Fab-scFv-Fc format antibody, the first Fc domain includes heterodimerization skew variants L368D/K370S and the second Fc domain includes heterodimerization skew variants S364K/E357Q; each of the first and second Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 1+1 Fab-scFv-Fc format antibody provided herein includes a (GKPGS)₄ charged scFv linker (SEQ ID NO:24). In some embodiments, the 1+1 Fab-scFv-Fc format antibody provided herein includes FcRn variants M428L/N434S, wherein numbering is according to EU numbering. In some embodiments, the scFv of the 1+1 Fab-scFv-Fc format antibody provided herein includes a charged scFv linker (including those shown in FIG. 6).

Any suitable CD28 binding domain can be included in subject 1+1 Fab-scFv-Fc format antibody, including any of the CD28 binding domains provided herein. In some embodiments, the CD28 binding domain is one of the following CD28 binding domains or a variant thereof: 1A7[CD28]_H1L1, 1A7[CD28]_H1.1_L1, 1A7[CD28]_H1_L1.71, 1A7[CD28]_H1.1_L1.71, 1A7[CD28]_H1.14_L1, IA7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]_H4L2 (FIGS. 16, 19 and 22). Additional VH and VL sequences of exemplary CD28 binding domains that can be used in the subject 1+1 Fab-scFv-Fc format antibody are depicted in FIGS. 16, 17, 18, 34 and 36.

In some embodiments of the 1+1 Fab-scFv-Fc format, the anti-CD28 ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 16, 19 and 22 or a variant thereof; and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 16, 19 and 22 or a variant thereof;
(i) a VH comprising a vhCDR1, a vhCDR2, and a vhCDR3 having an amino acid sequence of a vhCDR1, a vhCDR2, and a vhCDR3, respectively, of a VH depicted in FIG. 16, 17, or 34 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIG. 16, 18, or 36 or a variant thereof; or
(i) a VH comprising a vhCDR1, a vhCDR2, and a vhCDR3 having an amino acid sequence of a vhCDR1, a vhCDR2, and a vhCDR3, respectively, of a VH depicted in FIG. 34 or a variant thereof; and (ii) a VL comprising a vlCDR1, a vlCDR2, and a vlCDR3 having an amino acid sequence of a vlCDR1, a vlCDR2, and a vlCDR3, respectively, of the 1F11-1A3.315 L1 variable light domain (see FIG. 36A) or a variant thereof.

In some embodiments of the 1+1 Fab-scFv-Fc format, the anti-CD28 ABD has a VH and VL domain selected from the following:
(i) a VH having an amino acid sequence of a VH of any of the CD28 binding domains depicted in FIGS. 16, 19 and 22 or a variant thereof; and (ii) a VL having an amino acid sequence of the VL of the CD28 binding domain depicted in FIGS. 16, 19 and 22 or a variant thereof;
(i) a VH having an amino acid sequence of a VH depicted in FIG. 16, 17, or 34 or a variant thereof; and (ii) a VL having an amino acid sequence of the VL of a VL depicted in FIG. 16, 18, or 36 or a variant thereof; or
(i) a VH having an amino acid sequence of a VH depicted in FIG. 34 or a variant thereof; and (ii) a VL having an amino acid sequence of the 1F11-1A3.315 L1 variable light domain (see FIG. 36A) or a variant thereof.

Any suitable TROP2 binding domain can be included in subject 1+1 Fab-scFv-Fc format antibody, including any of the TROP2 binding domains provided herein. Exemplary TROP2 binding domains that can be used in the subject 1+1 Fab-scFv-Fc format antibody are depicted in FIG. 23. Additional VH and VL sequences of exemplary TROP2 binding domains that can be used in the subject 1+1 Fab-scFv-Fc format antibody are depicted in FIGS. 24-28, 36, ad 52-56.

In some embodiments of the 1+1 Fab-scFv-Fc format, the anti-TROP2 ABD has a VH and VL domain selected from the following:
(i) a VH having an amino acid sequence of a VH of any one of the TROP2 binding domains depicted in FIG. 23 or a variant thereof; and (ii) a VL having an amino acid sequence of the VL of the TROP2 binding domain depicted in FIG. 23 or a variant thereof;
(i) a VH having an amino acid sequence of a VH depicted in FIG. 24-28 or a variant thereof; and (ii) a VL having an amino acid sequence of the VL of a VL depicted in FIG. 24-28 or a variant thereof; or
(i) a VH having an amino acid sequence of a VH depicted in FIG. 36 or a variant thereof; and (ii) a VL having an amino acid sequence of the 1F11-1A3.315 L1 variable light domain (see FIG. 36A) or a variant thereof.

FIG. 10 shows some exemplary Fc domain sequences that are useful in the 1+1 Fab-scFv-Fc format antibodies. The “monomer 1” sequences depicted in FIG. 10 typically refer to the Fc domain of the “Fab-Fc heavy chain” and the “monomer 2” sequences refer to the Fc domain of the “scFv-Fc heavy chain.” In addition, FIGS. 12 and 13 provide exemplary CHi-hinge domains, CHi domains, and hinge domains that can be included in the first or second monomer of the 1+1 Fab-scFv-Fc format. Further, FIG. 14 provides useful CL sequences that can be used with this format.

4. 2+1 mAb-scFv Format

One heterodimeric antibody format that finds particular use in the subject bispecific anti-CD28×anti-TROP2 antibodies is the 2+1 mAb-scFv format shown in FIG. 15E. This antibody format includes three antigen binding domains: two Fab portions and an scFv that is attached to the C-terminal of one of the heavy chains. In some embodiments of this format, the Fab portions each bind TROP2, in this case, human TROP2 and the “extra” scFv domain binds CD28. That is, this mAb-scFv format is a trivalent antibody.

In these embodiments, the first chain or monomer comprises, from N- to C-terminal, VH1-CH1-hinge-CH2-CH3, the second monomer comprises, from N- to C-terminal, VH1-CH1-hinge-CH2-CH3-domain linker-scFv domain, where the scFv domain comprises a second VH (VH2), a second VL (VL2) and a scFv linker. The antibody also includes two light chains that each include a VL1-CL, wherein VL1 is a first variable light domain. The scFv domain comprises a second VH (VH2), a second VL (VL2) and a scFv linker. In some embodiments, the VH1 of the first monomer and the VL1 of one of the two light chains, and the VH1 of the second monomer and the VL1 of the other of the two light chains each form a first antigen binding domain (ABD). The VH2 and VL2 form a second ABD. In some embodiments, the first ABDs each bind to human TROP2, and the second ABD binds human CD28.

As for all the scFv domains herein, the scFv domain can be in either orientation, from N- to C-terminal, VH2-scFv linker-VL2 or VL2-scFv linker-VH2. Accordingly, the second monomer may comprise, from N- to C-terminal, VH1-CH1-hinge-CH2-CH3-domain linker-VH2-scFv linker-VL2 or VH1-CH1-hinge-CH2-CH3-domain linker-VL2-scFv linker-VH2.

In some embodiments, the first and second Fc domains of the 2+1 mAb-scFv format antibody are variant Fc domains that include heterodimerization skew variants (e.g., a set of amino acid substitutions as shown in FIGS. 3 and 9). Particularly useful heterodimerization skew variants include S364K/E357Q:L368D/K370S; L368D/K370S:S364K; L368E/K370S:S364K; T411T/E360E/Q362E:D 401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fe domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 4). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 mAb-scFv format antibody provided herein includes a charged scFv linker (including those shown in FIG. 6). In some embodiments, the 2+1 mAb-scFv format antibody provided herein includes FcRn variants M428L/N434S, wherein numbering is according to EU numbering.

In exemplary embodiments the 2+1 mAb-scFv format antibody includes a combination of amino acid modifications as depicted in FIG. 8. In such embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 mAb-scFv format antibody provided herein includes a (GKPGS)₄ charged scFv linker (SEQ ID NO:24). In some embodiments, 2+1 mAb-scFv format antibody provided herein includes FcRn variants M428L/N434S, wherein numbering is according to EU numbering.

In some embodiments, the scFv of the second monomer of the 2+1 mAb-scFv format antibody is a CD28 binding and the VH1 of the first and second monomer and the VL1 of the common light chain each form binding domains that bind TROP2. Any suitable TROP2 binding domain and CD28 binding domain can be included in the 2+1 mAb-scFv format antibody, including any of the TROP2 binding domains and CD28 binding domains and related VHs and VLs provided herein or a variant thereof (see, e.g., FIGS. 16-19, 22-28, 34, 36, and 52-56).

In some embodiments of the 2+1 mAb-scFv format, the anti-CD28 ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 16, 19 and 22 or a variant thereof; and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 16, 19 and 22 or a variant thereof; or
(i) a VH comprising a vhCDR1, a vhCDR2, and a vhCDR3 having an amino acid sequence of a vhCDR1, a vhCDR2, and a vhCDR3, respectively, of a VH depicted in FIG. 16, 17, or 34 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIG. 16, 18, or 36 or a variant thereof.

In some embodiments of the 2+1 mAb-scFv format, the anti-CD28 ABD has a VH and VL domain selected from the following:
(i) a VH having an amino acid sequence of a VH of any of the CD28 binding domains depicted in FIGS. 16, 19 and 22 or a variant thereof; and (ii) a VL having an amino acid sequence of the VL of the CD28 binding domain depicted in FIGS. 16, 19 and 22 or a variant thereof; or
(i) a VH having an amino acid sequence of a VH depicted in FIG. 16, 17, or 34 or a variant thereof; and (ii) a VL having an amino acid sequence of the VL of a VL depicted in FIG. 16, 18, or 36 or a variant thereof.

In some embodiments of the 2+1 mAb-scFv format, each of the anti-TROP2 ABDs has a VH and VL domain selected from the following:
(i) a VH having an amino acid sequence of a VH of any one of the TROP2 binding domains depicted in FIG. 23 or a variant thereof; and (ii) a VL having an amino acid sequence of the VL of the TROP2 binding domain depicted in FIG. 23 or a variant thereof; or
(i) a VH having an amino acid sequence of a VH depicted in FIG. 24-28 or a variant thereof; and (ii) a VL having an amino acid sequence of the VL of a VL depicted in FIG. 24-28 or a variant thereof.

FIG. 11 shows some exemplary Fc domain sequences that are useful in the 2+1 mAb-scFv format antibodies. In addition, FIGS. 12 and 13 provide exemplary CHi-hinge domains, CHi domains, and hinge domains that can be included in the first or second monomer of the 2+1 mAb-scFv format. Further, FIG. 14 provides useful CL sequences that can be used with this format.

An exemplary anti-CD28×anti-TROP2 antibody in the 2+1 mAb-scFv format is depicted in FIG. 31.

### 5. 2+1 Fab2-scFv-Fc Format

One heterodimeric antibody format that finds particular use in subject anti-CD28×anti-TROP2 antibodies provided herein is the 2+1 Fab2-scFv-Fc format (also referred to as “central-scFv format”) shown in FIG. 15B. This antibody format includes three antigen binding domains: two Fab portions and an scFv that is inserted between the VH-CH1 and CH2-CH3 regions of one of the monomers. In some embodiments of this format, the Fab portions each bind TROP2 and the “extra” scFv domain binds CD28. In some embodiments, the 2+1 Fab2-scFv-Fc format antibody is a trivalent antibody.

In some embodiments of the 2+1 Fab2-scFv-Fc format, a first monomer includes a standard heavy chain (i.e., VH1-CH1-hinge-CH2-CH3), wherein VH1 is a first variable heavy domain and CH2-CH3 is a first Fc domain. A second monomer includes another first variable heavy domain (VH1), a CH1 domain (and optional hinge), a second Fc domain, and an scFv that includes an scFv variable light domain (VL2), an scFv linker and a scFv variable heavy domain (VH2). The scFv is covalently attached between the C-terminus of the CH1 domain of the second monomer and the N-terminus of the second Fc domain using optional domain linkers (VH1-CH1-[optional linker]-VH2-scFv linker-VH2-[optional linker]-CH2-CH3, or the opposite orientation for the scFv, VH1-CH1-[optional linker]-VL2-scFv linker-VH2-[optional linker]-CH2-CH3). The optional linkers can be any suitable peptide linkers, including, for example, the domain linkers included in FIG. 7. This embodiment further utilizes a first and second common light chain that each include a first variable light domain (VL1) and a constant light domain (CL). The VH1-CH1 of the first and second monomers each interacts with the one of the two common light chains to form two identical Fabs. In some embodiments, the identical Fabs are TROP2 binding domains and the scFv is a CD28 binding domain. As for many of the embodiments herein, these constructs can include skew variants, pI variants, ablation variants, additional Fc variants, etc. as desired and described herein.

In some embodiments, the first and second Fc domains of the 2+1 Fab2-scFv-Fc format antibody are variant Fc domains that include heterodimerization skew variants (e.g., a set of amino acid substitutions as shown in FIGS. 3 and 9). Particularly useful heterodimerization skew variants include S364K/E357Q:L368D/K370S; L368D/K370S:S364K; L368E/K370S:S364K; T411T/E360E/Q362E:D 401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 4). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 Fab2-scFv-Fc format antibody provided herein includes a charged scFv linker (including those shown in FIG. 6). In some embodiments, the 2+1 Fab2-scFv-Fc format antibody provided herein includes FcRn variants M428L/N434S, wherein numbering is according to EU numbering.

In exemplary embodiments 2+1 Fab2-scFv-Fc format antibody includes a combination of amino acid modifications as depicted in FIG. 8. In such embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 Fab2-scFv-Fc format antibody provided herein includes a (GKPGS)₄ charged scFv linker (SEQ ID NO:24). In some embodiments, the 2+1 Fab2-scFv-Fc format antibody provided herein includes FcRn variants M428L/N434S, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/S267K, and the second Fc domain comprises amino acid variants S364K/E357Q/E233P/L234V/L235A/G236del/S267K, wherein numbering is according to EU numbering.

In some embodiments, the scFv of the second monomer of the 2+1 Fab2-scFv-Fc format antibody is a CD28 binding and the VH1 of the first and second monomer and the VL1 of the common light chain each form binding domains that bind TROP2. Any suitable CD28 binding domain can be included in subject 2+1 Fab2-scFv-Fc format antibody, including any of the CD28 binding domains provided herein. In some embodiments, the CD28 binding domain is one of the following CD28 binding domains or a variant thereof: 1A7[CD28]_H1L1, 1A7[CD28]_H1.1_L1, 1A7[CD28]_H1_L1.71, IA7[CD28]_H1.1_L1.71, 1A7[CD28]_H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]H4L2 (FIGS. 16, 19 and 22).

In some embodiments of the 2+1 Fab2-scFv-Fc format, the anti-CD28 ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 16, 19 and 22 or a variant thereof; and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 16, 19 and 22 or a variant thereof; or
(i) a VH comprising a vhCDR1, a vhCDR2, and a vhCDR3 having an amino acid sequence of a vhCDR1, a vhCDR2, and a vhCDR3, respectively, of a VH depicted in FIG. 16, 17, or 34 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIG. 16, 18, or 36 or a variant thereof.

In some embodiments of the 1+1 Fab-scFv-Fc format, the anti-CD28 ABD has a VH and VL domain selected from the following:
(i) a VH having an amino acid sequence of a VH of any of the CD28 binding domains depicted in FIGS. 16, 19 and 22 or a variant thereof; and (ii) a VL having an amino acid sequence of the VL of the CD28 binding domain depicted in FIGS. 16, 19 and 22 or a variant thereof; or
(i) a VH having an amino acid sequence of a VH depicted in FIG. 16, 17, or 34 or a variant thereof; and (ii) a VL having an amino acid sequence of the VL of a VL depicted in FIG. 16, 18, or 36 or a variant thereof.

In some embodiments, the VH1 of the first and second monomer and the VL1 of the common light chain of the 2+1 Fab2-scFv-Fc format antibody each form a binding domain that binds TROP2. Any suitable TROP2 binding domain can be included in subject 2+1 Fab2-scFv-Fc format antibody, including any of the TROP2 binding domains provided herein. Exemplary TROP2 binding domains that can be used in the subject 2+1 Fab2-scFv-Fc format antibody are depicted in FIG. 23. Additional VH and VL sequences of exemplary TROP2 binding domains that can be used in the subject 2+1 Fab2-scFv-Fc format antibody are depicted in FIGS. 24-28, 36, and 52-56.

In some embodiments of 2+1 Fab2-scFv-Fc format, the anti-TROP2 ABDs each include a VH and VL domain selected from the following:
(i) a VH having an amino acid sequence of a VH of any one of the TROP2 binding domains depicted in FIG. 23 or a variant thereof; and (ii) a VL having an amino acid sequence of the VL of the TROP2 binding domain depicted in FIG. 23 or a variant thereof; or
(i) a VH having an amino acid sequence of a VH depicted in FIG. 24-28 or a variant thereof; and (ii) a VL having an amino acid sequence of the VL of a VL depicted in FIG. 24-28 or a variant thereof.

FIG. 10 shows some exemplary Fc domain sequences that are useful in the 2+1 Fab2-scFv-Fc format antibodies. In addition, FIGS. 12 and 13 provide exemplary CH1-hinge domains, CH1 domains, and hinge domains that can be included in the first or second monomer of the 2+1 Fab2-scFv-Fc format. Further, FIG. 14 provides useful CL sequences that can be used with this format.

### 6. 2+1 Fab2-Fc×scFv-Fc Format

One heterodimeric antibody format that finds particular use in the subject anti-anti-CD28×anti-TROP2 antibodies provided herein is the 2+1 Fab2-Fc×scFv-Fc format (also referred to as “stacked bottle opener”) shown in FIG. 15N. This format includes a first monomer, a second monomer and two common light chains. The first monomer includes, from N- to C-terminus: a VH1-CH1-domain linker-VH1-CH1-hinge-CH2-CH3, wherein the VH1s are each a first variable heavy domain and the CH2-CH3 is a first variant Fc domain. The domain linker can be any useful domain linker (see, e.g., FIG. 7). The second monomer includes a single-chain Fv (“scFv”) covalently attached to a second variant Fc domain by a domain linker (scFv-domain linker-CH2-CH3). The two common light chains each include a VL1-CL wherein the VL1s are each a first variable light domain. The scFv of the second monomer includes a second variable heavy domain (VH2) attached to a second variable light domain (VL2) by an scFv linker. In this embodiment, the two VH1-CHls of the first monomer each interact with one of the two common light chains to form two identical first antigen binding domains, and the VH2 and VL2 form a second antigen binding domain. In some embodiments, each of the first antigen binding domains are TROP2 binding domains, and the second antigen binding domain is a CD28 binding domain. As for many of the embodiments herein, these constructs can include skew variants, pI variants, ablation variants, additional Fc variants, etc. as desired and described herein.

In some embodiments, the first and second Fc domains of the 2+1 Fab2-Fc×scFv-Fc format antibody are variant Fc domains that include heterodimerization skew variants (e.g., a set of amino acid substitutions as shown in FIGS. 3 and 9). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 4). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the 2+1 Fab2-Fc×scFv-Fc format antibody provided herein includes a charged scFv linker (including those shown in FIG. 6). In some embodiments, the 2+1 Fab2-scFv-Fc format antibody provided herein includes FcRn variants M428L/N434S, wherein numbering is according to EU numbering.

In exemplary embodiments, the 2+1 Fab2-Fc×scFv-Fc format antibody includes a combination of amino acid modifications as depicted in FIG. 8. In such embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 Fab2-scFv-Fc format antibody provided herein includes a (GKPGS)₄ charged scFv linker (SEQ ID NO:24). In some embodiments, the 2+1 Fab2-scFv-Fc format antibody provided herein includes FcRn variants M428L/N434S, wherein numbering is according to EU numbering.

In some embodiments, the CH1-hinge-CH2-CH3 of the first monomer comprises amino acid variants L368D/K370S/N208D/Q295E/N384D/Q418E/N421D/E233P/L234V/L235A/G236del/S267K, and the second Fc domain comprises amino acid variants S364K/E357Q/E233P/L234V/L235A/G236del/S267K, wherein numbering is according to EU numbering.

In some embodiments, the scFv of the second monomer of the 2+1 Fab2-Fc×scFv-Fc format antibody is a CD28 binding and the VH1 of the first and second monomer and the VL1 of the common light chain each form binding domains that bind TROP2. Any suitable CD28 binding domain can be included in subject 2+1 Fab2-scFv-Fc format antibody, including any of the CD28 binding domains provided herein. In some embodiments, the CD28 binding domain is one of the following CD28 binding domains or a variant thereof: 1A7[CD28]_H1L1, 1A7[CD28]_H1.1_L1, 1A7[CD28]_H1_L1.71, 1A7[CD28]_H1.1_L1.71, 1A7[CD28]_H1.14_L1, 1A7[CD28]_H1.14_L1.71, CD28.3[CD28]_H0L0, hCD28.3[CD28]_H1L1, 5.11A1[CD28]_H0L0, TGN1412_H1L1, 341VL34[CD28]_H1L1, 341VL36[CD28]_H1L1, 281VL4[CD28]_H1L1, HuTN228[CD28]_H1L1, PV1[CD28]_H0L0, m9.3[CD28]_H0L0, hu9.3[CD28]_H1L1, 9G2[CD28]_H0L0, 9G2[CD28]_H1L1, 2F10A3.140[CD28]_H1L1, and TN228[CD28]H4L2 (FIGS. 16, 19 and 22).

In some embodiments of the 2+1 Fab2-Fc×scFv-Fc format, the anti-CD28 ABD has a VH and VL domain selected from the following:
(i) a VH comprising a vhCDR1, a vhCDR2, and a vhCDR3 having an amino acid sequence of a vhCDR1, a vhCDR2, and a vhCDR3, respectively, of a VH of any of the CD28 binding domains depicted in FIGS. 16, 19 and 22 or a variant thereof; and (ii) a VL comprising a vlCDR1, a vlCDR2, and a vlCDR3 having an amino acid sequence of a vlCDR1, a vlCDR2, and a vlCDR3, respectively, of the VL of the CD28 binding domain depicted in FIGS. 16, 19 and 22 or a variant thereof; or
(i) a VH comprising a vhCDR1, a vhCDR2, and a vhCDR3 having an amino acid sequence of a vhCDR1, a vhCDR2, and a vhCDR3, respectively, of a VH depicted in FIG. 16, 17, or 34 or a variant thereof; and (ii) a VL comprising a vlCDR1, a vlCDR2, and a vlCDR3 having an amino acid sequence of a vlCDR1, a vlCDR2, and a vlCDR3, respectively, of a VL depicted in FIG. 16, 18, or 36 or a variant thereof.

In some embodiments of the 2+1 Fab2-Fc×scFv-Fc, the anti-CD28 ABD has a VH and VL domain selected from the following:
(i) a VH having an amino acid sequence of a VH of any of the CD28 binding domains depicted in FIGS. 16, 19 and 22 or a variant thereof; and (ii) a VL having an amino acid sequence of the VL of the CD28 binding domain depicted in FIGS. 16, 19 and 22 or a variant thereof; or
(i) a VH having an amino acid sequence of a VH depicted in FIG. 16, 17, or 34 or a variant thereof; and (ii) a VL having an amino acid sequence of the VL of a VL depicted in FIG. 16, 18, or 36 or a variant thereof.

In some embodiments, the VH1 of the first and second monomer and the VL1 of the common light chain of the 2+1 Fab2-Fc×scFv-Fc format antibody each form a binding domain that binds TROP2. Any suitable TROP2 binding domain can be included in subject 2+1 Fab2-Fc×scFv-Fc format antibody, including any of the TROP2 binding domains provided herein. Exemplary TROP2 binding domains that can be used in the subject 2+1 Fab2-Fc×scFv-Fc format antibody are depicted in FIG. 23. Additional VH and VL sequences of exemplary TROP2 binding domains that can be used in the subject 2+1 Fab2-Fc×scFv-Fc antibody are depicted in FIGS. 24-28, and 52-56.

In some embodiments of 2+1 Fab2-Fc×scFv-Fc format, the anti-TROP2 ABDs each include a VH and VL domain selected from the following:
(i) a VH having an amino acid sequence of a VH of any one of the TROP2 binding domains depicted in FIG. 23 or a variant thereof; and (ii) a VL having an amino acid sequence of the VL of the TROP2 binding domain depicted in FIG. 23 or a variant thereof; or
(i) a VH having an amino acid sequence of a VH depicted in FIG. 24-28 or a variant thereof; and (ii) a VL having an amino acid sequence of the VL of a VL depicted in FIG. 24-28 or a variant thereof.

FIG. 10 shows some exemplary Fc domain sequences that are useful in 2+1 Fab2-Fc×scFv-Fc format antibodies. In addition, FIGS. 12 and 13 provide exemplary CH1-hinge domains, CH1 domains, and hinge domains that can be included in the first or second monomer of the 2+1 Fab2-Fc×scFv-Fc format. Further, FIG. 14 provides useful CL sequences that can be used with this format.

7. Dual scFv Formats

One heterodimeric antibody format that finds particular use in the subject bispecific anti-CD28×anti-TROP2 antibodies is the dual scFv format, as are known in the art and shown in FIG. 15F. In this embodiment, the heterodimeric bispecific antibody is made up of two scFv-Fc monomers (both in either (vh-scFv linker-vl-[optional domain linker]-CH2-CH3) format or (vl-scFv linker-vh-[optional domain linker]-CH2-CH3) format, or with one monomer in one orientation and the other in the other orientation.

In this case, all ABDs are in the scFv format. Any suitable TROP2 binding domain and CD28 binding domain can be included in anti-CD28×anti-TROP2 antibody in the dual scFv format, including any of the TROP2 binding domains and CD28 binding domains provided herein.

In addition, the Fc domains of the dual scFv format comprise skew variants (e.g. a set of amino acid substitutions as shown in FIGS. 3 and 9, with particularly useful skew variants being selected from the group consisting of S364K/E357Q:L368D/K370S; L368D/K370S:S364K; L368E/K370S:S364K; T411T/E360E/Q362E:D401K; L368D/K370S:S364K/E357L, K370S:S 364K/E357Q, T366S/L368A/Y407V:T366W and T366S/L368A/Y407V/Y349C:T366W/S354C), optionally ablation variants (including those shown in FIG. 5), optionally charged scFv linkers (including those shown in FIG. 6) and the heavy chain comprises pI variants (including those shown in FIG. 4).

In some embodiments, the dual scFv format includes skew variants, pI variants, and ablation variants. Accordingly, some embodiments include formats that comprise: a) a first monomer that comprises the skew variants S364K/E357Q, the ablation variants E233P/L234V/L235A/G236del/S267K, and a scFv that binds a first antigen (VH1-scFv linker-VL1-[optional domain linker]-CH2-CH3 or VL1-scFv linker-VH1-[optional domain linker]-CH2-CH3) and b) a first monomer that comprises the skew variants L368D/K370S, the ablation variants E233P/L234V/L235A/G236del/S267K, and a scFv that binds a second antigen (VH1-scFv linker-VL1-[optional domain linker]-CH2-CH3 or VL1-scFv linker-VH1-[optional domain linker]-CH2-CH3). pI variants can be as outlined herein, but most common will be charged scFv linkers of opposite charge for each monomer. FcRn variants, particularly 428L/434S, can optionally be included.

Any suitable TROP2 binding domain and CD28 binding domain can be included in the dual scFv format, including any of the TROP2 binding domains and CD28 binding domains and related VHs and VLs provided herein or a variant thereof (see, e.g., FIGS. 16-19, 22-28, 34, 36 and 52-56).

### 8. One-Armed Central-scFv

One heterodimeric antibody format that finds particular use in subject anti-CD28×anti-TROP2 antibodies provided herein is the one-armed central-scFv format shown in FIG. 15J. In this embodiment, one monomer comprises just an Fc domain, while the other monomer includes a Fab domain (a first antigen binding domain), a scFv domain (a second antigen binding domain) and an Fc domain, where the scFv domain is inserted between the Fc domain and the Fc domain.

In this embodiment, one monomer comprises a first heavy chain comprising a first variable heavy domain, a CH1 domain and Fc domain, with a scFv comprising a scFv variable light domain, an scFv linker and a scFv variable heavy domain. The scFv is covalently attached between the C-terminus of the CH1 domain of the heavy constant domain and the N-terminus of the first Fc domain using domain linkers, in either orientation, VH1-CH1-[optional domain linker]-VH2-scFv linker-VL2-[optional domain linker]-CH2-CH3 or VH1-CH1-[optional domain linker]-VL2-scFv linker-VH2-[optional domain linker]-CH2-CH3. The second monomer comprises an Fc domain (CH2-CH3). This embodiment further utilizes a light chain comprising a variable light domain and a constant light domain, that associates with the heavy chain to form a Fab. As for many of the embodiments herein, these constructs include skew variants, pI variants, ablation variants, additional Fc variants, etc. as desired and described herein.

Any suitable TROP2 binding domain and CD28 binding domain can be included in the one-armed central-scFv format, including any of the TROP2 binding domains and CD28 binding domains and related VHs and VLs provided herein or a variant thereof (see, e.g., FIGS. 16-19, 22-28, 34, 36, and 52-56).

9. One-Armed scFv-mAb Format

One heterodimeric antibody format that finds particular use in subject anti-CD28×anti-TROP2 antibodies provided herein is the one-armed mAb-scFv format shown in FIG. 15G. This format includes: 1) a first monomer that comprises an “empty” Fc domain; 2) a second monomer that includes a first variable heavy domain (VH), a scFv domain (a second antigen binding domain) and an Fc domain, where the scFv domain is attached to the N-terminus of the first variable heavy domain; and 3) a light chain that includes a first variable light domain and a constant light domain. The first variable heavy domain and the first variable light domain form a first antigen binding domain and the scFv is a second antigen binding domain. In this format, one of the first antigen binding domain and second binding domain binds CD28, and the other antigen binding domain binds TROP2. As for many of the embodiments herein, these constructs include skew variants, pI variants, ablation variants, additional Fc variants, etc. as desired and described herein.

Any suitable TROP2 binding domain and CD28 binding domain can be included in the one-armed scFv-mAb format antibody, including any of the TROP2 binding domains and CD28 binding domains and related VHs and VLs provided herein or a variant thereof (see, e.g., FIGS. 16-19, 22-28, 34, 36, and 52-56).

10. scFv-mAb Format

One heterodimeric antibody format that finds particular use in subject anti-CD28×anti-TROP2 antibodies provided herein is the mAb-scFv format shown in FIG. 15H. In this embodiment, the format relies on the use of a N-terminal attachment of a scFv to one of the monomers, thus forming a third antigen binding domain, wherein the Fab portions of the two monomers each bind one target and the “extra” scFv domain binds a different target.

In this embodiment, the first monomer comprises a first heavy chain (comprising a variable heavy domain and a constant domain), with a N-terminally covalently attached scFv comprising a scFv variable light domain, an scFv linker and a scFv variable heavy domain in either orientation ((vhl-scFv linker-vl1-[optional domain linker]-vh2-CH1-hinge-CH2-CH3) or (with the scFv in the opposite orientation) ((vl1-scFv linker-vh1-[optional domain linker]-vh2-CH1-hinge-CH2-CH3)). The second monomer comprises a heavy chain VH2-CH1-hinge-CH2-CH3. This embodiment further utilizes a common light chain comprising a variable light domain and a constant light domain, that associates with the heavy chains to form two identical Fabs. As for many of the embodiments herein, these constructs include skew variants, pI variants, ablation variants, additional Fc variants, etc. as desired and described herein.

Any suitable TROP2 binding domain and CD28 binding domain can be included in the scFv-mAb antibody format, including any of the TROP2 binding domains and CD28 binding domains and related VHs and VLs provided herein or a variant thereof (see, e.g., FIGS. 16-19, 22-28, 34, 36, and 52-56).

11. mAb-Fv Format

One heterodimeric antibody format that finds particular use in subject anti-CD28×anti-TROP2 antibodies provided herein is the mAb-Fv format (FIG. 15K). In this embodiment, the format relies on the use of a C-terminal attachment of an “extra” variable heavy domain to one monomer and the C-terminal attachment of an “extra” variable light domain to the other monomer, thus forming a third antigen binding domain (i.e. an “extra” Fv domain), wherein the Fab portions of the two monomers bind CD28 and the “extra” Fv domain binds TROP2.

In this embodiment, the first monomer comprises a first heavy chain, comprising a first variable heavy domain and a first constant heavy domain comprising a first Fc domain, with a first variable light domain covalently attached to the C-terminus of the first Fc domain using a domain linker (vh1-CH1-hinge-CH2-CH3-[optional linker]-vl2). The second monomer comprises a second variable heavy domain, a second constant heavy domain comprising a second Fc domain, and a third variable heavy domain covalently attached to the C-terminus of the second Fc domain using a domain linker (vh1-CH1-hinge-CH2-CH3-[optional linker]-vh2. This embodiment further utilizes a common light chain comprising a variable light domain and a constant light domain, which associates with the heavy chains to form two identical Fabs that include two identical Fvs. The two C-terminally attached variable domains make up the “extra” third Fv. As for many of the embodiments herein, these constructs include skew variants, pI variants, ablation variants, additional Fc variants, etc. as desired and described herein.

Any suitable TROP2 binding domain and CD28 binding domain can be included in the mAb-Fv format, including any of the TROP2 binding domains and CD28 binding domains and related VHs and VLs provided herein or a variant thereof (see, e.g., FIGS. 16-19, 22-28, 34, 36 and 52-56).

### 12. Central-Fv Format

One heterodimeric antibody format that finds particular use in subject anti-CD28×anti-TROP2 antibodies provided herein is the central-Fv format shown in FIG. 15L. In this embodiment, the format relies on the use of an inserted Fv domain thus forming an “extra” third antigen binding domain, wherein the Fab portions of the two monomers bind TROP2 and the “extra” central-Fv domain binds CD28. The Fv domain is inserted between the Fc domain and the CH1-Fv region of the monomers, thus providing a third antigen binding domain, wherein each monomer contains a component of the Fv (e.g. one monomer comprises a variable heavy domain and the other a variable light domain of the “extra” central Fv domain).

In this embodiment, one monomer comprises a first heavy chain comprising a first variable heavy domain, a CH1 domain, and Fc domain and an additional variable light domain. The additional variable light domain is covalently attached between the C-terminus of the CH1 domain of the heavy constant domain and the N-terminus of the first Fc domain using domain linkers (vh1-CH1-[optional linker]-vl2-hinge-CH2-CH3). The other monomer comprises a first heavy chain comprising a first variable heavy domain, a CH1 domain and Fc domain and an additional variable heavy domain (vh1-CH1-[optional linker]-vh2-hinge-CH2-CH3). The additional variable heavy domain is covalently attached between the C-terminus of the CH1 domain of the heavy constant domain and the N-terminus of the first Fc domain using domain linkers. This embodiment utilizes a common light chain comprising a variable light domain and a constant light domain, that associates with the heavy chains to form two identical Fabs that each bind TROP2. The additional variable heavy domain and additional variable light domain form an “extra” central Fv that binds CD28. As for many of the embodiments herein, these constructs include skew variants, pI variants, ablation variants, additional Fc variants, etc. as desired and described herein.

Any suitable TROP2 binding domain and CD28 binding domain can be included in the central-Fv format, including any of the TROP2 binding domains and CD28 binding domains and related VHs and VLs provided herein or a variant thereof (see, e.g., FIGS. 16-19, 22-28, 34, 36, and 52-56).

### 13. Non-Heterodimeric Bispecific Antibodies

As will be appreciated by those in the art, the anti-CD28×anti-TROP2 antibodies provided herein can also be included in non-heterodimeric bispecific formats (see FIG. 15I). In this format, the anti-CD28×anti-TROP2 includes: 1) a first monomer comprising a VH1-CH1-hinge-CH2-CH3; 2) a second monomer comprising a VH2-CH1-hinge-CH2-CH3; 3) a first light chain comprising a VL1-CL; and 4) a second light chain comprising a VL2-CL. In such embodiments, the VH1 and VL1 form a first antigen binding domain and VH2 and VL2 form a second antigen binding domain. One of the first or second antigen binding domains binds CD28 and the other antigen binding domain binds TROP2.

Any suitable TROP2 binding domain and CD28 binding domain can be included in anti-CD28×anti-TROP2 antibody in the non-heterodimeric bispecific antibody format, including any of the TROP2 binding domains and CD28 binding domains and related VHs and VLs provided herein or a variant thereof (see, e.g., FIGS. 16-19, 22-28, 34, 36, and 52-56).

### 14. Trident Format

In some embodiments, the anti-CD28×anti-TROP2 antibodies provided herein are in the “Trident” format as generally described in WO2015/184203, hereby expressly incorporated by reference in its entirety and in particular for the Figures, Legends, definitions and sequences of “Heterodimer-Promoting Domains” or “HPDs”, including “K-coil” and “E-coil” sequences. Tridents rely on using two different HPDs that associate to form a heterodimeric structure as a component of the structure, see FIG. 15M. In this embodiment, the Trident format include a “traditional” heavy and light chain (e.g. VH1-CH1-hinge-CH2-CH3 and VL1-CL), a third chain comprising a first “diabody-type binding domain” or “DART®”, VH2-(linker)-VL3-HPD1 and a fourth chain comprising a second DART®, VH3-(linker)-(linker)-VL2-HPD2. The VH1 and VL1 form a first ABD, the VH2 and VL2 form a second ABD, and the VH3 and VL3 form a third ABD. In some cases, as is shown in FIG. 15M, the second and third ABDs bind the same antigen.

Any suitable TROP2 binding domain and CD28 binding domain can be included in the trident format, including any of the TROP2 binding domains and CD28 binding domains and related VHs and VLs provided herein or a variant thereof (see, e.g., FIGS. 16-19, 22-28, 34, 36, and 52-56).

### V. Nucleic Acids

In another aspect, provided herein are nucleic acid compositions encoding the anti-CD28×anti-TROP2 antibodies provided herein. A nucleic acid composition may refer to one or multiple polynucleotides.

As will be appreciated by those in the art, the nucleic acid compositions will depend on the format and scaffold of the heterodimeric protein. Thus, for example, when the format requires three amino acid sequences, such as for the 1+1 CLC, 2+1 CLC, 1+1 Fab-scFv-Fc, 2+1 mAb-scFv, or 2+1 Fab2-scFv-Fc formats, three polynucleotides can be incorporated into one or more expression vectors for expression. In exemplary embodiments, each polynucleotide is incorporated into a different expression vector.

As is known in the art, the nucleic acids encoding the components of the binding domains and antibodies disclosed herein can be incorporated into expression vectors as is known in the art, and depending on the host cells used to produce the heterodimeric antibodies of the invention. Generally the nucleic acids are operably linked to any number of regulatory elements (promoters, origin of replication, selectable markers, ribosomal binding sites, inducers, etc.). The expression vectors can be extra-chromosomal or integrating vectors.

The polynucleotides and/or expression vectors of the invention are then transformed into any number of different types of host cells as is well known in the art, including mammalian, bacterial, yeast, insect and/or fungal cells, with mammalian cells (e.g., CHO cells), finding use in many embodiments.

In some embodiments, polynucleotides encoding each monomer are each contained within a single expression vector, generally under different or the same promoter controls. In embodiments of particular use in the present invention, each of these polynucleotides are contained on different expression vectors. As shown herein and in U.S. 62/025,931, hereby incorporated by reference, different vector ratios can be used to drive heterodimer formation. That is, surprisingly, while the proteins comprise first monomer: second monomer:light chains (in the case of many of the embodiments herein that have three polypeptides comprising the heterodimeric antibody) in a 1:1:2 ratio, these are not the ratios that give the best results.

The antibodies provided herein are made by culturing host cells comprising the expression vector(s) as is well known in the art. Once produced, traditional antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the “1+1 Fab-scFv-Fc” heterodimer (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

### VI. Biological and Biochemical Functionality of the Anti-CD28×Anti-TROP2 Antibodies

Generally the anti-CD28×anti-TROP2 antibodies described herein are administered to patients with a TROP2-associated cancer, and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

### A. Antibody Compositions for In Vivo Administration

Embodiments of the invention relate to a pharmaceutical composition comprising any one of the anti-CD28×anti-TROP2 antibodies described herein and a pharmaceutically acceptable carrier. Formulations of the anti-CD28×anti-TROP2 antibodies described herein are prepared for storage by mixing an antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

### VII. Treatments

Once made, the compositions of the invention find use in a number of oncology applications, by treating cancer, generally by enhancing immune responses (e.g., T cell activation and proliferation), particularly when used with anti-cancer therapies such as anti-tumor bispecific antibodies. In some embodiments, the antibodies provided herein enhance immune responses (e.g., T cell activation and proliferation) by providing agonistic co-stimulation of T cells in the microenvironment of tumors expressing TROP2.

In some embodiments, the anti-CD28×anti-TROP2 bispecific antibodies provided herein are administered with an anti-tumor therapy including, for example, anti-tumor-associated antigen (TAA) bispecific antibodies.

### A. Anti-CD28×Anti-TROP2/Anti-TROP2 Bispecific Antibody

In some embodiments, the anti-CD28×anti-TROP2 antibodies provided herein are administered with an anti-TROP2 bispecific antibody that is a T-cell engaging bispecific antibody, such as those that bind to human CD3.

In classic T cell/APC interaction, there is a first signal provided by TCR reactivity with peptide-MHC (Signal 1) and a second signal provided by CD28 crosslinking by CD80/CD86 being expressed on APCs (Signal 2) which together fully activate T cells (see FIG. 29A). In contrast, only the first signal is provided in treatment with CD3 bispecific antibodies that target a tumor-associated antigen (TAA)(i.e., anti-CD3×anti-TROP2 bispecific antibodies).

Without being bound by any particular theory of operation, it is believed that the anti-CD28×anti-TROP2 bispecific antibodies provided herein can enhance the anti-tumor response of an anti-CD3×anti-TROP2 bispecific antibody by CD28 costimulation (see FIG. 29B). Thus, in one aspect, provided herein are methods of methods of treating a TROP2-associated cancer in a patient by administering the patient an anti-CD3×anti-TROP2 bispecific antibody and an anti-CD28×anti-TROP2 bispecific antibody provided herein.

Anti-CD3×anti-TROP2 antibodies that are useful for providing “signal 1” in combination with the subject anti-CD28×anti-TROP2 antibodies include those with any of the CD3 binding domains and TROP2 binding domains provided herein (see, e.g., FIGS. 23-28 and 32). Suitable antibody formats for such anti-CD3×anti-TROP2 antibodies, include, but are not limited to the antibody formats described herein (see, e.g., FIG. 15). In some embodiments, the anti-CD3×anti-TROP2 antibody and the anti-CD28×anti-TROP2 antibody used in combination bind to the same TROP2 epitope. In some embodiments, the anti-CD3×anti-TROP2 antibody and the anti-CD28×anti-TROP2 antibody used in combination bind to different TROP2 epitopes.

### B. Administrative Modalities

The antibodies provided herein administered to a subject, in accord with known methods, such as intravenous administration as a bolus or by continuous infusion over a period of time.

### C. Treatment Modalities

In the methods of the invention, therapy is used to provide a positive therapeutic response with respect to a disease or condition.

By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the bispecific antibodies used in the present invention depend on the disease or condition to be treated and may be determined by the persons skilled in the art.

### EXAMPLES

### Example 1: CD28 Binding Domains

Sequences for human, mouse, and cynomolgus CD28 are depicted in FIGS. 1A-1B and are useful for the development of cross-reactive CD28 antigen binding domains for ease of clinical development.

### 1A: Novel CD28 Binding Domains

An approach considered to avoid the superagonism associated with TGN1412 was to generate novel CD28 binding domains having lower affinity binding to CD28 and/or binding to a different CD28 epitope than TGN1412. In one campaign to generate such novel CD28 binding domains, in-house de novo phage libraries were panned against CD28.

### 1A(a): Phage-Derived Clone 1A7

It should be noted that this phage library utilized a human germline VL with diversity introduced into the LCDR3. The amino acid sequences for exemplary phage-derived clone 1A7 are depicted in FIG. 16.

Towards optimization of CD28 bsAbs, numerous 1A7 affinity variants were developed by engineering VH variants (illustrative sequences as depicted in FIGS. 17A-17F and as SEQ ID NOs: 117-185), VL variants (illustrative sequences as depicted in FIGS. 18A-181 and as SEQ ID NOs: 186-291), consensus sequences for which are depicted in FIGS. 20A-20B, and combinations thereof (illustrative sequences for which are depicted in FIG. 19A-19C). Monovalent affinity of illustrative variants, in the context of scFvs, are depicted in FIG. 21.

Additionally, there was a desire to utilize the 1A7 binding domain in common light chain formats. The phage library for discovering CD28 clone 1A7 utilized the same parental human germline VL as the phage library for discovering TROP2 binding domain 1F11-1A3.315 as will be described in Example 2A. Surprisingly, it was found that the variable light domain of clone 1A7 differed from the parental germline VL by only a single amino acid in the LCDR3; however, despite there being only a single amino acid difference, the VH of 1A7 does not pair well with the parental germline VL resulting in diminished binding. Accordingly, affinity-optimization library was generated with focus on substitutions only in the variable heavy domain of 1A7 (to ensure that ability of the germline VL to act as common light chain with the TROP2 binding domains from Example 2A was not perturbed). Amino acid sequences for exemplary affinity-optimized 1A7 variable domains are depicted in FIG. 34 and SEQ ID NOs: XXX-YYY. Monovalent KD of affinity-engineered 1A7 Fab variants were determined via Octet, data for which are depicted in FIG. 35. Note that the numbers may vary between experiment and batches. For example, in another set of experiments, the 1A7_H1.129_IGKV1-39_L1 domain showed affinity of 60-70 nM; while the 1A7_H1.118_IGKV1-39_L1 domain showed affinity of −400 nM. However, the trend remains the same e.g. 1A7_H1.129_IGKV1-39_L1 has tighter affinity than 1A7_H1.118_IGKV1-39_L1. Nonetheless, the KD in FIG. 35 will be used to describe the binding domains herein.

### 1B: Additional CD28 Binding Domains

Sequences for additional CD28 binding domains which may find use in the TROP2×CD28 bsAbs of the invention are depicted in FIGS. 22A-22H.

### Example 2: TROP2 Binding Domains

Sequences for human, mouse, and cynomolgus TROP2 are depicted in FIG. 2 and are useful for the development of cross-reactive TROP2 antigen binding domains for ease of clinical development.

### 2A: Novel TROP2 Binding Domains

To engineer TROP2×CD28 bsAbs, common light chain formats were explored, as bispecific antibody formats utilizing just Fab domains may be less immunogenic and stable than those utilizing scFv domains. A phage library utilizing a constant human germline VL (utilizing the same human germline VL as in Example 1A(a), except without any diversity) and diversity in the VH was used. The amino acid sequences for exemplary phage-derived TROP2 clone 1F11-1A3.315 are depicted in FIG. 36.

To tune binding affinity for TROP2 (and potencies of the bispecific molecules), an affinity ladder was engineered (illustrative sequences for which are depicted in FIG. 36). Initially, KD of affinity-engineered 1F11-1A3.315 variants were determined in a bivalent format, data for which are depicted in FIG. 37. Subsequently, monovalent KD were determined, and 1F11-1A3.315_H1L1 was 13 nM, 1F11-1A3.315_H1.10_L1 was 5 nM, and 1F11-1A3.315_H1.19_L1 was 2 nM. Although these are weaker (as these variants are monovalent), the trend is in line with bivalent i.e. H1L1<H1. 10_L1<H1.19_L1.

While the above affinity-maturation campaign sought to improve the binding affinity of the 1F11-1A3.315 clone, another avenue that was explored was weaker affinity TROP2 binding. Accordingly, additional phage-derived TROP2 clones were identified, sequences for which are depicted in FIGS. 52-56. KD were determined as follows: 1C9A4.313 (40 nM), 1B11A3.316 (54 nM), 1B2A4.312 (64 nM), 1A3A4.312 (80 nM), and 2C5A3.316 (345 nM).

### 2B: Additional TROP2 Binding Domains

Sequences for TROP2 binding domains which may find use in the TROP2×CD28 bsAbs of the invention are depicted in FIGS. 23-28.

### Example 3: Engineering TROP2×CD28 bsAbs

T cells require multiple signals for complete activation and differentiation. Signal 1, promoted by recognition of a peptide-MHC (pMHC) complex by the T cell receptor (TCR), is absolutely required for T cell activation. Signal 2, which synergizes with, and amplifies Signal 1, is typically provided by the interaction of the CD28 ligands CD80 and CD86 with CD28 itself. Although CD28 engagement alone is typically inert, when combined with Signal 1 activation, it promotes additional activation, survival, and proliferative signals, including IL2 secretion (see FIG. 29). As CD80 and CD86 are only naturally expressed by professional antigen-presenting cells (APC), the extent of CD28 costimulation in the tumor setting can be highly variable. By creating this novel class of tumor-targeted CD28 bispecific antibodies, CD80/CD86 engagement of CD28 can be mimicked, providing an artificial source of Signal 2. Notably, signal can either be provided by the natural TCR:pMHC recognition of tumor cells, or it can be provided by combination of the CD28 bispecific with a CD3 bispecific (which can mimic Signal 1). With these concepts in mind, TROP2×CD28 bsAbs were conceived, and a number of formats are contemplated for use, schematics for which are outlined in FIG. 15.

### 3A: 1+1 Fab-scFv-Fc Format

One exemplary format utilizing Fab domains and scFv is the 1+1 Fab-scFv-Fc format (depicted schematically in FIG. 15A) which comprises a first monomer comprising a single-chain Fv (“scFv”) with a first antigen binding specificity covalently attached to a first heterodimeric Fc domain i.e., scFv-domain linker-CH2-CH3, a second monomer comprising a heavy chain i.e., VH-CH1-hinge-CH2-CH3, wherein the CH2-CH3 is a second heterodimeric Fc domain complementary to the first heterodimeric Fc domain, and a light chain (LC) transfected separately so that a Fab domain having a second antigen binding specificity is formed with the variable heavy domain. Any number of heterodimerization approaches as is known in the art could find use in this (and other) bispecific formats, in combination with any number of approaches for purifying heterodimers from contaminating homodimers, including those depicted in FIG. 3. Any of the number of linkers as is known in the art may find use in linking the VH and VL domains of the scFv. Finally, it may be useful to maximize serum half-life of the bsAbs, and any of the number of half-life extending variants as is known in the art may find use in these bsAbs.

In particular, the 1+1 Fab-scFv-Fc bsAbs may utilize Backbones 1 or 11 in FIG. 10. The backbones utilize the L368D/K370S (on the HC): S364K/E357Q (on the scFv-Fc) heterodimeric Fc variants. The HC side further includes pI variants N208D/Q295E/N384D/Q418E/N421D to increase negative charge of the heavy chain. The scFv utilizes a positively charged (GKPGS)4 linker (SEQ ID NO: 24) between the VH and VL domains to increase positive charge of the scFv-Fc chain. Collectively, these two approaches enable easy purification of heterodimers from contaminating homodimers. The FcγR ablation variants utilized in this platform are the E233P/L234V/L235A/G236_/S267K substitutions on both the HC and the scFv-Fc monomers. In some cases, bsAbs includes the M428L/N434S half-life extension variants. Sequences for illustrative TROP2×CD28 bsAbs in the 1+1 Fab-scFv-Fc format are depicted in FIG. 30.

3B: 2+1 mAb-scFv Format

Another exemplary format utilizing Fab domains and scFv is the 2+1 mAb-scFv format (depicted schematically in FIG. 15E which comprises a first monomer comprising a first heavy chain covalently attached to a single-chain Fv (“scFv”) with a first antigen binding specificity i.e., VH-CH1-hinge-CH2-CH3-domain linker-scFv, wherein CH2-CH3 is a first heterodimeric Fc domain; a second monomer comprising a heavy chain i.e., VH-CH1-hinge-CH2-CH3, wherein the CH2-CH3 is a second heterodimeric Fc domain complementary to the first heterodimeric Fc domain, and a light chain (LC) transfected separately so that a Fab domain having a second antigen binding specificity is formed with the two VH domains. In particular, the 1+1 Fab-scFv-Fc bsAbs may utilize Backbones 1 or 11 in FIG. 10. The backbones utilize the L368D/K370S (on the HC): S364K/E357Q (on the HC-scFv side) heterodimeric Fc variants. The HC further includes pI variants N208D/Q295E/N384D/Q418E/N421D to increase negative charge of the heavy chain. The scFv utilizes a positively charged (GKPGS)4 linker (SEQ ID NO: 24) between the VH and VL domains to increase positive charge of the HC-scFv chain. Collectively, these two approaches enable easy purification of heterodimers from contaminating homodimers. The FcγR ablation variants utilized in this platform are the E233P/L234V/L235A/G236_/S267K substitutions on both the HC and the scFv-Fc monomers. In some cases, bsAbs includes the M428L/N434S half-life extension variants. Sequences for illustrative TROP2×CD28 bsAbs in the 2+1 mAb-scFv format are depicted in FIG. 31.

### 3C: 1+1 Common Light Chain (CLC) Format

One common light chain format is the 1+1 Common Light Chain (CLC) format (depicted schematically in FIG. 15A) which comprises a first monomer comprising VH1-CH1-hinge-CH2-CH3, a second monomer comprising VH2-CH1-hinge-CH2-CH3, and a third monomer comprising VL-CL. The VL pairs with the VH1 to form a binding domain with a first antigen binding specificity; and the VL pairs with the VH2 to form a binding domain with a second antigen binding specificity. Sequences for illustrative TROP2×CD28 bsAbs (based on binding domains as described here) in the 1+1 CLC format are depicted in FIG. 38.

### 3D: 2+1 Common Light Chain (CLC) Format

Another common light chain format is the 2+1 CLC format (depicted schematically in FIG. 15B) which comprises a first monomer comprising VH1-CH1-linker-VH1-CH1-hinge-CH2-CH3, a second monomer comprising VH2-CH1-hinge-CH2-CH3, and a third monomer comprising VL-CL. The VL pairs with the first and second VH1 to form binding domains with a first antigen binding specificity; and the VL pairs with the VH2 to form a binding domain with a second antigen binding specificity. Sequences for illustrative TROP2×CD28 bsAbs (based on binding domains as described here) in the 2+1 CLC format are depicted in FIG. 39.

Although Example 3A provides particularly useful backbones for 1+1 Fab-scFv-Fc which may be adapted for the other formats, any number of heterodimerization approaches as is known in the art could find use in the bispecific formats, in combination with any number of approaches for purifying heterodimers from contaminating homodimers, including those depicted in FIGS. 3-4. Any of the number of linkers as is known in the art may find use in linking the VH and VL domains of the scFv. Finally, it may be useful to maximize serum half-life of the bsAbs, and any of the number of half-life extending variants as is known in the art may find use in these bsAbs. Regardless of bsAb format, the CD28 bispecific antibodies are monovalent for CD28 and incorporate Fc variants engineered to ablate FcγR binding to avoid potential superagonism. Such Fc variants include those depicted in FIG. 5.

### Example 4: TROP2×CD28 bsAbs Induce Cytokine Secretion By T Cell in the Presence of a Signal

10,000 A431-ß2M-null cells (TROP2^{high}) expressing αCD3 scFv (as a surrogate for Signal 1) were plated overnight. A431 cells were then treated with a dose titration of TROP2×CD28 bsAbs or PBS control in the presence of purified T cells at an E:T ratio of 10:1 and 1:1. IL2 secretion was measured 1 day post T cell seeding using MSD assay (Meso Scale Discovery, Rockville, Md.). Data depicted in FIG. 33 show that TROP2×CD28 bsAbs having both monovalent and bivalent TROP2 binding were active in inducing cytokine secretion.

### Example 5: Combination with CD3 bsAbs

As described in Example 3, the TROP2×CD28 bsAbs are contemplated for combination with CD3 bsAbs. Such CD3 bsAbs may utilize the CD3 binding domains as depicted in FIG. 32. Additionally, the TROP2×CD28 bsAbs may be combined with TROP2×CD3 bsAbs (see e.g., U.S. Pat. Nos. 9,382,329, 10,245,321, and Liu H, Bai L, Huang L, et al. Bispecific antibody targeting TROP2×CD3 suppresses tumor growth of triple negative breast cancer. J Immunother Cancer. 2021; 9(10):e003468. doi: 10.1136/jitc-2021-003468 hereby incorporated by reference). They may also be combined with other CD3 bsAbs.

XENP44424 and XENP44505, TROP2×CD28 bsAbs based on the novel 1F11 TROP2 binding domain in the 1+1 Fab-scFv-Fc format (respectively with the CD28 scFv in VHVL or VLVH orientations) were incubated with 1 μg/ml illustrative B7H3×CD3 bsAb and the following cancer cell lines with varying cell densities at low 1:10 effector:target ratio: 647-V (TROP2high), A431 (TROP2med), MDA-MB-231 (TROP2low-med), and DU145 (TROP2low-med) to assess cytokine secretion by effector cells. Data as depicted in FIG. 41 show that both TROP2×CD28 bsAbs dose dependently combined productively with B7H3×CD3 bsAb to induce cytokine secretion. Notably, the orientation of the CD3 scFv did not affect activity. In a similar experiment, 1 μg/ml XENP44424 or XENP44505 was incubated with dose titration illustrative B7H3×CD3 bsAb. Data as depicted in Figure X is consistent with data as depicted in FIG. 41.

### Example 6: Tuning TROP2×CD28 bsAbs

TROP2×CD28 bsAbs were investigated in alternative formats, specifically 1+1 CLC and 2+1 CLC formats. In addition, in 2+1 CLC formats, the alternative orientations of the 2^{nd} TROP2 binding domain (i.e. stacked on top of the 1^{st} TROP2 binding domain or on top of the CD28 binding domain) were investigated. Additionally, the impact of tuning binding affinities of the TROP2 binding domains was investigated. Molecules embodying these various parameters were investigated in the following experiments.

In a first experiment, dose titration of TROP2×CD28 bsAbs were incubated with 1 μg/ml illustrative B7H3×CD3 bsAb and OVCAR5 (TROP2^{high}) or pp65-MDA_MB-231 cancer cells at 1:1 effector:target ratio. Data depicting IL2 secretion after 24 hours are shown in FIGS. 42-43 and 50-51 (IFNγ data not shown depict the same trend).

In a second experiment, dose titration of TROP2×CD28 bsAbs were incubated with 1 μ/ml illustrative B7H3×CD3 bsAb and DU145, MCF7, OvCAR5, and MDA-MB-231 cancer cells at 1:1 effector:target ratio. Data depicting IL2 secretion after 24 hours are shown in FIGS. 44-45. IFNγ data depicted in FIG. 48 show the same trend.

Collectively, the data show that tighter TROP2 affinity variants enabled higher potencies (for example, compare XENPs 44595, 44597, and 44599 in FIGS. 42, 44, and 50 which differ in only the TROP2 binding domain). Additionally, no TROP2 avidity effect was conveyed by the 2+1 CLC format (for example, comparing XENP44595 and XENP44601 in FIGS. 42-44). However in the 2+1 CLC format, the orientation of the 2^{nd} TROP2 binding domain did matter, as stacking the TROP2 binding domain on the CD28 binding domain as in XENP44869 and XENP44871 reduced potency (see FIGS. 42 and 50). Notably, the control RSV×CD28 bsAb XENP44623 and XENP44624 are inactive.

### Example 7: Combination with PD-1 Blockade

Checkpoint blockade (e.g. PD-1 blockade) may be a useful therapeutic modality to stack with engagement of T cell costimulatory receptors on TILs with agonistic antibodies as it would provide broad utility in solid tumors and circumvent CTLA4 inhibition of CD28 pathway. Accordingly, the combination of TROP2×CD28 bispecific antibody XENP44880 with XENP16432 (a bivalent anti-PD-1 mAb based on the variable regions of nivolumab) was investigated. Dose titration of TROP2×CD28 bsAb were incubated with illustrative PD-1 mAb and MDA-MB-231 cancer cells at 10:1 effector:target ratio. IL2 secretion after 24 hours are depicted in FIG. 46, and show that combining TROP2×CD28 bsAb combines productively with PD-1 blockade.

### Example 8: T Cell Expansion and Cytotoxicity

1 μg/ml TROP2×CD28 bsAb XENP44599 was incubated with cocultures of purified T cells and OVCAR5 cells and dose titration of illustrative B7H3×CD3 bsAb. T cell activation and proliferation and tumor cell killing are depicted in FIG. 47, and show that TROP2×CD28 improves T cell expansion and cytotoxicity of a CD3 T-cell engager.

### Example 9: Pharmacokinetics

Cynomolgus monkeys were dosed with TROP2×CD28 bsAbs having the 2 nM or 13 nM TROP2 binding domain (respectively XENP44599 and XENP44595). As depicted in Figure X, the bsAb having the higher TROP2 affinity clearly rapidly, due to TMDD; while on the other hand, the bsAb having the 13 nM TROP2 affinity demonstrated much better serum half-life.

### Example 10: Selectivity

While TROP2 is expressed highly on cancer cells, it is also expressed on normal cells. TROP2×CD28 bsAbs in the 1+1 CLC format having 2, 5, or 13 nM TROP2 binding domain and 21, 43, or 63 nM CD28 binding domain. OvCAR5 (TROP2^{high}) and OvCAR3 (TROP2^{low}) were incubated with T cells at 1:1 effector:target ratio, 1 μg/ml illustrative EpCAM×CD3 bsAb, and dose titration of the TROP2×CD28 bsAb, data for which are depicted in Figures X and Y showing potency and efficacy shift on TROP2^{high} vs. TROP2^{low} cell lines.

In another experiment, select TROP2×CD28 bsAbs were incubated with illustrative EpCAM×CD3 bsAb, purified T cells, and OVCAR5 cells to investigate cytotoxicity and T cell proliferation induced by the CD28 bsAbs. Data depicted in FIG. 60 show that each of the CD28 bsAbs dose-dependently induced target cell kill and T cell proliferation. The potency is consistent with the experiment above.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

1.-97. (canceled)

98. A bispecific antibody comprises a means for binding tumor-associated calcium signal transducer 2 (TROP2) and a means for binding CD28.

99. A bispecific antibody comprising:
a) a TROP2 binding domain comprising i) a first variable heavy domain (VH1), and ii) a first variable light domain (VL1); and
b) an anti-CD28 binding domain comprising i) a second variable heavy domain (VH2), and ii) a second variable light domain (VL2).

100. The bispecific antibody of claim 99, wherein VH1 and VL1 are selected from any of the TROP2 binding domain VHs and VLs of FIGS. 23-26 and 52-56.

101. The bispecific antibody according to claim 99, wherein VH2 and VL2 are selected from one of the following:
(1) a VH and VL of any of the CD28 binding domains from FIGS. 16, 19, and 23; or
(2) (i) a VH from FIG. 17 or a variant thereof, and (ii) a VL from FIG. 18.

102. The bispecific antibody of claim 99, wherein VH1 is selected from a variable heavy domain of FIG. 36, VH2 is a selected from a variable heavy domain of FIG. 34 or 52-56, and the VL1 and VL2 are each the 1F11-1A3.315[TROP2]_L1 variable light domain (see FIG. 36A).

103. A heterodimeric antibody comprising:
a) a first monomer comprising:
i) a single chain variable fragment (scFv); and
ii) a first Fc domain, wherein the scFv is covalently attached to the N-terminus of the first Fc domain using a domain linker;
b) a second monomer comprising, from N-terminal to C-terminal, a VH1-CH1-hinge-CH2-CH3, wherein VH1 is a first variable heavy domain and CH2-CH3 is a second Fc domain; and
c) a light chain comprising, from N-terminal to C-terminal, VL1-CL, wherein VL1 is a first variable light domain and CL is a constant light domain,
wherein the scFv comprises a second VH domain (VH2), a scFv linker, and a second variable light domain (VL2),
wherein the VH1 and the VL1 together form a first antigen binding domain (ABD) and the VH2 and the VL2 together form a second ABD, and
wherein one of the first ABD and second ABD is a CD28 binding domain and the other of the first ABD and second ABD is a tumor-associated calcium signal transducer 2 (TROP2) binding domain.

104. The heterodimeric antibody according to claim 103, wherein the first ABD is the TROP2 binding domain and the second ABD is the CD28 binding domain.

105. The heterodimeric antibody according to claim 104, wherein VH1 and VL1 are selected from:
(1) a VH and VL of any of the TROP2 binding domains from FIGS. 23-26 and 52-56; and
(2) a VH having an amino acid sequence of a VH depicted in FIG. 36 or a variant thereof, and a VL having an amino acid sequence of the 1F11-1A3.315 L1 variable light domain (FIG. 36A).

106. The heterodimeric antibody according to 104, wherein VH2 and VL2 are selected from one of the following:
(1) a VH and VL of any of the CD28 binding domains from FIGS. 16, 19, and 22; or
(2) (i) a VH from FIG. 16, 17, or 34; and (ii) a VL from FIG. 16, 18, or 36.

107. A nucleic acid composition comprising:
a) a first nucleic acid encoding the first monomer of claim 103;
b) a second nucleic acid encoding the second monomer of claim 103; and
c) a third nucleic acid encoding the light chain of claim 103.

108. An expression vector composition comprising:
a) a first expression vector comprising the first nucleic acid of claim 107;
b) a second expression vector comprising the second nucleic acid of claim 107; and
c) a third expression vector comprising the third nucleic acid of claim 107; respectively.

109. A host cell comprising the expression vector composition of claim 108.

110. A method of making a heterodimeric antibody comprising culturing the host cell of claim 109 under conditions wherein the heterodimeric antibody is expressed and recovering the heterodimeric antibody.

111. A heterodimeric antibody comprising:
a) a first monomer comprising, from N-terminal to C-terminal, VH1-CH1-hinge-CH2-CH3-domain linker-scFv,
wherein VH1 is a first variable heavy domain, and CH2-CH3 is a first Fc domain;
b) a second monomer comprising, from N-terminal to C-terminal, a VH1-CH1-hinge-CH2-CH3, wherein VH1 is a first variable heavy domain and CH2-CH3 is a second Fc domain; and
c) a first light chain comprising, from N-terminal to C-terminal, VL1-CL, wherein VL1 is a first variable light domain and CL is a constant light domain; and
d) a second light chain comprising, from N-terminal to C-terminal, VL1-CL, wherein VL1 is a first variable light domain and CL is a constant light domain,
wherein the scFv comprises a second VH domain (VH2), a scFv linker, and a second variable light domain (VL2),
wherein the VH1 of the first monomer and the VL1 of the first light chain and the VH1 of the second monomer and the VL1 of the second light chain each form a first antigen binding domain (ABD), and the VH2 and the VL2 form a second ABD, and
wherein one of the first and second ABDs bind human CD28 and the other of the first and second ABDs binds TROP2.

112. The heterodimeric antibody according to claim 111, wherein VH1 and VL1 are a VH and VL of any of the TROP2 binding domain VHs and VLs from FIGS. 23-26 and 52-56.

113. The heterodimeric antibody according to claim 111, wherein VH2 and VL2 are selected from one of the following:
(1) a VH and VL of any of the CD28 binding domains from FIGS. 16, 19, and 22; or
(2) (i) a VH from FIG. 16, 17, or 34, or a variant thereof, and (ii) a VL from FIG. 16, 18, or 36.

114. A nucleic acid composition comprising:
a) a first nucleic acid encoding the first monomer of claim 111;
b) a second nucleic acid encoding the second monomer of claim 111; and
c) a third nucleic acid encoding the light chain of claim 111.

115. An expression vector composition comprising:
a) a first expression vector comprising the first nucleic acid of claim 114;
b) a second expression vector comprising the second nucleic acid of claim 114; and
c) a third expression vector comprising the third nucleic acid of claim 114; respectively.

116. A host cell comprising the expression vector composition of claim 115.

117. A method of making a heterodimeric antibody comprising culturing the host cell of claim 116 under conditions wherein the heterodimeric antibody is expressed and recovering the heterodimeric antibody.

118. A multivalent antibody comprising:
a) a first monomer comprising, from N-terminal to C-terminal, a VH1-CH1-hinge-CH2-CH3, wherein VH1 is a first variable heavy domain and CH2-CH3 is a first Fc domain;
b) a second monomer comprising, from N-terminal to C-terminal, a VH2-CH1-hinge-CH2-CH3, wherein VH2 is a second variable heavy domain and CH2-CH3 is a second Fc domain;
c) a first common light chain and second common light chain that each comprise a VL-CL, wherein VL is a variable domain and CL is a constant light domain;
wherein the VH1 and the VL of the first common light chain form a first antigen binding domain (ABD) and the VH2 and the VL of the second common light chain together form a second ABD,
wherein the first common light chain and second common light chain have the same amino acid sequence, and
wherein one of the first ABD and second ABD is a CD28 binding domain and the other of the first ABD and second ABD is a tumor-associated calcium signal transducer 2 (TROP2) binding domain.

119. The multivalent antibody according to claim 118, wherein VH1 is a variable heavy domain from FIG. 36 or 52-56, VH2 is a variable heavy domain from FIG. 34, and the VL of first and second common light chain is the 1F11-1A3.315[TROP2]_L1 variable light domain (see FIG. 36A).

120. A nucleic acid composition comprising:
a) a first nucleic acid encoding the first monomer of claim 118;
b) a second nucleic acid encoding the second monomer of claim 118; and
c) a third nucleic acid encoding the first common light chain of claim 118.

121. An expression vector composition comprising:
a) a first expression vector comprising the first nucleic acid of claim 120;
b) a second expression vector comprising the second nucleic acid of claim 120; and
c) a third expression vector comprising the third nucleic acid of claim 120.

122. A host cell comprising the expression vector composition of claim 121.

123. A method of making a multivalent antibody comprising culturing the host cell of claim 122 under conditions wherein the multivalent antibody is expressed and recovering the multivalent antibody.

124. A multivalent antibody comprising:
a) a first monomer comprising, from N-terminal to C-terminal, a VH1-CH1-linker-VH1-CH1-hinge-CH2-CH3, wherein the VH1s are each a first variable heavy domain and CH2-CH3 is a first Fc domain;
b) a second monomer comprising, from N-terminal to C-terminal, a VH2-CH1-hinge-CH2-CH3, wherein VH2 is a second variable heavy domain and CH2-CH3 is a second Fc domain;
c) a first common light chain, a second common light chain, and third common light chain that each comprise a VL-CL, wherein VL is a variable domain and CL is a constant light domain;
wherein the VH1s of the first monomer each pair with a VL of the first common light chain or second common light to form two first antigen binding domains (ABDs) and the VH2 and the VL of the third common light chain together form a second ABD,
wherein the first common light chain, second common light chain, and third common light chain each have the same amino acid sequence, and
wherein the first ABDs are each a CD28 binding domain and the second ABD is a tumor-associated calcium signal transducer 2 (TROP2) binding domain, or the first ABDs are each a TROP2 binding domain and the second ABD is a CD28 binding domain.

125. The multivalent antibody according to claim 124, wherein the VH1s are each a variable heavy domain from FIG. 36 or 52-56, VH2 is a variable heavy domain from FIG. 34, and the VL of the first, second, and third common light chain is the 1F11-1A3.315[TROP2]_L1 variable light domain (see FIG. 36A).

126. A nucleic acid composition comprising:
a) a first nucleic acid encoding the first monomer of claim 124;
b) a second nucleic acid encoding the second monomer of claim 124; and
c) a third nucleic acid encoding the first common light chain of claim 124.

127. An expression vector composition comprising:
a) a first expression vector comprising the first nucleic acid of claim 126;
b) a second expression vector comprising the second nucleic acid of claim 126; and
c) a third expression vector comprising the third nucleic acid of claim 126.

128. A host cell comprising the expression vector composition of claim 127.

129. A method of making a multivalent antibody comprising culturing the host cell of claim 128 under conditions wherein the multivalent antibody is expressed and recovering the multivalent antibody.

130. A tumor-associated calcium signal transducer 2 (TROP2) binding domain comprising a variable heavy domain selected from SEQ ID NOs: 802, 810-840, 902, 910, 918, 926, and 934 and a variable light domain of SEQ ID NO:806


SEQUENCE LISTING (941 sequences)

SEQ ID NO: 1 - protein, 220 aa, Homo sapiens
  source 1..220: note = sp|P10747|CD28_HUMAN T-cell-specific surface glycoprotein CD28 OS=Homo sapiens OX=9606 GN=CD28 PE=1; SV = 1
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 134 aa, Homo sapiens
  source 1..134: note = sp|P10747|19-152
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 3 - protein, 218 aa, Mus musculus
  source 1..218: note = sp|P31041|CD28_MOUSE T-cell-specific surface glycoprotein CD28 OS=Mus musculus OX=10090 GN=Cd28 PE=1; SV = 2
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 4 - protein, 131 aa, Mus musculus
  source 1..131: note = sp|P31041|20-150
NKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKL

SEQ ID NO: 5 - protein, 220 aa, Macaca fascicularis
  source 1..220: note = tr|Q0PDN3|Q0PDN3_MACFA CD28 OS=Macaca fascicularis; OX = 9541 GN=CD28 PE=2 SV=1
MLRLLLALNLLPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWALVVVGGVLACYSLLVTVAFCIFWMRSKRSRLLHSDYMNMTPRRPGPTRKHYQPYAPPRDFAAYRS

SEQ ID NO: 6 - protein, 134 aa, Macaca fascicularis; identical to SEQ ID NO: 2
  source 1..134: note = tr|Q0PDN3|19-152

SEQ ID NO: 7 - protein, 584 aa, Macaca fascicularis
  source 1..584: note = XP_015308533.1 PREDICTED: CD276 antigen isoform X1 [Macaca fascicularis]
MKLSSDHVFPLFRKLQWLPAAFRIQFTPVSPSAGAAFHHGEPSCQLPHSKMLHRRGSPGMGVHVGAALGALWFCLTGALEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSITITPQRSPTGAVEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSVTITGQPMTFPPEALWVTVGLSVCLVALLVALAFVCWRKIKQSCEEENAGAEDQDGEGEGSKTALQPLKHSDSKEDDGQELA

SEQ ID NO: 8 - protein, 438 aa, Macaca fascicularis
  source 1..438: note = XP_015308533.1|79-516
LEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSITITPQRSPTGAVEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSVTITGQPMTFPPEA

SEQ ID NO: 9 - protein, 323 aa, Homo sapiens
  source 1..323: note = sp|P09758|TACD2_HUMAN Tumor-associated calcium signal transducer 2 OS=Homo sapiens OX=9606 GN=TACSTD2; PE = 1 SV=3
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 10 - protein, 248 aa, Homo sapiens
  source 1..248: note = sp|P09758|27-274
HTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLT

SEQ ID NO: 11 - protein, 317 aa, Mus musculus
  source 1..317: note = sp|Q8BGV3|TACD2_MOUSE Tumor-associated calcium signal transducer 2 OS=Mus musculus OX=10090 GN=Tacstd2; PE = 1 SV=1
MARGLDLAPLLLLLLAMATRFCTAQSNCTCPTNKMTVCDTNGPGGVCQCRAMGSQVLVDCSTLTSKCLLLKARMSARKSGRSLVMPSEHAILDNDGLYDPECDDKGRFKARQCNQTSVCWCVNSVGVRRTDKGDQSLRCDEVVRTHHILIELRHRPTDRAFNHSDLDSELRRLFQERYKLHPSFLSAVHYEEPTIQIELRQNASQKGLRDVDIADAAYYFERDIKGESLFMGRRGLDVQVRGEPLHVERTLIYYLDEKPPQFSMKRLTAGVIAVIAVVSVAVVAGVVVLVVTKRRKSGKYKKVELKELGEMRSEPSL

SEQ ID NO: 12 - protein, 246 aa, Mus musculus
  source 1..246: note = sp|Q8BGV3|25-270
QSNCTCPTNKMTVCDTNGPGGVCQCRAMGSQVLVDCSTLTSKCLLLKARMSARKSGRSLVMPSEHAILDNDGLYDPECDDKGRFKARQCNQTSVCWCVNSVGVRRTDKGDQSLRCDEVVRTHHILIELRHRPTDRAFNHSDLDSELRRLFQERYKLHPSFLSAVHYEEPTIQIELRQNASQKGLRDVDIADAAYYFERDIKGESLFMGRRGLDVQVRGEPLHVERTLIYYLDEKPPQFSMKRLTAG

SEQ ID NO: 13 - protein, 323 aa, Macaca fascicularis
  source 1..323: note = XP_005543292.2 tumor-associated calcium signal transducer 2 [Macaca fascicularis]
MARGPGLAPPPLRLPLLLLLLAAVTGHTAAQDNCTCPTNKMTVCSPDGPGGRCQCRALGSGVAVDCSTLTSKCLLLKARMSAPKNARTLVRPNEHALVDNDGLYDPDCDPEGRFKARQCNQTSVCWCVNSVGVRRTDKGDLSLRCDELVRTHHILIDLRHRPTASAFNHSDLDAELRRLFRERYRLHPKFVAAVHYEQPTIQIELRQNTSQKAAGDVDIGDAAYYFERDVKGESLFQGRGGLDLRVRGEPLQVERTLIYYLDEIPPKFSMKRLTAGLIAVIVVVVVALVAGVAVLVISNRRKSGKYKKVEIKELGELRKEPSL

SEQ ID NO: 14 - protein, 248 aa, Macaca fascicularis
  source 1..248: note = XP_005543292.2|27-274
HTAAQDNCTCPTNKMTVCSPDGPGGRCQCRALGSGVAVDCSTLTSKCLLLKARMSAPKNARTLVRPNEHALVDNDGLYDPDCDPEGRFKARQCNQTSVCWCVNSVGVRRTDKGDLSLRCDELVRTHHILIDLRHRPTASAFNHSDLDAELRRLFRERYRLHPKFVAAVHYEQPTIQIELRQNTSQKAAGDVDIGDAAYYFERDVKGESLFQGRGGLDLRVRGEPLQVERTLIYYLDEIPPKFSMKRLT

SEQ ID NO: 15 - protein, 15 aa, synthetic construct
GGGGSGGGGSGGGGS

SEQ ID NO: 16 - protein, 18 aa, synthetic construct
GSTSGSGKPGSGEGSTKG

SEQ ID NO: 17 - protein, 14 aa, synthetic construct
IRPRAIGGSKPRVA

SEQ ID NO: 18 - protein, 15 aa, synthetic construct
GKGGSGKGGSGKGGS

SEQ ID NO: 19 - protein, 15 aa, synthetic construct
GGKGSGGKGSGGKGS

SEQ ID NO: 20 - protein, 15 aa, synthetic construct
GGGKSGGGKSGGGKS

SEQ ID NO: 21 - protein, 15 aa, synthetic construct
GKGKSGKGKSGKGKS

SEQ ID NO: 22 - protein, 15 aa, synthetic construct
GGGKSGGKGSGKGGS

SEQ ID NO: 23 - protein, 15 aa, synthetic construct
GKPGSGKPGSGKPGS

SEQ ID NO: 24 - protein, 20 aa, synthetic construct
GKPGSGKPGSGKPGSGKPGS

SEQ ID NO: 25 - protein, 20 aa, synthetic construct
GKGKSGKGKSGKGKSGKGKS

SEQ ID NO: 26 - protein, 20 aa, synthetic construct
GGGGSGGGGSGGGGSGGGGS

SEQ ID NO: 27 - protein, 14 aa, synthetic construct
STAGDTHLGGEDFD

SEQ ID NO: 28 - protein, 15 aa, synthetic construct
GEGGSGEGGSGEGGS

SEQ ID NO: 29 - protein, 15 aa, synthetic construct
GGEGSGGEGSGGEGS

SEQ ID NO: 30 - protein, 15 aa, synthetic construct
GGGESGGGESGGGES

SEQ ID NO: 31 - protein, 15 aa, synthetic construct
GEGESGEGESGEGES

SEQ ID NO: 32 - protein, 15 aa, synthetic construct
GGGESGGEGSGEGGS

SEQ ID NO: 33 - protein, 20 aa, synthetic construct
GEGESGEGESGEGESGEGES

SEQ ID NO: 34 - protein, 19 aa, synthetic construct
PRGASKSGSASQTGSAPGS

SEQ ID NO: 35 - protein, 19 aa, synthetic construct
GTAAAGAGAAGGAAAGAAG

SEQ ID NO: 36 - protein, 19 aa, synthetic construct
GTSGSSGSGSGGSGSGGGG

SEQ ID NO: 37 - protein, 5 aa, synthetic construct
GGGGS

SEQ ID NO: 38 - protein, 10 aa, synthetic construct
GGGGSGGGGS

SEQ ID NO: 39 - protein, 25 aa, synthetic construct
GGGGSGGGGSGGGGSGGGGSGGGGS

SEQ ID NO: 40 - protein, 30 aa, synthetic construct
GGGGSGGGGSGGGGSGGGGSGGGGSGGGGS

SEQ ID NO: 41 - protein, 35 aa, synthetic construct
GGGGSGGGGSGGGGSGGGGSGGGGSGGGGSGGGGS

SEQ ID NO: 42 - protein, 5 aa, synthetic construct
GGGGA

SEQ ID NO: 43 - protein, 10 aa, synthetic construct
GGGGAGGGGA

SEQ ID NO: 44 - protein, 15 aa, synthetic construct
GGGGAGGGGAGGGGA

SEQ ID NO: 45 - protein, 20 aa, synthetic construct
GGGGAGGGGAGGGGAGGGGA

SEQ ID NO: 46 - protein, 25 aa, synthetic construct
GGGGAGGGGAGGGGAGGGGAGGGGA

SEQ ID NO: 47 - protein, 30 aa, synthetic construct
GGGGAGGGGAGGGGAGGGGAGGGGAGGGGA

SEQ ID NO: 48 - protein, 35 aa, synthetic construct
GGGGAGGGGAGGGGAGGGGAGGGGAGGGGAGGGGA

SEQ ID NO: 49 - protein, 30 aa, synthetic construct
DPALVHQRPAPPGGGGSGGGGSGGGGSGGG

SEQ ID NO: 50 - protein, 5 aa, synthetic construct
GKPGS

SEQ ID NO: 51 - protein, 25 aa, synthetic construct
GKPGSGKPGSGKPGSGKPGSGKPGS

SEQ ID NO: 52 - protein, 30 aa, synthetic construct
GKPGSGKPGSGKPGSGKPGSGKPGSGKPGS

SEQ ID NO: 53 - protein, 5 aa, synthetic construct
GGGES

SEQ ID NO: 54 - protein, 15 aa, synthetic construct
EPKSCDKTHTCPPCP

SEQ ID NO: 55 - protein, 9 aa, synthetic construct
KTHTCPPCP

SEQ ID NO: 56 - protein, 15 aa, synthetic construct
EPKSSDKTHTCPPCP

SEQ ID NO: 57 - protein, 19 aa, synthetic construct
GGGGSGGGGSKTHTCPPCP

SEQ ID NO: 58 - protein, 19 aa, synthetic construct
GKPGSGKPGSKTHTCPPCP

SEQ ID NO: 59 - protein, 14 aa, synthetic construct
GKPGSKTHTCPPCP

SEQ ID NO: 60 - protein, 5 aa, synthetic construct
EPKSC

SEQ ID NO: 61 - protein, 15 aa, synthetic construct
EPKSCGGGGSGGGGS

SEQ ID NO: 62 - protein, 15 aa, synthetic construct
EPKSCGKPGSGKPGS

SEQ ID NO: 63 - protein, 10 aa, synthetic construct
EPKSCGKPGS

SEQ ID NO: 64 - protein, 216 aa, synthetic construct
  source 1..216: note = Heterodimeric Fc Backbone 1 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 65 - protein, 216 aa, synthetic construct
  source 1..216: note = Heterodimeric Fc Backbone 1 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 216 aa, synthetic construct; identical to SEQ ID NO: 64
  source 1..216: note = Heterodimeric Fc Backbone 2 monomer 1 (-)

SEQ ID NO: 67 - protein, 216 aa, synthetic construct
  source 1..216: note = Heterodimeric Fc Backbone monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 216 aa, synthetic construct
  source 1..216: note = Heterodimeric Fc Backbone 3 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCEVSGFYPSDIAVEWESDGQPENNYKTTPPVLDSDGSFFLYSKLTVDKSRWEQGDVFSCSVMHEALHNHYTQKSLSLSPGK

SEQ ID NO: 69 - protein, 216 aa, synthetic construct; identical to SEQ ID NO: 67
  source 1..216: note = Heterodimeric Fc Backbone 3 monomer 2 (+)

SEQ ID NO: 70 - protein, 216 aa, synthetic construct
  source 1..216: note = Heterodimeric Fc Backbone 4 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTENEVSLTCLVKGFYPSDIAVEWESDGQPENNYKTTPPVLDSDGSFFLYSKLEVDKSRWEQGDVFSCSVMHEALHNHYTQKSLSLSPGK

SEQ ID NO: 71 - protein, 216 aa, synthetic construct
  source 1..216: note = Heterodimeric Fc Backbone 4 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSKGSFFLYSKLTVDKSRWQQGNVFSCSVMHEALHNHYTQKSLSLSPGK

SEQ ID NO: 72 - protein, 216 aa, synthetic construct
  source 1..216: note = Heterodimeric Fc Backbone 5 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 73 - protein, 216 aa, synthetic construct
  source 1..216: note = Heterodimeric Fc Backbone 5 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 216 aa, synthetic construct
  source 1..216: note = Heterodimeric Fc Backbone 6 monomer 1 (-)
APPVAGPSVFLFPPKPKDTLMISRTPEVTCVVVDVKHEDPEVKFNWYVDGVEVHNAKTKPREEEYASTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 75 - protein, 216 aa, synthetic construct
  source 1..216: note = Heterodimeric Fc Backbone 6 monomer 2 (+)
APPVAGPSVFLFPPKPKDTLMISRTPEVTCVVVDVKHEDPEVKFNWYVDGVEVHNAKTKPREEQYASTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 216 aa, synthetic construct
  source 1..216: note = Heterodimeric Fc Backbone 7 monomer 1 (-)
APPVAGPSVFLFPPKPKDTLMISRTPEVTCVVVDVKHEDPEVKFNWYVDGVEVHNAKTKPREEEYSSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 77 - protein, 216 aa, synthetic construct
  source 1..216: note = Heterodimeric Fc Backbone 7 monomer 2 (+)
APPVAGPSVFLFPPKPKDTLMISRTPEVTCVVVDVKHEDPEVKFNWYVDGVEVHNAKTKPREEQYSSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 217 aa, synthetic construct
  source 1..217: note = Heterodimeric Fc Backbone 8 monomer 1 (-)
APEFLGGPSVFLFPPKPKDTLMISRTPEVTCVVVDVSQEDPEVQFNWYVDGVEVHNAKTKPREEEFNSTYRVVSVLTVLHQDWLNGKEYKCKVSNKGLPSSIEKTISKAKGQPREPQVYTLPPSQEEMTKNQVSLTCDVSGFYPSDIAVEWESDGQPENNYKTTPPVLDSDGSFFLYSRLTVDKSRWEEGDVFSCSVMHEALHNHYTQKSLSLSLGK

SEQ ID NO: 79 - protein, 217 aa, synthetic construct
  source 1..217: note = Heterodimeric Fc Backbone 8 monomer 2 (+)
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEQMTKNQVKLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - protein, 216 aa, synthetic construct
  source 1..216: note = Heterodimeric Fc Backbone 9 monomer 1 (-)
APPVAGPSVFLFPPKPKDTLMISRTPEVTCVVVDVSHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 81 - protein, 216 aa, synthetic construct
  source 1..216: note = Heterodimeric Fc Backbone 9 monomer 2 (+)
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 82 - protein, 216 aa, synthetic construct
  source 1..216: note = Heterodimeric Fc Backbone 10 monomer 1 (-)
APPVAGPSVFLFPPKPKDTLMISRTPEVTCVVVDVKHEDPEVQFNWYVDGVEVHNAKTKPREEEFNSTFRVVSVLTVVHQDWLNGKEYKCKVSNKGLPAPIEKTISKTKGQPREPQVYTLPPSREEMTKNQVSLTCDVSGFYPSDIAVEWESDGQPENNYKTTPPMLDSDGSFFLYSKLTVDKSRWEQGDVFSCSVMHEALHNHYTQKSLSLSPGK

SEQ ID NO: 83 - protein, 216 aa, synthetic construct
  source 1..216: note = Heterodimeric Fc Backbone 10 monomer 2 (+)
APPVAGPSVFLFPPKPKDTLMISRTPEVTCVVVDVKHEDPEVQFNWYVDGVEVHNAKTKPREEQFNSTFRVVSVLTVVHQDWLNGKEYKCKVSNKGLPAPIEKTISKTKGQPREPQVYTLPPSREQMTKNQVKLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 84 - protein, 216 aa, synthetic construct
  source 1..216: note = Heterodimeric Fc Backbone 11 monomer 1 (-)
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 85 - protein, 216 aa, synthetic construct
  source 1..216: note = Heterodimeric Fc Backbone 11 monomer 2 (+)
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 86 - protein, 216 aa, synthetic construct
  source 1..216: note = Heterodimeric Fc Backbone 12 monomer 1 (-)
APPVAGPSVFLFPPKPKDTLMISRTPEVTCVVVDVKHEDPEVKFKWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 216 aa, synthetic construct; identical to SEQ ID NO: 64
  source 1..216: note = Heterodimeric Fc Backbone 12 monomer 2 (+)

SEQ ID NO: 88 - protein, 216 aa, synthetic construct
  source 1..216: note = Heterodimeric Fc Backbone 13 monomer 1
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 89 - protein, 216 aa, synthetic construct
  source 1..216: note = Heterodimeric Fc Backbone 13 monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 90 - protein, 216 aa, synthetic construct
  source 1..216: note = Heterodimeric Fc Backbone 14 monomer 1
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 91 - protein, 216 aa, synthetic construct
  source 1..216: note = Heterodimeric Fc Backbone 14 monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 92 - protein, 216 aa, synthetic construct
  source 1..216: note = Heterodimeric Fc Backbone 15 monomer 1
APPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCDVSGFYPSDIAVEWESDGQPENNYKTTPPVLDSDGSFFLYSKLTVDKSRWEQGDVFSCSVLHEALHAHYTQKSLSLSPGK

SEQ ID NO: 93 - protein, 216 aa, synthetic construct
  source 1..216: note = Heterodimeric Fc Backbone 15 monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSRDQLTKNQVKLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 94 - protein, 216 aa, synthetic construct; identical to SEQ ID NO: 64
  source 1..216: note = 2 + 1 mAb-scFv Heterodimeric Fc Backbone monomer 1

SEQ ID NO: 95 - protein, 215 aa, synthetic construct
  source 1..215: note = 2 + 1 mAb-scFv Heterodimeric Fc Backbone monomer 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 98 aa, synthetic construct
  source 1..98: note = IgG1 CH1(+)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 97 - protein, 98 aa, synthetic construct
  source 1..98: note = IgG1 CH1(-)
ASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKV

SEQ ID NO: 98 - protein, 98 aa, synthetic construct
  source 1..98: note = IgG2 CH1(+)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 99 - protein, 98 aa, synthetic construct
  source 1..98: note = IgG2 CH1(-)
ASTKGPSVFPLAPCSRSTSESTAALGCLVKDYFPEPVTVSWNSGALTSGVHTFPAVLQSSGLYSLSSVVTVPSSNFGTQTYTCNVDHKPSDTKVDKTV

SEQ ID NO: 100 - protein, 98 aa, synthetic construct
  source 1..98: note = IgG4 CH1(+)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 101 - protein, 98 aa, synthetic construct
  source 1..98: note = IgG4 CH1(-)
ASTKGPSVFPLAPCSRSTSESTAALGCLVKDYFPEPVTVSWNSGALTSGVHTFPAVLQSSGLYSLSSVVTVPSSSLGTKTYTCNVDHKPSDTKVDKRV

SEQ ID NO: 102 - protein, 15 aa, synthetic construct
  source 1..15: note = IgG1 hinge
EPKSCDKTHTCPPCP

SEQ ID NO: 103 - protein, 12 aa, synthetic construct
  source 1..12: note = IgG2 hinge
ERKCCVECPPCP

SEQ ID NO: 104 - protein, 12 aa, synthetic construct
  source 1..12: note = IgG4 hinge
ESKYGPPCPSCP

SEQ ID NO: 105 - protein, 107 aa, synthetic construct [sequence omitted]

SEQ ID NO: 106 - protein, 106 aa, synthetic construct
  source 1..106: note = Light Chain Constant Domain - Lambda
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 107 - protein, 121 aa, synthetic construct
  source 1..121: note = 1A7[CD28]_H1L1 Variable heavy (vh) domain
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSS

SEQ ID NO: 108 - protein, 5 aa, synthetic construct
  source 1..5: note = 1A7[CD28]_H1L1 vhCDR1
SYAMS

SEQ ID NO: 109 - protein, 17 aa, synthetic construct
  source 1..17: note = 1A7[CD28]_H1L1 vhCDR2
TISGSGDSTYYADSVKG

SEQ ID NO: 110 - protein, 12 aa, synthetic construct
  source 1..12: note = 1A7[CD28]_H1L1 vhCDR3
SGPGLRQVGFDY

SEQ ID NO: 111 - protein, 107 aa, synthetic construct
  source 1..107: note = 1A7[CD28]_H1L1 Variable light (vl) domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIK

SEQ ID NO: 112 - protein, 11 aa, synthetic construct
  source 1..11: note = 1A7[CD28]_H1L1 vlCDR1
RASQSISSYLN

SEQ ID NO: 113 - protein, 7 aa, synthetic construct
  source 1..7: note = 1A7[CD28]_H1L1 vlCDR2
AASSLQS

SEQ ID NO: 114 - protein, 9 aa, synthetic construct
  source 1..9: note = 1A7[CD28]_H1L1 vlCDR3
QQSYSTPFT

SEQ ID NO: 115 - protein, 450 aa, synthetic construct
  source 1..450: note = XENP28428 Heavy Chain
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 214 aa, synthetic construct
  source 1..214: note = XENP28428 Light Chain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 121 aa, synthetic construct
  source 1..121: note = 1A7[CD28]_H1.1
EVQLLESGGGLVQPGGSLRLSCAASGFTFSSYYMSWVRQAPGKGLEWVSTISGSGDSTYYADSVKGRFTISRDNSKNTLYLQMNSLRAEDTAVYYCAKSGPGLRQVGFDYWGQGTLVTVSS

SEQ ID NO: 118 - protein, 121 aa, synthetic construct
  source 1..121: note = 1A7[CD28]_H1.2
EVQLLESGGGLVQPGGSLRLSCAASGFTFSSYAMSWVRQAPGKGLEWVSTIDGSGDSTYYADSVKGRFTISRDNSKNTLYLQMNSLRAEDTAVYYCAKSGPGLRQVGFDYWGQGTLVTVSS

SEQ ID NO: 119 - protein, 121 aa, synthetic construct
  source 1..121: note = 1A7[CD28]_H1.3
EVQLLESGGGLVQPGGSLRLSCAASGFTFSSYAMSWVRQAPGKGLEWVSTIEGSGDSTYYADSVKGRFTISRDNSKNTLYLQMNSLRAEDTAVYYCAKSGPGLRQVGFDYWGQGTLVTVSS

SEQ ID NO: 120 - protein, 121 aa, synthetic construct
  source 1..121: note = 1A7[CD28]_H1.4
EVQLLESGGGLVQPGGSLRLSCAASGFTFSSYAMSWVRQAPGKGLEWVSTIYGSGDSTYYADSVKGRFTISRDNSKNTLYLQMNSLRAEDTAVYYCAKSGPGLRQVGFDYWGQGTLVTVSS

SEQ ID NO: 121 - protein, 121 aa, synthetic construct
  source 1..121: note = 1A7[CD28]_H1.5
EVQLLESGGGLVQPGGSLRLSCAASGFTFSSYAMSWVRQAPGKGLEWVSTISDSGDSTYYADSVKGRFTISRDNSKNTLYLQMNSLRAEDTAVYYCAKSGPGLRQVGFDYWGQGTLVTVSS

SEQ ID NO: 122 - protein, 121 aa, synthetic construct
  source 1..121: note = 1A7[CD28]_H1.6
EVQLLESGGGLVQPGGSLRLSCAASGFTFSSYAMSWVRQAPGKGLEWVSTISESGDSTYYADSVKGRFTISRDNSKNTLYLQMNSLRAEDTAVYYCAKSGPGLRQVGFDYWGQGTLVTVSS

SEQ ID NO: 123 - protein, 121 aa, synthetic construct
  source 1..121: note = 1A7[CD28]_H1.7
EVQLLESGGGLVQPGGSLRLSCAASGFTFSSYAMSWVRQAPGKGLEWVSTISGSGTSTYYADSVKGRFTISRDNSKNTLYLQMNSLRAEDTAVYYCAKSGPGLRQVGFDYWGQGTLVTVSS

SEQ ID NO: 124 - protein, 121 aa, synthetic construct
  source 1..121: note = 1A7[CD28]_H1.8
EVQLLESGGGLVQPGGSLRLSCAASGFTFSSYAMSWVRQAPGKGLEWVSTISGSGYSTYYADSVKGRFTISRDNSKNTLYLQMNSLRAEDTAVYYCAKSGPGLRQVGFDYWGQGTLVTVSS

SEQ ID NO: 125 - protein, 121 aa, synthetic construct
  source 1..121: note = 1A7[CD28]_H1.9
EVQLLESGGGLVQPGGSLRLSCAASGFTFSSYAMSWVRQAPGKGLEWVSTISGSGDYTYYADSVKGRFTISRDNSKNTLYLQMNSLRAEDTAVYYCAKSGPGLRQVGFDYWGQGTLVTVSS

SEQ ID NO: 126 - protein, 121 aa, synthetic construct
  source 1..121: note = 1A7[CD28]_H1.10
EVQLLESGGGLVQPGGSLRLSCAASGFTFSSYYMSWVRQAPGKGLEWVSTIDGSGDSTYYADSVKGRFTISRDNSKNTLYLQMNSLRAEDTAVYYCAKSGPGLRQVGFDYWGQGTLVTVSS

SEQ ID NO: 127 - protein, 121 aa, synthetic construct
  source 1..121: note = 1A7[CD28]_H1.11
EVQLLESGGGLVQPGGSLRLSCAASGFTFSSYYMSWVRQAPGKGLEWVSTIEGSGDSTYYADSVKGRFTISRDNSKNTLYLQMNSLRAEDTAVYYCAKSGPGLRQVGFDYWGQGTLVTVSS

SEQ ID NO: 128 - protein, 121 aa, synthetic construct
  source 1..121: note = 1A7[CD28]_H1.12
EVQLLESGGGLVQPGGSLRLSCAASGFTFSSYYMSWVRQAPGKGLEWVSTIYGSGDSTYYADSVKGRFTISRDNSKNTLYLQMNSLRAEDTAVYYCAKSGPGLRQVGFDYWGQGTLVTVSS

SEQ ID NO: 129 - protein, 121 aa, synthetic construct
  source 1..121: note = 1A7[CD28]_H1.13
EVQLLESGGGLVQPGGSLRLSCAASGFTFSSYYMSWVRQAPGKGLEWVSTISDSGDSTYYADSVKGRFTISRDNSKNTLYLQMNSLRAEDTAVYYCAKSGPGLRQVGFDYWGQGTLVTVSS

SEQ ID NO: 130 - protein, 121 aa, synthetic construct
  source 1..121: note = 1A7[CD28]_H1.14
EVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSS

SEQ ID NO: 131 - protein, 121 aa, synthetic construct
  source 1..121: note = 1A7[CD28]_H1.15
EVQLLESGGGLVQPGGSLRLSCAASGFTFSSYYMSWVRQAPGKGLEWVSTISGSGTSTYYADSVKGRFTISRDNSKNTLYLQMNSLRAEDTAVYYCAKSGPGLRQVGFDYWGQGTLVTVSS

SEQ ID NO: 132 - protein, 121 aa, synthetic construct
  source 1..121: note = 1A7[CD28]_H1.16
EVQLLESGGGLVQPGGSLRLSCAASGFTFSSYYMSWVRQAPGKGLEWVSTISGSGYSTYYADSVKGRFTISRDNSKNTLYLQMNSLRAEDTAVYYCAKSGPGLRQVGFDYWGQGTLVTVSS

SEQ ID NO: 133 - protein, 121 aa, synthetic construct
  source 1..121: note = 1A7[CD28]_H1.17
EVQLLESGGGLVQPGGSLRLSCAASGFTFSSYYMSWVRQAPGKGLEWVSTISGSGDYTYYADSVKGRFTISRDNSKNTLYLQMNSLRAEDTAVYYCAKSGPGLRQVGFDYWGQGTLVTVSS

SEQ ID NO: 134 - protein, 121 aa, synthetic construct
  source 1..121: note = 1A7[CD28]_H1.18
EVQLLESGGGLVQPGGSLRLSCAASGFTFSSYAMSWVRQAPGKGLEWVSTIDDSGDSTYYADSVKGRFTISRDNSKNTLYLQMNSLRAEDTAVYYCAKSGPGLRQVGFDYWGQGTLVTVSS

SEQ ID NO: 135 - protein, 121 aa, synthetic construct
  source 1..121: note = 1A7[CD28]_H1.19
EVQLLESGGGLVQPGGSLRLSCAASGFTFSSYAMSWVRQAPGKGLEWVSTIDESGDSTYYADSVKGRFTISRDNSKNTLYLQMNSLRAEDTAVYYCAKSGPGLRQVGFDYWGQGTLVTVSS

SEQ ID NO: 136 - protein, 121 aa, synthetic construct
  source 1..121: note = 1A7[CD28]_H1.20
EVQLLESGGGLVQPGGSLRLSCAASGFTFSSYAMSWVRQAPGKGLEWVSTIDGSGTSTYYADSVKGRFTISRDNSKNTLYLQMNSLRAEDTAVYYCAKSGPGLRQVGFDYWGQGTLVTVSS

SEQ ID NO: 137 - protein, 121 aa, synthetic construct
  source 1..121: note = 1A7[CD28]_H1.21
EVQLLESGGGLVQPGGSLRLSCAASGFTFSSYAMSWVRQAPGKGLEWVSTIDGSGYSTYYADSVKGRFTISRDNSKNTLYLQMNSLRAEDTAVYYCAKSGPGLRQVGFDYWGQGTLVTVSS

SEQ ID NO: 138 - protein, 121 aa, synthetic construct
  source 1..121: note = 1A7[CD28]_H1.22
EVQLLESGGGLVQPGGSLRLSCAASGFTFSSYAMSWVRQAPGKGLEWVSTIDGSGDYTYYADSVKGRFTISRDNSKNTLYLQMNSLRAEDTAVYYCAKSGPGLRQVGFDYWGQGTLVTVSS

SEQ ID NO: 139 - protein, 121 aa, synthetic construct
  source 1..121: note = 1A7[CD28]_H1.23
EVQLLESGGGLVQPGGSLRLSCAASGFTFSSYAMSWVRQAPGKGLEWVSTIEDSGDSTYYADSVKGRFTISRDNSKNTLYLQMNSLRAEDTAVYYCAKSGPGLRQVGFDYWGQGTLVTVSS

SEQ ID NO: 140 - protein, 121 aa, synthetic construct
  source 1..121: note = 1A7[CD28]_H1.24
EVQLLESGGGLVQPGGSLRLSCAASGFTFSSYAMSWVRQAPGKGLEWVSTIEESGDSTYYADSVKGRFTISRDNSKNTLYLQMNSLRAEDTAVYYCAKSGPGLRQVGFDYWGQGTLVTVSS

SEQ ID NO: 141 - protein, 121 aa, synthetic construct
  source 1..121: note = 1A7[CD28]_H1.25
EVQLLESGGGLVQPGGSLRLSCAASGFTFSSYAMSWVRQAPGKGLEWVSTIEGSGTSTYYADSVKGRFTISRDNSKNTLYLQMNSLRAEDTAVYYCAKSGPGLRQVGFDYWGQGTLVTVSS

SEQ ID NO: 142 - protein, 121 aa, synthetic construct
  source 1..121: note = 1A7[CD28]_H1.26
EVQLLESGGGLVQPGGSLRLSCAASGFTFSSYAMSWVRQAPGKGLEWVSTIEGSGYSTYYADSVKGRFTISRDNSKNTLYLQMNSLRAEDTAVYYCAKSGPGLRQVGFDYWGQGTLVTVSS

SEQ ID NO: 143 - protein, 121 aa, synthetic construct
  source 1..121: note = 1A7[CD28]_H1.27
EVQLLESGGGLVQPGGSLRLSCAASGFTFSSYAMSWVRQAPGKGLEWVSTIEGSGDYTYYADSVKGRFTISRDNSKNTLYLQMNSLRAEDTAVYYCAKSGPGLRQVGFDYWGQGTLVTVSS

SEQ ID NO: 144 - protein, 121 aa, synthetic construct
  source 1..121: note = 1A7[CD28]_H1.28
EVQLLESGGGLVQPGGSLRLSCAASGFTFSSYAMSWVRQAPGKGLEWVSTIYDSGDSTYYADSVKGRFTISRDNSKNTLYLQMNSLRAEDTAVYYCAKSGPGLRQVGFDYWGQGTLVTVSS

SEQ ID NO: 145 - protein, 121 aa, synthetic construct
  source 1..121: note = 1A7[CD28]_H1.29
EVQLLESGGGLVQPGGSLRLSCAASGFTFSSYAMSWVRQAPGKGLEWVSTIYESGDSTYYADSVKGRFTISRDNSKNTLYLQMNSLRAEDTAVYYCAKSGPGLRQVGFDYWGQGTLVTVSS

SEQ ID NO: 146 - protein, 121 aa, synthetic construct
  source 1..121: note = 1A7[CD28]_H1.30
EVQLLESGGGLVQPGGSLRLSCAASGFTFSSYAMSWVRQAPGKGLEWVSTIYGSGTSTYYADSVKGRFTISRDNSKNTLYLQMNSLRAEDTAVYYCAKSGPGLRQVGFDYWGQGTLVTVSS

SEQ ID NO: 147 - protein, 121 aa, synthetic construct
  source 1..121: note = 1A7[CD28]_H1.31
EVQLLESGGGLVQPGGSLRLSCAASGFTFSSYAMSWVRQAPGKGLEWVSTIYGSGYSTYYADSVKGRFTISRDNSKNTLYLQMNSLRAEDTAVYYCAKSGPGLRQVGFDYWGQGTLVTVSS

SEQ ID NO: 148 - protein, 121 aa, synthetic construct
  source 1..121: note = 1A7[CD28]_H1.32
EVQLLESGGGLVQPGGSLRLSCAASGFTFSSYAMSWVRQAPGKGLEWVSTIYGSGDYTYYADSVKGRFTISRDNSKNTLYLQMNSLRAEDTAVYYCAKSGPGLRQVGFDYWGQGTLVTVSS

SEQ ID NO: 149 - protein, 121 aa, synthetic construct
  source 1..121: note = 1A7[CD28]_H1.33
EVQLLESGGGLVQPGGSLRLSCAASGFTFSSYAMSWVRQAPGKGLEWVSTISDSGTSTYYADSVKGRFTISRDNSKNTLYLQMNSLRAEDTAVYYCAKSGPGLRQVGFDYWGQGTLVTVSS

SEQ ID NO: 150 - protein, 121 aa, synthetic construct
  source 1..121: note = 1A7[CD28]_H1.34
EVQLLESGGGLVQPGGSLRLSCAASGFTFSSYAMSWVRQAPGKGLEWVSTISDSGYSTYYADSVKGRFTISRDNSKNTLYLQMNSLRAEDTAVYYCAKSGPGLRQVGFDYWGQGTLVTVSS

SEQ ID NO: 151 - protein, 121 aa, synthetic construct
  source 1..121: note = 1A7[CD28]_H1.35
EVQLLESGGGLVQPGGSLRLSCAASGFTFSSYAMSWVRQAPGKGLEWVSTISDSGDYTYYADSVKGRFTISRDNSKNTLYLQMNSLRAEDTAVYYCAKSGPGLRQVGFDYWGQGTLVTVSS

SEQ ID NO: 152 - protein, 121 aa, synthetic construct
  source 1..121: note = 1A7[CD28]_H1.36
EVQLLESGGGLVQPGGSLRLSCAASGFTFSSYAMSWVRQAPGKGLEWVSTISESGTSTYYADSVKGRFTISRDNSKNTLYLQMNSLRAEDTAVYYCAKSGPGLRQVGFDYWGQGTLVTVSS

SEQ ID NO: 153 - protein, 121 aa, synthetic construct
  source 1..121: note = 1A7[CD28]_H1.37
EVQLLESGGGLVQPGGSLRLSCAASGFTFSSYAMSWVRQAPGKGLEWVSTISESGYSTYYADSVKGRFTISRDNSKNTLYLQMNSLRAEDTAVYYCAKSGPGLRQVGFDYWGQGTLVTVSS

SEQ ID NO: 154 - protein, 121 aa, synthetic construct
  source 1..121: note = 1A7[CD28]_H1.38
EVQLLESGGGLVQPGGSLRLSCAASGFTFSSYAMSWVRQAPGKGLEWVSTISESGDYTYYADSVKGRFTISRDNSKNTLYLQMNSLRAEDTAVYYCAKSGPGLRQVGFDYWGQGTLVTVSS

SEQ ID NO: 155 - protein, 121 aa, synthetic construct
  source 1..121: note = 1A7[CD28]_H1.39
EVQLLESGGGLVQPGGSLRLSCAASGFTFSSYAMSWVRQAPGKGLEWVSTISGSGTYTYYADSVKGRFTISRDNSKNTLYLQMNSLRAEDTAVYYCAKSGPGLRQVGFDYWGQGTLVTVSS

SEQ ID NO: 156 - protein, 121 aa, synthetic construct
  source 1..121: note = 1A7[CD28]_H1.40
EVQLLESGGGLVQPGGSLRLSCAASGFTFSSYAMSWVRQAPGKGLEWVSTISGSGYYTYYADSVKGRFTISRDNSKNTLYLQMNSLRAEDTAVYYCAKSGPGLRQVGFDYWGQGTLVTVSS

SEQ ID NO: 157 - protein, 121 aa, synthetic construct
  source 1..121: note = 1A7[CD28]_H1.41
EVQLLESGGGLVQPGGSLRLSCAASGFTFSSYYMSWVRQAPGKGLEWVSTIEGSGDYTYYADSVKGRFTISRDNSKNTLYLQMNSLRAEDTAVYYCAKSGPGLRQVGFDYWGQGTLVTVSS

SEQ ID NO: 158 - protein, 121 aa, synthetic construct
  source 1..121: note = 1A7[CD28]_H1.42
EVQLLESGGGLVQPGGSLRLSCAASGFTFSSYYMSWVRQAPGKGLEWVSTISESGDYTYYADSVKGRFTISRDNSKNTLYLQMNSLRAEDTAVYYCAKSGPGLRQVGFDYWGQGTLVTVSS

SEQ ID NO: 159 - protein, 121 aa, synthetic construct
  source 1..121: note = 1A7[CD28]_H1.43
EVQLLESGGGLVQPGGSLRLSCAASGFSFSGNYMTWVRQAPGKGLEWVATITADSDATYYADSVKGRFTISRDNSKNTLYLQMNSLRAEDTAVYYCAKSGPGLRQVGFDYWGQGTLVTVSS

SEQ ID NO: 160 - protein, 121 aa, synthetic construct
  source 1..121: note = 1A7[CD28]_H1.44
EVQLLESGGGLVQPGGSLRLSCAASGFTFSGYSMNWVRQAPGKGLEWVATIYANGSYTYYADSVKGRFTISRDNSKNTLYLQMNSLRAEDTAVYYCAKSGPGLRQVGFDYWGQGTLVTVSS

SEQ ID NO: 161 - protein, 121 aa, synthetic construct
  source 1..121: note = 1A7[CD28]_H1.45
EVQLLESGGGLVQPGGSLRLSCAASGFNFEEYSMNWVRQAPGKGLEWVATITYNGDYTYYADSVKGRFTISRDNSKNTLYLQMNSLRAEDTAVYYCAKSGPGLRQVGFDYWGQGTLVTVSS

SEQ ID NO: 162 - protein, 121 aa, synthetic construct
  source 1..121: note = 1A7[CD28]_H1.46
EVQLLESGGGLVQPGGSLRLSCAASGFSFRTYYMTWVRQAPGKGLEWVATITSDGDYTYYADSVKGRFTISRDNSKNTLYLQMNSLRAEDTAVYYCAKSGPGLRQVGFDYWGQGTLVTVSS

SEQ ID NO: 163 - protein, 121 aa, synthetic construct
  source 1..121: note = 1A7[CD28]_H1.47
EVQLLESGGGLVQPGGSLRLSCAASGFSFKGYSMNWVRQAPGKGLEWVATIYASSDSTYYADSVKGRFTISRDNSKNTLYLQMNSLRAEDTAVYYCAKSGPGLRQVGFDYWGQGTLVTVSS

SEQ ID NO: 164 - protein, 121 aa, synthetic construct
  source 1..121: note = 1A7[CD28]_H1.48
EVQLLESGGGLVQPGGSLRLSCAASGFSFGEYSMNWVRQAPGKGLEWVSTIYADGDYTYYADSVKGRFTISRDNSKNTLYLQMNSLRAEDTAVYYCAKSGPGLRQVGFDYWGQGTLVTVSS

SEQ ID NO: 165 - protein, 121 aa, synthetic construct
  source 1..121: note = 1A7[CD28]_H1.49
EVQLLESGGGLVQPGGSLRLSCAASGFTFTDYSMNWVRQAPGKGLEWVATIYADSSSTYYADSVKGRFTISRDNSKNTLYLQMNSLRAEDTAVYYCAKSGPGLRQVGFDYWGQGTLVTVSS

SEQ ID NO: 166 - protein, 121 aa, synthetic construct
  source 1..121: note = 1A7[CD28]_H1.50
EVQLLESGGGLVQPGGSLRLSCAASGFTFGGYSMNWVRQAPGKGLEWVATIYYDSTTTYYADSVKGRFTISRDNSKNTLYLQMNSLRAEDTAVYYCAKSGPGLRQVGFDYWGQGTLVTVSS

SEQ ID NO: 167 - protein, 121 aa, synthetic construct
  source 1..121: note = 1A7[CD28]_H1.51
EVQLLESGGGLVQPGGSLRLSCAASGFTFGAYSMNWVRQAPGKGLEWVSTIYNDGATTYYADSVKGRFTISRDNSKNTLYLQMNSLRAEDTAVYYCAKSGPGLRQVGFDYWGQGTLVTVSS

SEQ ID NO: 168 - protein, 121 aa, synthetic construct
  source 1..121: note = 1A7[CD28]_H1.52
EVQLLESGGGLVQPGGSLRLSCAASGFSFEAYSMNWVRQAPGKGLEWVATIYYDSSYTYYADSVKGRFTISRDNSKNTLYLQMNSLRAEDTAVYYCAKSGPGLRQVGFDYWGQGTLVTVSS

SEQ ID NO: 169 - protein, 121 aa, synthetic construct
  source 1..121: note = 1A7[CD28]_H1.53
EVQLLESGGGLVQPGGSLRLSCAASGFSFGSYYMSWVRQAPGKGLEWVASIYYGGYDTYYADSVKGRFTISRDNSKNTLYLQMNSLRAEDTAVYYCAKSGPGLRQVGFDYWGQGTLVTVSS

SEQ ID NO: 170 - protein, 121 aa, synthetic construct
  source 1..121: note = 1A7[CD28]_H1.54
EVQLLESGGGLVQPGGSLRLSCAASGFNFAEYSMSWVRQAPGKGLEWVATIYAGSDYTYYADSVKGRFTISRDNSKNTLYLQMNSLRAEDTAVYYCAKSGPGLRQVGFDYWGQGTLVTVSS

SEQ ID NO: 171 - protein, 121 aa, synthetic construct
  source 1..121: note = 1A7[CD28]_H1.55
EVQLLESGGGLVQPGGSLRLSCAASGFTFGAYSINWVRQAPGKGLEWVATIYYDGSYTYYADSVKGRFTISRDNSKNTLYLQMNSLRAEDTAVYYCAKSGPGLRQVGFDYWGQGTLVTVSS

SEQ ID NO: 172 - protein, 121 aa, synthetic construct
  source 1..121: note = 1A7[CD28]_H1.56
EVQLLESGGGLVQPGGSLRLSCAASGFTFSRYYINWVRQAPGKGLEWVASIYDGGADTYYADSVKGRFTISRDNSKNTLYLQMNSLRAEDTAVYYCAKSGPGLRQVGFDYWGQGTLVTVSS

SEQ ID NO: 173 - protein, 121 aa, synthetic construct
  source 1..121: note = 1A7[CD28]_H1.57
EVQLLESGGGLVQPGGSLRLSCAASGFSFGTYSINWVRQAPGKGLEWVSTIYYDGASTYYADSVKGRFTISRDNSKNTLYLQMNSLRAEDTAVYYCAKSGPGLRQVGFDYWGQGTLVTVSS

SEQ ID NO: 174 - protein, 121 aa, synthetic construct
  source 1..121: note = 1A7[CD28]_H1.58
EVQLLESGGGLVQPGGSLRLSCAASGFTFGKYSISWVRQAPGKGLEWVATIYNDGYYTYYADSVKGRFTISRDNSKNTLYLQMNSLRAEDTAVYYCAKSGPGLRQVGFDYWGQGTLVTVSS

SEQ ID NO: 175 - protein, 121 aa, synthetic construct
  source 1..121: note = 1A7[CD28]_H1.59
EVQLLESGGGLVQPGGSLRLSCAASGFSFEKYYISWVRQAPGKGLEWVASIYDGSYDTYYADSVKGRFTISRDNSKNTLYLQMNSLRAEDTAVYYCAKSGPGLRQVGFDYWGQGTLVTVSS

SEQ ID NO: 176 - protein, 121 aa, synthetic construct
  source 1..121: note = 1A7[CD28]_H1.60
EVQLLESGGGLVQPGGSLRLSCAASGFSFGTYSMNWVRQAPGKGLEWVATIDYDGSNTYYADSVKGRFTISRDNSKNTLYLQMNSLRAEDTAVYYCAKSGPGLRQVGFDYWGQGTLVTVSS

SEQ ID NO: 177 - protein, 121 aa, synthetic construct
  source 1..121: note = 1A7[CD28]_H1.61
EVQLLESGGGLVQPGGSLRLSCAASGFSFAAYSMSWVRQAPGKGLEWVATIYAGSDYTYYADSVKGRFTISRDNSKNTLYLQMNSLRAEDTAVYYCAKSGPGLRQVGFDYWGQGTLVTVSS

SEQ ID NO: 178 - protein, 121 aa, synthetic construct
  source 1..121: note = 1A7[CD28]_H1.62
EVQLLESGGGLVQPGGSLRLSCAASGFTFTTYSMTWVRQAPGKGLEWVATIYNDGYYTYYADSVKGRFTISRDNSKNTLYLQMNSLRAEDTAVYYCAKSGPGLRQVGFDYWGQGTLVTVSS

SEQ ID NO: 179 - protein, 121 aa, synthetic construct
  source 1..121: note = 1A7[CD28]_H1.63
EVQLLESGGGLVQPGGSLRLSCAASGFSFKDYSMNWVRQAPGKGLEWVATIYYDGTYTYYADSVKGRFTISRDNSKNTLYLQMNSLRAEDTAVYYCAKSGPGLRQVGFDYWGQGTLVTVSS

SEQ ID NO: 180 - protein, 121 aa, synthetic construct
  source 1..121: note = 1A7[CD28]_H1.64
EVQLLESGGGLVQPGGSLRLSCAASGFSFAAYSMNWVRQAPGKGLEWVATIYAGGAYTYYADSVKGRFTISRDNSKNTLYLQMNSLRAEDTAVYYCAKSGPGLRQVGFDYWGQGTLVTVSS

SEQ ID NO: 181 - protein, 121 aa, synthetic construct
  source 1..121: note = 1A7[CD28]_H1.65
EVQLLESGGGLVQPGGSLRLSCAASGFNFAEYYISWVRQAPGKGLEWVASIYNGGSDTYYADSVKGRFTISRDNSKNTLYLQMNSLRAEDTAVYYCAKSGPGLRQVGFDYWGQGTLVTVSS

SEQ ID NO: 182 - protein, 121 aa, synthetic construct
  source 1..121: note = 1A7[CD28]_H1.66
EVQLLESGGGLVQPGGSLRLSCAASGFSLGEYYMNWVRQAPGKGLEWVSTIDYDGTYTYYADSVKGRFTISRDNSKNTLYLQMNSLRAEDTAVYYCAKSGPGLRQVGFDYWGQGTLVTVSS

SEQ ID NO: 183 - protein, 121 aa, synthetic construct
  source 1..121: note = 1A7[CD28]_H1.67
EVQLLESGGGLVQPGGSLRLSCAASGFTFNEYYITWVRQAPGKGLEWVSSIYSSSYDTYYADSVKGRFTISRDNSKNTLYLQMNSLRAEDTAVYYCAKSGPGLRQVGFDYWGQGTLVTVSS

SEQ ID NO: 184 - protein, 121 aa, synthetic construct
  source 1..121: note = 1A7[CD28]_H1.68
EVQLLESGGGLVQPGGSLRLSCAASGFTFKDYYITWVRQAPGKGLEWVSSIYTSGYDTYYADSVKGRFTISRDNSKNTLYLQMNSLRAEDTAVYYCAKSGPGLRQVGFDYWGQGTLVTVSS

SEQ ID NO: 185 - protein, 121 aa, synthetic construct
  source 1..121: note = 1A7[CD28]_H1.69
EVQLLESGGGLVQPGGSLRLSCAASGFSFGKYSINWVRQAPGKGLEWVATIYSDGTDTYYADSVKGRFTISRDNSKNTLYLQMNSLRAEDTAVYYCAKSGPGLRQVGFDYWGQGTLVTVSS

SEQ ID NO: 186 - protein, 107 aa, synthetic construct
  source 1..107: note = 1A7[CD28]_L1.1
DIQMTQSPSSLSASVGDRVTITCRASQSISAYLNWYQQKPGKAPKLLIYAASSLQSGVPSRFSGSGSGTDFTLTISSLQPEDFATYYCQQSYSTPFTFGQGTKLEIK

SEQ ID NO: 187 - protein, 107 aa, synthetic construct
  source 1..107: note = 1A7[CD28]_L1.2
DIQMTQSPSSLSASVGDRVTITCRASQSISDYLNWYQQKPGKAPKLLIYAASSLQSGVPSRFSGSGSGTDFTLTISSLQPEDFATYYCQQSYSTPFTFGQGTKLEIK

SEQ ID NO: 188 - protein, 107 aa, synthetic construct
  source 1..107: note = 1A7[CD28]_L1.3
DIQMTQSPSSLSASVGDRVTITCRASQSISGYLNWYQQKPGKAPKLLIYAASSLQSGVPSRFSGSGSGTDFTLTISSLQPEDFATYYCQQSYSTPFTFGQGTKLEIK

SEQ ID NO: 189 - protein, 107 aa, synthetic construct
  source 1..107: note = 1A7[CD28]_L1.4
DIQMTQSPSSLSASVGDRVTITCRASQSISHYLNWYQQKPGKAPKLLIYAASSLQSGVPSRFSGSGSGTDFTLTISSLQPEDFATYYCQQSYSTPFTFGQGTKLEIK

SEQ ID NO: 190 - protein, 107 aa, synthetic construct
  source 1..107: note = 1A7[CD28]_L1.5
DIQMTQSPSSLSASVGDRVTITCRASQSISKYLNWYQQKPGKAPKLLIYAASSLQSGVPSRFSGSGSGTDFTLTISSLQPEDFATYYCQQSYSTPFTFGQGTKLEIK

SEQ ID NO: 191 - protein, 107 aa, synthetic construct
  source 1..107: note = 1A7[CD28]_L1.6
DIQMTQSPSSLSASVGDRVTITCRASQSISNYLNWYQQKPGKAPKLLIYAASSLQSGVPSRFSGSGSGTDFTLTISSLQPEDFATYYCQQSYSTPFTFGQGTKLEIK

SEQ ID NO: 192 - protein, 107 aa, synthetic construct
  source 1..107: note = 1A7[CD28]_L1.7
DIQMTQSPSSLSASVGDRVTITCRASQSISQYLNWYQQKPGKAPKLLIYAASSLQSGVPSRFSGSGSGTDFTLTISSLQPEDFATYYCQQSYSTPFTFGQGTKLEIK

SEQ ID NO: 193 - protein, 107 aa, synthetic construct
  source 1..107: note = 1A7[CD28]_L1.8
DIQMTQSPSSLSASVGDRVTITCRASQSISTYLNWYQQKPGKAPKLLIYAASSLQSGVPSRFSGSGSGTDFTLTISSLQPEDFATYYCQQSYSTPFTFGQGTKLEIK

SEQ ID NO: 194 - protein, 107 aa, synthetic construct
  source 1..107: note = 1A7[CD28]_L1.9
DIQMTQSPSSLSASVGDRVTITCRASQSISVYLNWYQQKPGKAPKLLIYAASSLQSGVPSRFSGSGSGTDFTLTISSLQPEDFATYYCQQSYSTPFTFGQGTKLEIK

SEQ ID NO: 195 - protein, 107 aa, synthetic construct
  source 1..107: note = 1A7[CD28]_L1.10
DIQMTQSPSSLSASVGDRVTITCRASQSISYYLNWYQQKPGKAPKLLIYAASSLQSGVPSRFSGSGSGTDFTLTISSLQPEDFATYYCQQSYSTPFTFGQGTKLEIK

SEQ ID NO: 196 - protein, 107 aa, synthetic construct
  source 1..107: note = 1A7[CD28]_L1.11
DIQMTQSPSSLSASVGDRVTITCRASQSISSALNWYQQKPGKAPKLLIYAASSLQSGVPSRFSGSGSGTDFTLTISSLQPEDFATYYCQQSYSTPFTFGQGTKLEIK

SEQ ID NO: 197 - protein, 107 aa, synthetic construct
  source 1..107: note = 1A7[CD28]_L1.12
DIQMTQSPSSLSASVGDRVTITCRASQSISSDLNWYQQKPGKAPKLLIYAASSLQSGVPSRFSGSGSGTDFTLTISSLQPEDFATYYCQQSYSTPFTFGQGTKLEIK

SEQ ID NO: 198 - protein, 107 aa, synthetic construct
  source 1..107: note = 1A7[CD28]_L1.13
DIQMTQSPSSLSASVGDRVTITCRASQSISSFLNWYQQKPGKAPKLLIYAASSLQSGVPSRFSGSGSGTDFTLTISSLQPEDFATYYCQQSYSTPFTFGQGTKLEIK

SEQ ID NO: 199 - protein, 107 aa, synthetic construct
  source 1..107: note = 1A7[CD28]_L1.14
DIQMTQSPSSLSASVGDRVTITCRASQSISSHLNWYQQKPGKAPKLLIYAASSLQSGVPSRFSGSGSGTDFTLTISSLQPEDFATYYCQQSYSTPFTFGQGTKLEIK

SEQ ID NO: 200 - protein, 107 aa, synthetic construct
  source 1..107: note = 1A7[CD28]_L1.15
DIQMTQSPSSLSASVGDRVTITCRASQSISSKLNWYQQKPGKAPKLLIYAASSLQSGVPSRFSGSGSGTDFTLTISSLQPEDFATYYCQQSYSTPFTFGQGTKLEIK

SEQ ID NO: 201 - protein, 107 aa, synthetic construct
  source 1..107: note = 1A7[CD28]_L1.16
DIQMTQSPSSLSASVGDRVTITCRASQSISSLLNWYQQKPGKAPKLLIYAASSLQSGVPSRFSGSGSGTDFTLTISSLQPEDFATYYCQQSYSTPFTFGQGTKLEIK

SEQ ID NO: 202 - protein, 107 aa, synthetic construct
  source 1..107: note = 1A7[CD28]_L1.17
DIQMTQSPSSLSASVGDRVTITCRASQSISSNLNWYQQKPGKAPKLLIYAASSLQSGVPSRFSGSGSGTDFTLTISSLQPEDFATYYCQQSYSTPFTFGQGTKLEIK

SEQ ID NO: 203 - protein, 107 aa, synthetic construct
  source 1..107: note = 1A7[CD28]_L1.18
DIQMTQSPSSLSASVGDRVTITCRASQSISSQLNWYQQKPGKAPKLLIYAASSLQSGVPSRFSGSGSGTDFTLTISSLQPEDFATYYCQQSYSTPFTFGQGTKLEIK

SEQ ID NO: 204 - protein, 107 aa, synthetic construct
  source 1..107: note = 1A7[CD28]_L1.19
DIQMTQSPSSLSASVGDRVTITCRASQSISSSLNWYQQKPGKAPKLLIYAASSLQSGVPSRFSGSGSGTDFTLTISSLQPEDFATYYCQQSYSTPFTFGQGTKLEIK

SEQ ID NO: 205 - protein, 107 aa, synthetic construct
  source 1..107: note = 1A7[CD28]_L1.20
DIQMTQSPSSLSASVGDRVTITCRASQSISSWLNWYQQKPGKAPKLLIYAASSLQSGVPSRFSGSGSGTDFTLTISSLQPEDFATYYCQQSYSTPFTFGQGTKLEIK

SEQ ID NO: 206 - protein, 107 aa, synthetic construct
  source 1..107: note = 1A7[CD28]_L1.21
DIQMTQSPSSLSASVGDRVTITCRASQSISSYLAWYQQKPGKAPKLLIYAASSLQSGVPSRFSGSGSGTDFTLTISSLQPEDFATYYCQQSYSTPFTFGQGTKLEIK

SEQ ID NO: 207 - protein, 107 aa, synthetic construct
  source 1..107: note = 1A7[CD28]_L1.22
DIQMTQSPSSLSASVGDRVTITCRASQSISSYLDWYQQKPGKAPKLLIYAASSLQSGVPSRFSGSGSGTDFTLTISSLQPEDFATYYCQQSYSTPFTFGQGTKLEIK

SEQ ID NO: 208 - protein, 107 aa, synthetic construct
  source 1..107: note = 1A7[CD28]_L1.23
DIQMTQSPSSLSASVGDRVTITCRASQSISSYLGWYQQKPGKAPKLLIYAASSLQSGVPSRFSGSGSGTDFTLTISSLQPEDFATYYCQQSYSTPFTFGQGTKLEIK

SEQ ID NO: 209 - protein, 107 aa, synthetic construct
  source 1..107: note = 1A7[CD28]_L1.24
DIQMTQSPSSLSASVGDRVTITCRASQSISSYLHWYQQKPGKAPKLLIYAASSLQSGVPSRFSGSGSGTDFTLTISSLQPEDFATYYCQQSYSTPFTFGQGTKLEIK

SEQ ID NO: 210 - protein, 107 aa, synthetic construct
  source 1..107: note = 1A7[CD28]_L1.25
DIQMTQSPSSLSASVGDRVTITCRASQSISSYLQWYQQKPGKAPKLLIYAASSLQSGVPSRFSGSGSGTDFTLTISSLQPEDFATYYCQQSYSTPFTFGQGTKLEIK

SEQ ID NO: 211 - protein, 107 aa, synthetic construct
  source 1..107: note = 1A7[CD28]_L1.26
DIQMTQSPSSLSASVGDRVTITCRASQSISSYLSWYQQKPGKAPKLLIYAASSLQSGVPSRFSGSGSGTDFTLTISSLQPEDFATYYCQQSYSTPFTFGQGTKLEIK

SEQ ID NO: 212 - protein, 107 aa, synthetic construct
  source 1..107: note = 1A7[CD28]_L1.27
DIQMTQSPSSLSASVGDRVTITCRASQSISSYLTWYQQKPGKAPKLLIYAASSLQSGVPSRFSGSGSGTDFTLTISSLQPEDFATYYCQQSYSTPFTFGQGTKLEIK

SEQ ID NO: 213 - protein, 107 aa, synthetic construct
  source 1..107: note = 1A7[CD28]_L1.28
DIQMTQSPSSLSASVGDRVTITCRASQSISSYLYWYQQKPGKAPKLLIYAASSLQSGVPSRFSGSGSGTDFTLTISSLQPEDFATYYCQQSYSTPFTFGQGTKLEIK

SEQ ID NO: 214 - protein, 107 aa, synthetic construct
  source 1..107: note = 1A7[CD28]_L1.29
DIQMTQSPSSLSASVGDRVTITCRASQSISSYLNWYQQKPGKAPKLLIYDASSLQSGVPSRFSGSGSGTDFTLTISSLQPEDFATYYCQQSYSTPFTFGQGTKLEIK

SEQ ID NO: 215 - protein, 107 aa, synthetic construct
  source 1..107: note = 1A7[CD28]_L1.30
DIQMTQSPSSLSASVGDRVTITCRASQSISSYLNWYQQKPGKAPKLLIYGASSLQSGVPSRFSGSGSGTDFTLTISSLQPEDFATYYCQQSYSTPFTFGQGTKLEIK

SEQ ID NO: 216 - protein, 107 aa, synthetic construct
  source 1..107: note = 1A7[CD28]_L1.31
DIQMTQSPSSLSASVGDRVTITCRASQSISSYLNWYQQKPGKAPKLLIYKASSLQSGVPSRFSGSGSGTDFTLTISSLQPEDFATYYCQQSYSTPFTFGQGTKLEIK

SEQ ID NO: 217 - protein, 107 aa, synthetic construct
  source 1..107: note = 1A7[CD28]_L1.32
DIQMTQSPSSLSASVGDRVTITCRASQSISSYLNWYQQKPGKAPKLLIYLASSLQSGVPSRFSGSGSGTDFTLTISSLQPEDFATYYCQQSYSTPFTFGQGTKLEIK

SEQ ID NO: 218 - protein, 107 aa, synthetic construct
  source 1..107: note = 1A7[CD28]_L1.33
DIQMTQSPSSLSASVGDRVTITCRASQSISSYLNWYQQKPGKAPKLLIYQASSLQSGVPSRFSGSGSGTDFTLTISSLQPEDFATYYCQQSYSTPFTFGQGTKLEIK

SEQ ID NO: 219 - protein, 107 aa, synthetic construct
  source 1..107: note = 1A7[CD28]_L1.34
DIQMTQSPSSLSASVGDRVTITCRASQSISSYLNWYQQKPGKAPKLLIYSASSLQSGVPSRFSGSGSGTDFTLTISSLQPEDFATYYCQQSYSTPFTFGQGTKLEIK

SEQ ID NO: 220 - protein, 107 aa, synthetic construct
  source 1..107: note = 1A7[CD28]_L1.35
DIQMTQSPSSLSASVGDRVTITCRASQSISSYLNWYQQKPGKAPKLLIYTASSLQSGVPSRFSGSGSGTDFTLTISSLQPEDFATYYCQQSYSTPFTFGQGTKLEIK

SEQ ID NO: 221 - protein, 107 aa, synthetic construct
  source 1..107: note = 1A7[CD28]_L1.36
DIQMTQSPSSLSASVGDRVTITCRASQSISSYLNWYQQKPGKAPKLLIYWASSLQSGVPSRFSGSGSGTDFTLTISSLQPEDFATYYCQQSYSTPFTFGQGTKLEIK

SEQ ID NO: 222 - protein, 107 aa, synthetic construct
  source 1..107: note = 1A7[CD28]_L1.37
DIQMTQSPSSLSASVGDRVTITCRASQSISSYLNWYQQKPGKAPKLLIYYASSLQSGVPSRFSGSGSGTDFTLTISSLQPEDFATYYCQQSYSTPFTFGQGTKLEIK

SEQ ID NO: 223 - protein, 107 aa, synthetic construct
  source 1..107: note = 1A7[CD28]_L1.38
DIQMTQSPSSLSASVGDRVTITCRASQSISSYLNWYQQKPGKAPKLLIYAASALQSGVPSRFSGSGSGTDFTLTISSLQPEDFATYYCQQSYSTPFTFGQGTKLEIK

SEQ ID NO: 224 - protein, 107 aa, synthetic construct
  source 1..107: note = 1A7[CD28]_L1.39
DIQMTQSPSSLSASVGDRVTITCRASQSISSYLNWYQQKPGKAPKLLIYAASDLQSGVPSRFSGSGSGTDFTLTISSLQPEDFATYYCQQSYSTPFTFGQGTKLEIK

SEQ ID NO: 225 - protein, 107 aa, synthetic construct
  source 1..107: note = 1A7[CD28]_L1.40
DIQMTQSPSSLSASVGDRVTITCRASQSISSYLNWYQQKPGKAPKLLIYAASKLQSGVPSRFSGSGSGTDFTLTISSLQPEDFATYYCQQSYSTPFTFGQGTKLEIK

SEQ ID NO: 226 - protein, 107 aa, synthetic construct
  source 1..107: note = 1A7[CD28]_L1.41
DIQMTQSPSSLSASVGDRVTITCRASQSISSYLNWYQQKPGKAPKLLIYAASNLQSGVPSRFSGSGSGTDFTLTISSLQPEDFATYYCQQSYSTPFTFGQGTKLEIK

SEQ ID NO: 227 - protein, 107 aa, synthetic construct
  source 1..107: note = 1A7[CD28]_L1.42
DIQMTQSPSSLSASVGDRVTITCRASQSISSYLNWYQQKPGKAPKLLIYAASQLQSGVPSRFSGSGSGTDFTLTISSLQPEDFATYYCQQSYSTPFTFGQGTKLEIK

SEQ ID NO: 228 - protein, 107 aa, synthetic construct
  source 1..107: note = 1A7[CD28]_L1.43
DIQMTQSPSSLSASVGDRVTITCRASQSISSYLNWYQQKPGKAPKLLIYAASTLQSGVPSRFSGSGSGTDFTLTISSLQPEDFATYYCQQSYSTPFTFGQGTKLEIK

SEQ ID NO: 229 - protein, 107 aa, synthetic construct
  source 1..107: note = 1A7[CD28]_L1.44
DIQMTQSPSSLSASVGDRVTITCRASQSISSYLNWYQQKPGKAPKLLIYAASYLQSGVPSRFSGSGSGTDFTLTISSLQPEDFATYYCQQSYSTPFTFGQGTKLEIK

SEQ ID NO: 230 - protein, 107 aa, synthetic construct
  source 1..107: note = 1A7[CD28]_L1.45
DIQMTQSPSSLSASVGDRVTITCRASQSISSYLNWYQQKPGKAPKLLIYAASSLASGVPSRFSGSGSGTDFTLTISSLQPEDFATYYCQQSYSTPFTFGQGTKLEIK

SEQ ID NO: 231 - protein, 107 aa, synthetic construct
  source 1..107: note = 1A7[CD28]_L1.46
DIQMTQSPSSLSASVGDRVTITCRASQSISSYLNWYQQKPGKAPKLLIYAASSLESGVPSRFSGSGSGTDFTLTISSLQPEDFATYYCQQSYSTPFTFGQGTKLEIK

SEQ ID NO: 232 - protein, 107 aa, synthetic construct
  source 1..107: note = 1A7[CD28]_L1.47
DIQMTQSPSSLSASVGDRVTITCRASQSISSYLNWYQQKPGKAPKLLIYAASSLFSGVPSRFSGSGSGTDFTLTISSLQPEDFATYYCQQSYSTPFTFGQGTKLEIK

SEQ ID NO: 233 - protein, 107 aa, synthetic construct
  source 1..107: note = 1A7[CD28]_L1.48
DIQMTQSPSSLSASVGDRVTITCRASQSISSYLNWYQQKPGKAPKLLIYAASSLHSGVPSRFSGSGSGTDFTLTISSLQPEDFATYYCQQSYSTPFTFGQGTKLEIK

SEQ ID NO: 234 - protein, 107 aa, synthetic construct
  source 1..107: note = 1A7[CD28]_L1.49
DIQMTQSPSSLSASVGDRVTITCRASQSISSYLNWYQQKPGKAPKLLIYAASSLISGVPSRFSGSGSGTDFTLTISSLQPEDFATYYCQQSYSTPFTFGQGTKLEIK

SEQ ID NO: 235 - protein, 107 aa, synthetic construct
  source 1..107: note = 1A7[CD28]_L1.50
DIQMTQSPSSLSASVGDRVTITCRASQSISSYLNWYQQKPGKAPKLLIYAASSLKSGVPSRFSGSGSGTDFTLTISSLQPEDFATYYCQQSYSTPFTFGQGTKLEIK

SEQ ID NO: 236 - protein, 107 aa, synthetic construct
  source 1..107: note = 1A7[CD28]_L1.51
DIQMTQSPSSLSASVGDRVTITCRASQSISSYLNWYQQKPGKAPKLLIYAASSLNSGVPSRFSGSGSGTDFTLTISSLQPEDFATYYCQQSYSTPFTFGQGTKLEIK

SEQ ID NO: 237 - protein, 107 aa, synthetic construct
  source 1..107: note = 1A7[CD28]_L1.52
DIQMTQSPSSLSASVGDRVTITCRASQSISSYLNWYQQKPGKAPKLLIYAASSLSSGVPSRFSGSGSGTDFTLTISSLQPEDFATYYCQQSYSTPFTFGQGTKLEIK

SEQ ID NO: 238 - protein, 107 aa, synthetic construct
  source 1..107: note = 1A7[CD28]_L1.53
DIQMTQSPSSLSASVGDRVTITCRASQSISSYLNWYQQKPGKAPKLLIYAASSLVSGVPSRFSGSGSGTDFTLTISSLQPEDFATYYCQQSYSTPFTFGQGTKLEIK

SEQ ID NO: 239 - protein, 107 aa, synthetic construct
  source 1..107: note = 1A7[CD28]_L1.54
DIQMTQSPSSLSASVGDRVTITCRASQSISSYLNWYQQKPGKAPKLLIYAASSLYSGVPSRFSGSGSGTDFTLTISSLQPEDFATYYCQQSYSTPFTFGQGTKLEIK

SEQ ID NO: 240 - protein, 107 aa, synthetic construct
  source 1..107: note = 1A7[CD28]_L1.55
DIQMTQSPSSLSASVGDRVTITCRASQSISSYLNWYQQKPGKAPKLLIYAASSLQAGVPSRFSGSGSGTDFTLTISSLQPEDFATYYCQQSYSTPFTFGQGTKLEIK

SEQ ID NO: 241 - protein, 107 aa, synthetic construct
  source 1..107: note = 1A7[CD28]_L1.56
DIQMTQSPSSLSASVGDRVTITCRASQSISSYLNWYQQKPGKAPKLLIYAASSLQDGVPSRFSGSGSGTDFTLTISSLQPEDFATYYCQQSYSTPFTFGQGTKLEIK

SEQ ID NO: 242 - protein, 107 aa, synthetic construct
  source 1..107: note = 1A7[CD28]_L1.57
DIQMTQSPSSLSASVGDRVTITCRASQSISSYLNWYQQKPGKAPKLLIYAASSLQGGVPSRFSGSGSGTDFTLTISSLQPEDFATYYCQQSYSTPFTFGQGTKLEIK

SEQ ID NO: 243 - protein, 107 aa, synthetic construct
  source 1..107: note = 1A7[CD28]_L1.58
DIQMTQSPSSLSASVGDRVTITCRASQSISSYLNWYQQKPGKAPKLLIYAASSLQHGVPSRFSGSGSGTDFTLTISSLQPEDFATYYCQQSYSTPFTFGQGTKLEIK

SEQ ID NO: 244 - protein, 107 aa, synthetic construct
  source 1..107: note = 1A7[CD28]_L1.59
DIQMTQSPSSLSASVGDRVTITCRASQSISSYLNWYQQKPGKAPKLLIYAASSLQKGVPSRFSGSGSGTDFTLTISSLQPEDFATYYCQQSYSTPFTFGQGTKLEIK

SEQ ID NO: 245 - protein, 107 aa, synthetic construct
  source 1..107: note = 1A7[CD28]_L1.60
DIQMTQSPSSLSASVGDRVTITCRASQSISSYLNWYQQKPGKAPKLLIYAASSLQQGVPSRFSGSGSGTDFTLTISSLQPEDFATYYCQQSYSTPFTFGQGTKLEIK

SEQ ID NO: 246 - protein, 107 aa, synthetic construct
  source 1..107: note = 1A7[CD28]_L1.61
DIQMTQSPSSLSASVGDRVTITCRASQSISSYLNWYQQKPGKAPKLLIYAASSLQTGVPSRFSGSGSGTDFTLTISSLQPEDFATYYCQQSYSTPFTFGQGTKLEIK

SEQ ID NO: 247 - protein, 107 aa, synthetic construct
  source 1..107: note = 1A7[CD28]_L1.62
DIQMTQSPSSLSASVGDRVTITCRASQSISSYLNWYQQKPGKAPKLLIYAASSLQVGVPSRFSGSGSGTDFTLTISSLQPEDFATYYCQQSYSTPFTFGQGTKLEIK

SEQ ID NO: 248 - protein, 107 aa, synthetic construct
  source 1..107: note = 1A7[CD28]_L1.63
DIQMTQSPSSLSASVGDRVTITCRASQSISSYLNWYQQKPGKAPKLLIYAASSLQYGVPSRFSGSGSGTDFTLTISSLQPEDFATYYCQQSYSTPFTFGQGTKLEIK

SEQ ID NO: 249 - protein, 107 aa, synthetic construct
  source 1..107: note = 1A7[CD28]_L1.64
DIQMTQSPSSLSASVGDRVTITCRASQSISSYLNWYQQKPGKAPKLLIYAASSLQSGVPSRFSGSGSGTDFTLTISSLQPEDFATYYCQQAYSTPFTFGQGTKLEIK

SEQ ID NO: 250 - protein, 107 aa, synthetic construct
  source 1..107: note = 1A7[CD28]_L1.65
DIQMTQSPSSLSASVGDRVTITCRASQSISSYLNWYQQKPGKAPKLLIYAASSLQSGVPSRFSGSGSGTDFTLTISSLQPEDFATYYCQQDYSTPFTFGQGTKLEIK

SEQ ID NO: 251 - protein, 107 aa, synthetic construct
  source 1..107: note = 1A7[CD28]_L1.66
DIQMTQSPSSLSASVGDRVTITCRASQSISSYLNWYQQKPGKAPKLLIYAASSLQSGVPSRFSGSGSGTDFTLTISSLQPEDFATYYCQQFYSTPFTFGQGTKLEIK

SEQ ID NO: 252 - protein, 107 aa, synthetic construct
  source 1..107: note = 1A7[CD28]_L1.67
DIQMTQSPSSLSASVGDRVTITCRASQSISSYLNWYQQKPGKAPKLLIYAASSLQSGVPSRFSGSGSGTDFTLTISSLQPEDFATYYCQQHYSTPFTFGQGTKLEIK

SEQ ID NO: 253 - protein, 107 aa, synthetic construct
  source 1..107: note = 1A7[CD28]_L1.68
DIQMTQSPSSLSASVGDRVTITCRASQSISSYLNWYQQKPGKAPKLLIYAASSLQSGVPSRFSGSGSGTDFTLTISSLQPEDFATYYCQQKYSTPFTFGQGTKLEIK

SEQ ID NO: 254 - protein, 107 aa, synthetic construct
  source 1..107: note = 1A7[CD28]_L1.69
DIQMTQSPSSLSASVGDRVTITCRASQSISSYLNWYQQKPGKAPKLLIYAASSLQSGVPSRFSGSGSGTDFTLTISSLQPEDFATYYCQQLYSTPFTFGQGTKLEIK

SEQ ID NO: 255 - protein, 107 aa, synthetic construct
  source 1..107: note = 1A7[CD28]_L1.70
DIQMTQSPSSLSASVGDRVTITCRASQSISSYLNWYQQKPGKAPKLLIYAASSLQSGVPSRFSGSGSGTDFTLTISSLQPEDFATYYCQQTYSTPFTFGQGTKLEIK

SEQ ID NO: 256 - protein, 107 aa, synthetic construct
  source 1..107: note = 1A7[CD28]_L1.71
DIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 257 - protein, 107 aa, synthetic construct
  source 1..107: note = 1A7[CD28]_L1.72
DIQMTQSPSSLSASVGDRVTITCRASQSISSYLNWYQQKPGKAPKLLIYAASSLQSGVPSRFSGSGSGTDFTLTISSLQPEDFATYYCQQYYSTPFTFGQGTKLEIK

SEQ ID NO: 258 - protein, 107 aa, synthetic construct
  source 1..107: note = 1A7[CD28]_L1.73
DIQMTQSPSSLSASVGDRVTITCRASQSISSYLNWYQQKPGKAPKLLIYAASSLQSGVPSRFSGSGSGTDFTLTISSLQPEDFATYYCQQSASTPFTFGQGTKLEIK

SEQ ID NO: 259 - protein, 107 aa, synthetic construct
  source 1..107: note = 1A7[CD28]_L1.74
DIQMTQSPSSLSASVGDRVTITCRASQSISSYLNWYQQKPGKAPKLLIYAASSLQSGVPSRFSGSGSGTDFTLTISSLQPEDFATYYCQQSDSTPFTFGQGTKLEIK

SEQ ID NO: 260 - protein, 107 aa, synthetic construct
  source 1..107: note = 1A7[CD28]_L1.75
DIQMTQSPSSLSASVGDRVTITCRASQSISSYLNWYQQKPGKAPKLLIYAASSLQSGVPSRFSGSGSGTDFTLTISSLQPEDFATYYCQQSFSTPFTFGQGTKLEIK

SEQ ID NO: 261 - protein, 107 aa, synthetic construct
  source 1..107: note = 1A7[CD28]_L1.76
DIQMTQSPSSLSASVGDRVTITCRASQSISSYLNWYQQKPGKAPKLLIYAASSLQSGVPSRFSGSGSGTDFTLTISSLQPEDFATYYCQQSHSTPFTFGQGTKLEIK

SEQ ID NO: 262 - protein, 107 aa, synthetic construct
  source 1..107: note = 1A7[CD28]_L1.77
DIQMTQSPSSLSASVGDRVTITCRASQSISSYLNWYQQKPGKAPKLLIYAASSLQSGVPSRFSGSGSGTDFTLTISSLQPEDFATYYCQQSKSTPFTFGQGTKLEIK

SEQ ID NO: 263 - protein, 107 aa, synthetic construct
  source 1..107: note = 1A7[CD28]_L1.78
DIQMTQSPSSLSASVGDRVTITCRASQSISSYLNWYQQKPGKAPKLLIYAASSLQSGVPSRFSGSGSGTDFTLTISSLQPEDFATYYCQQSLSTPFTFGQGTKLEIK

SEQ ID NO: 264 - protein, 107 aa, synthetic construct
  source 1..107: note = 1A7[CD28]_L1.79
DIQMTQSPSSLSASVGDRVTITCRASQSISSYLNWYQQKPGKAPKLLIYAASSLQSGVPSRFSGSGSGTDFTLTISSLQPEDFATYYCQQSQSTPFTFGQGTKLEIK

SEQ ID NO: 265 - protein, 107 aa, synthetic construct
  source 1..107: note = 1A7[CD28]_L1.80
DIQMTQSPSSLSASVGDRVTITCRASQSISSYLNWYQQKPGKAPKLLIYAASSLQSGVPSRFSGSGSGTDFTLTISSLQPEDFATYYCQQSVSTPFTFGQGTKLEIK

SEQ ID NO: 266 - protein, 107 aa, synthetic construct
  source 1..107: note = 1A7[CD28]_L1.81
DIQMTQSPSSLSASVGDRVTITCRASQSISSYLNWYQQKPGKAPKLLIYAASSLQSGVPSRFSGSGSGTDFTLTISSLQPEDFATYYCQQSWSTPFTFGQGTKLEIK

SEQ ID NO: 267 - protein, 107 aa, synthetic construct
  source 1..107: note = 1A7[CD28]_L1.82
DIQMTQSPSSLSASVGDRVTITCRASQSISSYLNWYQQKPGKAPKLLIYAASSLQSGVPSRFSGSGSGTDFTLTISSLQPEDFATYYCQQSYATPFTFGQGTKLEIK

SEQ ID NO: 268 - protein, 107 aa, synthetic construct
  source 1..107: note = 1A7[CD28]_L1.83
DIQMTQSPSSLSASVGDRVTITCRASQSISSYLNWYQQKPGKAPKLLIYAASSLQSGVPSRFSGSGSGTDFTLTISSLQPEDFATYYCQQSYDTPFTFGQGTKLEIK

SEQ ID NO: 269 - protein, 107 aa, synthetic construct
  source 1..107: note = 1A7[CD28]_L1.84
DIQMTQSPSSLSASVGDRVTITCRASQSISSYLNWYQQKPGKAPKLLIYAASSLQSGVPSRFSGSGSGTDFTLTISSLQPEDFATYYCQQSYGTPFTFGQGTKLEIK

SEQ ID NO: 270 - protein, 107 aa, synthetic construct
  source 1..107: note = 1A7[CD28]_L1.85
DIQMTQSPSSLSASVGDRVTITCRASQSISSYLNWYQQKPGKAPKLLIYAASSLQSGVPSRFSGSGSGTDFTLTISSLQPEDFATYYCQQSYHTPFTFGQGTKLEIK

SEQ ID NO: 271 - protein, 107 aa, synthetic construct
  source 1..107: note = 1A7[CD28]_L1.86
DIQMTQSPSSLSASVGDRVTITCRASQSISSYLNWYQQKPGKAPKLLIYAASSLQSGVPSRFSGSGSGTDFTLTISSLQPEDFATYYCQQSYKTPFTFGQGTKLEIK

SEQ ID NO: 272 - protein, 107 aa, synthetic construct
  source 1..107: note = 1A7[CD28]_L1.87
DIQMTQSPSSLSASVGDRVTITCRASQSISSYLNWYQQKPGKAPKLLIYAASSLQSGVPSRFSGSGSGTDFTLTISSLQPEDFATYYCQQSYNTPFTFGQGTKLEIK

SEQ ID NO: 273 - protein, 107 aa, synthetic construct
  source 1..107: note = 1A7[CD28]_L1.88
DIQMTQSPSSLSASVGDRVTITCRASQSISSYLNWYQQKPGKAPKLLIYAASSLQSGVPSRFSGSGSGTDFTLTISSLQPEDFATYYCQQSYQTPFTFGQGTKLEIK

SEQ ID NO: 274 - protein, 107 aa, synthetic construct
  source 1..107: note = 1A7[CD28]_L1.89
DIQMTQSPSSLSASVGDRVTITCRASQSISSYLNWYQQKPGKAPKLLIYAASSLQSGVPSRFSGSGSGTDFTLTISSLQPEDFATYYCQQSYTTPFTFGQGTKLEIK

SEQ ID NO: 275 - protein, 107 aa, synthetic construct
  source 1..107: note = 1A7[CD28]_L1.90
DIQMTQSPSSLSASVGDRVTITCRASQSISSYLNWYQQKPGKAPKLLIYAASSLQSGVPSRFSGSGSGTDFTLTISSLQPEDFATYYCQQSYVTPFTFGQGTKLEIK

SEQ ID NO: 276 - protein, 107 aa, synthetic construct
  source 1..107: note = 1A7[CD28]_L1.91
DIQMTQSPSSLSASVGDRVTITCRASQSISSYLNWYQQKPGKAPKLLIYAASSLQSGVPSRFSGSGSGTDFTLTISSLQPEDFATYYCQQSYYTPFTFGQGTKLEIK

SEQ ID NO: 277 - protein, 107 aa, synthetic construct
  source 1..107: note = 1A7[CD28]_L1.92
DIQMTQSPSSLSASVGDRVTITCRASQSISSYLNWYQQKPGKAPKLLIYAASSLQSGVPSRFSGSGSGTDFTLTISSLQPEDFATYYCQQSYSAPFTFGQGTKLEIK

SEQ ID NO: 278 - protein, 107 aa, synthetic construct
  source 1..107: note = 1A7[CD28]_L1.93
DIQMTQSPSSLSASVGDRVTITCRASQSISSYLNWYQQKPGKAPKLLIYAASSLQSGVPSRFSGSGSGTDFTLTISSLQPEDFATYYCQQSYSDPFTFGQGTKLEIK

SEQ ID NO: 279 - protein, 107 aa, synthetic construct
  source 1..107: note = 1A7[CD28]_L1.94
DIQMTQSPSSLSASVGDRVTITCRASQSISSYLNWYQQKPGKAPKLLIYAASSLQSGVPSRFSGSGSGTDFTLTISSLQPEDFATYYCQQSYSFPFTFGQGTKLEIK

SEQ ID NO: 280 - protein, 107 aa, synthetic construct
  source 1..107: note = 1A7[CD28]_L1.95
DIQMTQSPSSLSASVGDRVTITCRASQSISSYLNWYQQKPGKAPKLLIYAASSLQSGVPSRFSGSGSGTDFTLTISSLQPEDFATYYCQQSYSIPFTFGQGTKLEIK

SEQ ID NO: 281 - protein, 107 aa, synthetic construct
  source 1..107: note = 1A7[CD28]_L1.96
DIQMTQSPSSLSASVGDRVTITCRASQSISSYLNWYQQKPGKAPKLLIYAASSLQSGVPSRFSGSGSGTDFTLTISSLQPEDFATYYCQQSYSKPFTFGQGTKLEIK

SEQ ID NO: 282 - protein, 107 aa, synthetic construct
  source 1..107: note = 1A7[CD28]_L1.97
DIQMTQSPSSLSASVGDRVTITCRASQSISSYLNWYQQKPGKAPKLLIYAASSLQSGVPSRFSGSGSGTDFTLTISSLQPEDFATYYCQQSYSLPFTFGQGTKLEIK

SEQ ID NO: 283 - protein, 107 aa, synthetic construct
  source 1..107: note = 1A7[CD28]_L1.98
DIQMTQSPSSLSASVGDRVTITCRASQSISSYLNWYQQKPGKAPKLLIYAASSLQSGVPSRFSGSGSGTDFTLTISSLQPEDFATYYCQQSYSQPFTFGQGTKLEIK

SEQ ID NO: 284 - protein, 107 aa, synthetic construct
  source 1..107: note = 1A7[CD28]_L1.99
DIQMTQSPSSLSASVGDRVTITCRASQSISSYLNWYQQKPGKAPKLLIYAASSLQSGVPSRFSGSGSGTDFTLTISSLQPEDFATYYCQQSYSSPFTFGQGTKLEIK

SEQ ID NO: 285 - protein, 107 aa, synthetic construct
  source 1..107: note = 1A7[CD28]_L1.100
DIQMTQSPSSLSASVGDRVTITCRASQSISSYLNWYQQKPGKAPKLLIYAASSLQSGVPSRFSGSGSGTDFTLTISSLQPEDFATYYCQQSYSVPFTFGQGTKLEIK

SEQ ID NO: 286 - protein, 107 aa, synthetic construct
  source 1..107: note = 1A7[CD28]_L1.101
DIQMTQSPSSLSASVGDRVTITCRASQSISSYLNWYQQKPGKAPKLLIYAASSLQSGVPSRFSGSGSGTDFTLTISSLQPEDFATYYCQQSYSYPFTFGQGTKLEIK

SEQ ID NO: 287 - protein, 107 aa, synthetic construct
  source 1..107: note = 1A7[CD28]_L1.102
DIQMTQSPSSLSASVGDRVTITCRASQSISSYLNWYQQKPGKAPKLLIYAASSLQSGVPSRFSGSGSGTDFTLTISSLQPEDFATYYCQQSYSTPITFGQGTKLEIK

SEQ ID NO: 288 - protein, 107 aa, synthetic construct
  source 1..107: note = 1A7[CD28]_L1.103
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKLEIK

SEQ ID NO: 289 - protein, 107 aa, synthetic construct
  source 1..107: note = 1A7[CD28]_L1.104
DIQMTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKLEIK

SEQ ID NO: 290 - protein, 107 aa, synthetic construct
  source 1..107: note = 1A7[CD28]_L1.105
DIQMTQSPSSLSASVGDRVTITCRASQSISSYLTWYQQKPGKAPKLLIYAASSLQSGVPSRFSGSGSGTDFTLTISSLQPEDFATYYCQQVYSTPFTFGQGTKLEIK

SEQ ID NO: 291 - protein, 107 aa, synthetic construct
  source 1..107: note = 1A7[CD28]_L1.106
DIQMTQSPSSLSASVGDRVTITCRASQSISSYLTWYQQKPGKAPKLLIYAASSLQSGVPSRFSGSGSGTDFTLTISSLQPEDFATYYCQQSYNTPFTFGQGTKLEIK

SEQ ID NO: 292 - protein, 121 aa, synthetic construct; identical to SEQ ID NO: 117
  source 1..121: note = 1A7[CD28]_H1.1_L1 Variable Heavy (vh) Domain

SEQ ID NO: 293 - protein, 5 aa, synthetic construct
  source 1..5: note = 1A7[CD28]_H1.1_L1 vhCDR1
SYYMS

SEQ ID NO: 294 - protein, 17 aa, synthetic construct
  source 1..17: note = 1A7[CD28]_H1.1_L1 vhCDR2
TISGSGDSTYYADSVKG

SEQ ID NO: 295 - protein, 12 aa, synthetic construct
  source 1..12: note = 1A7[CD28]_H1.1_L1 vhCDR3
SGPGLRQVGFDY

SEQ ID NO: 296 - protein, 107 aa, synthetic construct; identical to SEQ ID NO: 111
  source 1..107: note = 1A7[CD28]_H1.1_L1 Variable Light (vl) Domain

SEQ ID NO: 297 - protein, 11 aa, synthetic construct
  source 1..11: note = 1A7[CD28]_H1.1_L1 vlCDR1
RASQSISSYLN

SEQ ID NO: 298 - protein, 7 aa, synthetic construct
  source 1..7: note = 1A7[CD28]_H1.1_L1 vlCDR2
AASSLQS

SEQ ID NO: 299 - protein, 9 aa, synthetic construct
  source 1..9: note = 1A7[CD28]_H1.1_L1 vlCDR3
QQSYSTPFT

SEQ ID NO: 300 - protein, 121 aa, synthetic construct; identical to SEQ ID NO: 107
  source 1..121: note = 1A7[CD28]_H1_L1.71 Variable Heavy (vh) Domain

SEQ ID NO: 301 - protein, 5 aa, synthetic construct
  source 1..5: note = 1A7[CD28]_H1_L1.71 vhCDR1
SYAMS

SEQ ID NO: 302 - protein, 17 aa, synthetic construct
  source 1..17: note = 1A7[CD28]_H1_L1.71 vhCDR2
TISGSGDSTYYADSVKG

SEQ ID NO: 303 - protein, 12 aa, synthetic construct
  source 1..12: note = 1A7[CD28]_H1_L1.71 vhCDR3
SGPGLRQVGFDY

SEQ ID NO: 304 - protein, 107 aa, synthetic construct; identical to SEQ ID NO: 256
  source 1..107: note = 1A7[CD28]_H1_L1.71 Variable Light (vl) Domain

SEQ ID NO: 305 - protein, 11 aa, synthetic construct
  source 1..11: note = 1A7[CD28]_H1_L1.71 vlCDR1
RASQSISSYLN

SEQ ID NO: 306 - protein, 7 aa, synthetic construct
  source 1..7: note = 1A7[CD28]_H1_L1.71 vlCDR2
AASSLQS

SEQ ID NO: 307 - protein, 9 aa, synthetic construct
  source 1..9: note = 1A7[CD28]_H1_L1.71 vlCDR3
QQVYSTPFT

SEQ ID NO: 308 - protein, 121 aa, synthetic construct; identical to SEQ ID NO: 117
  source 1..121: note = 1A7[CD28]_H1.1_L1.71 Variable Heavy (vh) Domain

SEQ ID NO: 309 - protein, 5 aa, synthetic construct
  source 1..5: note = 1A7[CD28]_H1.1_L1.71 vhCDR1
SYYMS

SEQ ID NO: 310 - protein, 17 aa, synthetic construct
  source 1..17: note = 1A7[CD28]_H1.1_L1.71 vhCDR2
TISGSGDSTYYADSVKG

SEQ ID NO: 311 - protein, 12 aa, synthetic construct
  source 1..12: note = 1A7[CD28]_H1.1_L1.71 vhCDR3
SGPGLRQVGFDY

SEQ ID NO: 312 - protein, 107 aa, synthetic construct; identical to SEQ ID NO: 256
  source 1..107: note = 1A7[CD28]_H1.1_L1.71 Variable Light (vl) Domain

SEQ ID NO: 313 - protein, 11 aa, synthetic construct
  source 1..11: note = 1A7[CD28]_H1.1_L1.71 vlCDR1
RASQSISSYLN

SEQ ID NO: 314 - protein, 7 aa, synthetic construct
  source 1..7: note = 1A7[CD28]_H1.1_L1.71 vlCDR2
AASSLQS

SEQ ID NO: 315 - protein, 9 aa, synthetic construct
  source 1..9: note = 1A7[CD28]_H1.1_L1.71 vlCDR3
QQVYSTPFT

SEQ ID NO: 316 - protein, 121 aa, synthetic construct; identical to SEQ ID NO: 130
  source 1..121: note = 1A7[CD28]_H1.14_L1 Variable Heavy (vh) Domain

SEQ ID NO: 317 - protein, 5 aa, synthetic construct
  source 1..5: note = 1A7[CD28]_H1.14_L1 vhCDR1
SYYMS

SEQ ID NO: 318 - protein, 17 aa, synthetic construct
  source 1..17: note = 1A7[CD28]_H1.14_L1 vhCDR2
TISESGDSTYYADSVKG

SEQ ID NO: 319 - protein, 12 aa, synthetic construct
  source 1..12: note = 1A7[CD28]_H1.14_L1 vhCDR3
SGPGLRQVGFDY

SEQ ID NO: 320 - protein, 107 aa, synthetic construct; identical to SEQ ID NO: 111
  source 1..107: note = 1A7[CD28]_H1.14_L1 Variable Light (vl) Domain

SEQ ID NO: 321 - protein, 11 aa, synthetic construct
  source 1..11: note = 1A7[CD28]_H1.14_L1 vlCDR1
RASQSISSYLN

SEQ ID NO: 322 - protein, 7 aa, synthetic construct
  source 1..7: note = 1A7[CD28]_H1.14_L1 vlCDR2
AASSLQS

SEQ ID NO: 323 - protein, 9 aa, synthetic construct
  source 1..9: note = 1A7[CD28]_H1.14_L1 vlCDR3
QQSYSTPFT

SEQ ID NO: 324 - protein, 121 aa, synthetic construct; identical to SEQ ID NO: 130
  source 1..121: note = 1A7[CD28]_H1.14_L1.71 Variable Heavy (vh) Domain

SEQ ID NO: 325 - protein, 5 aa, synthetic construct
  source 1..5: note = 1A7[CD28]_H1.14_L1.71 vhCDR1
SYYMS

SEQ ID NO: 326 - protein, 17 aa, synthetic construct
  source 1..17: note = 1A7[CD28]_H1.14_L1.71 vhCDR2
TISESGDSTYYADSVKG

SEQ ID NO: 327 - protein, 12 aa, synthetic construct
  source 1..12: note = 1A7[CD28]_H1.14_L1.71 vhCDR3
SGPGLRQVGFDY

SEQ ID NO: 328 - protein, 107 aa, synthetic construct; identical to SEQ ID NO: 256
  source 1..107: note = 1A7[CD28]_H1.14_L1.71 Variable Light (vl) Domain

SEQ ID NO: 329 - protein, 11 aa, synthetic construct
  source 1..11: note = 1A7[CD28]_H1.14_L1.71 vlCDR1
RASQSISSYLN

SEQ ID NO: 330 - protein, 7 aa, synthetic construct
  source 1..7: note = 1A7[CD28]_H1.14_L1.71 vlCDR2
AASSLQS

SEQ ID NO: 331 - protein, 9 aa, synthetic construct
  source 1..9: note = 1A7[CD28]_H1.14_L1.71 vlCDR3
QQVYSTPFT

SEQ ID NO: 332 - protein, 30 aa, synthetic construct
  source 1..30: note = 1A7 VH HFR1
  VARIANT 28: note = T, S, or N
  VARIANT 29: note = F or L
  VARIANT 30: note = S, E, R, K, G, T, A, or N
EVQLLESGGGLVQPGGSLRLSCAASGFXXX

SEQ ID NO: 333 - protein, 14 aa, synthetic construct
  source 1..14: note = 1A7 VH HFR2
  VARIANT 14: note = S or A
WVRQAPGKGLEWVX

SEQ ID NO: 334 - protein, 17 aa, synthetic construct
  source 1..17: note = 1A7 VH HCDR2
  VARIANT 1: note = T or S
  VARIANT 3: note = S, D, E, Y, or T
  VARIANT 4: note = G, D, E, A, Y, S, N, or T
  VARIANT 5: note = S, D, N, or G
  VARIANT 6: note = D, T, Y, S, or A
  VARIANT 7: note = D, T, Y, S, or A
  VARIANT 8: note = S, Y, A, T, D, or N
XIXXXXXXTYYADSVKG

SEQ ID NO: 335 - protein, 32 aa, synthetic construct
  source 1..32: note = 1A7 VH HFR3
RFTISRDNSKNTLYLQMNSLRAEDTAVYYCAK

SEQ ID NO: 336 - protein, 12 aa, synthetic construct
  source 1..12: note = 1A7 VH HCDR3
SGPGLRQVGFDY

SEQ ID NO: 337 - protein, 11 aa, synthetic construct
  source 1..11: note = 1A7 VH HFR4
WGQGTLVTVSS

SEQ ID NO: 338 - protein, 23 aa, synthetic construct
  source 1..23: note = 1A7 VL LFR1
DIQMTQSPSSLSASVGDRVTITC

SEQ ID NO: 339 - protein, 11 aa, synthetic construct
  source 1..11: note = 1A7 VL LCDR1
  VARIANT 8: note = S, A, D, G, H, K, N, Q, T, V, or Y
  VARIANT 9: note = Y, A, D, F, H, K, L, N, Q, S, or W
  VARIANT 11: note = N, A, D, G, H, Q, S, T, or Y
RASQSISXXLX

SEQ ID NO: 340 - protein, 15 aa, synthetic construct
  source 1..15: note = 1A7 VL LFR2
WYQQKPGKAPKLLIY

SEQ ID NO: 341 - protein, 32 aa, synthetic construct
  source 1..32: note = 1A7 VL LFR3
GVPSRFSGSGSGTDFTLTISSLQPEDFATYYC

SEQ ID NO: 342 - protein, 9 aa, synthetic construct
  source 1..9: note = 1A7 VL LCDR3
  VARIANT 3: note = S, A, D, F, H, K, L, T, V, or Y
  VARIANT 4: note = Y, A, D, F, H, K, L, Q, V, or W
  VARIANT 5: note = S, A, D, G, H, K, N, Q, T, V, or Y
  VARIANT 6: note = T, A, D, F, I, K, L, Q, S, V, or Y
  VARIANT 8: note = F, I, L, or W
QQXXXXPXT

SEQ ID NO: 343 - protein, 10 aa, synthetic construct
  source 1..10: note = 1A7 VL LFR4
FGQGTKLEIK

SEQ ID NO: 344 - protein, 121 aa, synthetic construct
  source 1..121: note = CD28.3[CD28]_H0L0 Variable heavy (vh) domain
EVKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSS

SEQ ID NO: 345 - protein, 5 aa, synthetic construct
  source 1..5: note = CD28.3[CD28]_H0L0 vhCDR1
EYIIH

SEQ ID NO: 346 - protein, 17 aa, synthetic construct
  source 1..17: note = CD28.3[CD28]_H0L0 vhCDR2
WFYPGSNDIQYNAKFKG

SEQ ID NO: 347 - protein, 12 aa, synthetic construct
  source 1..12: note = CD28.3[CD28]_H0L0 vhCDR3
RDDFSGYDALPY

SEQ ID NO: 348 - protein, 107 aa, synthetic construct
  source 1..107: note = CD28.3[CD28]_H0L0 Variable light (vl) domain
DIQMTQSPASLSVSVGETVTITCRTNENIYSNLAWYQQKQGKSPQLLIYAATHLVEGVPSRFSGSGSGTQYSLKITSLQSEDFGNYYCQHFWGTPCTFGGGTKLEIK

SEQ ID NO: 349 - protein, 11 aa, synthetic construct
  source 1..11: note = CD28.3[CD28]_H0L0 vlCDR1
RTNENIYSNLA

SEQ ID NO: 350 - protein, 7 aa, synthetic construct
  source 1..7: note = CD28.3[CD28]_H0L0 vlCDR2
AATHLVE

SEQ ID NO: 351 - protein, 9 aa, synthetic construct
  source 1..9: note = CD28.3[CD28]_H0L0 vlCDR3
QHFWGTPCT

SEQ ID NO: 352 - protein, 121 aa, synthetic construct
  source 1..121: note = hCD28.3[CD28]_H1L1 Variable heavy (vh) domain
QVQLQQSGAELKKPGASVKVSCKASGYTFTEYIIHWIKLRSGQGLEWIGWFYPGSNDIQYNAQFKGKATLTADKSSSTVYMELTGLTPEDSAVYFCARRDDFSGYDALPYWGQGTLVTVSA

SEQ ID NO: 353 - protein, 5 aa, synthetic construct
  source 1..5: note = hCD28.3[CD28]_H1L1 vhCDR1
EYIIH

SEQ ID NO: 354 - protein, 17 aa, synthetic construct
  source 1..17: note = hCD28.3[CD28]_H1L1 vhCDR2
WFYPGSNDIQYNAQFKG

SEQ ID NO: 355 - protein, 12 aa, synthetic construct
  source 1..12: note = hCD28.3[CD28]_H1L1 vhCDR3
RDDFSGYDALPY

SEQ ID NO: 356 - protein, 107 aa, synthetic construct
  source 1..107: note = hCD28.3[CD28]_H1L1 Variable light (vl) domain
DIQMTQSPSSLSASVGDRVTITCKTNENIYSNLAWYQQKDGKSPQLLIYAATHLVEGVPSRFSGSGSGTQYSLTISSLQPEDFGNYYCQHFWGTPCTFGGGTKLEIK

SEQ ID NO: 357 - protein, 11 aa, synthetic construct
  source 1..11: note = hCD28.3[CD28]_H1L1 vlCDR1
KTNENIYSNLA

SEQ ID NO: 358 - protein, 7 aa, synthetic construct
  source 1..7: note = hCD28.3[CD28]_H1L1 vlCDR2
AATHLVE

SEQ ID NO: 359 - protein, 9 aa, synthetic construct
  source 1..9: note = hCD28.3[CD28]_H1L1 vlCDR3
QHFWGTPCT

SEQ ID NO: 360 - protein, 120 aa, synthetic construct
  source 1..120: note = 5.11A1[CD28]_H0L0 Variable heavy (vh) domain
QVQLQQSGPELVKPGTSVRISCEASGYTFTSYYIHWVKQRPGQGLEWIGCIYPGNVNTNYNEKFKDKATLIVDTSSNTAYMQLSRMTSEDSAVYFCTRSHYGLDWNFDVWGAGTTVTVSS

SEQ ID NO: 361 - protein, 5 aa, synthetic construct
  source 1..5: note = 5.11A1[CD28]_H0L0 vhCDR1
SYYIH

SEQ ID NO: 362 - protein, 17 aa, synthetic construct
  source 1..17: note = 5.11A1[CD28]_H0L0 vhCDR2
CIYPGNVNTNYNEKFKD

SEQ ID NO: 363 - protein, 11 aa, synthetic construct
  source 1..11: note = 5.11A1[CD28]_H0L0 vhCDR3
SHYGLDWNFDV

SEQ ID NO: 364 - protein, 107 aa, synthetic construct
  source 1..107: note = 5.11A1[CD28]_H0L0 Variable light (vl) domain
DIQMNQSPSSLSASLGDTITITCHASQNIYVWLNWYQQKPGNIPKLLIYKASNLHTGVPSRFSGSGSGTGFTLTISSLQPEDIATYYCQQGQTYPYTFGGGTKLEIK

SEQ ID NO: 365 - protein, 11 aa, synthetic construct
  source 1..11: note = 5.11A1[CD28]_H0L0 vlCDR1
HASQNIYVWLN

SEQ ID NO: 366 - protein, 7 aa, synthetic construct
  source 1..7: note = 5.11A1[CD28]_H0L0 vlCDR2
KASNLHT

SEQ ID NO: 367 - protein, 9 aa, synthetic construct
  source 1..9: note = 5.11A1[CD28]_H0L0 vlCDR3
QQGQTYPYT

SEQ ID NO: 368 - protein, 120 aa, synthetic construct
  source 1..120: note = TGN1412_H1L1 Variable heavy (vh) domain
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 369 - protein, 5 aa, synthetic construct
  source 1..5: note = TGN1412_H1L1 vhCDR1
SYYIH

SEQ ID NO: 370 - protein, 17 aa, synthetic construct
  source 1..17: note = TGN1412_H1L1 vhCDR2
CIYPGNVNTNYNEKFKD

SEQ ID NO: 371 - protein, 11 aa, synthetic construct
  source 1..11: note = TGN1412_H1L1 vhCDR3
SHYGLDWNFDV

SEQ ID NO: 372 - protein, 107 aa, synthetic construct
  source 1..107: note = TGN1412_H1L1 Variable light (vl) domain
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 373 - protein, 11 aa, synthetic construct
  source 1..11: note = TGN1412_H1L1 vlCDR1
HASQNIYVWLN

SEQ ID NO: 374 - protein, 7 aa, synthetic construct
  source 1..7: note = TGN1412_H1L1 vlCDR2
KASNLHT

SEQ ID NO: 375 - protein, 9 aa, synthetic construct
  source 1..9: note = TGN1412_H1L1 vlCDR3
QQGQTYPYT

SEQ ID NO: 376 - protein, 127 aa, synthetic construct
  source 1..127: note = 341VL34[CD28]_H1L1 Variable heavy (vh) domain
EVQLVESGGGLVKPGGSLRLSCGGSGFTFNNAWMNWVRQAPGKGLEWVGRIKGKTDGGTADYAAPVKGRFTISRDYSKNTLYLQMNSLTTEDTAVYYCNTDLPYYYGSGRYSGMDVWGQGTTVTVSS

SEQ ID NO: 377 - protein, 5 aa, synthetic construct
  source 1..5: note = 341VL34[CD28]_H1L1 vhCDR1
NAWMN

SEQ ID NO: 378 - protein, 19 aa, synthetic construct
  source 1..19: note = 341VL34[CD28]_H1L1 vhCDR2
RIKGKTDGGTADYAAPVKG

SEQ ID NO: 379 - protein, 16 aa, synthetic construct
  source 1..16: note = 341VL34[CD28]_H1L1 vhCDR3
DLPYYYGSGRYSGMDV

SEQ ID NO: 380 - protein, 104 aa, synthetic construct
  source 1..104: note = 341VL34[CD28]_H1L1 Variable light (vl) domain
EIVLTQSPATLSLSPGERATLSCRASQSVSSYLAWYQQKPGQAPRLLIYDASNRATGIPARFSGSGSGTDFTLTISSLEPEDFAVYYCQQRSNTFGPGTKVDIK

SEQ ID NO: 381 - protein, 11 aa, synthetic construct
  source 1..11: note = 341VL34[CD28]_H1L1 vlCDR1
RASQSVSSYLA

SEQ ID NO: 382 - protein, 7 aa, synthetic construct
  source 1..7: note = 341VL34[CD28]_H1L1 vlCDR2
DASNRAT

SEQ ID NO: 383 - protein, 6 aa, synthetic construct
  source 1..6: note = 341VL34[CD28]_H1L1 vlCDR3
QQRSNT

SEQ ID NO: 384 - protein, 127 aa, synthetic construct
  source 1..127: note = 341VL36[CD28]_H1L1 Variable heavy (vh) domain
EVQLVESGGGLVKPGGSLRLSCGGSGFTFNNAWMNWVRQAPGKGLEWVGRIKGKTDGGTADYAAPVKGRFTISRDYSKNTLYLQMNSLKTEDTGVYYCTTYLPYYYGSERWSGMDVWGQGTTVTVSS

SEQ ID NO: 385 - protein, 5 aa, synthetic construct
  source 1..5: note = 341VL36[CD28]_H1L1 vhCDR1
NAWMN

SEQ ID NO: 386 - protein, 19 aa, synthetic construct
  source 1..19: note = 341VL36[CD28]_H1L1 vhCDR2
RIKGKTDGGTADYAAPVKG

SEQ ID NO: 387 - protein, 16 aa, synthetic construct
  source 1..16: note = 341VL36[CD28]_H1L1 vhCDR3
YLPYYYGSERWSGMDV

SEQ ID NO: 388 - protein, 104 aa, synthetic construct; identical to SEQ ID NO: 380
  source 1..104: note = 341VL36[CD28]_H1L1 Variable light (vl) domain

SEQ ID NO: 389 - protein, 11 aa, synthetic construct
  source 1..11: note = 341VL36[CD28]_H1L1 vlCDR1
RASQSVSSYLA

SEQ ID NO: 390 - protein, 7 aa, synthetic construct
  source 1..7: note = 341VL36[CD28]_H1L1 vlCDR2
DASNRAT

SEQ ID NO: 391 - protein, 6 aa, synthetic construct
  source 1..6: note = 341VL36[CD28]_H1L1 vlCDR3
QQRSNT

SEQ ID NO: 392 - protein, 126 aa, synthetic construct
  source 1..126: note = 281VL4[CD28]_H1L1 Variable heavy (vh) domain
EVQLVQSGSELKKPGSSVKVSCKASGGTSRSFAISWVRQAPGQGLEWMGGIIPIFGPANYAQKFQGRVTITADESTSTAYMELSSLRSEDTAVYYCARHAIAMGWGVITTNYFDSWGQGTMVTVSS

SEQ ID NO: 393 - protein, 5 aa, synthetic construct
  source 1..5: note = 281VL4[CD28]_H1L1 vhCDR1
SFAIS

SEQ ID NO: 394 - protein, 17 aa, synthetic construct
  source 1..17: note = 281VL4[CD28]_H1L1 vhCDR2
GIIPIFGPANYAQKFQG

SEQ ID NO: 395 - protein, 17 aa, synthetic construct
  source 1..17: note = 281VL4[CD28]_H1L1 vhCDR3
HAIAMGWGVITTNYFDS

SEQ ID NO: 396 - protein, 108 aa, synthetic construct
  source 1..108: note = 281VL4[CD28]_H1L1 Variable light (vl) domain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGQGTRLEIK

SEQ ID NO: 397 - protein, 12 aa, synthetic construct
  source 1..12: note = 281VL4[CD28]_H1L1 vlCDR1
RASQSVSSSYLA

SEQ ID NO: 398 - protein, 7 aa, synthetic construct
  source 1..7: note = 281VL4[CD28]_H1L1 vlCDR2
GASSRAT

SEQ ID NO: 399 - protein, 9 aa, synthetic construct
  source 1..9: note = 281VL4[CD28]_H1L1 vlCDR3
QQYGSSPIT

SEQ ID NO: 400 - protein, 121 aa, synthetic construct
  source 1..121: note = HuTN228[CD28]_H1L1 Variable heavy (vh) domain
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSS

SEQ ID NO: 401 - protein, 5 aa, synthetic construct
  source 1..5: note = HuTN228[CD28]_H1L1 vhCDR1
SYGVH

SEQ ID NO: 402 - protein, 16 aa, synthetic construct
  source 1..16: note = HuTN228[CD28]_H1L1 vhCDR2
VIWPGGGTNFNSALMS

SEQ ID NO: 403 - protein, 13 aa, synthetic construct
  source 1..13: note = HuTN228[CD28]_H1L1 vhCDR3
DRAYGNYLYAMDY

SEQ ID NO: 404 - protein, 111 aa, synthetic construct
  source 1..111: note = HuTN228[CD28]_H1L1 Variable light (vl) domain
DIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIK

SEQ ID NO: 405 - protein, 15 aa, synthetic construct
  source 1..15: note = HuTN228[CD28]_H1L1 vlCDR1
RASESVEYYVTSLMQ

SEQ ID NO: 406 - protein, 7 aa, synthetic construct
  source 1..7: note = HuTN228[CD28]_H1L1 vlCDR2
AASNVDS

SEQ ID NO: 407 - protein, 9 aa, synthetic construct
  source 1..9: note = HuTN228[CD28]_H1L1 vlCDR3
QQSRKVPFT

SEQ ID NO: 408 - protein, 124 aa, synthetic construct
  source 1..124: note = PV1[CD28]_H0L0 Variable heavy (vh) domain
QVQLKQSGAELVKPGASVKISCKTSGYTFTDGYMNWVEQKPGQGLEWIGRIDPDSGNTRYNQKFQGKATLTRDKSSSTVYMDLRSLTSEDSAVYYCARDGTFYGTYGYWYFDFWGQGTQVTVSS

SEQ ID NO: 409 - protein, 5 aa, synthetic construct
  source 1..5: note = PV1[CD28]_H0L0 vhCDR1
DGYMN

SEQ ID NO: 410 - protein, 17 aa, synthetic construct
  source 1..17: note = PV1[CD28]_H0L0 vhCDR2
RIDPDSGNTRYNQKFQG

SEQ ID NO: 411 - protein, 15 aa, synthetic construct
  source 1..15: note = PV1[CD28]_H0L0 vhCDR3
DGTFYGTYGYWYFDF

SEQ ID NO: 412 - protein, 113 aa, synthetic construct
  source 1..113: note = PV1[CD28]_H0L0 Variable light (vl) domain
DIVMTQSPYSLAVSAGEKVTMSCRSSQSLYYSGIKKNLLAWYQQKPGQSPKLLIYFTSTRLPGVPDRFTGSGSGTDYTLTITSVQAEDMGHYFCQQGISTPLTFGDGTKLEIR

SEQ ID NO: 413 - protein, 17 aa, synthetic construct
  source 1..17: note = PV1[CD28]_H0L0 vlCDR1
RSSQSLYYSGIKKNLLA

SEQ ID NO: 414 - protein, 7 aa, synthetic construct
  source 1..7: note = PV1[CD28]_H0L0 vlCDR2
FTSTRLP

SEQ ID NO: 415 - protein, 9 aa, synthetic construct
  source 1..9: note = PV1[CD28]_H0L0 vlCDR3
QQGISTPLT

SEQ ID NO: 416 - protein, 120 aa, synthetic construct
  source 1..120: note = m9.3[CD28]_H0L0 Variable heavy (vh) domain
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSS

SEQ ID NO: 417 - protein, 5 aa, synthetic construct
  source 1..5: note = m9.3[CD28]_H0L0 vhCDR1
DYGVH

SEQ ID NO: 418 - protein, 16 aa, synthetic construct
  source 1..16: note = m9.3[CD28]_H0L0 vhCDR2
VIWAGGGTNYNSALMS

SEQ ID NO: 419 - protein, 12 aa, synthetic construct
  source 1..12: note = m9.3[CD28]_H0L0 vhCDR3
DKGYSYYYSMDY

SEQ ID NO: 420 - protein, 111 aa, synthetic construct
  source 1..111: note = m9.3[CD28]_H0L0 Variable light (vl) domain
DIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIK

SEQ ID NO: 421 - protein, 15 aa, synthetic construct
  source 1..15: note = m9.3[CD28]_H0L0 vlCDR1
RASESVEYYVTSLMQ

SEQ ID NO: 422 - protein, 7 aa, synthetic construct
  source 1..7: note = m9.3[CD28]_H0L0 vlCDR2
AASNVES

SEQ ID NO: 423 - protein, 9 aa, synthetic construct
  source 1..9: note = m9.3[CD28]_H0L0 vlCDR3
QQSRKVPYT

SEQ ID NO: 424 - protein, 120 aa, synthetic construct
  source 1..120: note = hu9.3[CD28]_H1L1 Variable heavy (vh) domain
EVQLVQSGGGLVQPGGSLRLSCAGSGFTFSDYGVHWVRQAPGKGLEWVSAIWAGGGTNYASSVMGRFTISRDNAKNSLYLQMNSLRAEDMAVYYCARDKGYSYYYSMDYWGQGTLVTVSS

SEQ ID NO: 425 - protein, 5 aa, synthetic construct
  source 1..5: note = hu9.3[CD28]_H1L1 vhCDR1
DYGVH

SEQ ID NO: 426 - protein, 16 aa, synthetic construct
  source 1..16: note = hu9.3[CD28]_H1L1 vhCDR2
AIWAGGGTNYASSVMG

SEQ ID NO: 427 - protein, 12 aa, synthetic construct
  source 1..12: note = hu9.3[CD28]_H1L1 vhCDR3
DKGYSYYYSMDY

SEQ ID NO: 428 - protein, 111 aa, synthetic construct
  source 1..111: note = hu9.3[CD28]_H1L1 Variable light (vl) domain
DIVMTQSPDSLAVSLGERATINCRASESVEYYVTSLMAWYQQKPGQPPKLLIYAASNVESGVPDRFSGSGSGTNFSLTISSLQAEDVAVYYCQQSRKVPYTFGQGTKLEIK

SEQ ID NO: 429 - protein, 15 aa, synthetic construct
  source 1..15: note = hu9.3[CD28]_H1L1 vlCDR1
RASESVEYYVTSLMA

SEQ ID NO: 430 - protein, 7 aa, synthetic construct
  source 1..7: note = hu9.3[CD28]_H1L1 vlCDR2
AASNVES

SEQ ID NO: 431 - protein, 9 aa, synthetic construct
  source 1..9: note = hu9.3[CD28]_H1L1 vlCDR3
QQSRKVPYT

SEQ ID NO: 432 - protein, 127 aa, synthetic construct
  source 1..127: note = 9G2[CD28]_H0L0 Variable heavy (vh) domain
EVQLVESGGGLVQPGRSLKLSCAASGFTFSNYYMAWVRQAPTKGLEWVASITNTGGSTYYRDSVKGRFTISRDNAKSTLYLQMDSLRSEDTATYYCTRGLIYYYDGRNYYDYVMDAWGQGASVTVSS

SEQ ID NO: 433 - protein, 5 aa, synthetic construct
  source 1..5: note = 9G2[CD28]_H0L0 vhCDR1
NYYMA

SEQ ID NO: 434 - protein, 17 aa, synthetic construct
  source 1..17: note = 9G2[CD28]_H0L0 vhCDR2
SITNTGGSTYYRDSVKG

SEQ ID NO: 435 - protein, 18 aa, synthetic construct
  source 1..18: note = 9G2[CD28]_H0L0 vhCDR3
GLIYYYDGRNYYDYVMDA

SEQ ID NO: 436 - protein, 107 aa, synthetic construct
  source 1..107: note = 9G2[CD28]_H0L0 Variable light (vl) domain
DIQMTQSPASLSASLGETVSIECLASEGISNSLAWYQQKPGKSPQLLIYGASSLQDGVPSRFSGSGSGTQYSLKISGMQPEDEGVYYCQQGYKYPLTFGSGTKLEIK

SEQ ID NO: 437 - protein, 11 aa, synthetic construct
  source 1..11: note = 9G2[CD28]_H0L0 vlCDR1
LASEGISNSLA

SEQ ID NO: 438 - protein, 7 aa, synthetic construct
  source 1..7: note = 9G2[CD28]_H0L0 vlCDR2
GASSLQD

SEQ ID NO: 439 - protein, 9 aa, synthetic construct
  source 1..9: note = 9G2[CD28]_H0L0 vlCDR3
QQGYKYPLT

SEQ ID NO: 440 - protein, 127 aa, synthetic construct
  source 1..127: note = 9G2[CD28]_H1L1 Variable heavy (vh) domain
EVQLLESGGGLVQPGGSLRLSCAASGFTFSNYYMAWVRQAPGKGLEWVSSITNTGGSTYYRDSVKGRFTISRDNSKSTLYLQMNSLRAEDTAVYYCTRGLIYYYDGRNYYDYVMDAWGQGTTVTVSS

SEQ ID NO: 441 - protein, 5 aa, synthetic construct
  source 1..5: note = 9G2[CD28]_H1L1 vhCDR1
NYYMA

SEQ ID NO: 442 - protein, 17 aa, synthetic construct
  source 1..17: note = 9G2[CD28]_H1L1 vhCDR2
SITNTGGSTYYRDSVKG

SEQ ID NO: 443 - protein, 18 aa, synthetic construct
  source 1..18: note = 9G2[CD28]_H1L1 vhCDR3
GLIYYYDGRNYYDYVMDA

SEQ ID NO: 444 - protein, 107 aa, synthetic construct
  source 1..107: note = 9G2[CD28]_H1L1 Variable light (vl) domain
DIQMTQSPSSLSASVGDRVTITCRASEGISNSLAWYQQKPGKSPKLLIYGASSLQDGVPSRFSGSGSGTDYTLTISSLQPEDFATYYCQQGYKYPLTFGSGTKVEIK

SEQ ID NO: 445 - protein, 11 aa, synthetic construct
  source 1..11: note = 9G2[CD28]_H1L1 vlCDR1
RASEGISNSLA

SEQ ID NO: 446 - protein, 7 aa, synthetic construct
  source 1..7: note = 9G2[CD28]_H1L1 vlCDR2
GASSLQD

SEQ ID NO: 447 - protein, 9 aa, synthetic construct
  source 1..9: note = 9G2[CD28]_H1L1 vlCDR3
QQGYKYPLT

SEQ ID NO: 448 - protein, 121 aa, synthetic construct
  source 1..121: note = 2F10A3.140[CD28]_H1L1 Variable heavy (vh) domain
EVQLLESGGGLVQPGGSLRLSCAASGFSFGGNSMSWVRQAPGKGLEWVATISDNSYSTYYADSVKGRFTISRDNSKNTLYLQMNSLRAEDTAVYYCAKSGPGLRQVGFDYWGQGTLVTVSS

SEQ ID NO: 449 - protein, 5 aa, synthetic construct
  source 1..5: note = 2F10A3.140[CD28]_H1L1 vhCDR1
GNSMS

SEQ ID NO: 450 - protein, 17 aa, synthetic construct
  source 1..17: note = 2F10A3.140[CD28]_H1L1 vhCDR2
TISDNSYSTYYADSVKG

SEQ ID NO: 451 - protein, 12 aa, synthetic construct
  source 1..12: note = 2F10A3.140[CD28]_H1L1 vhCDR3
SGPGLRQVGFDY

SEQ ID NO: 452 - protein, 107 aa, synthetic construct; identical to SEQ ID NO: 111
  source 1..107: note = 2F10A3.140[CD28]_H1L1 Variable light (vl) domain

SEQ ID NO: 453 - protein, 11 aa, synthetic construct
  source 1..11: note = 2F10A3.140[CD28]_H1L1 vlCDR1
RASQSISSYLN

SEQ ID NO: 454 - protein, 7 aa, synthetic construct
  source 1..7: note = 2F10A3.140[CD28]_H1L1 vlCDR2
AASSLQS

SEQ ID NO: 455 - protein, 9 aa, synthetic construct
  source 1..9: note = 2F10A3.140[CD28]_H1L1 vlCDR3
QQSYSTPFT

SEQ ID NO: 456 - protein, 121 aa, synthetic construct
  source 1..121: note = TN228[CD28]_H4L2 Variable heavy (vh) domain
QVQLQESGPGLVKPSETLSLTCTVSGFSLTSYGVHWVRQPPGKGLEWIGVIWPGGGTNYNSALKSRVTISEDTSKSQVSLKLSSVTAADTAVYYCARDRAYGNYLYAMDYWGQGTLVTVSS

SEQ ID NO: 457 - protein, 5 aa, synthetic construct
  source 1..5: note = TN228[CD28]_H4L2 vhCDR1
SYGVH

SEQ ID NO: 458 - protein, 16 aa, synthetic construct
  source 1..16: note = TN228[CD28]_H4L2 vhCDR2
VIWPGGGTNYNSALKS

SEQ ID NO: 459 - protein, 13 aa, synthetic construct
  source 1..13: note = TN228[CD28]_H4L2 vhCDR3
DRAYGNYLYAMDY

SEQ ID NO: 460 - protein, 111 aa, synthetic construct
  source 1..111: note = TN228[CD28]_H4L2 Variable light (vl) domain
EIVLTQSPATLSLSPGERATLSCRASESVEYYVTSLMQWYQQKPGQAPRLLIYAASNVDSGVPARFSGSGSGTDFTLTISSLEPEDFAVYYCQQSRKVPFTFGGGTKVEIK

SEQ ID NO: 461 - protein, 15 aa, synthetic construct
  source 1..15: note = TN228[CD28]_H4L2 vlCDR1
RASESVEYYVTSLMQ

SEQ ID NO: 462 - protein, 7 aa, synthetic construct
  source 1..7: note = TN228[CD28]_H4L2 vlCDR2
AASNVDS

SEQ ID NO: 463 - protein, 9 aa, synthetic construct
  source 1..9: note = TN228[CD28]_H4L2 vlCDR3
QQSRKVPFT

SEQ ID NO: 464 - protein, 120 aa, synthetic construct
  source 1..120: note = RS7[TROP2] Variable Heavy (vh) Domain
VKLQESGPELKKPGETVKISCKASGYTFTNYGMNWVKQAPGKGLKWMGWINTYTGEPTYTDDFKGRFAFSLETSATTAYLQINNLKSEDMATYFCARGGFGSSYWYFDVWGQGTTVTVSS

SEQ ID NO: 465 - protein, 107 aa, synthetic construct
  source 1..107: note = RS7[TROP2] Variable Light (vl) Domain
DIQLTQSHKFMSTSVGDRVSITCKASQDVSIAVAWYQQKPGQSPKLLIYSASYRYTGVPDRFTGSGSGTDFTFTISSVQAEDLAVYYCQQHYITPLTFGAGTKLELK

SEQ ID NO: 466 - protein, 121 aa, synthetic construct
  source 1..121: note = hRS7[TROP2] Variable Heavy (vh) Domain
SVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 467 - protein, 107 aa, synthetic construct
  source 1..107: note = hRS7[TROP2] Variable Light (vl) Domain
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 468 - protein, 121 aa, synthetic construct
  source 1..121: note = hRS7v2[TROP2] Variable Heavy (vh) Domain
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 469 - protein, 107 aa, synthetic construct; identical to SEQ ID NO: 467
  source 1..107: note = hRS7v2[TROP2] Variable Light (vl) Domain

SEQ ID NO: 470 - protein, 121 aa, synthetic construct
  source 1..121: note = AR47A6.4.2[TROP2] Variable Heavy (vh) Domain
QIQLVQSGPELKKPGETVKISCKASGYTFTNYGMNWVKQAPGKGLKWMGWINTKTGEPTYAEEFKGRFAFSLETSASTAYLQINNLKKEDTATYFCGRGGYGSSYWYFDVWGAGTTVTVSS

SEQ ID NO: 471 - protein, 107 aa, synthetic construct
  source 1..107: note = AR47A6.4.2[TROP2] Variable Light (vl) Domain
DIQMTQSPASLSASVGETVTITCRASENIYSYLAWYQQKQGKSPQLLVYNAKTLAEGVPSRFSGSGSGTQFSLKINSLRPEDFGSYYCQHHYGSPLTFGAGTKLELR

SEQ ID NO: 472 - protein, 121 aa, synthetic construct
  source 1..121: note = huAR476A6.4.2 HV2/KV3[TROP2] Variable Heavy (vh) Domain
QIQLVQSGHEVKKPGASVKVSCKASGYTFTNYGMNWVRQAPGQGLEWMGWINTKTGEPTYAEEFKGRFVFSLETSASTAYLQISSLKAEDTAMYFCGRGGYGSSYWYFDVWGQGTTVTVSS

SEQ ID NO: 473 - protein, 107 aa, synthetic construct
  source 1..107: note = huAR476A6.4.2 HV2/KV3[TROP2] Variable Light (vl) Domain
DIQMTQSPSSLSASVGDRVTITCKASQDVSIAVAWYQQKPGKAPKVLIYSASYRYTGVPDRFSGSGSGTDFTFTISSLQPEDIAVYYCQQHYITPLTFGGGTKVEIK

SEQ ID NO: 474 - protein, 121 aa, synthetic construct; identical to SEQ ID NO: 472
  source 1..121: note = huAR476A6.4.2 HV2/KV4[TROP2] Variable Heavy (vh) Domain

SEQ ID NO: 475 - protein, 107 aa, synthetic construct
  source 1..107: note = huAR476A6.4.2 HV2/KV4[TROP2] Variable Light (vl) Domain
DIQMTQSPSSLSASVGDRVTITCKASQDVSIAVAWYQQKPGKAPKVLIYSASYRYTGVPSRFSGSGSGTDFTFTISSLQPEDIAVYYCQQHYITPLTFGGGTKVEIK

SEQ ID NO: 476 - protein, 121 aa, synthetic construct
  source 1..121: note = huAR476A6.4.2 HV3/KV3[TROP2] Variable Heavy (vh) Domain
QIQLVQSGHEVKKPGASVKVSCKASGYTFTNYGMNWVRQAPGQGLEWMGWINTKTGEPTYAEEFKGRFVFSLETSASTAYLQISSLKAEDMAMYFCGRGGYGSSYWYFDVWGQGTTVTVSS

SEQ ID NO: 477 - protein, 107 aa, synthetic construct; identical to SEQ ID NO: 473
  source 1..107: note = huAR476A6.4.2 HV3/KV3[TROP2] Variable Light (vl) Domain

SEQ ID NO: 478 - protein, 121 aa, synthetic construct; identical to SEQ ID NO: 476
  source 1..121: note = huAR476A6.4.2 HV2/KV4[TROP2] Variable Heavy (vh) Domain

SEQ ID NO: 479 - protein, 109 aa, synthetic construct
  source 1..109: note = 4F6[TROP2] Variable Heavy (vh) Domain
DVQLEQFGAELVRPGTSVKMSCKAAGYTFTNYWIGWVKQRPGHGLEWIGDIYPGGGYTNYNEKFKGKATLTADTSSSTAYMQLSSLTFEDFAIYYCARGTGGGDYWGQG

SEQ ID NO: 480 - protein, 93 aa, synthetic construct
  source 1..93: note = 4F6[TROP2] Variable Light (vl) Domain
IVITQSPASLSMAIGEKVSIRCITSTDIDDDMNWYQQKPGEPPKLLISEGNTLRPGVPSRFSSSGYGTDFVFTIENMLSEDVADYYCLQSDNL

SEQ ID NO: 481 - protein, 109 aa, synthetic construct; identical to SEQ ID NO: 479
  source 1..109: note = 2G10[TROP2] Variable Heavy (vh) Domain

SEQ ID NO: 482 - protein, 93 aa, synthetic construct
  source 1..93: note = 2G10[TROP2] Variable Light (vl) Domain
IVITQSPASLSMAIGEKVTIRCITSTDIDDDMNWYQQKPGEPPKLLISEGNTLRPGVPSRFSSSGYGTDFVFTIENMLSEDVADYYCLQSDNL

SEQ ID NO: 483 - protein, 109 aa, synthetic construct; identical to SEQ ID NO: 479
  source 1..109: note = 2EF[TROP2] Variable Heavy (vh) Domain

SEQ ID NO: 484 - protein, 98 aa, synthetic construct
  source 1..98: note = 2EF[TROP2] Variable Light (vl) Domain
DIVMTQSPASLAVSLGQRATISCRASQSVSTSSYSYMHWYQQKPGQPPKLLIKYASNLECGVPARFSGSGCGTDFTLNIHPVEEEDSATYYCQHSREI

SEQ ID NO: 485 - protein, 115 aa, synthetic construct
  source 1..115: note = K5-70[TROP2] Variable Heavy (vh) Domain
QVQLQQPGAELVRPGASVKLSCKASGYTFTIYWINWVKQRPGQGLEWIGNIYPSDSYTNYNQKFKDKATLTVDKSSSTAYMQLSSPTSEDSAVYYCTRTSMADYWGQGTTLTVSS

SEQ ID NO: 486 - protein, 107 aa, synthetic construct
  source 1..107: note = K5-70[TROP2] Variable Light (vl) Domain
DILLTQSPAILSVSPGERVSFSCRASQSIGTSIHWYQQRTNGSPRLLIKYASESISGIPSRFSGSGSGTDFTLSINSVESEDIADYYCQQSNSWPFTFGSGTKLEIK

SEQ ID NO: 487 - protein, 115 aa, synthetic construct
  source 1..115: note = K5-107[TROP2] Variable Heavy (vh) Domain
QVQLQQPGSELVRPGASVKLSCKASGYTFTSYWMHWVKQRHGQGLEWIGNIYPGGGYTNYDEKFKSKGTLTVDTSSSTAYMHLSSLTSEDSAVYYCTRSSVFDYWGQGTTLTVSS

SEQ ID NO: 488 - protein, 107 aa, synthetic construct
  source 1..107: note = K5-107[TROP2] Variable Light (vl) Domain
DILLTQSPAILSVSPGERVSFSCRASQNIGTSIHWFQQRTNGSPRLLIKYASESISGIPSRFSGSGSGTDFTLSINSVESEDIADYYCQQSNSWPFTFGSGTKLEIK

SEQ ID NO: 489 - protein, 113 aa, synthetic construct
  source 1..113: note = K5-116-2-1[TROP2] Variable Heavy (vh) Domain
QVQLQQPGAELVRPGASVKLSCKASGYTFTSYWITWVKQRPGQGLEWIGNIYPSDSYTNYNQKFRDKATLTVDKSSSTAYMQLSSPTSEDSAVYYCSALFDYWGQGTTLTVSS

SEQ ID NO: 490 - protein, 107 aa, synthetic construct
  source 1..107: note = K5-116-2-1[TROP2] Variable Light (vl) Domain
DILLTQSPAILSVSPGERVSFSCRASQSIGTSIHWYQQRTNGSPRLLIKYASESISGIPSRFSGSGSGTDFILSINSVESEDIADYYCQQSNSWPFTFGSGTKLEIK

SEQ ID NO: 491 - protein, 122 aa, synthetic construct
  source 1..122: note = T6-16[TROP2] Variable Heavy (vh) Domain
EVQLQQSGPELVKPGASVKISCKASGYTFTDYNMHWVKQSHGKNLEWIGYIYPYNGGTGYNQRFKSRATMTVDKSSSTAYMELRSLTSDDSAVYYCAREDYGSSPSYAMDYWGQGTSVIVSS

SEQ ID NO: 492 - protein, 111 aa, synthetic construct
  source 1..111: note = T6-16[TROP2] Variable Light (vl) Domain
DVVMTQTPLSLPVSLGDQASISCRSSQSLVHGNGNTYLHWYLQKPGQSPKLLIYKVSNRFSGVPDRFSGSGSGTDFTLKISRVEAEDLGVYFCSQTTHVPTFGSGTKLEIK

SEQ ID NO: 493 - protein, 116 aa, synthetic construct
  source 1..116: note = KM4097[TROP2] Variable Heavy (vh) Domain
QVQLQQSGPELVRPGTSVRISCKASGYTFTIYWLGWVKQRPGHGLEWIGNIFPGSAYINYNEKFKGKATLTADTSSSTAYMQLSSLTSEDSAVYFCAREGSNSGYWGQGTTLTVSS

SEQ ID NO: 494 - protein, 113 aa, synthetic construct
  source 1..113: note = KM4097[TROP2] Variable Light (vl) Domain
DIVMTQSPSSLSVSAGEKVTMTCKSSQSLLNSGNQQNYLAWYQQKPGQPPKLLIYGASTRESGVPDRFTGSGSGTDFTLTINSVQAEDLAVYYCQSDHIYPYTFGGGTKLEIK

SEQ ID NO: 495 - protein, 116 aa, synthetic construct
  source 1..116: note = HV0LV0[TROP2] Variable Heavy (vh) Domain
QVQLVQSGAEVKKPGASVKVSCKASGYTFTIYWLGWVRQAPGQGLEWMGNIFPGSAYINYNEKFKGRVTITADTSTSTAYMELSSLRSEDTAVYYCAREGSNSGYWGQGTLVTVSS

SEQ ID NO: 496 - protein, 113 aa, synthetic construct
  source 1..113: note = HV0LV0[TROP2] Variable Light (vl) Domain
DIVMTQSPDSLAVSLGERATINCKSSQSLLNSGNQQNYLAWYQQKPGQPPKLLIYGASTRESGVPDRFSGSGSGTDFTLTISSLQAEDVAVYYCQSDHIYPYTFGQGTKLEIK

SEQ ID NO: 497 - protein, 116 aa, synthetic construct
  source 1..116: note = HV3aLV0[TROP2] Variable Heavy (vh) Domain
QVQLVQSGPEVKKPGASVKVSCKASGYTFTIYWLGWVRQAPGQGLEWMGNIFPGSAYINYNEKFKGKVTITADTSTSTAYMELSSLRSEDTAVYFCAREGSNSGYWGQGTLVTVSS

SEQ ID NO: 498 - protein, 113 aa, synthetic construct; identical to SEQ ID NO: 496
  source 1..113: note = HV3aLV0[TROP2] Variable Light (vl) Domain

SEQ ID NO: 499 - protein, 116 aa, synthetic construct
  source 1..116: note = HV4LV0[TROP2] Variable Heavy (vh) Domain
QVQLVQSGPEVKKPGASVKVSCKASGYTFTIYWLGWVKQAPGQGLEWIGNIFPGSAYINYNEKFKGRVTITADTSTSTAYMELSSLRSEDTAVYFCAREGSNSGYWGQGTLVTVSS

SEQ ID NO: 500 - protein, 113 aa, synthetic construct; identical to SEQ ID NO: 496
  source 1..113: note = HV4LV0[TROP2] Variable Light (vl) Domain

SEQ ID NO: 501 - protein, 116 aa, synthetic construct
  source 1..116: note = HV5LV0[TROP2] Variable Heavy (vh) Domain
QVQLVQSGAEVKKPGASVKVSCKASGYTFTIYWLGWVKQAPGQGLEWIGNIFPGSAYINYNEKFKGKATITADTSTSTAYMELSSLRSEDTAVYFCAREGSNSGYWGQGTLVTVSS

SEQ ID NO: 502 - protein, 113 aa, synthetic construct; identical to SEQ ID NO: 496
  source 1..113: note = HV5LV0[TROP2] Variable Light (vl) Domain

SEQ ID NO: 503 - protein, 116 aa, synthetic construct
  source 1..116: note = HV10LV2[TROP2] Variable Heavy (vh) Domain
QVQLVQSGPEVVKPGASVKISCKASGYTFTIYWLGWVKQAPGQGLEWIGNIFPGSAYINYNEKFKGKATLTADTSTSTAYMELSSLRSEDTAVYFCAREGSNSGYWGQGTLLTVSS

SEQ ID NO: 504 - protein, 113 aa, synthetic construct
  source 1..113: note = HV10LV2[TROP2] Variable Light (vl) Domain
DIVMTQSPDSLAVSLGERVTINCKSSQSLLNSGNQQNYLAWYQQKPGQPPKLLIYGASTRESGVPDRFSGSGSGTDFTLTISSVQAEDVAVYYCQSDHIYPYTFGQGTKLEIK

SEQ ID NO: 505 - protein, 116 aa, synthetic construct; identical to SEQ ID NO: 503
  source 1..116: note = HV10LV4[TROP2] Variable Heavy (vh) Domain

SEQ ID NO: 506 - protein, 113 aa, synthetic construct
  source 1..113: note = HV10LV4[TROP2] Variable Light (vl) Domain
DIVMTQSPDSLAVSAGERVTMNCKSSQSLLNSGNQQNYLAWYQQKPGQPPKLLIYGASTRESGVPDRFSGSGSGTDFTLTISSVQAEDVAVYYCQSDHIYPYTFGQGTKLEIK

SEQ ID NO: 507 - protein, 119 aa, synthetic construct
  source 1..119: note = 7E6[TROP2] Variable Heavy (vh) Domain
QVQLKESGPGLVAPSQSLSITCTVSGFSLTSYGVHWVRQPPGKGLEWLGVIWTGGSTDYNSALMSRLSINKDNSKSQVFLKMNSLQTDDTAMYYCARDGDYDRYTMDYWGQGTSVTVSS

SEQ ID NO: 508 - protein, 111 aa, synthetic construct
  source 1..111: note = 7E6[TROP2] Variable Light (vl) Domain
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPYTFGGGTKLEIK

SEQ ID NO: 509 - protein, 119 aa, synthetic construct
  source 1..119: note = h7E6[TROP2] Variable Heavy (vh) Domain
QVQLQESGPGLVKPSETLSLTCTVSGGSISSYGVHWIRQPPGKGLEWIGVIWTGGSTDYNSALMSRVTISVDTSKNQFSLKLSSVTAADTAVYYCARDGDYDRYTMDYWGQGTLVTVSS

SEQ ID NO: 510 - protein, 111 aa, synthetic construct
  source 1..111: note = h7E6[TROP2] Variable Light (vl) Domain
DIVMTQSPDSLAVSLGERATINCRASKSVSTSGYSYMHWYQQKPGQPPKLLIYLASNLESGVPDRFSGSGSGTDFTLTISSLQAEDVAVYYCQHSRELPYTFGQGTKLEIK

SEQ ID NO: 511 - protein, 119 aa, synthetic construct
  source 1..119: note = h7E6_SVG[TROP2] Variable Heavy (vh) Domain
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGDYDRYTMDYWGQGTLVTVSS

SEQ ID NO: 512 - protein, 111 aa, synthetic construct; identical to SEQ ID NO: 510
  source 1..111: note = h7E6_SVG[TROP2] Variable Light (vl) Domain

SEQ ID NO: 513 - protein, 119 aa, synthetic construct; identical to SEQ ID NO: 511
  source 1..119: note = h7E6_SVGL[TROP2] Variable Heavy (vh) Domain

SEQ ID NO: 514 - protein, 111 aa, synthetic construct
  source 1..111: note = h7E6_SVGL[TROP2] Variable Light (vl) Domain
DIVMTQSPDSLAVSLGERATINCRASKSVSTSLYSYMHWYQQKPGQPPKLLIYLASNLESGVPDRFSGSGSGTDFTLTISSLQAEDVAVYYCQHSRELPYTFGQGTKLEIK

SEQ ID NO: 515 - protein, 119 aa, synthetic construct; identical to SEQ ID NO: 511
  source 1..119: note = h7E6_SVGN[TROP2] Variable Heavy (vh) Domain

SEQ ID NO: 516 - protein, 111 aa, synthetic construct
  source 1..111: note = h7E6_SVGN[TROP2] Variable Light (vl) Domain
DIVMTQSPDSLAVSLGERATINCRASKSVSTSNYSYMHWYQQKPGQPPKLLIYLASNLESGVPDRFSGSGSGTDFTLTISSLQAEDVAVYYCQHSRELPYTFGQGTKLEIK

SEQ ID NO: 517 - protein, 115 aa, synthetic construct
  source 1..115: note = 6G11[TROP2] Variable Heavy (vh) Domain
QVQLQQPGAELVRPGASVKLSCKASGYTFTSYWINWVKQRPGHGLEWIGNIYPSDSYSNYNQKFKDKATLTVDKSSSTAYMQVSSPTSEDSAVYYCTYGSSFDYWGQGTTVTVSS

SEQ ID NO: 518 - protein, 107 aa, synthetic construct
  source 1..107: note = 6G11[TROP2] Variable Light (vl) Domain
DILLTQSPAILSVSPGERVSFSCRASQTIGTSIHWYQQRTNGSPRLLIKYASESISGIPSRFSGSGSGTDFTLSINSVESEDIADYYCQQSNSWPFTFGSGTKLEIK

SEQ ID NO: 519 - protein, 115 aa, synthetic construct
  source 1..115: note = h6G11[TROP2] Variable Heavy (vh) Domain
QVQLVQSGAEVKKPGASVKVSCKASGYTFTSYWINWVRQAPGQGLEWMGNIYPSDSYSNYNQKFKDRVTMTRDTSTSTVYMELSSLRSEDTAVYYCARGSSFDYWGQGTLVTVSS

SEQ ID NO: 520 - protein, 107 aa, synthetic construct
  source 1..107: note = h6G11[TROP2] Variable Light (vl) Domain
EIVLTQSPATLSLSPGERATLSCRASQTIGTSIHWYQQKPGQAPRLLIYYASESISGIPARFSGSGSGTDFTLTISSLEPEDFAVYYCQQSNSWPFTFGQGTKLEIK

SEQ ID NO: 521 - protein, 115 aa, synthetic construct
  source 1..115: note = h6G11_FKG_SF[TROP2] Variable Heavy (vh) Domain
QVQLVQSGAEVKKPGASVKVSCKASGYTFTSYWINWVRQAPGQGLEWMGNIFPSDSYSNYNKKFKDRVTMTRDTSTSTVYMELSSLRSEDTAVYYCARGSGFDYWGQGTLVTVSS

SEQ ID NO: 522 - protein, 107 aa, synthetic construct
  source 1..107: note = h6G11_FKG_SF[TROP2] Variable Light (vl) Domain
EIVLTQSPATLSLSPGERATLSCRASQTIGTSIHWYQQKPGQAPRLLIYYASESISGIPARFSGSGSGTDFTLTISSLEPEDFAVYYCSQSFSWPFTFGQGTKLEIK

SEQ ID NO: 523 - protein, 115 aa, synthetic construct
  source 1..115: note = huK5-70[TROP2] Variable Heavy (vh) Domain
QVQLVQSGAEVKKPGASVKVSCKASGYTFTIYWINWVRQAPGQRLEWIGNIYPSDSYTNYNQKFKDKATLTVDTSASTAYMELSSLRSEDTAVYYCTRTSMADYWGQGTLVTVSS

SEQ ID NO: 524 - protein, 107 aa, synthetic construct
  source 1..107: note = huK5-70[TROP2] Variable Light (vl) Domain
EIVLTQSPATLSLSPGERATLSCRASQSIGTSIHWYQQKPGQAPRLLIKYASESISGIPARFSGSGSGTDFTLTISSLEPEDFAVYYCQQSNSWPFTFGQGTKVEIK

SEQ ID NO: 525 - protein, 115 aa, synthetic construct
  source 1..115: note = huK5-70-2[TROP2] Variable Heavy (vh) Domain
QVQLVQSGAEVKKPGASVKVSCKASGYTFTIYWINWVRQAPGQGLEWIGNIYPSDSYTNYNQKFKDKATLTVDTSASTAYMELSSLRSEDTAVYYCTRTSMADYWGQGTLVTVSS

SEQ ID NO: 526 - protein, 107 aa, synthetic construct; identical to SEQ ID NO: 524
  source 1..107: note = huK5-70-2[TROP2] Variable Light (vl) Domain

SEQ ID NO: 527 - protein, 122 aa, synthetic construct
  source 1..122: note = huT6-16-1[TROP2] Variable Heavy (vh) Domain
QVQLVQSGAEVKKPGASVKVSCKASGYTFTDYNMHWVRQAPGQGLEWIGYIYPYNGGTGYNQRFKSRATMTVDKSTSTAYMELRSLRSDDTAVYYCAREDYGSSPSYAMDYWGQGTMVTVSS

SEQ ID NO: 528 - protein, 111 aa, synthetic construct
  source 1..111: note = huT6-16-1[TROP2] Variable Light (vl) Domain
DIVMTQSPLSLPVTPGEPASISCRSSQSLVHGNGNTYLHWYLQKPGQSPQLLIYKVSNRFSGVPDRFSGSGSGTDFTLKISRVEAEDVGVYYCSQTTHVPTFGGGTKVEIK

SEQ ID NO: 529 - protein, 122 aa, synthetic construct
  source 1..122: note = huT6-16-2[TROP2] Variable Heavy (vh) Domain
QVQLVQSGAEVKKPGASVKVSCKASGYTFTDYNMHWVRQAPGQGLEWIGYIYPYNGGTGYNQRFKSRATMTVDTSTSTAYMELRSLRSDDTAVYYCAREDYGSSPSYAMDYWGQGTMVTVSS

SEQ ID NO: 530 - protein, 111 aa, synthetic construct; identical to SEQ ID NO: 528
  source 1..111: note = huT6-16-2[TROP2] Variable Light (vl) Domain

SEQ ID NO: 531 - protein, 121 aa, synthetic construct
  source 1..121: note = TINA[TROP2] Variable Heavy (vh) Domain
QIQLVQSGPELKKPGETVRISCKASGYTFTTAGMQWVQKMPGKGLKWIGWINTHSGVPKYAEDFKGRFAFSLETSASTAYLQISNLKNEDTTTYFCARSGFGSSYWYFDVWGAGTAVTVSS

SEQ ID NO: 532 - protein, 107 aa, synthetic construct
  source 1..107: note = TINA[TROP2] Variable Light (vl) Domain
DIVMTQSHKFMSTSVGDRVSITCKASQDVSTAVAWYQQKPGQSPKLLIYSASYRYTGVPDRFTGSGSGTAFTFTISSVQAEDLAVYYCQQHYITPLTFGAGTKLELK

SEQ ID NO: 533 - protein, 116 aa, synthetic construct
  source 1..116: note = 2EF[TROP2] Variable Heavy (vh) Domain
QVQLQQSGAELVRPGTSVKMSCKAAGYTFTNYWIGWVKQRPGHGLEWIGDIYPGGGYTNYNEKFKGKATLTADTSSSTAYMQLSSLTSEDSAIYYCARGTGGGDYWGQGTLVTVSA

SEQ ID NO: 534 - protein, 111 aa, synthetic construct
  source 1..111: note = 2EF[TROP2] Variable Light (vl) Domain
DIVLTQSPASLAVSLGQRATISCRASQSVSTSSYSYMHWYQQKPGQPPKLLIKYASNLESGVPARFSGSGSGTDFTLNIHPVEEEDTATYYCQHSWEIPYTFGGGTKLEIK

SEQ ID NO: 535 - protein, 119 aa, synthetic construct
  source 1..119: note = 2G10[TROP2] Variable Heavy (vh) Domain
QGQMQQSGAELVKPGASVKLSCKTSGFTFSSSYISWLKQKPRQSLEWIAWIYAGTGGTSYNQKFTGKAQLTVDTSSSTAYMQLSSLTSEDSAIYYCARHNPRYYAMDYWGQGTSVTVSS

SEQ ID NO: 536 - protein, 107 aa, synthetic construct
  source 1..107: note = 2G10[TROP2] Variable Light (vl) Domain
ETTVTQSPASLSMAIGEKVTIRCITSTDIDDDMNWYQQKPGEPPKLLISEGNTLRPGVPSRFSSSGYGTDFVFTIENMLSEDVADYYCLQSDNLPYTFGGGTKLEIK

SEQ ID NO: 537 - protein, 116 aa, synthetic construct
  source 1..116: note = Hu2EF-7[TROP2] Variable Heavy (vh) Domain
QVQLVQSGAEVKKPGASVKVSCKASGYTFTNYWIGWVKQAPGQGLEWIGDIYPGGGYTNYNEKFKGRATLTADTSASTAYMELSSLRSEDTAVYYCARGTGGGDYWGQGTLVTVSS

SEQ ID NO: 538 - protein, 111 aa, synthetic construct
  source 1..111: note = Hu2EF-7[TROP2] Variable Light (vl) Domain
DIVLTQSPDSLAVSLGERATINCRASQSVSTSSYSYMHWYQQKPGQPPKLLIKYASNLESGVPDRFSGSGSGTDFTLTISSLQAEDVAVYYCQHSWEIPYTFGGGTKLEIK

SEQ ID NO: 539 - protein, 119 aa, synthetic construct
  source 1..119: note = Hu2G10-5[TROP2] Variable Heavy (vh) Domain
QVQLVQSGAEVKKPGASVKVSCKASGFTFSSSYISWLRQAPGQRLEWIAWIYAGTGGTSYNQKFTGKATLTVDTSASTAYMELSSLRSEDTAVYYCARHNPRYYAMDYWGQGTTVTVSS

SEQ ID NO: 540 - protein, 107 aa, synthetic construct
  source 1..107: note = Hu2G10-5[TROP2] Variable Light (vl) Domain
DTQMTQSPSSLSASVGDRVTITCITSTDIDDDMNWYQQKPGKAPKLLISEGNTLRPGVPSRFSGSGYGTDFTFTISSLQPEDIATYYCLQSDNLPYTFGGGTKVEIK

SEQ ID NO: 541 - protein, 119 aa, synthetic construct
  source 1..119: note = Hu2G10-6[TROP2] Variable Heavy (vh) Domain
QVQLVQSGAEVKKPGASVKVSCKASGFTFSSSYISWLRQAPGQRLEWIAWIYAGTGGTSYNQKFTGRVTLTVDTSASTAYMELSSLRSEDTAVYYCARHNPRYYAMDYWGQGTTVTVSS

SEQ ID NO: 542 - protein, 107 aa, synthetic construct; identical to SEQ ID NO: 540
  source 1..107: note = Hu2G10-6[TROP2] Variable Light (vl) Domain

SEQ ID NO: 543 - protein, 121 aa, synthetic construct
  source 1..121: note = BAT-806[TROP2] Variable Heavy (vh) Domain
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGTLVTVSS

SEQ ID NO: 544 - protein, 107 aa, synthetic construct; identical to SEQ ID NO: 467
  source 1..107: note = BAT-806[TROP2] Variable Light (vl) Domain

SEQ ID NO: 545 - protein, 116 aa, synthetic construct
  source 1..116: note = trop2-ab7[TROP2] Variable Heavy (vh) Domain
QVQLQQSGPEVVKPGASVKMSCKASGYRFTDYVINWVKQRSGQGLEWIGQIYPGSDTFHYNKNFKGKATLTADKSSNTAYMQLNSLTSEDSAVYFCARFFEGFAYWGQGTLVTVSA

SEQ ID NO: 546 - protein, 107 aa, synthetic construct
  source 1..107: note = trop2-ab7[TROP2] Variable Light (vl) Domain
DIKMTQSPSSMYAFLGERVTITCKASQGINNYLSWFHLKPGKSPKSLIYRANRLVDGVPSRFSGSGSGQDYSLTISSLDYEDVGIYYCLQYDEFPLTFGGGTKLELK

SEQ ID NO: 547 - protein, 116 aa, synthetic construct
  source 1..116: note = Trop2-Ab8TROP2[TROP2] Variable Heavy (vh) Domain
QVQLQQSGPEVVKPGASVKMSCKASGYRFTDYVINWVKQRTGQGLEWIGQIYPGSDSFHYNKNFKGKATLTADKSSNTAYMQLNSLTSEDSAVYFCARFFEGLAYWGQGTLVTVSA

SEQ ID NO: 548 - protein, 107 aa, synthetic construct
  source 1..107: note = Trop2-Ab8TROP2[TROP2] Variable Light (vl) Domain
DIKMTQSPSSMYAFLGERVTITCKASQGINNYLSWFQLKPGKSPKSLIYRANRLVDGVPSRFSGSGSGQDYSLTISSLDYEDMGIYYCLQYDEFPLTFGGGTKLELK

SEQ ID NO: 549 - protein, 116 aa, synthetic construct
  source 1..116: note = Trop2-Ab22[TROP2] Variable Heavy (vh) Domain
QVQLQQSGPELVKPGASVKMSCKASGYTFTDYVINWVKQRPGQGLEWVGQIYPGSDGTYYNEPFKGEATLTADKSSNTAYMQLSSLTSEDSAVYFCARFFEGFAYWGQGTLVTVSA

SEQ ID NO: 550 - protein, 107 aa, synthetic construct
  source 1..107: note = Trop2-Ab22[TROP2] Variable Light (vl) Domain
DIKMTQSPSSMFASLGERVTITCKASQDINWYLSWFQQKPGKSPKTLIYRANRLVDGVPSRFSGSGSGQDYSLTISSLDYEDMGVYYCLQYEEFPLTFGAGTKLELK

SEQ ID NO: 551 - protein, 116 aa, synthetic construct; identical to SEQ ID NO: 547
  source 1..116: note = Trop2-Ab40TROP2[TROP2] Variable Heavy (vh) Domain

SEQ ID NO: 552 - protein, 107 aa, synthetic construct
  source 1..107: note = Trop2-Ab40TROP2[TROP2] Variable Light (vl) Domain
DIKMTQSPSSMYAFLGERVTITCTASQGINNYLSWFQLKPGKSPKSLIYRANRLVDGVPSRFSGSGSGQDYSLTISSLDYEDMGIYYCLQYDEFPLTFGGGTKLELK

SEQ ID NO: 553 - protein, 115 aa, synthetic construct
  source 1..115: note = Trop2-Ab46TROP2[TROP2] Variable Heavy (vh) Domain
QVQLQQPGAELVRPGASVRLSCKSSGYTFTSYWINWVKQRPGQGLEWIGNIFPSHSYTNYNQKFRDKATLTVDKSSSTAYLQLSSPTSDDSAVYYCTRGSGFDYWGQGTALTVSS

SEQ ID NO: 554 - protein, 108 aa, synthetic construct
  source 1..108: note = Trop2-Ab46TROP2[TROP2] Variable Light (vl) Domain
DILLTQSPAILSVSPGERVSFSCRASQNIGTSIHWYQQRTNGSPRLLISYASESISGIPSRFSGSGSGTDFTLLIINSVESEDIADYYCQHSYSWPFTFGSGTKLEVK

SEQ ID NO: 555 - protein, 114 aa, synthetic construct
  source 1..114: note = Trop2-Ab50TROP2[TROP2] Variable Heavy (vh) Domain
QVQLQQSGAELVRPGTSVKISCKASGYTFTNYWLDWIKQRPGQGLEWIGNIFPGGDYTNYNEEFRGKATLTADTSSRTAFMQLSSLTSEDSAVYFCARSSYDYWGQGTLVTVSA

SEQ ID NO: 556 - protein, 107 aa, synthetic construct
  source 1..107: note = Trop2-Ab50TROP2[TROP2] Variable Light (vl) Domain
DILLTQSPAILSVSPGERVSFSCRASQNIGTSIHWYQQRTNGSPRLLIKYSSESISGIPSRFSGSGSGTDFSLTINSVESEDIGDYYCQHSYTWPFTFGSGTKLEIK

SEQ ID NO: 557 - protein, 116 aa, synthetic construct
  source 1..116: note = Trop2-Ab51TROP2[TROP2] Variable Heavy (vh) Domain
QVQLQQSGPEVVKPGASVKMSCRASGYRFTDYVINWVKQRTGQGLEWIGQIYPGSDSFHYNKNFKGKATLTADKSSNTAYMQLNSLTSEDSAVYFCARFFEGFAYWGQGTLVTVSA

SEQ ID NO: 558 - protein, 107 aa, synthetic construct; identical to SEQ ID NO: 548
  source 1..107: note = Trop2-Ab51TROP2[TROP2] Variable Light (vl) Domain

SEQ ID NO: 559 - protein, 119 aa, synthetic construct
  source 1..119: note = 1B3[TROP2] Variable Heavy (vh) Domain
QVQLQQSGAELVRPGVSVKISCKGSGYTFTDYSINWVKQSHAKSLEWIGVISTYYGDASYNQKFKGKATMTVDKSSSTAYMELARLTSEDSAIYYCARYGDGYNQFDYWGQGTTLTVSS

SEQ ID NO: 560 - protein, 111 aa, synthetic construct
  source 1..111: note = 1B3[TROP2] Variable Light (vl) Domain
DIVLTQSPASLAVSLGQRATISCRVSESVDSYGNNFMHWYQQKPGQPPKLLIYRASNLESGVPARFSGSGSGTDFTLTINPMEADDVATYYCQQSYEDPYTFGGGTKLEIK

SEQ ID NO: 561 - protein, 118 aa, synthetic construct
  source 1..118: note = 15D9[TROP2] Variable Heavy (vh) Domain
QVQLQQSGAELVRPGVSVKISCKGSGYTFTDYSIHWVKQSHAKSLEWIGVISSYYGDATYNQKFKGKATMTVDKSSSTAYMELARLTSEDSAIYYCALLGAYNYFDYWGQGTTLTVSS

SEQ ID NO: 562 - protein, 111 aa, synthetic construct
  source 1..111: note = 15D9[TROP2] Variable Light (vl) Domain
DIVLTQSPASLAVSLGQRATISCRASESVDSYGNSFMHWYQQKPGQPPKLLVYRASNLESGIPARFSGSGSRTDFTLTINPVEADDVATYYCQQSYEDPPTFGGGTKLEIK

SEQ ID NO: 563 - protein, 118 aa, synthetic construct
  source 1..118: note = 15A10[TROP2] Variable Heavy (vh) Domain
EVQLEQSGPGLVQPSQSLSITCTVSGFSLTRYGVHWVRQSPGRSLEWLGVIWSGGNTDYNAAFISRLTITKDNSKSQVFFKMNSLQPNDTGVYYCAREDRYVGEFAYWGQGTLVTVSA

SEQ ID NO: 564 - protein, 111 aa, synthetic construct
  source 1..111: note = 15A10[TROP2] Variable Light (vl) Domain
DIVLTQSTASLAVSLGQRATISCRASESVDSYGNSFIHWYQQKPGQPPKLLIYRASNLESGIPARFSGSGSRTDFTLTINPVEADDVATYYCQQSNEDPRTFGGGTKLEIK

SEQ ID NO: 565 - protein, 118 aa, synthetic construct
  source 1..118: note = 17B6[TROP2] Variable Heavy (vh) Domain
QVQLQQSGAELVRPGVSVKISCKGSGYTFTDYGMNWVKQSHAKSLEWIGVISTFYGDANYNQKFKGKATMTVDKSSSTAYMELARLTSEDSAIYYCARRGNYDSMDYWGQGTSVTVSS

SEQ ID NO: 566 - protein, 111 aa, synthetic construct
  source 1..111: note = 17B6[TROP2] Variable Light (vl) Domain
DIVLTQSPVFLAVSLGQRATISCRASESVDSYGNSFMHWYQQKPGQPPKLLIYRASNLESGIPARFSGSGSRTDFTLTINPVEADDVETYYCQQSYEDPPTFGGGTKLEIE

SEQ ID NO: 567 - protein, 118 aa, synthetic construct
  source 1..118: note = 17D1[TROP2] Variable Heavy (vh) Domain
QVQLQQSGAELVRPGVSVKISCKGSGYTFTDYSIHWVKQSHAKSLEWIGLISTYYGDATYTQKFKGKATMTVDKSSSTAYMELARLTSEDSAIYYCALLGAYNYFDYWGQGTTLTVSS

SEQ ID NO: 568 - protein, 111 aa, synthetic construct
  source 1..111: note = 17D1[TROP2] Variable Light (vl) Domain
DIVLTQSPASLAVSLGQRATISCRASESVDSYGNSFMHWYQQKPGQPPKLLVYRASNLESGIPARFSGSGSRTDFTLTINPVEADDIATYYCQQSYEDPPTFGGGTKLEIK

SEQ ID NO: 569 - protein, 121 aa, synthetic construct
  source 1..121: note = M1[TROP2] Variable Heavy (vh) Domain
QVQLQQPGAELVRPGASVKLSCRASGYTFTNYWMNWVKQRPEQGLEWIGRIDPNDSETHYNQKFKDRAILTVDKASNTAYMQLSGLTSEDSAVHYCARSGFGSTYWFFDVWGAGTTVTVSS

SEQ ID NO: 570 - protein, 107 aa, synthetic construct
  source 1..107: note = M1[TROP2] Variable Light (vl) Domain
DIVMTQSHKFMSTSVGDRVSITCKASQDVSTAVAWYQQKPGQSPKLLIYSASYRYTGVPDRFAGSGYGTDFTFTISSVQTEDLTVYHCQQHYSTPLTFGPGTRLELK

SEQ ID NO: 571 - protein, 121 aa, synthetic construct
  source 1..121: note = HU1[TROP2] Variable Heavy (vh) Domain
QVQLVQSGAEVKKPGSSVKVSCKASGYTFTNYWMNWVRQAPGKGLEWVGRIDPNDSETHYNQKFKDRFTITRDNSKNTLYLQMNSLRAEDTAVYYCARSGFGSTYWFFDVWGQGTTVTVSS

SEQ ID NO: 572 - protein, 107 aa, synthetic construct
  source 1..107: note = HU1[TROP2] Variable Light (vl) Domain
AIRMTQSPFSLSASVGDRVTITCKASQDVSTAVAWYLQKPGQSPQLLIYSASYRYTRIPPRFSGSGYGTDFTLTINNIESEDAAYYFCQQHYSTPLTFGQGTRLEIK

SEQ ID NO: 573 - protein, 121 aa, synthetic construct
  source 1..121: note = HU2[TROP2] Variable Heavy (vh) Domain
EVQLLESGGGLVQPGGSLRLSCAASGFTFSNYWMNWVRQAPGKGLEWIGRIDPNDSETHYNQKFKDRVTISVDKSKNQFSLKLSSVTAADTAVYYCARSGFGSTYWFFDVWGQGTTVTVSS

SEQ ID NO: 574 - protein, 107 aa, synthetic construct
  source 1..107: note = HU2[TROP2] Variable Light (vl) Domain
ETTLTQSPAFMSATPGDKVNISCKASQDVSTAVAWYLQKPGQSPQLLIYSASYRYTGVPDRFSGSGSGTDFTLTISSLQAEDVAVYYCQQHYSTPLTFGQGTRLEIK

SEQ ID NO: 575 - protein, 121 aa, synthetic construct
  source 1..121: note = HU3[TROP2] Variable Heavy (vh) Domain
EVQLVESGGGLVQPGGSLRLSCAASGFTFSNYWMNWVRQAPGKGLEWMGRIDPNDSETHYNQKFKDRVTISVDKSKNQFSLKLSSVTAADTAVYYCARSGFGSTYWFFDVWGQGTMVTVSS

SEQ ID NO: 576 - protein, 107 aa, synthetic construct
  source 1..107: note = HU3[TROP2] Variable Light (vl) Domain
DIVMTQTPLSLPVTPGEPASISCKASQDVSTAVAWYLQKPGQSPQLLIYSASYRYTGVPSRFSGSGSGTDFTLTINSLEAEDAATYYCQQHYSTPLTFGQGTRLEIK

SEQ ID NO: 577 - protein, 121 aa, synthetic construct
  source 1..121: note = HU4[TROP2] Variable Heavy (vh) Domain
EVQLVESGGGLVQPGGSLRLSCAASGFTFSNYWMNWVRQAPGKGLEWIGRIDPNDSETHYNQKFKDRVTMTRDTSTSTVYMELSSLRSEDTAVYYCARSGFGSTYWFFDVWGQGTMVTVSS

SEQ ID NO: 578 - protein, 107 aa, synthetic construct
  source 1..107: note = HU4[TROP2] Variable Light (vl) Domain
DIVMTQSPDSLAVSLGERATINCKASQDVSTAVAWYQQKPGQAPRLLIYSASYRYTGVPSRFSGSGSGTDFTLTINSLEAEDAATYYCQQHYSTPLTFGQGTRLEIK

SEQ ID NO: 579 - protein, 121 aa, synthetic construct
  source 1..121: note = HU5[TROP2] Variable Heavy (vh) Domain
EVQLVESGGGLVQPGGSLRLSCAASGFTVSNYWMNWVRQAPGKGLEWIGRIDPNDSETHYNQKFKDRVTMTRDTSTSTVYMELSSLRSEDTAVYYCARSGFGSTYWFFDVWGQGTTVTVSS

SEQ ID NO: 580 - protein, 107 aa, synthetic construct
  source 1..107: note = HU5[TROP2] Variable Light (vl) Domain
DIQMTQSPSSLSASVGDRVTITCKASQDVSTAVAWYQQKPGKAPKLFIYSASYRYTGVPDRFSGSGSGTDFTLKISRVEAEDVGVYYCQQHYSTPLTFGQGTRLEIK

SEQ ID NO: 581 - protein, 121 aa, synthetic construct
  source 1..121: note = HU6[TROP2] Variable Heavy (vh) Domain
EVQLLESGGGLVQPGGSLRLSCAASGFTVSNYWMNWVRQAPGKGLEWMGRIDPNDSETHYNQKFKDRVTISVDKSKNQFSLKLSSVTAADTAVYYCARSGFGSTYWFFDVWGQGTTVTVSS

SEQ ID NO: 582 - protein, 107 aa, synthetic construct
  source 1..107: note = HU6[TROP2] Variable Light (vl) Domain
DVVMTQSPLSLPVTLGQPASISCKASQDVSTAVAWYQQKPGKAPKLFIYSASYRYTGVPSRFSGSGSGTDFTLTISSLQPEDFATYYCQQHYSTPLTFGQGTRLEIK

SEQ ID NO: 583 - protein, 121 aa, synthetic construct
  source 1..121: note = HU7[TROP2] Variable Heavy (vh) Domain
EVQLVESGGGLIQPGGSLKLSCAASGFTVSNYWMNWVRQAPGKGLEWMGRIDPNDSETHYNQKFKDRVTMTRDTSTSTVYMELSSLRSEDTAVYYCARSGFGSTYWFFDVWGQGTTVTVSS

SEQ ID NO: 584 - protein, 107 aa, synthetic construct
  source 1..107: note = HU7[TROP2] Variable Light (vl) Domain
AIRMTQSPFSLSASVGDRVTITCKASQDVSTAVAWYLQKPGQSPQLLIYSASYRYTGVPDRFSGSGSGTDFTLKISRVEAEDVGVYYCQQHYSTPLTFGQGTRLEIK

SEQ ID NO: 585 - protein, 121 aa, synthetic construct
  source 1..121: note = HU8[TROP2] Variable Heavy (vh) Domain
QVQLVQSGAEVKKPGASVKVSCKASGYTFTNYWMNWVRQAPGKGLEWMGRIDPNDSETHYNQKFKDRVTMTRDTSTSTVYMELSSLRSEDTAVYYCARSGFGSTYWFFDVWGQGTTVTVSS

SEQ ID NO: 586 - protein, 107 aa, synthetic construct
  source 1..107: note = HU8[TROP2] Variable Light (vl) Domain
AIRMTQSPFSLSASVGDRVTITCKASQDVSTAVAWYLQKPGQSPQLLIYSASYRYTGVPSRFSGSGSGTDFTLKISRVEAEDVGVYYCQQHYSTPLTFGQGTRLEIK

SEQ ID NO: 587 - protein, 121 aa, synthetic construct
  source 1..121: note = HU9[TROP2] Variable Heavy (vh) Domain
EVQLVESGGGLIQPGGSLRLSCAASGFTVSNYWMNWVRQAPGQGLEWMGRIDPNDSETHYNQKFKDRLTITKDTSKNQVVLTMTNMDPVDTATYYCARSGFGSTYWFFDVWGQGTTVTVSS

SEQ ID NO: 588 - protein, 107 aa, synthetic construct
  source 1..107: note = HU9[TROP2] Variable Light (vl) Domain
DIVMTQTPLSLPVTPGEPASISCKASQDVSTAVAWYLQKPGQSPQLLIYSASYRYTGVPDRFSGSGSGTDFTLTISSLQAEDVAVYYCQQHYSTPLTFGQGTRLEIK

SEQ ID NO: 589 - protein, 116 aa, synthetic construct; identical to SEQ ID NO: 495
  source 1..116: note = HV0_VH[TROP2] Variable Heavy (vh) Domain

SEQ ID NO: 590 - protein, 116 aa, synthetic construct; identical to SEQ ID NO: 497
  source 1..116: note = HV3a_VH[TROP2] Variable Heavy (vh) Domain

SEQ ID NO: 591 - protein, 116 aa, synthetic construct; identical to SEQ ID NO: 499
  source 1..116: note = HV4_VH[TROP2] Variable Heavy (vh) Domain

SEQ ID NO: 592 - protein, 116 aa, synthetic construct; identical to SEQ ID NO: 501
  source 1..116: note = HV5_VH[TROP2] Variable Heavy (vh) Domain

SEQ ID NO: 593 - protein, 116 aa, synthetic construct; identical to SEQ ID NO: 503
  source 1..116: note = HV10_VH[TROP2] Variable Heavy (vh) Domain

SEQ ID NO: 594 - protein, 113 aa, synthetic construct; identical to SEQ ID NO: 496
  source 1..113: note = LV0_VL[TROP2] Variable Light (vl) Domain

SEQ ID NO: 595 - protein, 113 aa, synthetic construct; identical to SEQ ID NO: 504
  source 1..113: note = LV2_VL[TROP2] Variable Light (vl) Domain

SEQ ID NO: 596 - protein, 113 aa, synthetic construct; identical to SEQ ID NO: 506
  source 1..113: note = LV4_VL[TROP2] Variable Light (vl) Domain

SEQ ID NO: 597 - protein, 121 aa, synthetic construct
  source 1..121: note = hTINA1 H1_VH[TROP2] Variable Heavy (vh) Domain
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 598 - protein, 121 aa, synthetic construct
  source 1..121: note = hTINA1 H2_VH[TROP2] Variable Heavy (vh) Domain
QVQLVQSGAEVKKPGASVKVSCKASGYTFTTAGMQWVRQAPGQGLEWMGWINTHSGVPKYAEDFKGRVTISLDTSTSTAYLQLSSLKSEDTAVYYCARSGFGSSYWYFDVWGQGTLVTVSS

SEQ ID NO: 599 - protein, 121 aa, synthetic construct
  source 1..121: note = hTINA1 H3_VH[TROP2] Variable Heavy (vh) Domain
QIQLVQSGAEVKKPGESVKVSCKASGYTFTTAGMQWVQQMPGKGLEWMGWINTHSGVPKYAEDFKGRVTFSLDTSTSTAYLQLSSLKSEDTAVYYCARSGFGSSYWYFDVWGQGTLVTVSS

SEQ ID NO: 600 - protein, 107 aa, synthetic construct
  source 1..107: note = hTINA1 L1_VL[TROP2] Variable Light (vl) Domain
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 601 - protein, 107 aa, synthetic construct
  source 1..107: note = hTINA1 L2_VL[TROP2] Variable Light (vl) Domain
DIVMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 602 - protein, 107 aa, synthetic construct
  source 1..107: note = hTINA1 L3_VL[TROP2] Variable Light (vl) Domain
DIVMTQSPSSLSASVGDRVTITCKASQDVSTAVAWYQQKPGKQPKLLIYSASYRYTGVPSRFSGSGSGTDFTLTISSLQPEDFAVYYCQQHYITPLTFGQGTKLEIK

SEQ ID NO: 603 - protein, 116 aa, synthetic construct
  source 1..116: note = Hu2EF VH4_VH[TROP2] Variable Heavy (vh) Domain
QVQLVQSGAEVKKPGASVKVSCKASGYTFTNYWIGWVRQAPGQGLEWIGDIYPGGGYTNYNEKFKGRATLTADTSASTAYMELSSLRSEDTAVYYCARGTGGGDYWGQGTLVTVSS

SEQ ID NO: 604 - protein, 116 aa, synthetic construct
  source 1..116: note = Hu2EF VH5_VH[TROP2] Variable Heavy (vh) Domain
QVQLVQSGAEVKKPGASVKVSCKASGYTFTNYWIGWVRQAPGQGLEWIGDIYPGGGYTNYNEKFKGRATLTADTSTSTAYMELSSLRSEDTAVYYCARGTGGGDYWGQGTLVTVSS

SEQ ID NO: 605 - protein, 116 aa, synthetic construct; identical to SEQ ID NO: 537
  source 1..116: note = Hu2EF VH7_VH[TROP2] Variable Heavy (vh) Domain

SEQ ID NO: 606 - protein, 111 aa, synthetic construct
  source 1..111: note = Hu2EF VL1_VL[TROP2] Variable Light (vl) Domain
DIVMTQSPDSLAVSLGERATINCRASQSVSTSSYSYMHWYQQKPGQPPKLLIKYASNLESGVPDRFSGSGSGTDFTLTISSLQAEDVAVYYCQHSWEIPYTFGQGTKLEIK

SEQ ID NO: 607 - protein, 111 aa, synthetic construct; identical to SEQ ID NO: 538
  source 1..111: note = Hu2EF VL2_VL[TROP2] Variable Light (vl) Domain

SEQ ID NO: 608 - protein, 119 aa, synthetic construct; identical to SEQ ID NO: 539
  source 1..119: note = Hu2G10 VH1_VH[TROP2] Variable Heavy (vh) Domain

SEQ ID NO: 609 - protein, 119 aa, synthetic construct; identical to SEQ ID NO: 541
  source 1..119: note = Hu2G10 VH2_VH[TROP2] Variable Heavy (vh) Domain

SEQ ID NO: 610 - protein, 107 aa, synthetic construct
  source 1..107: note = Hu2G10 VL1_VL[TROP2] Variable Light (vl) Domain
DIQMTQSPSSLSASVGDRVTITCITSTDIDDDMNWYQQKPGKAPKLLISEGNTLRPGVPSRFSGSGYGTDFTFTISSLQPEDIATYYCLQSDNLPYTFGGGTKVEIK

SEQ ID NO: 611 - protein, 107 aa, synthetic construct
  source 1..107: note = Hu2G10 VL2_VL[TROP2] Variable Light (vl) Domain
DIQMTQSPSSLSASVGDRVTITCITSTDIDDDMNWYQQKPGKAPKLLISEGNTLRPGVPSRFSGSGSGTDFTFTISSLQPEDIATYYCLQSDNLPYTFGGGTKVEIK

SEQ ID NO: 612 - protein, 107 aa, synthetic construct; identical to SEQ ID NO: 540
  source 1..107: note = Hu2G10 VL3_VL[TROP2] Variable Light (vl) Domain

SEQ ID NO: 613 - protein, 119 aa, synthetic construct
  source 1..119: note = DP002 HC1_VH[TROP2] Variable Heavy (vh) Domain
QVQLVQSGAEVKKPGASVKVSCKASGYTFTDYSINWVRQAPGQGLEWMGVISTYYGDASYNQKFKGRVTMTTDTSTSTAYMELRSLRSDDTAVYYCARYGDGYNQFDYWGQGTMVTVSS

SEQ ID NO: 614 - protein, 119 aa, synthetic construct
  source 1..119: note = DP002 HC2_VH[TROP2] Variable Heavy (vh) Domain
QVQLVQSGAEVKKPGASVKVSCKASGYTFTDYSINWVRQAPGQGLEWMGVISTYYGDASYNQKFKGRVTMTRDTSTSTVYMELSSLRSEDTAVYYCARYGDGYNQFDYWGQGTLVTVSS

SEQ ID NO: 615 - protein, 119 aa, synthetic construct
  source 1..119: note = DP002 HC3_VH[TROP2] Variable Heavy (vh) Domain
QVQLVQSGAEVKKPGASVKVSCKASGYTFTDYSINWVRQAPGQRLEWMGVISTYYGDASYNQKFKGRVTITRDTSASTAYMELSSLRSEDTAVYYCARYGDGYNQFDYWGQGTLVTVSS

SEQ ID NO: 616 - protein, 119 aa, synthetic construct
  source 1..119: note = DP002 HC4_VH[TROP2] Variable Heavy (vh) Domain
QVQLVQSGAEVKKPGSSVKVSCKASGYTFTDYSINWVRQAPGQGLEWMGVISTYYGDASYNQKFKGRVTITADESTSTAYMELSSLRSEDTAVYYCARYGDGYNQFDYWGQGTLVTVSS

SEQ ID NO: 617 - protein, 119 aa, synthetic construct
  source 1..119: note = DP002 HC5_VH[TROP2] Variable Heavy (vh) Domain
EVQLVQSGAEVKKPGESLKISCKGSGYTFTDYSINWVRQMPGKGLEWMGVISTYYGDASYNQKFKGQVTISADKSISTAYLQWSSLKASDTAMYYCARYGDGYNQFDYWGQGTLVTVSS

SEQ ID NO: 618 - protein, 111 aa, synthetic construct
  source 1..111: note = DP002 LC1_VL[TROP2] Variable Light (vl) Domain
DIVMTQSPDSLAVSLGERATINCRVSESVDSYGNNFMHWYQQKPGQPPKLLIYRASNLESGVPDRFSGSGSGTDFTLTISSLQAEDVAVYYCQQSYEDPYTFGQGTKLEIK

SEQ ID NO: 619 - protein, 111 aa, synthetic construct
  source 1..111: note = DP002 LC2_VL[TROP2] Variable Light (vl) Domain
DVVMTQSPLSLPVTLGQPASISCRVSESVDSYGNNFMHWFQQRPGQSPRRLIYRASNLESGVPDRFSGSGSGTDFTLKISRVEAEDVGVYYCQQSYEDPYTFGGGTKVEIK

SEQ ID NO: 620 - protein, 111 aa, synthetic construct
  source 1..111: note = DP002 LC3_VL[TROP2] Variable Light (vl) Domain
DIVMTQSPLSLPVTPGEPASISCRVSESVDSYGNNFMHWYLQKPGQSPQLLIYRASNLESGVPDRFSGSGSGTDFTLKISRVEAEDVGVYYCQQSYEDPYTFGGGTKVEIK

SEQ ID NO: 621 - protein, 111 aa, synthetic construct
  source 1..111: note = DP002 LC4_VL[TROP2] Variable Light (vl) Domain
EIVLTQSPATLSLSPGERATLSCRVSESVDSYGNNFMHWYQQKPGQAPRLLIYRASNLESGIPARFSGSGSGTDFTLTISSLEPEDFAVYYCQQSYEDPYTFGGGTKVEIK

SEQ ID NO: 622 - protein, 111 aa, synthetic construct
  source 1..111: note = DP002 LC5_VL[TROP2] Variable Light (vl) Domain
DIQMTQSPSSLSASVGDRVTITCRVSESVDSYGNNFMHWYQQKPGKAPKLLIYRASNLESGVPSRFSGSGSGTDFTFTISSLQPEDIATYYCQQSYEDPYTFGGGTKLEIK

SEQ ID NO: 623-625 - skipped (3 entries)

SEQ ID NO: 626 - protein, 450 aa, synthetic construct
  source 1..450: note = XENP41995 Chain 1 - sacituzumab[Trop2]_H0_IgG1_pI(- )_Isosteric_A_/PVA_/S267K/L368D/K370S
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 627 - protein, 712 aa, synthetic construct
  source 1..712: note = XENP41995 Chain 2 - sacituzumab[Trop2]_H0_IgG1_PVA_ /S267K/S364K/E357Q/K447del_(G4S)3_1A7[CD28]_L1.71_H1.14_sc Fv(GKPGS)4
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKGKPGSGKPGSGKPGSGKPGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSS

SEQ ID NO: 628 - protein, 214 aa, synthetic construct
  source 1..214: note = XENP41995 Chain 3 - sacituzumab[Trop2]_L0
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 629 - protein, 254 aa, synthetic construct
  source 1..254: note = CD3 High - [anti-CD3]_H1.30_L1.47_scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 630 - protein, 125 aa, synthetic construct
  source 1..125: note = CD3 High - [anti-CD3]_H1.30_L1.47_scFv Variable Heavy (vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 631 - protein, 5 aa, synthetic construct
  source 1..5: note = CD3 High - [anti-CD3]_H1.30_L1.47_scFv vhCDR1
TYAMN

SEQ ID NO: 632 - protein, 19 aa, synthetic construct
  source 1..19: note = CD3 High - [anti-CD3]_H1.30_L1.47_scFv vhCDR2
RIRSKYNNYATYYADSVKG

SEQ ID NO: 633 - protein, 14 aa, synthetic construct
  source 1..14: note = CD3 High - [anti-CD3]_H1.30_L1.47_scFv vhCDR3
HGNFGDSYVSWFAY

SEQ ID NO: 634 - protein, 109 aa, synthetic construct
  source 1..109: note = CD3 High - [anti-CD3]_H1.30_L1.47_scFv Variable Light (vl) Domain
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 635 - protein, 14 aa, synthetic construct
  source 1..14: note = CD3 High - [anti-CD3]_H1.30_L1.47_scFv vlCDR1
GSSTGAVTTSNYAN

SEQ ID NO: 636 - protein, 7 aa, synthetic construct
  source 1..7: note = CD3 High - [anti-CD3]_H1.30_L1.47_scFv vlCDR2
GTNKRAP

SEQ ID NO: 637 - protein, 9 aa, synthetic construct
  source 1..9: note = CD3 High - [anti-CD3]_H1.30_L1.47_scFv vlCDR3
ALWYSNHWV

SEQ ID NO: 638 - protein, 20 aa, synthetic construct
  source 1..20: note = CD3 High - [anti-CD3]_H1.30_L1.47_scFv Linker
GKPGSGKPGSGKPGSGKPGS

SEQ ID NO: 639 - protein, 254 aa, synthetic construct
  source 1..254: note = CD3 High-Int #1 - [anti-CD3]_H1.32_L1.47_scFv scFv
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 640 - protein, 125 aa, synthetic construct
  source 1..125: note = CD3 High-Int #1 - [anti-CD3]_H1.32_L1.47_scFv Variable Heavy (vh) Domain
EVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSS

SEQ ID NO: 641 - protein, 5 aa, synthetic construct
  source 1..5: note = CD3 High-Int #1 - [anti-CD3]_H1.32_L1.47_scFv vhCDR1
TYAMN

SEQ ID NO: 642 - protein, 19 aa, synthetic construct
  source 1..19: note = CD3 High-Int #1 - [anti-CD3]_H1.32_L1.47_scFv vhCDR2
RIRSKANNYATYYADSVKG

SEQ ID NO: 643 - protein, 14 aa, synthetic construct
  source 1..14: note = CD3 High-Int #1 - [anti-CD3]_H1.32_L1.47_scFv vhCDR3
HGNFGDSYVSWFAY

SEQ ID NO: 644 - protein, 109 aa, synthetic construct; identical to SEQ ID NO: 634
  source 1..109: note = CD3 High-Int #1 - [anti-CD3]_H1.32_L1.47_scFv Variable Light (vl) Domain

SEQ ID NO: 645 - protein, 14 aa, synthetic construct
  source 1..14: note = CD3 High-Int #1 - [anti-CD3]_H1.32_L1.47_scFv vlCDR1
GSSTGAVTTSNYAN

SEQ ID NO: 646 - protein, 7 aa, synthetic construct
  source 1..7: note = CD3 High-Int #1 - [anti-CD3]_H1.32_L1.47_scFv vlCDR2
GTNKRAP

SEQ ID NO: 647 - protein, 9 aa, synthetic construct
  source 1..9: note = CD3 High-Int #1 - [anti-CD3]_H1.32_L1.47_scFv vlCDR3
ALWYSNHWV

SEQ ID NO: 648 - protein, 20 aa, synthetic construct
  source 1..20: note = CD3 High-Int #1 - [anti-CD3]_H1.32_L1.47_scFv Linker
GKPGSGKPGSGKPGSGKPGS

SEQ ID NO: 649 - protein, 254 aa, synthetic construct
  source 1..254: note = CD3 High-Int #2 - [anti-CD3]_H1.89_L1.47_scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 650 - protein, 125 aa, synthetic construct
  source 1..125: note = CD3 High-Int #2 - [anti-CD3]_H1.89_L1.47_scFv Variable Heavy (vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSS

SEQ ID NO: 651 - protein, 5 aa, synthetic construct
  source 1..5: note = CD3 High-Int #2 - [anti-CD3]_H1.89_L1.47_scFv vhCDR1
TYAMN

SEQ ID NO: 652 - protein, 19 aa, synthetic construct
  source 1..19: note = CD3 High-Int #2 - [anti-CD3]_H1.89_L1.47_scFv vhCDR2
RIRSKYNNYATYYADSVKG

SEQ ID NO: 653 - protein, 14 aa, synthetic construct
  source 1..14: note = CD3 High-Int #2 - [anti-CD3]_H1.89_L1.47_scFv vhCDR3
HGNFGDEYVSWFAY

SEQ ID NO: 654 - protein, 109 aa, synthetic construct; identical to SEQ ID NO: 634
  source 1..109: note = CD3 High-Int #2 - [anti-CD3]_H1.89_L1.47_scFv Variable Light (vl) Domain

SEQ ID NO: 655 - protein, 14 aa, synthetic construct
  source 1..14: note = CD3 High-Int #2 - [anti-CD3]_H1.89_L1.47_scFv vlCDR1
GSSTGAVTTSNYAN

SEQ ID NO: 656 - protein, 7 aa, synthetic construct
  source 1..7: note = CD3 High-Int #2 - [anti-CD3]_H1.89_L1.47_scFv vlCDR2
GTNKRAP

SEQ ID NO: 657 - protein, 9 aa, synthetic construct
  source 1..9: note = CD3 High-Int #2 - [anti-CD3]_H1.89_L1.47_scFv vlCDR3
ALWYSNHWV

SEQ ID NO: 658 - protein, 20 aa, synthetic construct
  source 1..20: note = CD3 High-Int #2 - [anti-CD3]_H1.89_L1.47_scFv Linker
GKPGSGKPGSGKPGSGKPGS

SEQ ID NO: 659 - protein, 254 aa, synthetic construct
  source 1..254: note = CD3 High-Int - [anti-CD3]_H1.90_L1.47_scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 660 - protein, 125 aa, synthetic construct
  source 1..125: note = CD3 High-Int - [anti-CD3]_H1.90_L1.47_scFv Variable Heavy (vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFAYWGQGTLVTVSS

SEQ ID NO: 661 - protein, 5 aa, synthetic construct
  source 1..5: note = CD3 High-Int - [anti-CD3]_H1.90_L1.47_scFv vhCDR1
TYAMN

SEQ ID NO: 662 - protein, 19 aa, synthetic construct
  source 1..19: note = CD3 High-Int - [anti-CD3]_H1.90_L1.47_scFv vhCDR2
RIRSKYNNYATYYADSVKG

SEQ ID NO: 663 - protein, 14 aa, synthetic construct
  source 1..14: note = CD3 High-Int - [anti-CD3]_H1.90_L1.47_scFv vhCDR3
HGNFGDPYVSWFAY

SEQ ID NO: 664 - protein, 109 aa, synthetic construct; identical to SEQ ID NO: 634
  source 1..109: note = CD3 High-Int - [anti-CD3]_H1.90_L1.47_scFv Variable Light (vl) Domain

SEQ ID NO: 665 - protein, 14 aa, synthetic construct
  source 1..14: note = CD3 High-Int - [anti-CD3]_H1.90_L1.47_scFv vlCDR1
GSSTGAVTTSNYAN

SEQ ID NO: 666 - protein, 7 aa, synthetic construct
  source 1..7: note = CD3 High-Int - [anti-CD3]_H1.90_L1.47_scFv vlCDR2
GTNKRAP

SEQ ID NO: 667 - protein, 9 aa, synthetic construct
  source 1..9: note = CD3 High-Int - [anti-CD3]_H1.90_L1.47_scFv vlCDR3
ALWYSNHWV

SEQ ID NO: 668 - protein, 20 aa, synthetic construct
  source 1..20: note = CD3 High-Int - [anti-CD3]_H1.90_L1.47_scFv Linker
GKPGSGKPGSGKPGSGKPGS

SEQ ID NO: 669 - protein, 254 aa, synthetic construct
  source 1..254: note = Anti-CD3-Intermediate - [anti-CD3]_H1.33_L1.47_scFv scFv
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 670 - protein, 125 aa, synthetic construct
  source 1..125: note = Anti-CD3-Intermediate - [anti-CD3]_H1.33_L1.47_scFv Variable Heavy (vh) Domain
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSS

SEQ ID NO: 671 - protein, 5 aa, synthetic construct
  source 1..5: note = Anti-CD3-Intermediate - [anti-CD3]_H1.33_L1.47_scFv vhCDR1
TYAMN

SEQ ID NO: 672 - protein, 19 aa, synthetic construct
  source 1..19: note = Anti-CD3-Intermediate - [anti-CD3]_H1.33_L1.47_scFv vhCDR2
RIRSKYNNYATYYADSVKG

SEQ ID NO: 673 - protein, 14 aa, synthetic construct
  source 1..14: note = Anti-CD3-Intermediate - [anti-CD3]_H1.33_L1.47_scFv vhCDR3
HGNFGDSYVSWFDY

SEQ ID NO: 674 - protein, 109 aa, synthetic construct; identical to SEQ ID NO: 634
  source 1..109: note = Anti-CD3-Intermediate - [anti-CD3]_H1.33_L1.47_scFv Variable Light (vl) Domain

SEQ ID NO: 675 - protein, 14 aa, synthetic construct
  source 1..14: note = Anti-CD3-Intermediate - [anti-CD3]_H1.33_L1.47_scFv vlCDR1
GSSTGAVTTSNYAN

SEQ ID NO: 676 - protein, 7 aa, synthetic construct
  source 1..7: note = Anti-CD3-Intermediate - [anti-CD3]_H1.33_L1.47_scFv vlCDR2
GTNKRAP

SEQ ID NO: 677 - protein, 9 aa, synthetic construct
  source 1..9: note = Anti-CD3-Intermediate - [anti-CD3]_H1.33_L1.47_scFv vlCDR3
ALWYSNHWV

SEQ ID NO: 678 - protein, 20 aa, synthetic construct
  source 1..20: note = Anti-CD3-Intermediate - [anti-CD3]_H1.33_L1.47_scFv Linker
GKPGSGKPGSGKPGSGKPGS

SEQ ID NO: 679 - protein, 254 aa, synthetic construct
  source 1..254: note = CD3 High-Int - [anti-CD3]_H1.31_L1.47_scFv scFv
EVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 680 - protein, 125 aa, synthetic construct
  source 1..125: note = CD3 High-Int - [anti-CD3]_H1.31_L1.47_scFv Variable Heavy (vh) Domain
EVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 681 - protein, 5 aa, synthetic construct
  source 1..5: note = CD3 High-Int - [anti-CD3]_H1.31_L1.47_scFv vhCDR1
TYAMS

SEQ ID NO: 682 - protein, 19 aa, synthetic construct
  source 1..19: note = CD3 High-Int - [anti-CD3]_H1.31_L1.47_scFv vhCDR2
RIRSKYNNYATYYADSVKG

SEQ ID NO: 683 - protein, 14 aa, synthetic construct
  source 1..14: note = CD3 High-Int - [anti-CD3]_H1.31_L1.47_scFv vhCDR3
HGNFGDSYVSWFAY

SEQ ID NO: 684 - protein, 109 aa, synthetic construct; identical to SEQ ID NO: 634
  source 1..109: note = CD3 High-Int - [anti-CD3]_H1.31_L1.47_scFv Variable Light (vl) Domain

SEQ ID NO: 685 - protein, 14 aa, synthetic construct
  source 1..14: note = CD3 High-Int - [anti-CD3]_H1.31_L1.47_scFv vlCDR1
GSSTGAVTTSNYAN

SEQ ID NO: 686 - protein, 7 aa, synthetic construct
  source 1..7: note = CD3 High-Int - [anti-CD3]_H1.31_L1.47_scFv vlCDR2
GTNKRAP

SEQ ID NO: 687 - protein, 9 aa, synthetic construct
  source 1..9: note = CD3 High-Int - [anti-CD3]_H1.31_L1.47_scFv vlCDR3
ALWYSNHWV

SEQ ID NO: 688 - protein, 20 aa, synthetic construct
  source 1..20: note = CD3 High-Int - [anti-CD3]_H1.31_L1.47_scFv Linker
GKPGSGKPGSGKPGSGKPGS

SEQ ID NO: 689 - protein, 254 aa, synthetic construct
  source 1..254: note = CD3 High[VL-VH] - [anti-CD3]_L1.47_ H1.30_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 690 - protein, 109 aa, synthetic construct; identical to SEQ ID NO: 634
  source 1..109: note = CD3 High[VL-VH] - [anti-CD3]_L1.47_ H1.30_scFv Variable Light (vl) Domain

SEQ ID NO: 691 - protein, 14 aa, synthetic construct
  source 1..14: note = CD3 High[VL-VH] - [anti-CD3]_L1.47_ H1.30_scFv vlCDR1
GSSTGAVTTSNYAN

SEQ ID NO: 692 - protein, 7 aa, synthetic construct
  source 1..7: note = CD3 High[VL-VH] - [anti-CD3]_L1.47_ H1.30_scFv vlCDR2
GTNKRAP

SEQ ID NO: 693 - protein, 9 aa, synthetic construct
  source 1..9: note = CD3 High[VL-VH] - [anti-CD3]_L1.47_ H1.30_scFv vlCDR3
ALWYSNHWV

SEQ ID NO: 694 - protein, 125 aa, synthetic construct; identical to SEQ ID NO: 630
  source 1..125: note = CD3 High[VL-VH] - [anti-CD3]_L1.47_ H1.30_scFv Variable Heavy (vh) Domain

SEQ ID NO: 695 - protein, 5 aa, synthetic construct
  source 1..5: note = CD3 High[VL-VH] - [anti-CD3]_L1.47_ H1.30_scFv vhCDR1
TYAMN

SEQ ID NO: 696 - protein, 19 aa, synthetic construct
  source 1..19: note = CD3 High[VL-VH] - [anti-CD3]_L1.47_ H1.30_scFv vhCDR2
RIRSKYNNYATYYADSVKG

SEQ ID NO: 697 - protein, 14 aa, synthetic construct
  source 1..14: note = CD3 High[VL-VH] - [anti-CD3]_L1.47_ H1.30_scFv vhCDR3
HGNFGDSYVSWFAY

SEQ ID NO: 698 - protein, 20 aa, synthetic construct
  source 1..20: note = CD3 High[VL-VH] - [anti-CD3]_L1.47_ H1.30_scFv Linker
GKPGSGKPGSGKPGSGKPGS

SEQ ID NO: 699 - protein, 254 aa, synthetic construct
  source 1..254: note = CD3 High-Int #1[VL-VH] - [anti-CD3]_L1.47_ H1.32_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKANNYATYYADSVKGRFTISRDDSKNTLYLQMNSLRAEDTAVYYCVRHGNFGDSYVSWFAYWGQGTLVTVSS

SEQ ID NO: 700 - protein, 109 aa, synthetic construct; identical to SEQ ID NO: 634
  source 1..109: note = CD3 High-Int #1[VL-VH] - [anti-CD3]_L1.47_ H1.32_scFv Variable Light (vl) Domain

SEQ ID NO: 701 - protein, 14 aa, synthetic construct
  source 1..14: note = CD3 High-Int #1[VL-VH] - [anti-CD3]_L1.47_ H1.32_scFv vlCDR1
GSSTGAVTTSNYAN

SEQ ID NO: 702 - protein, 7 aa, synthetic construct
  source 1..7: note = CD3 High-Int #1[VL-VH] - [anti-CD3]_L1.47_ H1.32_scFv vlCDR2
GTNKRAP

SEQ ID NO: 703 - protein, 9 aa, synthetic construct
  source 1..9: note = CD3 High-Int #1[VL-VH] - [anti-CD3]_L1.47_ H1.32_scFv vlCDR3
ALWYSNHWV

SEQ ID NO: 704 - protein, 125 aa, synthetic construct; identical to SEQ ID NO: 640
  source 1..125: note = CD3 High-Int #1[VL-VH] - [anti-CD3]_L1.47_ H1.32_scFv Variable Heavy (vh) Domain

SEQ ID NO: 705 - protein, 5 aa, synthetic construct
  source 1..5: note = CD3 High-Int #1[VL-VH] - [anti-CD3]_L1.47_ H1.32_scFv vhCDR1
TYAMN

SEQ ID NO: 706 - protein, 19 aa, synthetic construct
  source 1..19: note = CD3 High-Int #1[VL-VH] - [anti-CD3]_L1.47_ H1.32_scFv vhCDR2
RIRSKANNYATYYADSVKG

SEQ ID NO: 707 - protein, 14 aa, synthetic construct
  source 1..14: note = CD3 High-Int #1[VL-VH] - [anti-CD3]_L1.47_ H1.32_scFv vhCDR3
HGNFGDSYVSWFAY

SEQ ID NO: 708 - protein, 20 aa, synthetic construct
  source 1..20: note = CD3 High-Int #1[VL-VH] - [anti-CD3]_L1.47_ H1.32_scFv Linker
GKPGSGKPGSGKPGSGKPGS

SEQ ID NO: 709 - protein, 254 aa, synthetic construct
  source 1..254: note = CD3 High-Int #2[VL-VH] - [anti-CD3]_L1.47_ H1.89_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDEYVSWFAYWGQGTLVTVSS

SEQ ID NO: 710 - protein, 109 aa, synthetic construct; identical to SEQ ID NO: 634
  source 1..109: note = CD3 High-Int #2[VL-VH] - [anti-CD3]_L1.47_ H1.89_scFv Variable Light (vl) Domain

SEQ ID NO: 711 - protein, 14 aa, synthetic construct
  source 1..14: note = CD3 High-Int #2[VL-VH] - [anti-CD3]_L1.47_ H1.89_scFv vlCDR1
GSSTGAVTTSNYAN

SEQ ID NO: 712 - protein, 7 aa, synthetic construct
  source 1..7: note = CD3 High-Int #2[VL-VH] - [anti-CD3]_L1.47_ H1.89_scFv vlCDR2
GTNKRAP

SEQ ID NO: 713 - protein, 9 aa, synthetic construct
  source 1..9: note = CD3 High-Int #2[VL-VH] - [anti-CD3]_L1.47_ H1.89_scFv vlCDR3
ALWYSNHWV

SEQ ID NO: 714 - protein, 125 aa, synthetic construct; identical to SEQ ID NO: 650
  source 1..125: note = CD3 High-Int #2[VL-VH] - [anti-CD3]_L1.47_ H1.89_scFv Variable Heavy (vh) Domain

SEQ ID NO: 715 - protein, 5 aa, synthetic construct
  source 1..5: note = CD3 High-Int #2[VL-VH] - [anti-CD3]_L1.47_ H1.89_scFv vhCDR1
TYAMN

SEQ ID NO: 716 - protein, 19 aa, synthetic construct
  source 1..19: note = CD3 High-Int #2[VL-VH] - [anti-CD3]_L1.47_ H1.89_scFv vhCDR2
RIRSKYNNYATYYADSVKG

SEQ ID NO: 717 - protein, 14 aa, synthetic construct
  source 1..14: note = CD3 High-Int #2[VL-VH] - [anti-CD3]_L1.47_ H1.89_scFv vhCDR3
HGNFGDEYVSWFAY

SEQ ID NO: 718 - protein, 20 aa, synthetic construct
  source 1..20: note = CD3 High-Int #2[VL-VH] - [anti-CD3]_L1.47_ H1.89_scFv Linker
GKPGSGKPGSGKPGSGKPGS

SEQ ID NO: 719 - protein, 254 aa, synthetic construct
  source 1..254: note = CD3 High-Int[VL-VH] - [anti-CD3]_L1.47_ H1.90_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDPYVSWFAYWGQGTLVTVSS

SEQ ID NO: 720 - protein, 109 aa, synthetic construct; identical to SEQ ID NO: 634
  source 1..109: note = CD3 High-Int[VL-VH] - [anti-CD3]_L1.47_ H1.90_scFv Variable Light (vl) Domain

SEQ ID NO: 721 - protein, 14 aa, synthetic construct
  source 1..14: note = CD3 High-Int[VL-VH] - [anti-CD3]_L1.47_ H1.90_scFv vlCDR1
GSSTGAVTTSNYAN

SEQ ID NO: 722 - protein, 7 aa, synthetic construct
  source 1..7: note = CD3 High-Int[VL-VH] - [anti-CD3]_L1.47_ H1.90_scFv vlCDR2
GTNKRAP

SEQ ID NO: 723 - protein, 9 aa, synthetic construct
  source 1..9: note = CD3 High-Int[VL-VH] - [anti-CD3]_L1.47_ H1.90_scFv vlCDR3
ALWYSNHWV

SEQ ID NO: 724 - protein, 125 aa, synthetic construct; identical to SEQ ID NO: 660
  source 1..125: note = CD3 High-Int[VL-VH] - [anti-CD3]_L1.47_ H1.90_scFv Variable Heavy (vh) Domain

SEQ ID NO: 725 - protein, 5 aa, synthetic construct
  source 1..5: note = CD3 High-Int[VL-VH] - [anti-CD3]_L1.47_ H1.90_scFv vhCDR1
TYAMN

SEQ ID NO: 726 - protein, 19 aa, synthetic construct
  source 1..19: note = CD3 High-Int[VL-VH] - [anti-CD3]_L1.47_ H1.90_scFv vhCDR2
RIRSKYNNYATYYADSVKG

SEQ ID NO: 727 - protein, 14 aa, synthetic construct
  source 1..14: note = CD3 High-Int[VL-VH] - [anti-CD3]_L1.47_ H1.90_scFv vhCDR3
HGNFGDPYVSWFAY

SEQ ID NO: 728 - protein, 20 aa, synthetic construct
  source 1..20: note = CD3 High-Int[VL-VH] - [anti-CD3]_L1.47_ H1.90_scFv Linker
GKPGSGKPGSGKPGSGKPGS

SEQ ID NO: 729 - protein, 254 aa, synthetic construct
  source 1..254: note = Anti-CD3-Intermediate[VL-VH] - [anti-CD3]_L1.47_ H1.33_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFDYWGQGTLVTVSS

SEQ ID NO: 730 - protein, 109 aa, synthetic construct; identical to SEQ ID NO: 634
  source 1..109: note = Anti-CD3-Intermediate[VL-VH] - [anti-CD3]_L1.47_ H1.33_scFv Variable Light (vl) Domain

SEQ ID NO: 731 - protein, 14 aa, synthetic construct
  source 1..14: note = Anti-CD3-Intermediate[VL-VH] - [anti-CD3]_L1.47_ H1.33_scFv vlCDR1
GSSTGAVTTSNYAN

SEQ ID NO: 732 - protein, 7 aa, synthetic construct
  source 1..7: note = Anti-CD3-Intermediate[VL-VH] - [anti-CD3]_L1.47_ H1.33_scFv vlCDR2
GTNKRAP

SEQ ID NO: 733 - protein, 9 aa, synthetic construct
  source 1..9: note = Anti-CD3-Intermediate[VL-VH] - [anti-CD3]_L1.47_ H1.33_scFv vlCDR3
ALWYSNHWV

SEQ ID NO: 734 - protein, 125 aa, synthetic construct; identical to SEQ ID NO: 670
  source 1..125: note = Anti-CD3-Intermediate[VL-VH] - [anti-CD3]_L1.47_ H1.33_scFv Variable Heavy (vh) Domain

SEQ ID NO: 735 - protein, 5 aa, synthetic construct
  source 1..5: note = Anti-CD3-Intermediate[VL-VH] - [anti-CD3]_L1.47_ H1.33_scFv vhCDR1
TYAMN

SEQ ID NO: 736 - protein, 19 aa, synthetic construct
  source 1..19: note = Anti-CD3-Intermediate[VL-VH] - [anti-CD3]_L1.47_ H1.33_scFv vhCDR2
RIRSKYNNYATYYADSVKG

SEQ ID NO: 737 - protein, 14 aa, synthetic construct
  source 1..14: note = Anti-CD3-Intermediate[VL-VH] - [anti-CD3]_L1.47_ H1.33_scFv vhCDR3
HGNFGDSYVSWFDY

SEQ ID NO: 738 - protein, 20 aa, synthetic construct
  source 1..20: note = Anti-CD3-Intermediate[VL-VH] - [anti-CD3]_L1.47_ H1.33_scFv Linker
GKPGSGKPGSGKPGSGKPGS

SEQ ID NO: 739 - protein, 254 aa, synthetic construct
  source 1..254: note = CD3 High-Int[VL-VH] - [anti-CD3]_L1.47_ H1.31_scFv scFv
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGKPGSGKPGSGKPGSGKPGSEVQLVESGGGLVQPGGSLRLSCAASGFTFSTYAMSWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 740 - protein, 109 aa, synthetic construct; identical to SEQ ID NO: 634
  source 1..109: note = CD3 High-Int[VL-VH] - [anti-CD3]_L1.47_ H1.31_scFv Variable Light (vl) Domain

SEQ ID NO: 741 - protein, 14 aa, synthetic construct
  source 1..14: note = CD3 High-Int[VL-VH] - [anti-CD3]_L1.47_ H1.31_scFv vlCDR1
GSSTGAVTTSNYAN

SEQ ID NO: 742 - protein, 7 aa, synthetic construct
  source 1..7: note = CD3 High-Int[VL-VH] - [anti-CD3]_L1.47_ H1.31_scFv vlCDR2
GTNKRAP

SEQ ID NO: 743 - protein, 9 aa, synthetic construct
  source 1..9: note = CD3 High-Int[VL-VH] - [anti-CD3]_L1.47_ H1.31_scFv vlCDR3
ALWYSNHWV

SEQ ID NO: 744 - protein, 125 aa, synthetic construct; identical to SEQ ID NO: 680
  source 1..125: note = CD3 High-Int[VL-VH] - [anti-CD3]_L1.47_ H1.31_scFv Variable Heavy (vh) Domain

SEQ ID NO: 745 - protein, 5 aa, synthetic construct
  source 1..5: note = CD3 High-Int[VL-VH] - [anti-CD3]_L1.47_ H1.31_scFv vhCDR1
TYAMS

SEQ ID NO: 746 - protein, 19 aa, synthetic construct
  source 1..19: note = CD3 High-Int[VL-VH] - [anti-CD3]_L1.47_ H1.31_scFv vhCDR2
RIRSKYNNYATYYADSVKG

SEQ ID NO: 747 - protein, 14 aa, synthetic construct
  source 1..14: note = CD3 High-Int[VL-VH] - [anti-CD3]_L1.47_ H1.31_scFv vhCDR3
HGNFGDSYVSWFAY

SEQ ID NO: 748 - protein, 20 aa, synthetic construct
  source 1..20: note = CD3 High-Int[VL-VH] - [anti-CD3]_L1.47_ H1.31_scFv Linker
GKPGSGKPGSGKPGSGKPGS

SEQ ID NO: 749 - protein, 4 aa, synthetic construct
YMNM

SEQ ID NO: 750 - protein, 5 aa, synthetic construct
GSGGS

SEQ ID NO: 751 - protein, 5 aa, synthetic construct
GGGGS

SEQ ID NO: 752 - protein, 4 aa, synthetic construct
GGGS

SEQ ID NO: 753 - protein, 450 aa, synthetic construct; identical to SEQ ID NO: 626
  source 1..450: note = XENP41988 Chain 1 - sacituzumab[Trop2]_H0_IgG1_pI(- )_Isosteric_A_/PVA_/S267K/L368D/K370S

SEQ ID NO: 754 - protein, 479 aa, synthetic construct
  source 1..479: note = XENP41988 Chain 2 - 1A7[CD28]_L1.71_H1.14_scFv(GKPG S)4_Fc(216)_IgG1_C220S/PVA_/S267K/S364K/E357Q
DIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKGKPGSGKPGSGKPGSGKPGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 755 - protein, 214 aa, synthetic construct; identical to SEQ ID NO: 628
  source 1..214: note = XENP41988 Chain 3 - sacituzumab[Trop2]_L0

SEQ ID NO: 756 - protein, 449 aa, synthetic construct
  source 1..449: note = XENP44424 Chain 1 - 1F11-1A3.315[Trop2]_H1_IgG1_pI( -)_Isosteric_A_PVA_/S267K/L368D/K370S/M428L/N434S
QVQLVQSGAEVKKPGASVKVSCKASGYTFTRYYMHWVRQAPGQGLEWMGIINPGGGSTGYAQKFQGRVTMTRDTSTSTVYMELSSLRSEDTAVYYCARDDVVAADVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 757 - protein, 479 aa, synthetic construct
  source 1..479: note = XENP44424 Chain 2 - 1A7[CD28]_H1.14_L1.71_scFv(GKPG S)4_Fc(216)_IgG1_C220S/PVA_/S267K/S364K/E357Q/M428L/N434S
EVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 758 - protein, 214 aa, synthetic construct
  source 1..214: note = XENP44424 Chain 3 - 1F11-1A3.315[Trop2]_L1
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 759 - protein, 449 aa, synthetic construct; identical to SEQ ID NO: 756
  source 1..449: note = XENP44505 Chain 1 - 1F11-1A3.315[Trop2]_H1_IgG1_pI( -)_Isosteric_A_PVA_/S267K/L368D/K370S/M428L/N434S

SEQ ID NO: 760 - protein, 479 aa, synthetic construct
  source 1..479: note = XENP44505 Chain 2 - 1A7[CD28]_L1.71_H1.14_scFv(GKPG S)4_Fc(216)_IgG1_C220S/PVA_/S267K/S364K/E357Q/M428L/N434S
DIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIKGKPGSGKPGSGKPGSGKPGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 761 - protein, 214 aa, synthetic construct; identical to SEQ ID NO: 758
  source 1..214: note = XENP44505 Chain 3 - 1F11-1A3.315[Trop2]_L1

SEQ ID NO: 762 - protein, 121 aa, synthetic construct
  source 1..121: note = 1A7[CD28]_H1.103
EVQLLESGGGLVQPGGSLRLSCAASGFTFSSYYMSWVRQAPGKGLEWVSTISERMDSTYYADSVKGRFTISRDNSKNTLYLQMNSLRAEDTAVYYCAKSGPGLRQVGFDYWGQGTLVTVSS

SEQ ID NO: 763 - protein, 121 aa, synthetic construct
  source 1..121: note = 1A7[CD28]_H1.104
EVQLLESGGGLVQPGGSLRLSCAASGFTFSSYYMSWVRQAPGKGLEWVSTISERGDPTYYADSVKGRFTISRDNSKNTLYLQMNSLRAEDTAVYYCAKSGPGLRQVGFDYWGQGTLVTVSS

SEQ ID NO: 764 - protein, 121 aa, synthetic construct
  source 1..121: note = 1A7[CD28]_H1.105
EVQLLESGGGLVQPGGSLRLSCAASGFTFSSYYMSWVRQAPGKGLEWVSTISERLDSTYYADSVKGRFTISRDNSKNTLYLQMNSLRAEDTAVYYCAKSGPGLRQVGFDYWGQGTLVTVSS

SEQ ID NO: 765 - protein, 121 aa, synthetic construct
  source 1..121: note = 1A7[CD28]_H1.106
EVQLLESGGGLVQPGGSLRLSCAASGFTFSSYYMSWVRQAPGKGLEWVSTISERGDSTYYADSVKGRFTISRDNSKNTLYLQMNSLRAEDTAVYYCAKSGPGLRQVGFDYWGQGTLVTVSS

SEQ ID NO: 766 - protein, 121 aa, synthetic construct
  source 1..121: note = 1A7[CD28]_H1.107
EVQLLESGGGLVQPGGSLRLSCAASGFTFSSYYMSWVRQAPGKGLEWVSTISESSDVTYYADSVKGRFTISRDNSKNTLYLQMNSLRAEDTAVYYCAKSGPGLRQVGFDYWGQGTLVTVSS

SEQ ID NO: 767 - protein, 121 aa, synthetic construct
  source 1..121: note = 1A7[CD28]_H1.108
EVQLLESGGGLVQPGGSLRLSCAASGFTFSSYYMSWVRQAPGKGLEWVSTISERNDSTYYADSVKGRFTISRDNSKNTLYLQMNSLRAEDTAVYYCAKSGPGLRQVGFDYWGQGTLVTVSS

SEQ ID NO: 768 - protein, 121 aa, synthetic construct
  source 1..121: note = 1A7[CD28]_H1.109
EVQLLESGGGLVQPGGSLRLSCAASGFTFSSYYMSWVRQAPGKGLEWVSTISERRDSTYYADSVKGRFTISRDNSKNTLYLQMNSLRAEDTAVYYCAKSGPGLRQVGFDYWGQGTLVTVSS

SEQ ID NO: 769 - protein, 121 aa, synthetic construct
  source 1..121: note = 1A7[CD28]_H1.110
EVQLLESGGGLVQPGGSLRLSCAASGFTFSSYYMSWVRQAPGKGLEWVWTISERGDSTYYADSVKGRFTISRDNSKNTLYLQMNSLRAEDTAVYYCAKSGPGLRQVGFDYWGQGTLVTVSS

SEQ ID NO: 770 - protein, 121 aa, synthetic construct
  source 1..121: note = 1A7[CD28]_H1.111
EVQLLESGGGLVQPGGSLRLSCAASGFTFSSYYMSWVRQAPGKGLEWVSTISERSDSTYYADSVKGRFTISRDNSKNTLYLQMNSLRAEDTAVYYCAKSGPGLRQVGFDYWGQGTLVTVSS

SEQ ID NO: 771 - protein, 121 aa, synthetic construct
  source 1..121: note = 1A7[CD28]_H1.112
EVQLLESGGGLVQPGGSLRLSCAASGFTFSSYYMSWVRQAPGKGLEWVSTISERQDSTYYADSVKGRFTISRDNSKNTLYLQMNSLRAEDTAVYYCAKSGPGLRQVGFDYWGQGTLVTVSS

SEQ ID NO: 772 - protein, 121 aa, synthetic construct
  source 1..121: note = 1A7[CD28]_H1.113
EVQLLESGGGLVQPGGSLRLSCAASGFTFSSYYMSWVRQAPGKGLEWVSTISERTDSTYYADSVKGRFTISRDNSKNTLYLQMNSLRAEDTAVYYCAKSGPGLRQVGFDYWGQGTLVTVSS

SEQ ID NO: 773 - protein, 121 aa, synthetic construct
  source 1..121: note = 1A7[CD28]_H1.114
EVQLLESGGGLVQPGGSLRLSCAASGFTFSSYYMSWVRQAPGKGLEWVSTIAERGDSTYYADSVKGRFTISRDNSKNTLYLQMNSLRAEDTAVYYCAKSGPGLRQVGFDYWGQGTLVTVSS

SEQ ID NO: 774 - protein, 121 aa, synthetic construct
  source 1..121: note = 1A7[CD28]_H1.115
EVQLLESGGGLVQPGGSLRLSCAASGFTFSSYYMSWVRQAPGKGLEWVMTISESGDYTYYADSVKGRFTISRDNSKNTLYLQMNSLRAEDTAVYYCAKSGPGLRQVGFDYWGQGTLVTVSS

SEQ ID NO: 775 - protein, 121 aa, synthetic construct
  source 1..121: note = 1A7[CD28]_H1.116
EVQLLESGGGLVQPGGSLRLSCAASGFTFSSYYMSWVRQAPGKGLEWVSTISERKDSTYYADSVKGRFTISRDNSKNTLYLQMNSLRAEDTAVYYCAKSGPGLRQVGFDYWGQGTLVTVSS

SEQ ID NO: 776 - protein, 121 aa, synthetic construct
  source 1..121: note = 1A7[CD28]_H1.117
EVQLLESGGGLVQPGGSLRLSCAASGFTFSSYYMSWVRQAPGKGLEWVSTISERHDSTYYADSVKGRFTISRDNSKNTLYLQMNSLRAEDTAVYYCAKSGPGLRQVGFDYWGQGTLVTVSS

SEQ ID NO: 777 - protein, 121 aa, synthetic construct
  source 1..121: note = 1A7[CD28]_H1.118
EVQLLESGGGLVQPGGSLRLSCAASGFTFSSYYMSWVRQAPGKGLEWVSTIAESGDPTYYADSVKGRFTISRDNSKNTLYLQMNSLRAEDTAVYYCAKSGPGLRQVGFDYWGQGTLVTVSS

SEQ ID NO: 778 - protein, 121 aa, synthetic construct
  source 1..121: note = 1A7[CD28]_H1.119
EVQLLESGGGLVQPGGSLRLSCAASGFTFSSYYMSWVRQAPGKGLEWVSTIVERGDVTYYADSVKGRFTISRDNSKNTLYLQMNSLRAEDTAVYYCAKSGPGLRQVGFDYWGQGTLVTVSS

SEQ ID NO: 779 - protein, 121 aa, synthetic construct
  source 1..121: note = 1A7[CD28]_H1.120
EVQLLESGGGLVQPGGSLRLSCAASGFTFSSYYMSWVRQAPGKGLEWVSTISERYDSTYYADSVKGRFTISRDNSKNTLYLQMNSLRAEDTAVYYCAKSGPGLRQVGFDYWGQGTLVTVSS

SEQ ID NO: 780 - protein, 121 aa, synthetic construct; identical to SEQ ID NO: 158
  source 1..121: note = 1A7[CD28]_H1.121

SEQ ID NO: 781 - protein, 121 aa, synthetic construct
  source 1..121: note = 1A7[CD28]_H1.122
EVQLLESGGGLVQPGGSLRLSCAASGFTFSSYYMSWVRQAPGKGLEWVTTISESGDPTYYADSVKGRFTISRDNSKNTLYLQMNSLRAEDTAVYYCAKSGPGLRQVGFDYWGQGTLVTVSS

SEQ ID NO: 782 - protein, 121 aa, synthetic construct
  source 1..121: note = 1A7[CD28]_H1.123
EVQLLESGGGLVQPGGSLRLSCAASGFTFSSYYMSWVRQAPGKGLEWVSTISENLDSTYYADSVKGRFTISRDNSKNTLYLQMNSLRAEDTAVYYCAKSGPGLRQVGFDYWGQGTLVTVSS

SEQ ID NO: 783 - protein, 121 aa, synthetic construct
  source 1..121: note = 1A7[CD28]_H1.124
EVQLLESGGGLVQPGGSLRLSCAASGFTFSSYYMSWVRQAPGKGLEWVSTISESADVTYYADSVKGRFTISRDNSKNTLYLQMNSLRAEDTAVYYCAKSGPGLRQVGFDYWGQGTLVTVSS

SEQ ID NO: 784 - protein, 121 aa, synthetic construct
  source 1..121: note = 1A7[CD28]_H1.125
EVQLLESGGGLVQPGGSLRLSCAASGFTFSSYYMSWVRQAPGKGLEWVSTISEHGLSTYYADSVKGRFTISRDNSKNTLYLQMNSLRAEDTAVYYCAKSGPGLRQVGFDYWGQGTLVTVSS

SEQ ID NO: 785 - protein, 121 aa, synthetic construct
  source 1..121: note = 1A7[CD28]_H1.126
EVQLLESGGGLVQPGGSLRLSCAASGFTFSSYYMSWVRQAPGKGLEWVSTISERDDSTYYADSVKGRFTISRDNSKNTLYLQMNSLRAEDTAVYYCAKSGPGLRQVGFDYWGQGTLVTVSS

SEQ ID NO: 786 - protein, 121 aa, synthetic construct
  source 1..121: note = 1A7[CD28]_H1.127
EVQLLESGGGLVQPGGSLRLSCAASGFTFSSYYMSWVRQAPGKGLEWVSTISENRDSTYYADSVKGRFTISRDNSKNTLYLQMNSLRAEDTAVYYCAKSGPGLRQVGFDYWGQGTLVTVSS

SEQ ID NO: 787 - protein, 121 aa, synthetic construct
  source 1..121: note = 1A7[CD28]_H1.128
EVQLLESGGGLVQPGGSLRLSCAASGFTFSSYYMSWVRQAPGKGLEWVSTISERFDSTYYADSVKGRFTISRDNSKNTLYLQMNSLRAEDTAVYYCAKSGPGLRQVGFDYWGQGTLVTVSS

SEQ ID NO: 788 - protein, 121 aa, synthetic construct
  source 1..121: note = 1A7[CD28]_H1.129
EVQLLESGGGLVQPGGSLRLSCAASGFTFSSYYMSWVRQAPGKGLEWVSTISERADSTYYADSVKGRFTISRDNSKNTLYLQMNSLRAEDTAVYYCAKSGPGLRQVGFDYWGQGTLVTVSS

SEQ ID NO: 789 - protein, 121 aa, synthetic construct
  source 1..121: note = 1A7[CD28]_H1.130
EVQLLESGGGLVQPGGSLRLSCAASGFTFSSYYMSWVRQAPGKGLEWVSTISERGDVTYYADSVKGRFTISRDNSKNTLYLQMNSLRAEDTAVYYCAKSGPGLRQVGFDYWGQGTLVTVSS

SEQ ID NO: 790 - protein, 121 aa, synthetic construct
  source 1..121: note = 1A7[CD28]_H1.131
EVQLLESGGGLVQPGGSLRLSCAASGFTFSSYYMSWVRQAPGKGLEWVSTISEREDSTYYADSVKGRFTISRDNSKNTLYLQMNSLRAEDTAVYYCAKSGPGLRQVGFDYWGQGTLVTVSS

SEQ ID NO: 791 - protein, 121 aa, synthetic construct
  source 1..121: note = 1A7[CD28]_H1.132
EVQLLESGGGLVQPGGSLRLSCAASGFTFSSYYMSWVRQAPGKGLEWVSTISESSMSTYYADSVKGRFTISRDNSKNTLYLQMNSLRAEDTAVYYCAKSGPGLRQVGFDYWGQGTLVTVSS

SEQ ID NO: 792 - protein, 121 aa, synthetic construct
  source 1..121: note = 1A7[CD28]_H1.133
EVQLLESGGGLVQPGGSLRLSCAASGFTFSSYYMSWVRQAPGKGLEWVSTISESVDPTYYADSVKGRFTISRDNSKNTLYLQMNSLRAEDTAVYYCAKSGPGLRQVGFDYWGQGTLVTVSS

SEQ ID NO: 793 - protein, 121 aa, synthetic construct
  source 1..121: note = 1A7[CD28]_H1.134
EVQLLESGGGLVQPGGSLRLSCAASGFTFSSYYMSWVRQAPGKGLEWVMTISERGDSTYYADSVKGRFTISRDNSKNTLYLQMNSLRAEDTAVYYCAKSGPGLRQVGFDYWGQGTLVTVSS

SEQ ID NO: 794 - protein, 121 aa, synthetic construct
  source 1..121: note = 1A7[CD28]_H1.135
EVQLLESGGGLVQPGGSLRLSCAASGFTFSSYYMSWVRQAPGKGLEWVSTISESWDHTYYADSVKGRFTISRDNSKNTLYLQMNSLRAEDTAVYYCAKSGPGLRQVGFDYWGQGTLVTVSS

SEQ ID NO: 795 - protein, 121 aa, synthetic construct
  source 1..121: note = 1A7[CD28]_H1.136
EVQLLESGGGLVQPGGSLRLSCAASGFTFSSYYMSWVRQAPGKGLEWVSTISERIDSTYYADSVKGRFTISRDNSKNTLYLQMNSLRAEDTAVYYCAKSGPGLRQVGFDYWGQGTLVTVSS

SEQ ID NO: 796 - protein, 121 aa, synthetic construct
  source 1..121: note = 1A7[CD28]_H1.137
EVQLLESGGGLVQPGGSLRLSCAASGFTFSSYYMSWVRQAPGKGLEWVVTISERGDSTYYADSVKGRFTISRDNSKNTLYLQMNSLRAEDTAVYYCAKSGPGLRQVGFDYWGQGTLVTVSS

SEQ ID NO: 797 - protein, 121 aa, synthetic construct
  source 1..121: note = 1A7[CD28]_H1.138
EVQLLESGGGLVQPGGSLRLSCAASGFTFSSYYMSWVRQAPGKGLEWVTTISERGDSTYYADSVKGRFTISRDNSKNTLYLQMNSLRAEDTAVYYCAKSGPGLRQVGFDYWGQGTLVTVSS

SEQ ID NO: 798 - protein, 30 aa, synthetic construct
  source 1..30: note = 1A7 VH HFR1
EVQLLESGGGLVQPGGSLRLSCAASGFTFS

SEQ ID NO: 799 - protein, 5 aa, synthetic construct
  source 1..5: note = 1A7 VH HCDR1
SYYMS

SEQ ID NO: 800 - protein, 14 aa, synthetic construct
  source 1..14: note = 1A7 VH HFR2
  VARIANT 14: note = S, W, M, T, or V
WVRQAPGKGLEWVX

SEQ ID NO: 801 - protein, 17 aa, synthetic construct
  source 1..17: note = 1A7 VH HCDR2
  VARIANT 3: note = A, V, or S
  VARIANT 5: note = S, N, H, or R
  VARIANT 6: note = G, L, S, N, R, Q, T, K, H, Y, A, D, F, E, V, W, I, or M
  VARIANT 7: note = L, M, or D
  VARIANT 8: note = S, P, V, Y, or H
TIXEXXXXTYYADSVKG

SEQ ID NO: 802 - protein, 120 aa, synthetic construct
  source 1..120: note = 1F11-1A3.315[TROP2]_H1L1 Variable heavy (vh) domain
QVQLVQSGAEVKKPGASVKVSCKASGYTFTRYYMHWVRQAPGQGLEWMGIINPGGGSTGYAQKFQGRVTMTRDTSTSTVYMELSSLRSEDTAVYYCARDDVVAADVFDYWGQGTLVTVSS

SEQ ID NO: 803 - protein, 10 aa, synthetic construct
  source 1..10: note = 1F11-1A3.315[TROP2]_H1L1 vhCDR1
GYTFTRYYMH

SEQ ID NO: 804 - protein, 17 aa, synthetic construct
  source 1..17: note = 1F11-1A3.315[TROP2]_H1L1 vhCDR2
IINPGGGSTGYAQKFQG

SEQ ID NO: 805 - protein, 11 aa, synthetic construct
  source 1..11: note = 1F11-1A3.315[TROP2]_H1L1 vhCDR3
DDVVAADVFDY

SEQ ID NO: 806 - protein, 107 aa, synthetic construct
  source 1..107: note = 1F11-1A3.315[TROP2]_H1L1 Variable light (vl) domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 807 - protein, 11 aa, synthetic construct
  source 1..11: note = 1F11-1A3.315[TROP2]_H1L1 vlCDR1
RASQSISSYLN

SEQ ID NO: 808 - protein, 7 aa, synthetic construct
  source 1..7: note = 1F11-1A3.315[TROP2]_H1L1 vlCDR2
AASSLQS

SEQ ID NO: 809 - protein, 9 aa, synthetic construct
  source 1..9: note = 1F11-1A3.315[TROP2]_H1L1 vlCDR3
QQSYSTPYT

SEQ ID NO: 810 - protein, 120 aa, synthetic construct
  source 1..120: note = 1F11-1A3.315[Trop2]_H1.1
QVQLVQSGAEVKKPGASVKVSCKASGYTLTRYYMHWVRQAPGQGLEWMGIINPGGGSTGYAQKFQGRVTMTRDTSTSTVYMELSSLRSEDTAVYYCARDDVVAADVFDYWGQGTLVTVSS

SEQ ID NO: 811 - protein, 120 aa, synthetic construct
  source 1..120: note = 1F11-1A3.315[Trop2]_H1.2
QVQLVQSGAEVKKPGASVKVSCKASGYTFTRYYMHWVRQAPGQGLEWMGIIEPGGGSTGYAQKFQGRVTMTRDTSTSTVYMELSSLRSEDTAVYYCARDDVVAADVFDYWGQGTLVTVSS

SEQ ID NO: 812 - protein, 120 aa, synthetic construct
  source 1..120: note = 1F11-1A3.315[Trop2]_H1.3
QVQLVQSGAEVKKPGASVKVSCKASGYTFTRYYMHWVRQAPGQGLEWMGIINPGGGSTTYAQKFQGRVTMTRDTSTSTVYMELSSLRSEDTAVYYCARDDVVAADVFDYWGQGTLVTVSS

SEQ ID NO: 813 - protein, 120 aa, synthetic construct
  source 1..120: note = 1F11-1A3.315[Trop2]_H1.4
QVQLVQSGAEVKKPGASVKVSCKASGYTFTRYYMHWVRQAPGQGLEWMGIINPGGGSTGYAQKFQGRVTMTRDTSTSTVYMELSSLRSEDTAVYYCARDDVTAADVFDYWGQGTLVTVSS

SEQ ID NO: 814 - protein, 120 aa, synthetic construct
  source 1..120: note = 1F11-1A3.315[Trop2]_H1.5
QVQLVQSGAEVKKPGASVKVSCKASGYTFTRYYMHWVRQAPGQGLEWMGIINPGGGSTGYAQKFQGRVTMTRDTSTSTVYMELSSLRSEDTAVYYCARDDVVKADVFDYWGQGTLVTVSS

SEQ ID NO: 815 - protein, 120 aa, synthetic construct
  source 1..120: note = 1F11-1A3.315[Trop2]_H1.6
QVQLVQSGAEVKKPGASVKVSCKASGYTLTRYYMHWVRQAPGQGLEWMGIIEPGGGSTGYAQKFQGRVTMTRDTSTSTVYMELSSLRSEDTAVYYCARDDVVAADVFDYWGQGTLVTVSS

SEQ ID NO: 816 - protein, 120 aa, synthetic construct
  source 1..120: note = 1F11-1A3.315[Trop2]_H1.7
QVQLVQSGAEVKKPGASVKVSCKASGYTLTRYYMHWVRQAPGQGLEWMGIINPGGGSTTYAQKFQGRVTMTRDTSTSTVYMELSSLRSEDTAVYYCARDDVVAADVFDYWGQGTLVTVSS

SEQ ID NO: 817 - protein, 120 aa, synthetic construct
  source 1..120: note = 1F11-1A3.315[Trop2]_H1.8
QVQLVQSGAEVKKPGASVKVSCKASGYTLTRYYMHWVRQAPGQGLEWMGIINPGGGSTGYAQKFQGRVTMTRDTSTSTVYMELSSLRSEDTAVYYCARDDVTAADVFDYWGQGTLVTVSS

SEQ ID NO: 818 - protein, 120 aa, synthetic construct
  source 1..120: note = 1F11-1A3.315[Trop2]_H1.9
QVQLVQSGAEVKKPGASVKVSCKASGYTLTRYYMHWVRQAPGQGLEWMGIINPGGGSTGYAQKFQGRVTMTRDTSTSTVYMELSSLRSEDTAVYYCARDDVVKADVFDYWGQGTLVTVSS

SEQ ID NO: 819 - protein, 120 aa, synthetic construct
  source 1..120: note = 1F11-1A3.315[Trop2]_H1.10
QVQLVQSGAEVKKPGASVKVSCKASGYTFTRYYMHWVRQAPGQGLEWMGIIEPGGGSTTYAQKFQGRVTMTRDTSTSTVYMELSSLRSEDTAVYYCARDDVVAADVFDYWGQGTLVTVSS

SEQ ID NO: 820 - protein, 120 aa, synthetic construct
  source 1..120: note = 1F11-1A3.315[Trop2]_H1.11
QVQLVQSGAEVKKPGASVKVSCKASGYTFTRYYMHWVRQAPGQGLEWMGIIEPGGGSTGYAQKFQGRVTMTRDTSTSTVYMELSSLRSEDTAVYYCARDDVTAADVFDYWGQGTLVTVSS

SEQ ID NO: 821 - protein, 120 aa, synthetic construct
  source 1..120: note = 1F11-1A3.315[Trop2]_H1.12
QVQLVQSGAEVKKPGASVKVSCKASGYTFTRYYMHWVRQAPGQGLEWMGIIEPGGGSTGYAQKFQGRVTMTRDTSTSTVYMELSSLRSEDTAVYYCARDDVVKADVFDYWGQGTLVTVSS

SEQ ID NO: 822 - protein, 120 aa, synthetic construct
  source 1..120: note = 1F11-1A3.315[Trop2]_H1.13
QVQLVQSGAEVKKPGASVKVSCKASGYTFTRYYMHWVRQAPGQGLEWMGIINPGGGSTTYAQKFQGRVTMTRDTSTSTVYMELSSLRSEDTAVYYCARDDVTAADVFDYWGQGTLVTVSS

SEQ ID NO: 823 - protein, 120 aa, synthetic construct
  source 1..120: note = 1F11-1A3.315[Trop2]_H1.14
QVQLVQSGAEVKKPGASVKVSCKASGYTFTRYYMHWVRQAPGQGLEWMGIINPGGGSTTYAQKFQGRVTMTRDTSTSTVYMELSSLRSEDTAVYYCARDDVVKADVFDYWGQGTLVTVSS

SEQ ID NO: 824 - protein, 120 aa, synthetic construct
  source 1..120: note = 1F11-1A3.315[Trop2]_H1.15
QVQLVQSGAEVKKPGASVKVSCKASGYTFTRYYMHWVRQAPGQGLEWMGIINPGGGSTGYAQKFQGRVTMTRDTSTSTVYMELSSLRSEDTAVYYCARDDVTKADVFDYWGQGTLVTVSS

SEQ ID NO: 825 - protein, 120 aa, synthetic construct
  source 1..120: note = 1F11-1A3.315[Trop2]_H1.16
QVQLVQSGAEVKKPGASVKVSCKASGYTLTRYYMHWVRQAPGQGLEWMGIIEPGGGSTGYAQKFQGRVTMTRDTSTSTVYMELSSLRSEDTAVYYCARDDVTAADVFDYWGQGTLVTVSS

SEQ ID NO: 826 - protein, 120 aa, synthetic construct
  source 1..120: note = 1F11-1A3.315[Trop2]_H1.17
QVQLVQSGAEVKKPGASVKVSCKASGYTLTRYYMHWVRQAPGQGLEWMGIIEPGGGSTGYAQKFQGRVTMTRDTSTSTVYMELSSLRSEDTAVYYCARDDVVKADVFDYWGQGTLVTVSS

SEQ ID NO: 827 - protein, 120 aa, synthetic construct
  source 1..120: note = 1F11-1A3.315[Trop2]_H1.18
QVQLVQSGAEVKKPGASVKVSCKASGYTLTRYYMHWVRQAPGQGLEWMGIIEPGGGSTTYAQKFQGRVTMTRDTSTSTVYMELSSLRSEDTAVYYCARDDVTAADVFDYWGQGTLVTVSS

SEQ ID NO: 828 - protein, 120 aa, synthetic construct
  source 1..120: note = 1F11-1A3.315[Trop2]_H1.19
QVQLVQSGAEVKKPGASVKVSCKASGYTLTRYYMHWVRQAPGQGLEWMGIIEPGGGSTTYAQKFQGRVTMTRDTSTSTVYMELSSLRSEDTAVYYCARDDVVKADVFDYWGQGTLVTVSS

SEQ ID NO: 829 - protein, 120 aa, synthetic construct
  source 1..120: note = 1F11-1A3.315[Trop2]_H1.20
QVQLVQSGAEVKKPGASVKVSCKASGYTLTRYYMHWVRQAPGQGLEWMGIIEPGGGSTSYAQKFQGRVTMTRDTSTSTVYMELSSLRSEDTAVYYCARDDVTAADVFDYWGQGTLVTVSS

SEQ ID NO: 830 - protein, 120 aa, synthetic construct
  source 1..120: note = 1F11-1A3.315[Trop2]_H1.21
QVQLVQSGAEVKKPGASVKVSCKASGYTLTRYYMHWVRQAPGQGLEWMGIIEPGGGSTSYAQKFQGRVTMTRDTSTSTVYMELSSLRSEDTAVYYCARDDVVKADVFDYWGQGTLVTVSS

SEQ ID NO: 831 - protein, 120 aa, synthetic construct
  source 1..120: note = 1F11-1A3.315[Trop2]_H1.22
QVQLVQSGAEVKKPGASVKVSCKASGYTLTRYYMHWVRQAPGQGLEWMGIIEPGGGSTSYAQKFQGRVTMTRDTSTSTVYMELSSLRSEDTAVYYCARDDVTKADVFDYWGQGTLVTVSS

SEQ ID NO: 832 - protein, 120 aa, synthetic construct
  source 1..120: note = 1F11-1A3.315[Trop2]_H1.23
QVQLVQSGAEVKKPGASVKVSCKASGYTFTRYYVHWVRQAPGQGLEWMGIINPGGGSTGYAQKFQGRVTMTRDTSTSTVYMELSSLRSEDTAVYYCARDDVVAADVFDYWGQGTLVTVSS

SEQ ID NO: 833 - protein, 120 aa, synthetic construct
  source 1..120: note = 1F11-1A3.315[Trop2]_H1.24
QVQLVQSGAEVKKPGASVKVSCKASGYTFTRYYMHWVRQAPGQGLEWMGIINPGGGSTDYAQKFQGRVTMTRDTSTSTVYMELSSLRSEDTAVYYCARDDVVAADVFDYWGQGTLVTVSS

SEQ ID NO: 834 - protein, 120 aa, synthetic construct
  source 1..120: note = 1F11-1A3.315[Trop2]_H1.25
QVQLVQSGAEVKKPGASVKVSCKASGYTFTRYYMHWVRQAPGQGLEWMGIINPGGGSTLYAQKFQGRVTMTRDTSTSTVYMELSSLRSEDTAVYYCARDDVVAADVFDYWGQGTLVTVSS

SEQ ID NO: 835 - protein, 120 aa, synthetic construct
  source 1..120: note = 1F11-1A3.315[Trop2]_H1.26
QVQLVQSGAEVKKPGASVKVSCKASGYTFTRYYMHWVRQAPGQGLEWMGIINPGGGSTGYAQKFQGRVTMTRDTSTSTVYMELSSLRSEDTAVYYCARDDVMAADVFDYWGQGTLVTVSS

SEQ ID NO: 836 - protein, 120 aa, synthetic construct
  source 1..120: note = 1F11-1A3.315[Trop2]_H1.27
QVQLVQSGAEVKKPGASVKVSCKASGYTLTRYYMHWVRQAPGQGLEWMGIIEPGGGSTDYAQKFQGRVTMTRDTSTSTVYMELSSLRSEDTAVYYCARDDVTAADVFDYWGQGTLVTVSS

SEQ ID NO: 837 - protein, 120 aa, synthetic construct
  source 1..120: note = 1F11-1A3.315[Trop2]_H1.28
QVQLVQSGAEVKKPGASVKVSCKASGYTLTRYYMHWVRQAPGQGLEWMGIIEPGGGSTLYAQKFQGRVTMTRDTSTSTVYMELSSLRSEDTAVYYCARDDVTAADVFDYWGQGTLVTVSS

SEQ ID NO: 838 - protein, 120 aa, synthetic construct
  source 1..120: note = 1F11-1A3.315[Trop2]_H1.29
QVQLVQSGAEVKKPGASVKVSCKASGYTLTRYYVHWVRQAPGQGLEWMGIIEPGGGSTDYAQKFQGRVTMTRDTSTSTVYMELSSLRSEDTAVYYCARDDVTAADVFDYWGQGTLVTVSS

SEQ ID NO: 839 - protein, 120 aa, synthetic construct
  source 1..120: note = 1F11-1A3.315[Trop2]_H1.30
QVQLVQSGAEVKKPGASVKVSCKASGYTLTRYYVHWVRQAPGQGLEWMGIIEPGGGSTLYAQKFQGRVTMTRDTSTSTVYMELSSLRSEDTAVYYCARDDVTAADVFDYWGQGTLVTVSS

SEQ ID NO: 840 - protein, 120 aa, synthetic construct
  source 1..120: note = 1F11-1A3.315[Trop2]_H1.31
QVQLVQSGAEVKKPGASVKVSCKASGYTLTRYYVHWVRQAPGQGLEWMGIIEPGGGSTDYAQKFQGRVTMTRDTSTSTVYMELSSLRSEDTAVYYCARDDVVKADVFDYWGQGTLVTVSS

SEQ ID NO: 841 - protein, 25 aa, synthetic construct [sequence omitted]

SEQ ID NO: 842 - protein, 10 aa, synthetic construct
  source 1..10: note = 1F11-1A3.315 VH HCDR1
  VARIANT 4: note = L or F
  VARIANT 9: note = M or V
GYTXTRYYXH

SEQ ID NO: 843 - protein, 14 aa, synthetic construct
  source 1..14: note = 1F11-1A3.315 VH HFR2
WVRQAPGQGLEWMG

SEQ ID NO: 844 - protein, 17 aa, synthetic construct
  source 1..17: note = 1F11-1A3.315 VH HCDR2
  VARIANT 3: note = N or E
  VARIANT 10: note = G, T, S, D, or L
IIXPGGGSTXYAQKFQG

SEQ ID NO: 845 - protein, 32 aa, synthetic construct
  source 1..32: note = 1F11-1A3.315 VH HFR3
RVTMTRDTSTSTVYMELSSLRSEDTAVYYCAR

SEQ ID NO: 846 - protein, 11 aa, synthetic construct
  source 1..11: note = 1F11-1A3.315 VH HCDR3
  VARIANT 4: note = V, T, or M
  VARIANT 5: note = A or K
DDVXXADVFDY

SEQ ID NO: 847 - protein, 11 aa, synthetic construct
  source 1..11: note = 1F11-1A3.315 VH HFR4
WGQGTLVTVSS

SEQ ID NO: 848 - protein, 450 aa, synthetic construct
  source 1..450: note = XENP44595 Chain 1 - 1A7[CD28]_H1.129_IgG1_pI(-)_Iso steric_A_PVA_/S267K/L368D/K370S/M428L/N434S
EVQLLESGGGLVQPGGSLRLSCAASGFTFSSYYMSWVRQAPGKGLEWVSTISERADSTYYADSVKGRFTISRDNSKNTLYLQMNSLRAEDTAVYYCAKSGPGLRQVGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 849 - protein, 449 aa, synthetic construct
  source 1..449: note = XENP44595 Chain 2 - 1F11-1A3.315[Trop2]_H1_IgG1_PVA _/S267K/S364K/E357Q/M428L/N434S
QVQLVQSGAEVKKPGASVKVSCKASGYTFTRYYMHWVRQAPGQGLEWMGIINPGGGSTGYAQKFQGRVTMTRDTSTSTVYMELSSLRSEDTAVYYCARDDVVAADVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 850 - protein, 214 aa, synthetic construct; identical to SEQ ID NO: 758
  source 1..214: note = XENP44595 Chain 3 - IGKV1-39_L1

SEQ ID NO: 851 - protein, 450 aa, synthetic construct
  source 1..450: note = XENP44596 Chain 1 - 1A7[CD28]_H1.106_IgG1_pI(-)_Iso steric_A_PVA_/S267K/L368D/K370S/M428L/N434S
EVQLLESGGGLVQPGGSLRLSCAASGFTFSSYYMSWVRQAPGKGLEWVSTISERGDSTYYADSVKGRFTISRDNSKNTLYLQMNSLRAEDTAVYYCAKSGPGLRQVGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 852 - protein, 449 aa, synthetic construct; identical to SEQ ID NO: 849
  source 1..449: note = XENP44596 Chain 2 - 1F11-1A3.315[Trop2]_H1_IgG1_PVA _/S267K/S364K/E357Q/M428L/N434S

SEQ ID NO: 853 - protein, 214 aa, synthetic construct; identical to SEQ ID NO: 758
  source 1..214: note = XENP44596 Chain 3 - IGKV1-39_L1

SEQ ID NO: 854 - protein, 450 aa, synthetic construct; identical to SEQ ID NO: 848
  source 1..450: note = XENP44597 Chain 1 - 1A7[CD28]_H1.129_IgG1_pI(-)_Iso steric_A_PVA_/S267K/L368D/K370S/M428L/N434S

SEQ ID NO: 855 - protein, 449 aa, synthetic construct
  source 1..449: note = XENP44597 Chain 2 - 1F11-1A3.315[Trop2]_H1.10_IgG1_ PVA_/S267K/S364K/E357Q/M428L/N434S
QVQLVQSGAEVKKPGASVKVSCKASGYTFTRYYMHWVRQAPGQGLEWMGIIEPGGGSTTYAQKFQGRVTMTRDTSTSTVYMELSSLRSEDTAVYYCARDDVVAADVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 856 - protein, 214 aa, synthetic construct; identical to SEQ ID NO: 758
  source 1..214: note = XENP44597 Chain 3 - IGKV1-39_L1

SEQ ID NO: 857 - protein, 450 aa, synthetic construct; identical to SEQ ID NO: 851
  source 1..450: note = XENP44598 Chain 1 - 1A7[CD28]_H1.106_IgG1_pI(-)_Iso steric_A_PVA_/S267K/L368D/K370S/M428L/N434S

SEQ ID NO: 858 - protein, 449 aa, synthetic construct; identical to SEQ ID NO: 855
  source 1..449: note = XENP44598 Chain 2 - 1F11-1A3.315[Trop2]_H1.10_IgG1_ PVA_/S267K/S364K/E357Q/M428L/N434S

SEQ ID NO: 859 - protein, 214 aa, synthetic construct; identical to SEQ ID NO: 758
  source 1..214: note = XENP44598 Chain 3 - IGKV1-39_L1

SEQ ID NO: 860 - protein, 450 aa, synthetic construct; identical to SEQ ID NO: 848
  source 1..450: note = XENP44599 Chain 1 - 1A7[CD28]_H1.129_IgG1_pI(-)_Iso steric_A_PVA_/S267K/L368D/K370S/M428L/N434S

SEQ ID NO: 861 - protein, 449 aa, synthetic construct
  source 1..449: note = XENP44599 Chain 2 - 1F11-1A3.315[Trop2]_H1.19_IgG1_ PVA_/S267K/S364K/E357Q/M428L/N434S
QVQLVQSGAEVKKPGASVKVSCKASGYTLTRYYMHWVRQAPGQGLEWMGIIEPGGGSTTYAQKFQGRVTMTRDTSTSTVYMELSSLRSEDTAVYYCARDDVVKADVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 862 - protein, 214 aa, synthetic construct; identical to SEQ ID NO: 758
  source 1..214: note = XENP44599 Chain 3 - IGKV1-39_L1

SEQ ID NO: 863 - protein, 450 aa, synthetic construct; identical to SEQ ID NO: 851
  source 1..450: note = XENP44600 Chain 1 - 1A7[CD28]_H1.106_IgG1_pI(-)_Iso steric_A_PVA_/S267K/L368D/K370S/M428L/N434S

SEQ ID NO: 864 - protein, 449 aa, synthetic construct; identical to SEQ ID NO: 861
  source 1..449: note = XENP44600 Chain 2 - 1F11-1A3.315[Trop2]_H1.19_IgG1_ PVA_/S267K/S364K/E357Q/M428L/N434S

SEQ ID NO: 865 - protein, 214 aa, synthetic construct; identical to SEQ ID NO: 758
  source 1..214: note = XENP44600 Chain 3 - IGKV1-39_L1

SEQ ID NO: 866 - protein, 450 aa, synthetic construct
  source 1..450: note = XENP49313 Chain 1 - 1A7[CD28]_H1.118_IgG1_pI(-)_Iso steric_A_PVA_/S267K/L368D/K370S/M428L/N434S
EVQLLESGGGLVQPGGSLRLSCAASGFTFSSYYMSWVRQAPGKGLEWVSTIAESGDPTYYADSVKGRFTISRDNSKNTLYLQMNSLRAEDTAVYYCAKSGPGLRQVGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 867 - protein, 449 aa, synthetic construct; identical to SEQ ID NO: 849
  source 1..449: note = XENP49313 Chain 2 - 1F11-1A3.315[Trop2]_H1_IgG1_PVA _/S267K/S364K/E357Q/M428L/N434S

SEQ ID NO: 868 - protein, 214 aa, synthetic construct; identical to SEQ ID NO: 758
  source 1..214: note = XENP49313 Chain 3 - IGKV1-39_L1

SEQ ID NO: 869 - protein, 450 aa, synthetic construct; identical to SEQ ID NO: 866
  source 1..450: note = XENP49314 Chain 1 - 1A7[CD28]_H1.118_IgG1_pI(-)_Iso steric_A_PVA_/S267K/L368D/K370S/M428L/N434S

SEQ ID NO: 870 - protein, 449 aa, synthetic construct; identical to SEQ ID NO: 855
  source 1..449: note = XENP49314 Chain 2 - 1F11-1A3.315[Trop2]_H1.10_IgG1_ PVA_/S267K/S364K/E357Q/M428L/N434S

SEQ ID NO: 871 - protein, 214 aa, synthetic construct; identical to SEQ ID NO: 758
  source 1..214: note = XENP49314 Chain 3 - IGKV1-39_L1

SEQ ID NO: 872 - protein, 450 aa, synthetic construct; identical to SEQ ID NO: 866
  source 1..450: note = XENP49315 Chain 1 - 1A7[CD28]_H1.118_IgG1_pI(-)_Iso steric_A_PVA_/S267K/L368D/K370S/M428L/N434S

SEQ ID NO: 873 - protein, 449 aa, synthetic construct; identical to SEQ ID NO: 861
  source 1..449: note = XENP49315 Chain 2 - 1F11-1A3.315[Trop2]_H1.19_IgG1_ PVA_/S267K/S364K/E357Q/M428L/N434S

SEQ ID NO: 874 - protein, 214 aa, synthetic construct; identical to SEQ ID NO: 758
  source 1..214: note = XENP49315 Chain 3 - IGKV1-39_L1

SEQ ID NO: 875 - protein, 450 aa, synthetic construct; identical to SEQ ID NO: 848
  source 1..450: note = XENP44601 Chain 1 - 1A7[CD28]_H1.129_IgG1_pI(-)_Iso steric_A_PVA_/S267K/L368D/K370S/M428L/N434S

SEQ ID NO: 876 - protein, 682 aa, synthetic construct
  source 1..682: note = XENP44601 Chain 2 - 1F11-1A3.315[Trop2]_H1_(GKPGS)2 _1F11-1A3.315[Trop2]_H1_IgG1_PVA_/S267K/S364K/E357Q/M428L/ N434S
QVQLVQSGAEVKKPGASVKVSCKASGYTFTRYYMHWVRQAPGQGLEWMGIINPGGGSTGYAQKFQGRVTMTRDTSTSTVYMELSSLRSEDTAVYYCARDDVVAADVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGKPGSGKPGSQVQLVQSGAEVKKPGASVKVSCKASGYTFTRYYMHWVRQAPGQGLEWMGIINPGGGSTGYAQKFQGRVTMTRDTSTSTVYMELSSLRSEDTAVYYCARDDVVAADVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 877 - protein, 214 aa, synthetic construct; identical to SEQ ID NO: 758
  source 1..214: note = XENP44601 Chain 3 - IGKV1-39_L1

SEQ ID NO: 878 - protein, 450 aa, synthetic construct; identical to SEQ ID NO: 851
  source 1..450: note = XENP44602 Chain 1 - 1A7[CD28]_H1.106_IgG1_pI(-)_Iso steric_A_PVA_/S267K/L368D/K370S/M428L/N434S

SEQ ID NO: 879 - protein, 682 aa, synthetic construct; identical to SEQ ID NO: 876
  source 1..682: note = XENP44602 Chain 2 - 1F11-1A3.315[Trop2]_H1_(GKPGS)2 _1F11-1A3.315[Trop2]_H1_IgG1_PVA_/S267K/S364K/E357Q/M428L/ N434S

SEQ ID NO: 880 - protein, 214 aa, synthetic construct; identical to SEQ ID NO: 758
  source 1..214: note = XENP44602 Chain 3 - IGKV1-39_L1

SEQ ID NO: 881 - protein, 449 aa, synthetic construct; identical to SEQ ID NO: 756
  source 1..449: note = XENP44869 Chain 1 - 1F11-1A3.315[Trop2]_H1_IgG1_pI( -)_Isosteric_A_PVA_/S267K/L368D/K370S/M428L/N434S

SEQ ID NO: 882 - protein, 683 aa, synthetic construct
  source 1..683: note = XENP44869 Chain 2 - 1F11-1A3.315[Trop2]_H1_(GKPGS)2 _1A7[CD28]_H1.129_IgG1_PVA_/S267K/S364K/E357Q/M428L/N434S
QVQLVQSGAEVKKPGASVKVSCKASGYTFTRYYMHWVRQAPGQGLEWMGIINPGGGSTGYAQKFQGRVTMTRDTSTSTVYMELSSLRSEDTAVYYCARDDVVAADVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGKPGSGKPGSEVQLLESGGGLVQPGGSLRLSCAASGFTFSSYYMSWVRQAPGKGLEWVSTISERADSTYYADSVKGRFTISRDNSKNTLYLQMNSLRAEDTAVYYCAKSGPGLRQVGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 883 - protein, 214 aa, synthetic construct; identical to SEQ ID NO: 758
  source 1..214: note = XENP44869 Chain 3 - IGKV1-39_L1

SEQ ID NO: 884 - protein, 449 aa, synthetic construct; identical to SEQ ID NO: 756
  source 1..449: note = XENP44870 Chain 1 - 1F11-1A3.315[Trop2]_H1_IgG1_pI( -)_Isosteric_A_PVA_/S267K/L368D/K370S/M428L/N434S

SEQ ID NO: 885 - protein, 683 aa, synthetic construct
  source 1..683: note = XENP44870 Chain 2 - 1F11-1A3.315[Trop2]_H1_(GKPGS)2 _1A7[CD28]_H1.106_IgG1_PVA_/S267K/S364K/E357Q/M428L/N434S
QVQLVQSGAEVKKPGASVKVSCKASGYTFTRYYMHWVRQAPGQGLEWMGIINPGGGSTGYAQKFQGRVTMTRDTSTSTVYMELSSLRSEDTAVYYCARDDVVAADVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGKPGSGKPGSEVQLLESGGGLVQPGGSLRLSCAASGFTFSSYYMSWVRQAPGKGLEWVSTISERGDSTYYADSVKGRFTISRDNSKNTLYLQMNSLRAEDTAVYYCAKSGPGLRQVGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 886 - protein, 214 aa, synthetic construct; identical to SEQ ID NO: 758
  source 1..214: note = XENP44870 Chain 3 - IGKV1-39_L1

SEQ ID NO: 887 - protein, 449 aa, synthetic construct
  source 1..449: note = XENP44871 Chain 1 - 1F11-1A3.315[Trop2]_H1.19_IgG1_ pI(-)_Isosteric_A_PVA_/S267K/L368D/K370S/M428L/N434S
QVQLVQSGAEVKKPGASVKVSCKASGYTLTRYYMHWVRQAPGQGLEWMGIIEPGGGSTTYAQKFQGRVTMTRDTSTSTVYMELSSLRSEDTAVYYCARDDVVKADVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVLHEALHSHYTQKSLSLSPGK

SEQ ID NO: 888 - protein, 683 aa, synthetic construct
  source 1..683: note = XENP44871 Chain 2 - 1F11-1A3.315[Trop2]_H1.19_(GKPG S)2_1A7[CD28]_H1.129_IgG1_PVA_/S267K/S364K/E357Q/M428L/N43 4S
QVQLVQSGAEVKKPGASVKVSCKASGYTLTRYYMHWVRQAPGQGLEWMGIIEPGGGSTTYAQKFQGRVTMTRDTSTSTVYMELSSLRSEDTAVYYCARDDVVKADVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGKPGSGKPGSEVQLLESGGGLVQPGGSLRLSCAASGFTFSSYYMSWVRQAPGKGLEWVSTISERADSTYYADSVKGRFTISRDNSKNTLYLQMNSLRAEDTAVYYCAKSGPGLRQVGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 889 - protein, 214 aa, synthetic construct; identical to SEQ ID NO: 758
  source 1..214: note = XENP44871 Chain 3 - IGKV1-39_L1

SEQ ID NO: 890 - protein, 449 aa, synthetic construct; identical to SEQ ID NO: 887
  source 1..449: note = XENP44872 Chain 1 - 1F11-1A3.315[Trop2]_H1.19_IgG1_ pI(-)_Isosteric_A_PVA_/S267K/L368D/K370S/M428L/N434S

SEQ ID NO: 891 - protein, 683 aa, synthetic construct
  source 1..683: note = XENP44872 Chain 2 - 1F11-1A3.315[Trop2]_H1.19_(GKPG S)2_1A7[CD28]_H1.106_IgG1_PVA_/S267K/S364K/E357Q/M428L/N43 4S
QVQLVQSGAEVKKPGASVKVSCKASGYTLTRYYMHWVRQAPGQGLEWMGIIEPGGGSTTYAQKFQGRVTMTRDTSTSTVYMELSSLRSEDTAVYYCARDDVVKADVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGKPGSGKPGSEVQLLESGGGLVQPGGSLRLSCAASGFTFSSYYMSWVRQAPGKGLEWVSTISERGDSTYYADSVKGRFTISRDNSKNTLYLQMNSLRAEDTAVYYCAKSGPGLRQVGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 892 - protein, 214 aa, synthetic construct; identical to SEQ ID NO: 758
  source 1..214: note = XENP44872 Chain 3 - IGKV1-39_L1

SEQ ID NO: 893 - protein, 450 aa, synthetic construct; identical to SEQ ID NO: 848
  source 1..450: note = XENP44623 Chain 1 - 1A7[CD28]_H1.129_IgG1_pI(-)_Iso steric_A_PVA_/S267K/L368D/K370S/M428L/N434S

SEQ ID NO: 894 - protein, 449 aa, synthetic construct
  source 1..449: note = XENP44623 Chain 2 - Numax[RSV]_H1_IgG1_PVA_/S267K/S364K/E357Q/M428L/N434S
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 895 - protein, 214 aa, synthetic construct; identical to SEQ ID NO: 758
  source 1..214: note = XENP44623 Chain 3 - IGKV1-39_L1

SEQ ID NO: 896 - protein, 450 aa, synthetic construct; identical to SEQ ID NO: 851
  source 1..450: note = XENP44624 Chain 1 - 1A7[CD28]_H1.106_IgG1_pI(-)_Iso steric_A_PVA_/S267K/L368D/K370S/M428L/N434S

SEQ ID NO: 897 - protein, 449 aa, synthetic construct; identical to SEQ ID NO: 894
  source 1..449: note = XENP44624 Chain 2 - Numax[RSV]_H1_IgG1_PVA_/S267K/S364K/E357Q/M428L/N434S

SEQ ID NO: 898 - protein, 214 aa, synthetic construct; identical to SEQ ID NO: 758
  source 1..214: note = XENP44624 Chain 3 - IGKV1-39_L1

SEQ ID NO: 899 - protein, 450 aa, synthetic construct; identical to SEQ ID NO: 848
  source 1..450: note = XENP44880 Chain 1 - 1A7[CD28]_H1.129_IgG1_pI(-)_Iso steric_A_PVA_/S267K/L368D/K370S/M428L/N434S

SEQ ID NO: 900 - protein, 682 aa, synthetic construct
  source 1..682: note = XENP44880 Chain 2 - 1F11-1A3.315[Trop2]_H1.19_(GKPG S)2_Numax[RSV]_H1_IgG1_PVA_/S267K/S364K/E357Q/M428L/N434S
QVQLVQSGAEVKKPGASVKVSCKASGYTLTRYYMHWVRQAPGQGLEWMGIIEPGGGSTTYAQKFQGRVTMTRDTSTSTVYMELSSLRSEDTAVYYCARDDVVKADVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGKPGSGKPGSQVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 901 - protein, 214 aa, synthetic construct; identical to SEQ ID NO: 758
  source 1..214: note = XENP44880 Chain 3 - IGKV1-39_L1

SEQ ID NO: 902 - protein, 122 aa, synthetic construct
  source 1..122: note = 2C5A3.316[TROP2]_H1L1 Variable heavy (vh) domain
QVQLVQSGAEVKKPGSSVKVSCKASGGTFTTSGISWVRQAPGQGLEWMGGIVPLLGTVNYAQKFQGRVTITADESTSTAYMELSSLRSEDTAVYYCARSGAATDARYGLDYWGQGTLVTVSS

SEQ ID NO: 903 - protein, 5 aa, synthetic construct
  source 1..5: note = 2C5A3.316[TROP2]_H1L1 vhCDR1
TSGIS

SEQ ID NO: 904 - protein, 17 aa, synthetic construct
  source 1..17: note = 2C5A3.316[TROP2]_H1L1 vhCDR2
GIVPLLGTVNYAQKFQG

SEQ ID NO: 905 - protein, 13 aa, synthetic construct
  source 1..13: note = 2C5A3.316[TROP2]_H1L1 vhCDR3
SGAATDARYGLDY

SEQ ID NO: 906 - protein, 107 aa, synthetic construct; identical to SEQ ID NO: 806
  source 1..107: note = 2C5A3.316[TROP2]_H1L1 Variable light (vl) domain

SEQ ID NO: 907 - protein, 11 aa, synthetic construct
  source 1..11: note = 2C5A3.316[TROP2]_H1L1 vlCDR1
RASQSISSYLN

SEQ ID NO: 908 - protein, 7 aa, synthetic construct
  source 1..7: note = 2C5A3.316[TROP2]_H1L1 vlCDR2
AASSLQS

SEQ ID NO: 909 - protein, 9 aa, synthetic construct
  source 1..9: note = 2C5A3.316[TROP2]_H1L1 vlCDR3
QQSYSTPYT

SEQ ID NO: 910 - protein, 121 aa, synthetic construct
  source 1..121: note = 1A3A4.312[TROP2]_H1L1 Variable heavy (vh) domain
QVQLVQSGAEVKKPGASVKVSCKASGYTFTRHYMHWVRQAPGQGLEWMGIINPGGGATSYAQKFQGRVTMTRDTSTSTVYMELSSLRSEDTAVYYCAREDYAGKWWGMDYWGQGTLVTVSS

SEQ ID NO: 911 - protein, 5 aa, synthetic construct
  source 1..5: note = 1A3A4.312[TROP2]_H1L1 vhCDR1
RHYMH

SEQ ID NO: 912 - protein, 17 aa, synthetic construct
  source 1..17: note = 1A3A4.312[TROP2]_H1L1 vhCDR2
IINPGGGATSYAQKFQG

SEQ ID NO: 913 - protein, 12 aa, synthetic construct
  source 1..12: note = 1A3A4.312[TROP2]_H1L1 vhCDR3
EDYAGKWWGMDY

SEQ ID NO: 914 - protein, 107 aa, synthetic construct; identical to SEQ ID NO: 806
  source 1..107: note = 1A3A4.312[TROP2]_H1L1 Variable light (vl) domain

SEQ ID NO: 915 - protein, 11 aa, synthetic construct
  source 1..11: note = 1A3A4.312[TROP2]_H1L1 vlCDR1
RASQSISSYLN

SEQ ID NO: 916 - protein, 7 aa, synthetic construct
  source 1..7: note = 1A3A4.312[TROP2]_H1L1 vlCDR2
AASSLQS

SEQ ID NO: 917 - protein, 9 aa, synthetic construct
  source 1..9: note = 1A3A4.312[TROP2]_H1L1 vlCDR3
QQSYSTPYT

SEQ ID NO: 918 - protein, 123 aa, synthetic construct
  source 1..123: note = 1B2A4.312[TROP2]_H1L1 Variable heavy (vh) domain
QVQLVQSGAEVKKPGASVKVSCKASGYTFTGSYMHWVRQAPGQGLEWMGIINPSGESTVYAQKFQGRVTMTRDTSTSTVYMELSSLRSEDTAVYYCARDPGGSWPFSYALDYWGQGTLVTVSS

SEQ ID NO: 919 - protein, 5 aa, synthetic construct
  source 1..5: note = 1B2A4.312[TROP2]_H1L1 vhCDR1
GSYMH

SEQ ID NO: 920 - protein, 17 aa, synthetic construct
  source 1..17: note = 1B2A4.312[TROP2]_H1L1 vhCDR2
IINPSGESTVYAQKFQG

SEQ ID NO: 921 - protein, 14 aa, synthetic construct
  source 1..14: note = 1B2A4.312[TROP2]_H1L1 vhCDR3
DPGGSWPFSYALDY

SEQ ID NO: 922 - protein, 107 aa, synthetic construct; identical to SEQ ID NO: 806
  source 1..107: note = 1B2A4.312[TROP2]_H1L1 Variable light (vl) domain

SEQ ID NO: 923 - protein, 11 aa, synthetic construct
  source 1..11: note = 1B2A4.312[TROP2]_H1L1 vlCDR1
RASQSISSYLN

SEQ ID NO: 924 - protein, 7 aa, synthetic construct
  source 1..7: note = 1B2A4.312[TROP2]_H1L1 vlCDR2
AASSLQS

SEQ ID NO: 925 - protein, 9 aa, synthetic construct
  source 1..9: note = 1B2A4.312[TROP2]_H1L1 vlCDR3
QQSYSTPYT

SEQ ID NO: 926 - protein, 119 aa, synthetic construct
  source 1..119: note = 1B11A3.316[TROP2]_H1L1 Variable heavy (vh) domain
QVQLVQSGAEVKKPGASVKVSCKASGYTFTRYYMHWVRQAPGQGLEWMGIINPGGGSTIYAQKFQGRVTMTRDTSTSTVYMELSSLRSEDTAVYYCARDDVIVDAFDYWGQGTLVTVSS

SEQ ID NO: 927 - protein, 5 aa, synthetic construct
  source 1..5: note = 1B11A3.316[TROP2]_H1L1 vhCDR1
RYYMH

SEQ ID NO: 928 - protein, 17 aa, synthetic construct
  source 1..17: note = 1B11A3.316[TROP2]_H1L1 vhCDR2
IINPGGGSTIYAQKFQG

SEQ ID NO: 929 - protein, 10 aa, synthetic construct
  source 1..10: note = 1B11A3.316[TROP2]_H1L1 vhCDR3
DDVIVDAFDY

SEQ ID NO: 930 - protein, 107 aa, synthetic construct; identical to SEQ ID NO: 806
  source 1..107: note = 1B11A3.316[TROP2]_H1L1 Variable light (vl) domain

SEQ ID NO: 931 - protein, 11 aa, synthetic construct
  source 1..11: note = 1B11A3.316[TROP2]_H1L1 vlCDR1
RASQSISSYLN

SEQ ID NO: 932 - protein, 7 aa, synthetic construct
  source 1..7: note = 1B11A3.316[TROP2]_H1L1 vlCDR2
AASSLQS

SEQ ID NO: 933 - protein, 9 aa, synthetic construct
  source 1..9: note = 1B11A3.316[TROP2]_H1L1 vlCDR3
QQSYSTPYT

SEQ ID NO: 934 - protein, 120 aa, synthetic construct
  source 1..120: note = 1C9A4.313[TROP2]_H1L1 Variable heavy (vh) domain
QVQLVQSGAEVKKPGSSVKVSCKASGGTFSNYAIGWVRQAPGQGLEWMGGIVPIFGVADYAQKFQGRVTITADESTSTAYMELSSLRSEDTAVYYCAREGWYLLNYFDYWGQGTLVTVSS

SEQ ID NO: 935 - protein, 5 aa, synthetic construct
  source 1..5: note = 1C9A4.313[TROP2]_H1L1 vhCDR1
NYAIG

SEQ ID NO: 936 - protein, 17 aa, synthetic construct
  source 1..17: note = 1C9A4.313[TROP2]_H1L1 vhCDR2
GIVPIFGVADYAQKFQG

SEQ ID NO: 937 - protein, 11 aa, synthetic construct
  source 1..11: note = 1C9A4.313[TROP2]_H1L1 vhCDR3
EGWYLLNYFDY

SEQ ID NO: 938 - protein, 107 aa, synthetic construct; identical to SEQ ID NO: 806
  source 1..107: note = 1C9A4.313[TROP2]_H1L1 Variable light (vl) domain

SEQ ID NO: 939 - protein, 11 aa, synthetic construct
  source 1..11: note = 1C9A4.313[TROP2]_H1L1 vlCDR1
RASQSISSYLN

SEQ ID NO: 940 - protein, 7 aa, synthetic construct
  source 1..7: note = 1C9A4.313[TROP2]_H1L1 vlCDR2
AASSLQS

SEQ ID NO: 941 - protein, 9 aa, synthetic construct
  source 1..9: note = 1C9A4.313[TROP2]_H1L1 vlCDR3
QQSYSTPYT